(19) United States
(12) Patent Application Publication
(10) Pub. No.: US 2013/0109757 A1
(43) Pub. Date: May 2, 2013

(54) PROCESS FOR THE PREPARATION OF PYRROLINES FROM GAMMA-NITROKETONES. USE OF THE GAMMA-NITROKETONES AS PESTICIDAL AGENTS

(71) Applicants: Wahed Ahmed Moradi, Monheim (DE); Thomas Norbert Müller, Monheim (DE); Tetsuya Murata, Izumi-shi (JP); Mamoru Hatazawa, Ibaraki (JP); Peter Bruechner, Dusseldorf (DE); Eiichi Shimojo, Osaka (JP); Teruyuki Ichihara, Tochigi (JP); Masashi Ataka, Saitama (JP); Katsuhiko Shibuya, Tochigi (JP); Ulrich Görgens, Ratingen (DE)

(72) Inventors: Wahed Ahmed Moradi, Monheim (DE); Thomas Norbert Müller, Monheim (DE); Tetsuya Murata, Izumi-shi (JP); Mamoru Hatazawa, Ibaraki (JP); Peter Bruechner, Dusseldorf (DE); Eiichi Shimojo, Osaka (JP); Teruyuki Ichihara, Tochigi (JP); Masashi Ataka, Saitama (JP); Katsuhiko Shibuya, Tochigi (JP); Ulrich Görgens, Ratingen (DE)

(73) Assignee: BAYER INTELLECTUAL PROPERTY GMBH, Monheim (DE)

(21) Appl. No.: 13/641,061

(22) PCT Filed: Apr. 11, 2011
(86) PCT No.: PCT/EP2011/055639

§ 371 Date: Dec. 26, 2012

(30) Foreign Application Priority Data
Apr. 13, 2010 (JP) 2010-092182

(51) Int. Cl. A01N 37/18; C07D 207/20

(52) U.S. Cl. CPC A01N 37/18; C07D 207/20
USPC 514/629; 548/565; 564/222

(57) ABSTRACT

The invention is directed to method for the preparation of pyrrolines of the general formula (I)

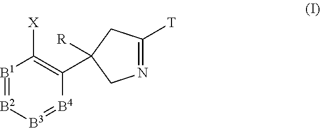

by catalytic hydration of a nitroketone of the general formula (II)

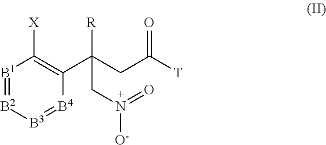

employing a transition metal catalyst and gaseous hydrogen at an elevated pressure in a solvent, optionally in the presence of at least one additive selected among Lewis acids, Brønstedt acids, organic sulfur-containing compounds, organic or inorganic bases, and water scavengers, wherein in formulae (I) and (II) B¹, B², B³, B⁴, X, R, and T are as defined in the specification, and to a nitroketone of formula (II) to be used as intermediate in the process according to the invention and as pesticidal agent.

The present invention relates to a method (process) for the synthesis of substituted pyrrolines as well as to specific γ-nitroketones which can be used as starting materials in the process according to the invention. Pyrrolines are biological active and can thus be used as pesticides (cf. WO 2009/097992, WO 2009/112275).

Several methods for the manufacturing of pyrrolines are known (cf. Cheruku, Srinivasa et al. Tetrahedron Letters 44 (2003), 3701-3703 and Moffett and White, J. Org. Chem. 17 (1952) 407-413)). One method is the reductive cyclization of γ-nitroketones using zinc powder together with HCO₂H-EtOH (1:1) which results in pyrroline N-oxides and pyrrolines (cf. Cheruku, Srinivasa et al. Tetrahedron Letters 44 (2003), 3701-3703).

The reductive cyclization of a bromine-substituted pyrrole-γ-nitroketone is described by Laha, K. Joydev et al. in J. Org. Chem. 2006, 71, pp. 4092-4102 (therein named as compound 8) using zinc dust and HCO₂NH₄ in THF at room temperature as given in the following reaction scheme 1. Notably, both zinc and the ammonium formiate are used in a 15-fold excess based on the molar amount of the starting material 8.

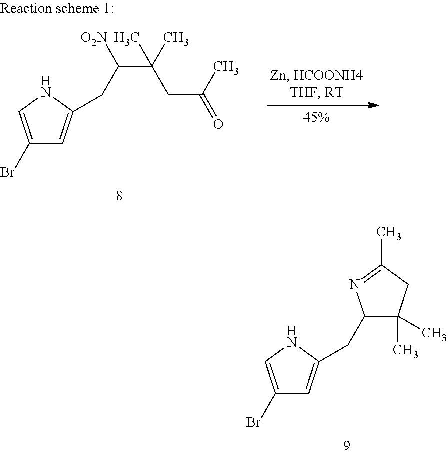 [SMILES: CC(=O)CC(C)(C)C(Cc1cc(Br)c[nH]1)[N+](=O)[O-] ; CC1=NC(Cc2cc(Br)c[nH]2)C(C)(C)C1]

This reaction resulted in the desired product 9 with a yield of 45% (see also WO 2007/64842).

It is emphasized in this article that if the reaction continues over more than 5 hours, a significant formation of a side product occurs. Besides the fact that a large excess of reducing agent is needed, which is—at least from an economical and ecological stand point—disadvantageous, the yield for the desired product is quite poor.

WO 2010/149506 describes another method for the reductive cyclization of a γ-nitroketone, namely 4-[3-(3,5-dichloro-phenyl)-4,4,4-trifluoro-3-nitromethylbutyryl]-2-methyl-N-thietan-3-yl-benzamide in DMF with zinc powder and HCl at 80° C. for 4 hours. After work-up the desired product, namely 4-[4-(3,5-dichloro-phenyl)-4-trifluoromethyl-4,5-dihydropyrrol-2-yl]-2-methyl-N-thietan-3-yl-benzamide was obtained in a yield of only 17%.

Another known method for reductive cyclization is the catalytic hydrogenation. The treatment of γ-nitroketones with nickel catalyst (Raney™ 2800 nickel) in ethanol at room temperature under a hydrogen atmosphere at atmospheric pressure is described in Cheruku, Srinivasa et al. Tetrahedron Letters 44 (2003), 3701-3703. General conditions under which the reductive cyclization by catalytic hydrogentation of alkylnitroketones can take place are described by Nishimura in “Handbook of Heterogeneous Catalytic Hydrogenation for Organic Synthesis”, pp. 353-358, John Wiley and Sons, New York, 2001.

The reductive cyclization is, however, in general problematic when γ-nitroketones are used which carry halogen substituent(s). Such compounds are easily dehalogenated during the catalytic hydrogenation. The tendency of a halogen-containing compound to dehalogenate during catalytic hydrogenation is higher for bromine- than for chlorine-containing compounds and higher for two- or morefold substituted compounds than for onefold substituted compounds. (cf. Nishimura in “Handbook of Heterogeneous Catalytic Hydrogenation for Organic Synthesis”, pp. 623-637, John Wiley and Sons, New York, 2001).

This renders the reductive cyclization via catalytic hydrogenation of substituted γ-nitroketones costly and inefficient and thus not suitable for being used on an industrial scale.

Although Li et al. report in Chem. Commun. 2009, pp. 2136-2138 about a Raney™ nickel-mediated hydrogenation of a substituted γ-nitroketone 3c to a compound 4 (reaction scheme 2) in 80% yield, Li et al. do not disclose any experimental data nor reaction conditions and thus rendering the disclosure not workable. As demonstrated herein, the inventors failed to reproduce the high yield under the conditions as foreshadowed by Li et al. using the compounds of formula (II) of the present invention.

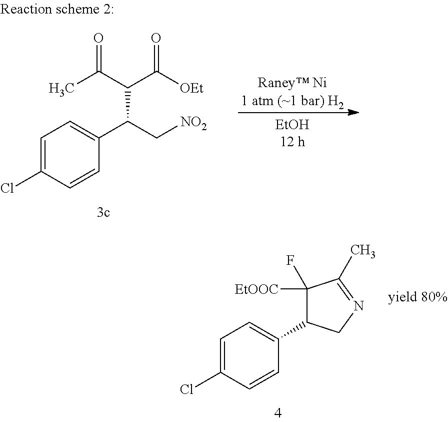

When looking for a new and efficient synthesis route to manufacture certain pyrrolines by using the reductive cyclization of a γ-nitroketone, the inventors found an excellent method for the preparation of pyrrolines of the general formula (I) through catalytic hydrogenation

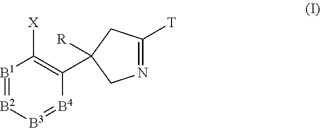

by using a nitroketone of the general formula (II)

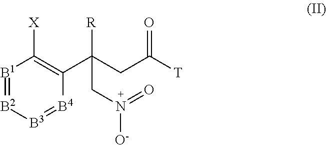

wherein in the formulae (I) and (II) B¹, B², B³ and B⁴, X, R, and T are as defined herein, which is efficient, cost-effective and can be used on a large scale.

Thus the invention is directed to a method for the preparation of pyrrolines of the general formula (I)

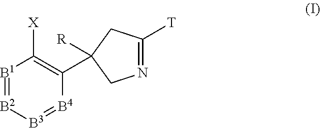

by catalytic hydration of a nitroketone of the general formula (II)

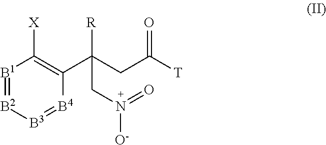

employing a transition metal catalyst and gaseous hydrogen at an elevated pressure in a suitable solvent, optionally in the presence of at least one additive selected among Lewis acids, Brønstedt acids, organic sulfur-containing compounds, organic or inorganic bases, and water scavengers
wherein in the formulae (I) and (II)
T is one of the following chemical groupings (T1), (T2), (T3), (T4) or (T5)

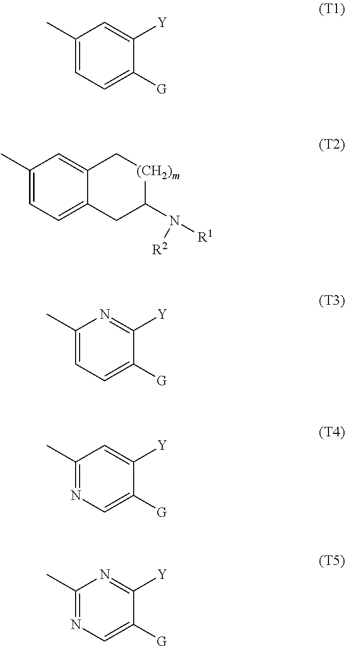

wherein
Y is hydrogen, halogen, nitro, cyano, amino, hydroxy, mercapto, alkyl, haloalkyl, cycloalkyl, halocycloalkyl, alkoxy, haloalkoxy, alkylthio, alkylsulfinyl, alkylsulfonyl, haloalkylthio, haloalkylsulfinyl, haloalkylsulfonyl, alkylsulfonyloxy, haloalkylsulfonyloxy, mono- or di-alkylaminosulfonyl, mono- or di-haloalkylamino-sulfonyl, mono- or di-alkylamino, acylamino, alkoxycarbonylamino, haloalkoxycarbonylamino, alkylsulfonylamino, haloalkylsulfonylamino, trialkylsilyl, alkoxyamino, haloalkoxyamino, alkoxyiminoalkyl, haloalkoxyiminoalkyl, alkylsulfinylimino, alkylsulfinyliminoalkyl, alkylsulfinyliminoalkylcarbonyl, alkylsulfoxyimino, alkylsulfoxyiminoalkyl, alkoxycarbonyl, alkylcarbonyl, aminocarbonyl, mono- or di-alkylaminocarbonyl, aminothiocarbonyl or mono- or di-alkylaminothiocarbonyl, and each group from alkyl to di-alkylaminothiocarbonyl among the definitions of Y may be optionally substituted;
G is a saturated or unsaturated 5- to 6-membered heterocyclic group which may be substituted, or
G is halogen, cyano, alkyl, alkoxycarbonyl, carboxy, or CH₃S, or
G is one of the following groups (G10a) or (G10b):

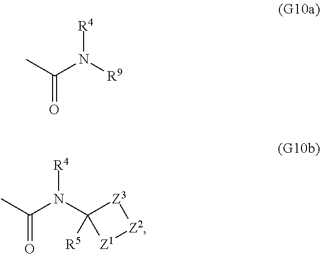

G is one of the following groups (G11) to (G16):

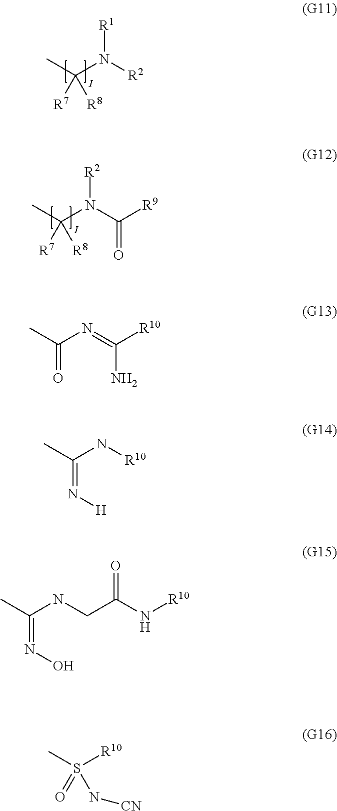

R¹ is hydrogen, cyano, formyl, thioformyl, alkylcarbonyl, haloalkylcarbonyl, alkyl-thiocarbonyl, haloalkyl-thiocarbonyl, mono- or di-alkylaminocarbonyl, mono- or di-alkylamino-thiocarbonyl, alkoxyaminocarbonyl, alkoxyamino-thiocarbonyl, alkoxycarbonyl, alkoxyalkylcarbonyl, alkoxy-thiocarbonyl, alkylthio-carbonyl, alkylthio-thiocarbonyl, alkylsulfonyl, haloalkylsulfonyl, cycloalkylcarbonyl, alkenylcarbonyl, alkynylcarbonyl, cycloalkyl-alkylcarbonyl, alkylthioalkylcarbonyl, alkylsulfinylalkylcarbonyl, alkylsulfonylalkylcarbonyl, alkylcarbonylalkylcarbonyl, cycloalkylaminocarbonyl, alkenylaminocarbonyl, alkynylaminocarbonyl, —CH₂—R³, —C(O)R³ or —C(S)R³, and each group from alkylcarbonyl to alkynylaminocarbonyl among the definitions of R¹ may be substituted;
R² is hydrogen, amino, hydroxy, cyano, alkyl, haloalkyl, cycloalkyl, alkenyl, alkynyl, alkylimino, alkoxy, alkylcarbonyl, alkylcarbonylamino, alkoxyalkyl, cyanoalkyl, alkoxycarbonylalkyl, —CH₂—R³, —C(O)R³ or —C(S)R³, and each group from alkyl to alkylcarbonylamino among the definitions of R² may be substituted; or
R¹ and R² together with the N atom to which they are bound, may form a 3- to 6-membered heterocyclic ring which may be substituted and may further comprise N, O or S;
R³ is phenyl which may be substituted, a 5- to 6-membered heterocyclic ring which may be substituted, optionally substituted alkyl, amino, or mono- or diaalkylamino;
m is 1 or 2;
R⁴ and R⁵ independently are hydrogen, halogen, alkyl which may be substituted or haloalkyl which may be substituted;
Z¹, Z² and Z³ independently is —CR⁴R⁵—, —C(O)—, —C(N—OR⁶)—, —NR⁶—, —S(O)ₙ—, —S(N—R⁶)— or —S(O)(N—R⁶)—, or oxygen with the proviso that Z¹, Z² and Z³ do not simultaneously represent —CR⁴R⁵— or do not simultaneously represent oxygen;
n is 0, 1 or 2;
R⁶ is hydrogen, cyano, nitro, alkyl, haloalkyl, cycloalkylalkyl, alkylcarbonyl, haloalkylcarbonyl, alkoxycarbonyl, haloalkoxycarbonyl, alkylsulfonyl, haloalkylsulfonyl, aralkyl (arylalkyl) or alkyl which is substituted with a heterocycle, and each group from alkyl to alkyl which is substituted with a heterocycle among the definitions of R⁶ may be substituted;
R⁷ and R⁸ each independently are hydrogen, cyano, alkyl, cycloalkyl, haloalkyl, cyclohaloalkyl, alkenyl, alkynyl, haloalkenyl, haloalkynyl, alkoxycarbony, or alkoxythiocarbonyl, and each of the groups from alkyl to alkoxythiocarbonyl among the definition of R⁷ and R⁸ may be substituted; or
R⁹ is hydrogen, alkyl, alkoxy, haloalkyl, mono- or di-alkylamino, alkoxyamino, alkoxyalkyl, alkylhio, cycloalkyl, alkenyl, alkynyl, cycloalkylalkyl, alkylthioalkyl, alkylsulfinylalkyl, alkylsulfonylalkyl, alkylcarbonylalkyl, cycloalkylamino, alkenylamino, alkynylamino, —CH₂R³, —C(O)R³ or —C(S)R³, and each group from alkyl to alkynylamino among the definitions of R⁹ may be substituted;
R¹⁰ is hydrogen, hydroxy, alkyl, haloalkyl, cycloalkyl, alkenyl, alkinyl, alkoxy, alkylcarbonyl, alkoxycarbonyl, alkoxyalkyl, cyanoalkyl, alkoxycarbonylalkyl, —CH₂R³, —C(O)R³ or —C(S)R³ and each group from alkyl to alkoxycarbonylalkyl among the definitions of R¹⁰ may be substituted;
l stands for 1, 2 or 3;
R is alkyl which may be substituted or haloalkyl which may be substituted;
X is hydrogen, halogen, nitro, cyano, hydroxy, mercapto, amino, alkyl, haloalkyl, alkoxy, haloalkoxy, alkylthio, alkylsulfinyl, alkylsulfonyl, haloalkylthio, haloalkylsulfinyl, haloalkylsulfonyl, acylamino, alkoxycarbonylamino, haloalkoxycarbonylamino, alkoxyimino, haloalkoxyimino, alkylsulfonylamino or sulfur pentafluoride, and each group from alkyl to alkylsulfonylamino among the definitions of X may be optionally substituted; and
B¹, B², B³ and B⁴ independently is C—X or N.

In an embodiment (M-1), the invention is directed to the method according to the invention, wherein the pressure is in the range from 2 to 100 bar, preferably in the range from 3.5 bar to 100 bar, more preferably in the range from 5 to 50 bar, most preferably in the range from 10 to 30 bar.

In an embodiment (M-2), the invention is directed to the method according to embodiment (M-1), wherein the transition metal catalyst used contains at least one metal selected from platinum, palladium, cobalt or nickel.

In an embodiment (M-3), the invention is directed to the method according to the invention, wherein Raney-Nickel is used as transition metal catalyst in the presence of the additive, preferably a sulfur-containing compound.

In an embodiment (M-4), the invention is directed to the method according to embodiment (M-3), wherein the additive is selected from thiophene, tetrahydrothiophene and 2,2′-thiobisethanol.

Each of the nitroketones represented by formula (II) has an asymmetric carbon. Thus, the nitroketones represented by formula (II) and specified herein include also the optical isomers of the respective compound.

The invention is also directed to nitroketone compounds of formula (II) as defined herein and their use as starting materials in the method according to the invention.

The invention is further directed to the nitroketone compounds of formula (II) as defined herein being useful as insecticidal agents for combating harmful invertebrate pests, such as insects which occure in the agriculture or insects which occure in the veterinary field (such as endo- or ectoparasites).

The invention is moreover directed to the use of the nitroketone compounds of formula (II) as defined herein for the preparation of a pyrroline of formula (I).

The invention is further directed to a pyrroline of formula (I) which is manufactured with the method according to the invention.

Preferred nitroketone compounds of formula (II) which are used or employed according to the invention are compounds of general formula (II)

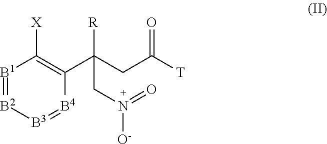

wherein
T is one of the following chemical groupings (T1), (T2), (T3), (T4) or (T5)

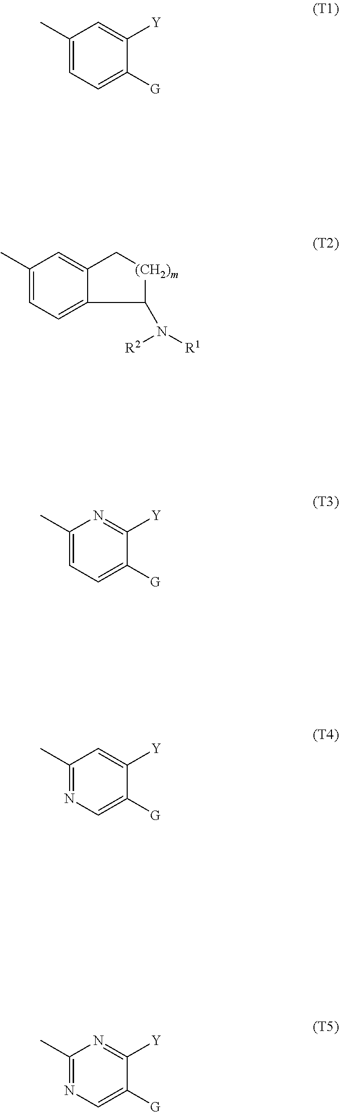

wherein
Y is hydrogen, halogen, nitro, cyano, amino, hydroxy, mercapto, C₁₋₁₂ alkyl, C₁₋₁₂ haloalkyl, C₃₋₈ cycloalkyl, C₃₋₈ halocycloalkyl, C₁₋₁₂ alkoxy, C₁₋₁₂ haloalkoxy, C₁₋₁₂ alkylthio, C₁₋₁₂ alkylsulfinyl, C₁₋₁₂ alkylsulfonyl, C₁₋₁₂ haloalkylthio, C₁₋₁₂ haloalkylsulfinyl, C₁₋₁₂ haloalkylsulfonyl, C₁₋₁₂ alkylsulfonyloxy, C₁₋₁₂ haloalkylsulfonyloxy, mono-(C₁₋₁₂) or di-(C₂₋₂₄) alkylaminosulfonyl, mono-(C₁₋₁₂) or di-(C₂₋₂₄) haloalkylaminosulfonyl, mono-(C₁₋₁₂) or di(C₂₋₂₄) alkylamino, C₁₋₁₂ alkyl-carbonylamino, C₃₋₈ cycloalkyl-carbonylamino, benzoylamino, C₁₋₁₂ alkoxy-carbonylamino, C₁₋₁₂ haloalkoxy-carbonylamino, C₁₋₁₂ alkylsulfonylamino, C₁₋₁₂ haloalkylsulfonylamino, C₃₋₃₆ trialkylsilyl, C₁₋₁₂ alkoxyamino, C₁₋₁₂ haloalkoxyamino, C₁₋₁₂ alkoxyimino-C₁₋₁₂ alkyl, C₁₋₁₂ haloalkoxyimino-C₁₋₁₂ alkyl, C₁₋₁₂ alkylsulfinylimino, C₁₋₁₂ alkylsulfinylimino-C₁₋₁₂ alkyl, C₁₋₁₂ alkylsulfinylimino-C₁₋₁₂ alkyl-carbonyl, C₁₋₁₂ alkylsulfoxyimino, C₁₋₁₂ alkylsulfoxyimino-C₁₋₁₂ alkyl, C₁₋₁₂ alkoxy-carbonyl, C₁₋₁₂ alkyl-carbonyl, aminocarbonyl, mono-(C₁₋₁₂) or di-(C₂₋₂₄) alkyl-aminocarbonyl, aminothiocarbonyl or mono-(C₁₋₁₂) or di-(C₂₋₂₄) alkylaminothio-carbonyl, and each group from C₁₋₁₂ alkyl to di-(C₂₋₂₄) alkylaminothio-carbonyl among the definitions of Y may be optionally substituted; preferably Y is hydrogen, halogen, nitro, cyano, amino, hydroxy, mercapto, C₁₋₆ alkyl, C₁₋₆ haloalkyl, C₃₋₆ cycloalkyl, C₃₋₆ halocycloalkyl, C₁₋₆ alkoxy, C₁₋₆ haloalkoxy, C₁₋₆ alkylthio, C₁₋₆ alkylsulfinyl, C₁₋₆ alkylsulfonyl, C₁₋₆ haloalkylthio, C₁₋₆ haloalkylsulfinyl, C₁₋₆ haloalkylsulfonyl, C₁₋₆ alkylsulfonyloxy, C₁₋₆ haloalkylsulfonyloxy, mono-(C₁₋₆) or di-(C₂₋₁₂) alkylaminosulfonyl, mono-(C₁₋₆) or di-(C₂₋₁₂) haloalkylaminosulfonyl, mono-(C₁₋₆) or di-(C₂₋₁₂) alkylamino, C₁₋₆ alkyl-carbonylamino, C₃₋₆ cycloalkyl-carbonylamino, benzoylamino, C₁₋₆ alkoxy-carbonylamino, C₁₋₆ haloalkoxy-carbonylamino, C₁₋₆ alkylsulfonylamino, C₁₋₆ haloalkylsulfonylamino, C₃₋₁₈ trialkylsilyl, C₁₋₆ alkoxyamino, C₁₋₆ haloalkoxyamino, C₁₋₆ alkoxyimino-C₁₋₆ alkyl, C₁₋₆ haloalkoxyimino-C₁₋₆ alkyl, C₁₋₆ alkylsulfinylimino, C₁₋₆ alkylsulfinylimino-C₁₋₆ alkyl, C₁₋₆ alkylsulfinylimino-C₁₋₆ alkyl-carbonyl, C₁₋₆ alkylsulfoxyimino, C₁₋₆ alkylsulfoxyimino-C₁₋₆ alkyl, C₁₋₆ alkoxy-carbonyl, C₁₋₆ alkyl-carbonyl, aminocarbonyl, mono-(C₁₋₆) or di-(C₂₋₁₂) alkyl-aminocarbonyl, aminothiocarbonyl or mono-(C₁₋₆) or di-(C₂₋₁₂) alkylaminothio-carbonyl, and each group from C₁₋₆ alkyl to di-(C₂₋₁₂) alkylaminothio-carbonyl among the definitions of Y may be optionally substituted;
G is a saturated or unsaturated 5- to 6-membered heterocyclic group which may be substituted, preferably G is one of the following heterocyclic groups (G1) to (G9):

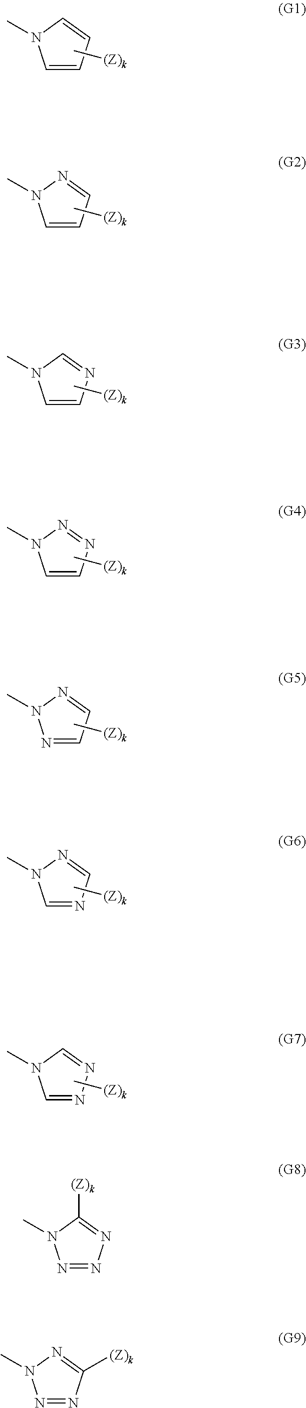

more preferably G is one of the groups (G2), (G6), (G8), or (G9), most preferably G is (G2) or (G6), wherein in the groups (G1) to (G9), the substituent (Z) is C₁₋₆ alkyl, C₁₋₆ haloalkyl, halogen, (preferably fluorine, chlorine, bromine), cyano or nitro, and k is 0, 1, 2, 3 or 4, or
G is halogen, cyano, C₁₋₁₂ alkyl, C₁₋₁₂ alkoxy-carbonyl, carboxy or CH₃S, preferably G is halogen, cyano, C₁₋₆ alkyl, C₁₋₆alkoxy-carbonyl, carboxy or CH₃S, or
G is one of the following groups (G10a) or (G10b):

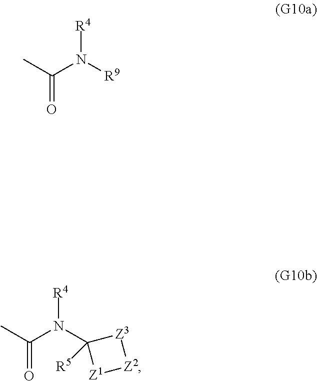

or
G is one of the following groups (G11) to (G16):

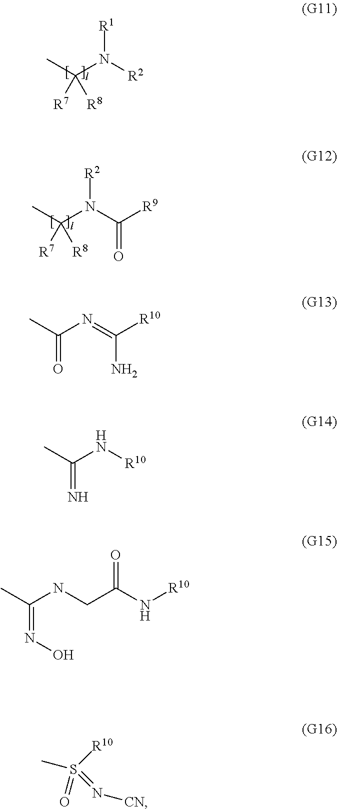

preferably G is a group (G11) or (G12),
R¹ is hydrogen, cyano, formyl, thioformyl, C₁₋₁₂ alkyl-carbonyl, C₁₋₁₂ haloalkyl-carbonyl, C₁₋₁₂ alkyl-thiocarbonyl, C₁₋₁₂ haloalkyl-thiocarbonyl, mono-(C₁₋₁₂) or di-(C₂₋₂₄) alkyl-aminocarbonyl, mono-(C₁₋₁₂) or di-(C₂₋₂₄) alkylamino-thiocarbonyl, C₁₋₁₂ alkoxy-aminocarbonyl, C₁₋₁₂ alkoxyamino-thiocarbonyl, C₁₋₁₂ alkoxy-carbonyl, C₁₋₁₂ alkoxy-C₁₋₁₂ alkyl-carbonyl, C₁₋₁₂ alkoxy-thiocarbonyl, C₁₋₁₂ alkylthio-carbonyl, C₁₋₁₂ alkylthio-thiocarbonyl, C₁₋₁₂ alkylsulfonyl, C₁₋₁₂ haloalkylsulfonyl, C₃₋₈ cycloalkyl-carbonyl, C₂₋₆ alkenyl-carbonyl, C₂₋₆ alkynyl-carbonyl, C₃₋₈ cycloalkyl-C₁₋₄ alkyl-carbonyl, C₁₋₁₂ alkylthio-C₁₋₁₂ alkyl-carbonyl, C₁₋₁₂ alkylsulfinyl-C₁₋₁₂alkyl-carbonyl, C₁₋₁₂ alkylsulfonyl-C₁₋₁₂alkyl-carbonyl, C₁₋₁₂ alkylcarbonyl-C₁₋₁₂ alkyl-carbonyl, C₃₋₈ cycloalkylamino-carbonyl, C₂₋₆ alkenylamino-carbonyl, C₂₋₆ alkynylamino-carbonyl, —C(O)R³ or —C(S)R³, and each group from C₁₋₁₂ alkyl-carbonyl to C₂₋₆ alkynylamino-carbonyl among the definitions of R¹ may be optionally substituted; preferably R¹ is hydrogen, cyano, carbonyl, thiocarbonyl, C₁₋₆ alkyl-carbonyl, C₁₋₆ haloalkyl-carbonyl, C₁₋₆ alkyl-thiocarbonyl, C₁₋₆ haloalkyl-thiocarbonyl, mono-(C₁₋₆) or di-(C₂₋₁₂) alkyl-aminocarbonyl, mono-(C₁₋₆) or di-(C₂₋₁₂) alkylamino-thiocarbonyl, C₁₋₆ alkoxy-aminocarbonyl, C₁₋₆ alkoxyamino-thiocarbonyl, C₁₋₆ alkoxy-carbonyl, C₁₋₆ alkoxy-C₁₋₆ alkyl-carbonyl, C₁₋₆ alkoxy-thiocarbonyl, C₁₋₆ alkylthio-carbonyl, C₁₋₆ alkylthio-thiocarbonyl, C₁₋₆ alkylsulfonyl, C₁₋₆ haloalkylsulfonyl, C₃₋₆ cycloalkyl-carbonyl, C₂₋₄ alkenyl-carbonyl, C₂₋₄ alkynyl-carbonyl, C₃₋₆ cycloalkyl-C₁₋₂ alkyl-carbonyl, C₁₋₆ alkylthio-C₁₋₆ alkyl-carbonyl, C₁₋₆ alkylsulfinyl-C₁₋₆ alkyl-carbonyl, C₁₋₆ alkylsulfonyl-C₁₋₆ alkyl-carbonyl, C₁₋₆ alkylcarbonyl-C₁₋₆ alkyl-carbonyl, C₃₋₆ cycloalkylamino-carbonyl, C₂₋₄ alkenylamino-carbonyl, C₂₋₄ alkynylamino-carbonyl, —CH₂—R³, —C(O)R³ or —C(S)R³, and each group from C₁₋₆ alkyl to C₁₋₆ alkynylamino-carbonyl among the definitions of R¹ may be optionally substituted;
R² is hydrogen, amino, hydroxy, cyano, C₁₋₁₂ alkyl, C₁₋₁₂ haloalkyl, C₃₋₈ cycloalkyl, C₂₋₆ alkenyl, C₂₋₆ alkynyl, C₁₋₁₂ alkylimino, C₁₋₁₂ alkoxy, C₁₋₁₂ alkyl-carbonyl, C₁₋₁₂ alkyl-carbonylamino, C₁₋₁₂ alkoxy-C₁₋₁₂ alkyl, C₁₋₁₂ cyanoalkyl, C₁₋₁₂ alkoxycarbonyl-C₁₋₁₂ alkyl, —CH₂—R³, —C(O)R³ or —C(S)R³, and each group from C₁₋₁₂ alkyl to C₁₋₁₂ alkyl-carbonylamino among the definitions of R² may be optionally substituted; preferably R² is hydrogen, amino, hydroxy, cyano, C₁₋₆ alkyl, C₁₋₆ haloalkyl, C₃₋₆ cycloalkyl, C₂₋₄ alkenyl, C₂₋₄ alkynyl, C₁₋₆ alkylimino, C₁₋₆ alkoxy, C₁₋₆ alkyl-carbonyl, C₁₋₆ alkyl-carbonylamino, C₁₋₆ alkoxy-C₁₋₆ alkyl, C₁₋₆ cyanoalkyl, C₁₋₆ alkoxycarbonyl-C₁₋₆ alkyl, —CH₂—R³, —C(O)R³ or —C(S)R³, and each group from C₁₋₆ alkyl to C₁₋₆ alkyl-carbonylamino among the definitions of R² may be optionally substituted; or
R¹ and R², together with the N atom to which they are bound, may form a 3- to 6-membered heterocyclic ring which may be substituted and may further comprise N, O or S; preferably R¹ and R², together with the N atom to which they are bound, may form a 3- to 6-membered heterocyclic ring which may be substituted and may further comprise N, O or S;
R³ is phenyl which may be substituted, a 5- to 6-membered heterocyclic group which may be substituted and comprises at least one of N, O and S, optionally substituted C₁₋₁₂ alkyl, amino, mono- or di(C₁₋₁₂)alkylamino; preferably optionally substituted phenyl, pyridyl, pyrimidinyl, or a group (G1) to (G9) as defined herein, or an optionally substituted C₁₋₆ alkyl, amino, mono- or di(C₁₋₁₂)alkylamino group;
m is 1 or 2;
R⁴ and R⁵ independently are hydrogen, halogen, C₁₋₁₂ alkyl which may be substituted or C₁₋₁₂ haloalkyl which may be substituted; preferably R⁴ and R⁵ independently are hydrogen, halogen, C₁₋₆ alkyl which may be substituted or C₁₋₆haloalkyl which may be substituted;
Z¹, Z² and Z³ independently are —CR⁴R⁵—, —C(O)—, —C(N—OR⁶)—, —NR⁶—, —S(O)ₙ—, —S(N—R⁶)—, —S(O)(N—R⁶)—, or oxygen, with the proviso that Z¹, Z² and Z³ do not simultaneously represent —CR⁴R⁵—, or do not simultaneously represent oxygen;
n is 0, 1 or 2;
R⁶ is hydrogen, cyano, nitro, C₁₋₁₂ alkyl, C₁₋₁₂ haloalkyl, C₃₋₈ cycloalkyl-C₁₋₄ alkyl, C₁₋₁₂ alkyl-carbonyl, C₁₋₁₂ haloalkyl-carbonyl, C₁₋₁₂ alkoxy-carbonyl, C₁₋₁₂ haloalkoxy-carbonyl, C₁₋₁₂ alkylsulfonyl, C₁₋₁₂ haloalkylsulfonyl, benzyl which may be substituted, phenethyl which may be substituted or C₁₋₄ alkyl which is substituted with a 6-membered heterocycle comprising 1 to 3 nitrogen, and each group from C₁₋₁₂ alkyl to C₁₋₄ alkyl which is substituted with a 6-membered heterocycle comprising 1 to 3 nitrogen among the definitions of R⁶ may be optionally substituted; preferably R⁶ is hydrogen, cyano, nitro, C₁₋₆ alkyl, C₁₋₆ haloalkyl, C₃₋₆ cycloalkyl-C₁₋₂ alkyl, C₁₋₆ alkyl-carbonyl, C₁₋₆ haloalkyl-carbonyl, C₁₋₆ alkoxy-carbonyl, C₁₋₆ haloalkoxy-carbonyl, C₁₋₆ alkylsulfonyl, C₁₋₆ haloalkylsulfonyl, benzyl which may be substituted, phenethyl which may be substituted or C₁₋₄ alkyl which is substituted with a 6-membered heterocycle comprising 1 to 3 nitrogen, and each group from C₁₋₆ alkyl to C₁₋₄ alkyl which is substituted with a 6-membered heterocycle comprising 1 to 3 nitrogen among the definitions of R⁶ may be optionally substituted;
R⁷ and R⁸ each independently is hydrogen, cyano, C₁₋₁₂ alkyl, C₃₋₈ cycloalkyl, C₁₋₁₂ haloalkyl, C₃₋₈ cyclohaloalkyl, C₂₋₁₂ alkenyl, C₂₋₁₂ alkynyl, C₂₋₁₂ haloalkenyl, C₂₋₁₂ haloalkynyl, C₁₋₁₂ alkoxy-carbonyl, C₁₋₁₂ alkoxy-thiocarbonyl, and each of the groups from C₁₋₁₂ alkyl to C₁₋₁₂ alkoxy-thiocarbonyl among the groups of R⁷ and R⁸ may be substituted; preferably R⁷ and R⁸ each independently is hydrogen, cyano, C₁₋₆ alkyl, C₃₋₆ cycloalkyl, C₁₋₆ haloalkyl, C₃₋₆ cyclohaloalkyl, C₂₋₆ alkenyl, C₂₋₆ alkynyl, C₂₋₁₂ haloalkenyl, C₂₋₆ haloalkynyl, C₁₋₆ alkoxy-carbonyl, C₁₋₆ alkoxy-thiocarbonyl, and each of the groups from C₁₋₆ alkyl to C₁₋₆ alkoxy-thiocarbonyl among the groups of R⁷ and R⁸ may be substituted; or
R⁷ and R⁸ together with the carbon atom to which they are bound may form a 3 to 6-membered carbocyclic ring;
R⁹ is hydrogen, C₁₋₁₂ alkyl, C₁₋₁₂ alkoxy, C₁₋₁₂ haloalkyl, mono- or di-(C₁₋₁₂)alkylamino, C₁₋₁₂ alkoxyamino, C₁₋₁₂alkoxy(C₁₋₁₂)alkyl, C₁₋₁₂ alkylhio, C₃₋₈ cycloalkyl, C₂₋₁₂ alkenyl, C₂₋₁₂alkynyl, C₃₋₈ cycloalkylalkyl, C₁₋₁₂ alkylthio(C₁₋₁₂)alkyl, C₁₋₁₂ alkylsulfinyl(C₁₋₁₂)alkyl, C₁₋₁₂ alkylsulfonyl(C₁₋₁₂)alkyl, C₁₋₁₂alkylcarbonyl(C₁₋₁₂)alkyl, C₃₋₈ cycloalkylamino, C₂₋₁₂ alkenylamino, C₂₋₁₂ alkynylamino, —CH₂R³, —C(O)R³ or —C(S)R³, and each group from C₁₋₁₂ alkyl to C₂₋₁₂ alkynylamino among the definitions of R⁹ may be substituted; preferably R⁹ is hydrogen, C₁₋₆ alkyl, C₁₋₆ alkoxy, C₁₋₆ haloalkyl, mono- or di-(C₁₋₆)alkylamino, C₁₋₆ alkoxyamino, C₁₋₆ alkoxy(C₁₋₆)alkyl, C₁₋₆ alkylhio, C₃₋₆ cycloalkyl, C₂₋₆ alkenyl, C₂₋₆ alkynyl, C₃₋₆ cycloalkylalkyl, C₁₋₆ alkylthio(C₁₋₆)alkyl, C₁₋₆ alkylsulfinyl(C₁₋₆)alkyl, C₁₋₆ alkylsulfonyl(C₁₋₆)alkyl, C₁₋₆ alkylcarbonyl(C₁₋₆)alkyl, C₃₋₆ cycloalkylamino, C₂₋₆ alkenylamino, C₂₋₆ alkynylamino, —CH₂R³, —C(O)R³ or —C(S)R³, and each group from C₁₋₆ alkyl to C₂₋₆ alkynylamino among the definitions of R⁹ may be substituted; more preferably R⁹ is C₁₋₆ alkyl, C₁₋₆ alkoxy, C₁₋₆ haloalkyl, mono- or di-(C₁₋₆)alkylamino, C₁₋₆ alkoxy(C₁₋₆)alkyl, C₁₋₆ alkylhio, C₃₋₆ cycloalkyl, C₂₋₆ alkenyl, C₂₋₆ alkynyl, C₃₋₆ cycloalkylalkyl, C₁₋₆ alkylthio(C₁₋₆)alkyl, C₁₋₆ alkylsulfinyl(C₁₋₆)alkyl, C₁₋₆ alkylsulfonyl(C₁₋₆)alkyl, and each group from C₁₋₆ alkyl to C₁₋₆ alkylsulfonyl(C₁₋₆)alkyl among the definitions of R⁹ may be substituted; most preferably R⁹ is C₁₋₆ alkyl, C₁₋₆ alkoxy, C₁₋₆ haloalkyl, C₁₋₆alkoxy(C₁₋₆)alkyl, C₁₋₆ alkylhio, and each group from C₁₋₆ alkyl to C₁₋₆ alkylthio among the definitions of R⁹ may be substituted;
R¹⁰ is hydrogen, hydroxy, C₁₋₁₂alkyl, C₁₋₁₂haloalkyl, C₃₋₈ cycloalkyl, C₂₋₁₂ alkenyl, C₂₋₁₂ alkinyl, C₁₋₁₂ alkoxy, C₁₋₁₂ alkylcarbonyl, C₁₋₁₂ alkoxycarbonyl, C₁₋₁₂ alkoxy(C₁₋₁₂)alkyl, C₁₋₁₂ cyanoalkyl, C₁₋₁₂ alkoxycarbonyl(C₁₋₁₂)alkyl, —CH₂R³, —C(O)R³ or —C(S)R³ and each group from C₁₋₁₂ alkyl to C₁₋₁₂ alkoxycarbonyl(C₁₋₁₂)alkyl among the definitions of R¹⁰ may be substituted; preferably R¹⁹ is hydrogen, hydroxy, C₁₋₆ alkyl, C₁₋₆ haloalkyl, C₃₋₆ cycloalkyl, C₂₋₆ alkenyl, C₂₋₆ alkinyl, C₁₋₆ alkoxy, C₁₋₆ alkylcarbonyl, C₁₋₆ alkoxycarbonyl, C₁₋₆ alkoxy(C₁₋₆)alkyl, C₁₋₆ cyanoalkyl, C₁₋₆ alkoxycarbonyl(C₁₋₆)alkyl, —CH₂R³, —C(O)R³ or —C(S)R³ and each group from C₁₋₆ alkyl to C₁₋₆ alkoxycarbonyl(C₁₋₆)alkyl among the definitions of R¹⁰ may be substituted; more preferably R¹⁰ is hydrogen, hydroxy, C₁₋₆alkyl, C₁₋₆haloalkyl, C₃₋₆ cycloalkyl, C₂₋₆ alkenyl, C₂₋₆ alkinyl, C₁₋₆alkoxy, C₁₋₆ alkylcarbonyl, C₁₋₆ cyanoalkyl, —CH₂R³, and each group from C₁₋₆ alkyl to C₁₋₆ alkylcarbonyl among the definitions of R¹⁰ may be substituted;
l stands for 1, 2 or 3;
R is C₁₋₁₂ alkyl which may be substituted or C₁₋₁₂haloalkyl which may be substituted; preferably R is C₁₋₆ alkyl which may be substituted or C₁₋₆ haloalkyl which may be substituted; most preferred R is CF₃;
X is hydrogen, halogen, nitro, cyano, hydroxy, mercapto, amino, C₁₋₁₂ alkyl, C₁₋₁₂ haloalkyl, C₁₋₁₂ alkoxy, C₁₋₁₂ haloalkoxy, C₁₋₁₂ alkylthio, C₁₋₁₂ alkylsulfinyl, C₁₋₁₂ alkylsulfonyl, C₁₋₁₂ haloalkylthio, C₁₋₁₂ haloalkylsulfinyl, C₁₋₁₂ haloalkylsulfonyl, C₁₋₁₂ alkylcarbonylamino, benzoylamino, C₁₋₁₂ alkoxy-carbonylamino, C₁₋₁₂ haloalkoxy-carbonylamino, C₁₋₁₂ alkoxyimino, C₁₋₁₂ haloalkoxyimino, C₁₋₁₂ alkylsulfonylamino or sulfur pentafluoride, and each group from C₁₋₁₂ alkyl to C₁₋₁₂ alkylsulfonylamino among the definitions of X may be optionally substituted; preferably X is hydrogen, halogen, nitro, cyano, hydroxy, mercapto, amino, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, C₁₋₆ haloalkylthio, C₁₋₆ haloalkylsulfinyl, C₁₋₆ haloalkylsulfonyl, C₁₋₆ alkylcarbonylamino, benzoylamino, C₁₋₆ alkoxy-carbonylamino, C₁₋₆ haloalkoxy-carbonylamino, C₁₋₆ alkoxyimino, C₁₋₆ haloalkoxyimino, C₁₋₆ alkylsulfonylamino or sulfur pentafluoride, and each group from C₁₋₆ alkyl to C₁₋₆ alkylsulfonylamino among the definitions of X may be optionally substituted; most preferred X is hydrogen, fluorine, chlorine, bromine, cyano, and CF₃; and
B¹, B², B³ and B⁴ independently are C—X or N; preferably B¹, B², B³ and B⁴ stand for C—X, or B¹, B³ and B⁴ stand for C—X while B² stands for nitrogen.

### EMBODIMENT [A]

### Preferred Nitroketones to be Used or Employed According to the Invention are Those of Formula (II)

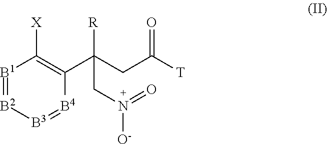 [0076] wherein
R is CF₃;
B¹, B², B³ and B⁴ independently of each other stand for C—X or nitrogen; preferably B¹, B², B³ and B⁴ stand for C—X, or B¹, B³ and B⁴ stand for C—X while B² stands for nitrogen;
X independently of each other is hydrogen, halogen, cyano or C₁₋₆haloalkyl; preferably X independently of each other is hydrogen, fluorine, chlorine, bromine, cyano, and CF₃;
T is one of the following chemical groups (T1), (T2), (T3), (T4) or (T5)

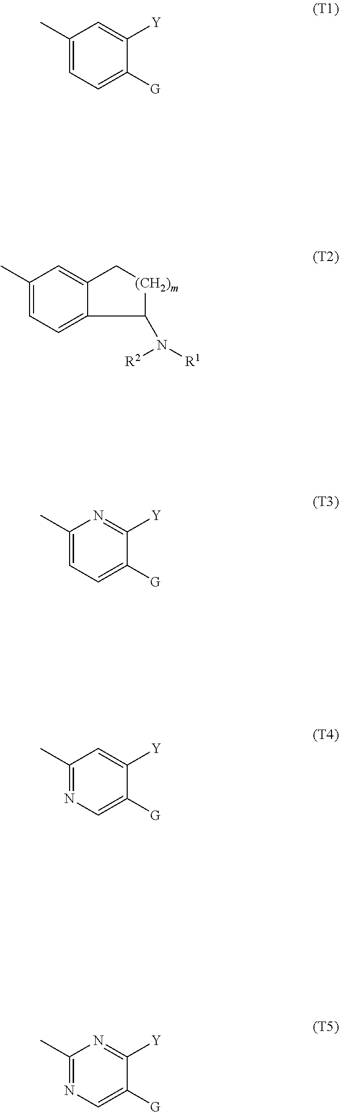

preferably T is a group (T1) or (T2),
wherein in the chemical groupings (T1) to (T5)
Y is hydrogen, halogen, nitro, cyano, amino, hydroxy, C₁₋₁₂ alkyl, C₁₋₁₂ haloalkyl, C₁₋₁₂ alkoxy, or C₁₋₁₂ haloalkoxy; preferably Y is hydrogen, halogen (in particular fluorine, chlorine, bromine), cyano, nitro, C₁₋₆ alkyl, C₁₋₆ haloalkyl, and C₁₋₆ alkoxy, or C₁₋₆ haloalkoxy;
m is 1 or 2, preferably m is 1;
R¹ is hydrogen, formyl, C₁₋₁₂ alkyl-carbonyl, C₁₋₁₂ haloalkyl-carbonyl, C₁₋₁₂ alkyl-thiocarbonyl, C₁₋₁₂ haloalkyl-thiocarbonyl, mono-(C₁₋₁₂) or di-(C₂₋₂₄) alkyl-aminocarbonyl, mono-(C₁₋₁₂) or di-(C₂₋₂₄) alkylamino-thiocarbonyl, C₁₋₁₂ alkoxy-carbonyl, C₁₋₁₂ alkoxy-C₁₋₁₂ alkyl-carbonyl, C₁₋₁₂ alkylsulfonyl, C₁₋₁₂ alkylsulfinyl, C₁₋₁₂ haloalkylsulfonyl, C₃₋₈ cycloalkyl-carbonyl, C₂₋₆ alkenyl-carbonyl, C₂₋₆ alkynyl-carbonyl, C₃₋₈ cycloalkyl-C₁₋₄ alkyl-carbonyl, C₁₋₁₂ alkylthio-C₁₋₁₂ alkyl-carbonyl, C₁₋₁₂ alkylsulfinyl-C₁₋₁₂alkyl-carbonyl, C₁₋₁₂ alkylsulfonyl-C₁₋₁₂alkyl-carbonyl, C₃₋₈ cycloalkylamino-carbonyl, C₂₋₆ alkenylamino-carbonyl, C₂₋₆ alkynylamino-carbonyl; preferably R¹ is hydrogen, C₁₋₆ alkyl-carbonyl, C₁₋₆ haloalkyl-carbonyl, C₁₋₆ alkyl-thio carbonyl, mono-(C₁₋₆) or di-(C₁₋₆)alkyl-aminocarbonyl, C₁₋₆ alkoxy-carbonyl, C₁₋₆ alkoxy-C₁₋₆ alkyl-carbonyl, C₁₋₆ alkylsulfonyl, C₁₋₆ alkylsulfinyl, C₁₋₆ haloalkylsulfonyl, C₃₋₆ cycloalkyl-carbonyl, C₂₋₆ alkenyl-carbonyl, C₁₋₆ alkylthio-C₁₋₆ alkyl-carbonyl, C₃₋₈ cycloalkyl-C₁₋₄ alkyl-carbonyl, C₃₋₆ cycloalkylamino-carbonyl;
R² is hydrogen, hydroxy, C₁₋₁₂ alkyl, C₂₋₆ alkenyl, C₁₋₁₂ alkoxy, C₁₋₁₂ alkyl-carbonyl, C₁₋₁₂ alkoxy-carbonyl, C₁₋₁₂ alkoxy-carbonyl-C₁₋₁₂ alkyl, cyano-C₁₋₁₂ alkyl, CH₂—R³; preferably R² is C₁₋₆ alkyl, C₁₋₆ alkoxy, C₁₋₆ alkyl-carbonyl, —CH₂—R³;
R³ is phenyl which may be substituted, a 5- to 6-membered heterocyclic group which may be substituted, optionally substituted C₁₋₁₂ alkyl, amino, mono- or di(C₁₋₁₂)alkylamino; preferably optionally substituted phenyl, pyridyl, pyrimidinyl, or a group (G1) to (G9) as defined herein, or an optionally substituted C₁₋₆ alkyl, amino, mono- or di(C₁₋₁₂)alkylamino group;
G is a halogen (in particular fluorine, chlorine or bromine), cyano, C₁₋₆ alkyl, C₁₋₆ alkoxycarbonyl, carboxy or CH₃S, or
G is a 5-membered heterocycle selected from a group consisting of the (G1) to (G9)

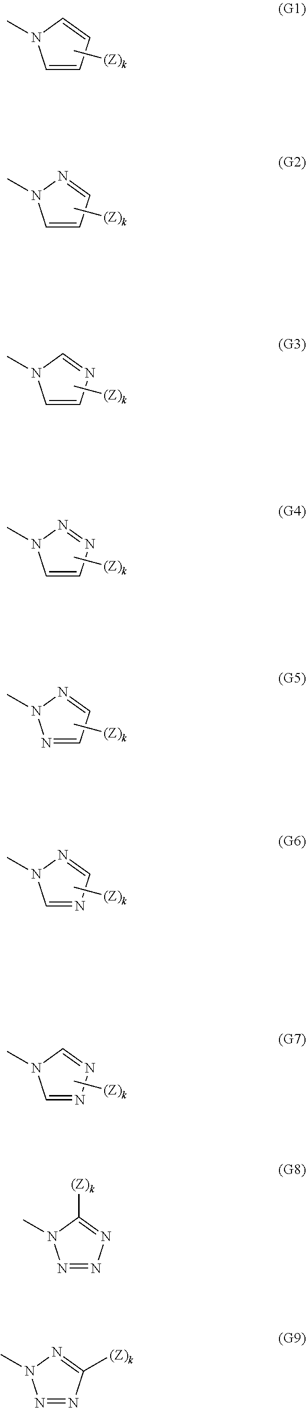

among the groups (G1) to (G9), groups (G2), (G6), (G8) and (G9) are preferred, more preferred are (G2) or (G6),
wherein in groups (G1) to (G9)
   (Z) is C₁₋₆ alkyl, C₁₋₆ halogenalkyl, halogen, cyano, or nitro; preferably (Z) is methyl, ethyl, trifluormethyl, chlorine, bromine or cyano, and
   k is 0, 1, 2, 3 or 4, or
G is one of the following groups (G10a) or (G10b):

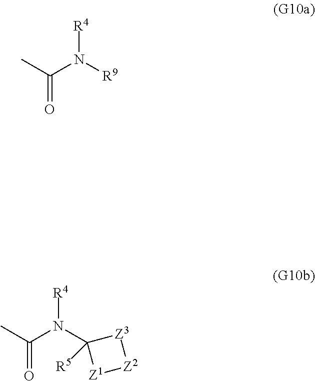

Z¹, Z² and Z³ independently of each other are —CR⁴R⁵—, —C(O)—, —C(N—OR⁶)—, —NR⁶—, —S(O)ₙ—, —S(N—R⁶)—, —S(O)(N—R⁶)—, or oxygen; preferably Z¹, Z² and Z³ independently of each other are —CR⁴R⁵—, —S(O)ₙ—, or oxygen, both with the proviso that Z¹, Z² and Z³ are not simultaneously —CR⁴R⁵— or oxygen; more preferably Z¹, Z³ are —CR⁴R⁵— (in particular CH₂), and Z² is oxygen or group —S(O)ₙ—;
n is 0, 1 or 2;
R⁴ and R⁵ independently of each other are hydrogen, halogen, C₁₋₁₂ alkyl or C₁₋₁₂haloalkyl; preferably R⁴ and R⁵ independently of each other are hydrogen or C₁₋₆ alkyl;
R⁶ is hydrogen, cyano, nitro, C₁₋₁₂ alkyl, C₁₋₁₂ haloalkyl, C₃₋₈ cycloalkyl-C₁₋₄ alkyl, C₁₋₁₂ alkyl-carbonyl, C₁₋₁₂ haloalkyl-carbonyl, C₁₋₁₂ alkoxy-carbonyl, C₁₋₁₂ haloalkoxy-carbonyl, C₁₋₁₂ alkylsulfonyl, C₁₋₁₂ haloalkylsulfonyl, benzyl which may be substituted, phenethyl which may be substituted or C₁₋₄ alkyl which is substituted with a 6-membered heterocycle comprising 1 to 3 nitrogen, and each group from C₁₋₁₂ alkyl to C₁₋₄ alkyl which is substituted with a 6-membered heterocycle comprising 1 to 3 nitrogen among the definitions of R⁶ may be optionally substituted;
G is one of the following groups (G11) to (G16):

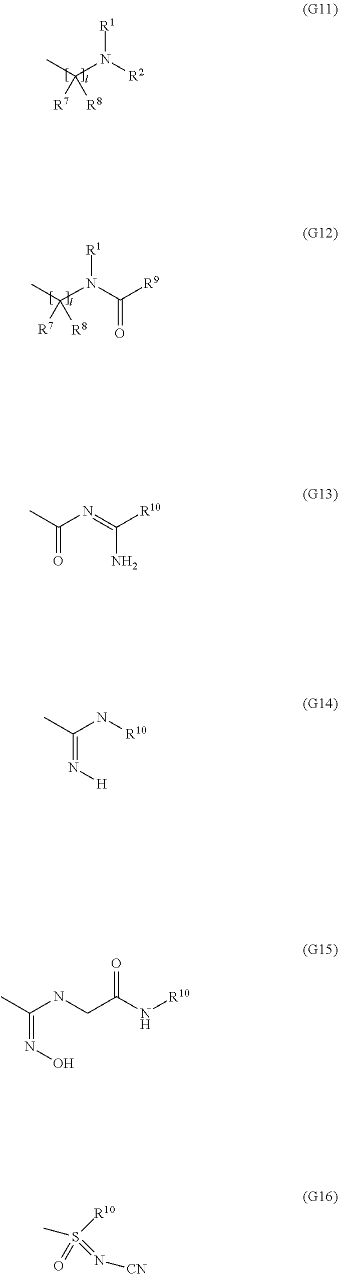

among the groups (G11) to (G16), groups (G11) and G12) are preferred,
wherein in groups (G11) to (G16):
   R⁷ and R⁸ each independently is hydrogen cyano, C₁₋₆ alkyl, C₃₋₆ cycloalkyl, C₁₋₆ haloalkyl, C₃₋₆ cyclohaloalkyl, C₂₋₆ alkenyl, C₂₋₆ alkynyl, C₂₋₁₂ haloalkenyl, C₂₋₆ haloalkynyl, C₁₋₆ alkoxy-carbonyl, C₁₋₆ alkoxy-thiocarbonyl and each of the groups from C₁₋₆ alkyl to C₁₋₆ alkoxy-thiocarbonyl among the groups of R⁷ and R⁸ may be substituted, or
   R⁷ and R⁸ together with the carbon atom to which they are bound may form a 3 to 6-membered carbocyclic ring;
   R⁹ is hydrogen, C₁₋₆ alkyl, C₁₋₆ alkoxy, C₁₋₆ haloalkyl, mono- or di-(C₁₋₆)alkylamino, C₁₋₆ alkoxyamino, C₁₋₆alkoxy(C₁₋₆)alkyl, C₁₋₆ alkylhio, C₃₋₆ cycloalkyl, C₂₋₆ alkenyl, C₂₋₆ alkynyl, C₃₋₆cycloalkylalkyl, C₁₋₆ alkylthio C₁₋₆ alkyl, C₁₋₆ alkylsulfinyl(C₁₋₆)alkyl, C₁₋₆ alkylsulfonyl(C₁₋₆)alkyl, C₁₋₆alkylcarbonyl(C₁₋₆)alkyl, C₃₋₆ cycloalkylamino, C₂₋₆ alkenylamino, C₂₋₆ alkynylamino, —CH₂R³, —C(O)R³ or —C(S)R³, and each group from C₁₋₆ alkyl to C₂₋₆ alkynylamino among the definitions of R⁹ may be substituted; preferably R⁹ is C₁₋₆ alkyl, C₁₋₆ alkoxy, C₁₋₆ haloalkyl, mono- or di-(C₁₋₆)alkylamino, C₁₋₆ alkoxy(C₁₋₆)alkyl, C₁₋₆ alkylhio, C₃₋₆ cycloalkyl, C₂₋₆ alkenyl, C₂₋₆ alkynyl, C₃₋₆cycloalkylalkyl, C₁₋₆ alkylthio C₁₋₆ alkyl, C₁₋₆ alkylsulfinyl(C₁₋₆)alkyl, C₁₋₆ alkylsulfonyl(C₁₋₆)alkyl; more preferably R⁹ is C₁₋₆ alkyl, C₁₋₆ alkoxy, C₁₋₆ haloalkyl, C₁₋₆ alkoxy(C₁₋₆)alkyl, C₁₋₆ alkylhio which groups may be substituted;
   R¹⁰ is hydrogen, hydroxy, C₁₋₆ alkyl, C₁₋₆ haloalkyl, C₃₋₆ cycloalkyl, C₂₋₆ alkenyl, C₂₋₆ alkinyl, C₁₋₆ alkoxy, C₁₋₆ alkylcarbonyl, C₁₋₆ alkoxycarbonyl, C₁₋₆ alkoxy(C₁₋₆)alkyl, C₁₋₆ cyanoalkyl, C₁₋₆ alkoxycarbonyl (C₁₋₆)alkyl, —CH₂R³, —C(O)R³ or —C(S)R³ and each group from C₁₋₆ alkyl to C₁₋₆ alkoxycarbonyl (C₁₋₆)alkyl among the definitions of R¹⁰ may be substituted; preferably R¹⁰ is hydrogen, hydroxy, C₁₋₆ alkyl, C₁₋₆ haloalkyl, C₃₋₆ cycloalkyl, C₂₋₆ alkenyl, C₂₋₆ alkinyl, C₁₋₆ alkoxy, C₁₋₆ alkylcarbonyl, C₁₋₆ cyanoalkyl, —CH₂R³ which groups may be substituted; and
   l stands for 1, 2 or 3.

If G is (G13) or (G14), it is preferred that R¹⁰ is hydroxyl, C₁₋₁₂ alkoxy, or C₁₋₁₂ alkyl, preferably hydroxyl, C₁₋₆ alkoxy, or C₁₋₁₂ alkyl.

In Embodiment [A] compounds having one of the following general structures (A-IIa) or (A-IIb) are preferred:

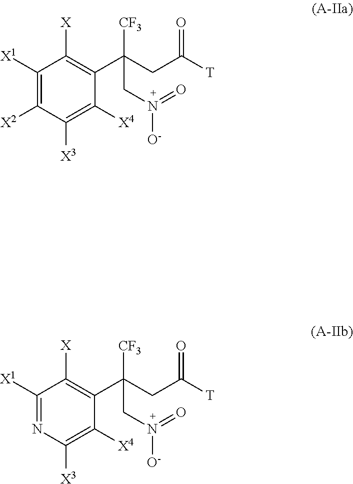

wherein X¹, X², X³ and X⁴ have the meaning as defined for X in embodiment A and T is as generally defined herein for group T.

### EMBODIMENT [B]

Preferred are nitroketones of formula (II) to be used or employed according to the invention (e.g in the manufacturing method or for combating invertebrate pests) having one of the following general structures (II-a) to (II-o), wherein X¹, X², X³ and X⁴ are as defined for X herein, and all other groups, such as R², R⁷, R⁸, R⁹, Y, G, (Z), and k are as defined and given herein.

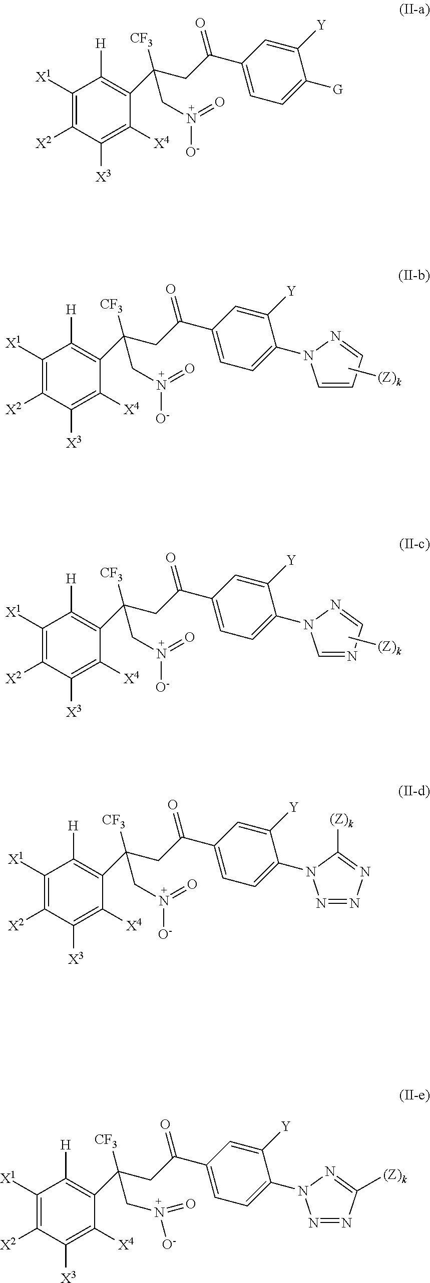

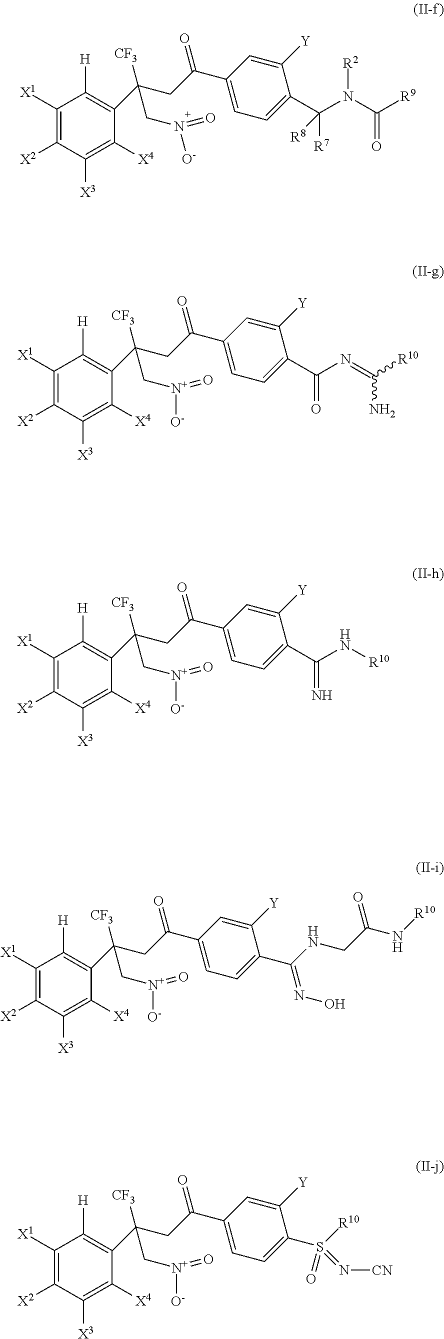

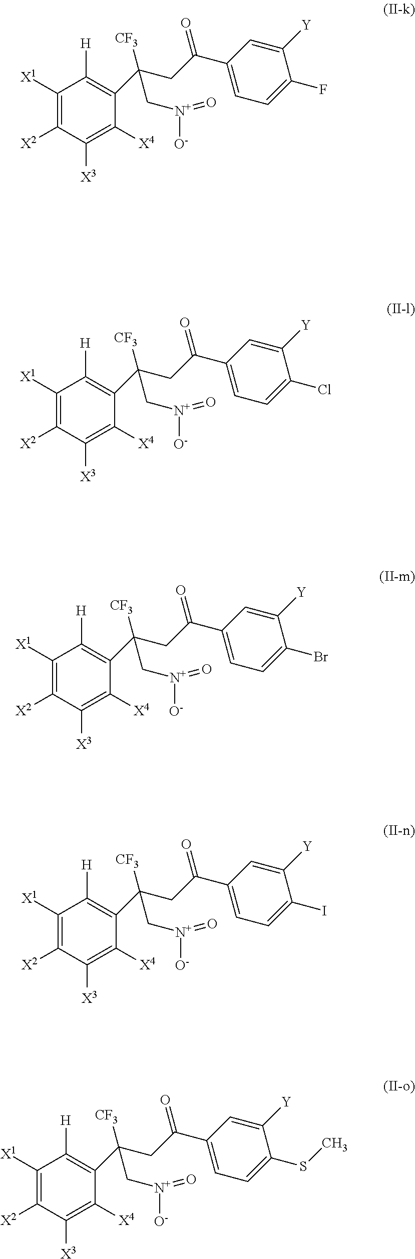

### EMBODIMENT [C]

Preferred are nitroketones of formula (II) to be used or employed according to the invention (e.g in the manufacturing method or for combating invertebrate pests) having one of the following general structures (II-p) to (II-ad) wherein X¹, X², X³ and X⁴ are as defined for X herein, and all other groups, such as R², R⁷, R⁸, R⁹, Y, G, (Z), and k are as defined and given herein.

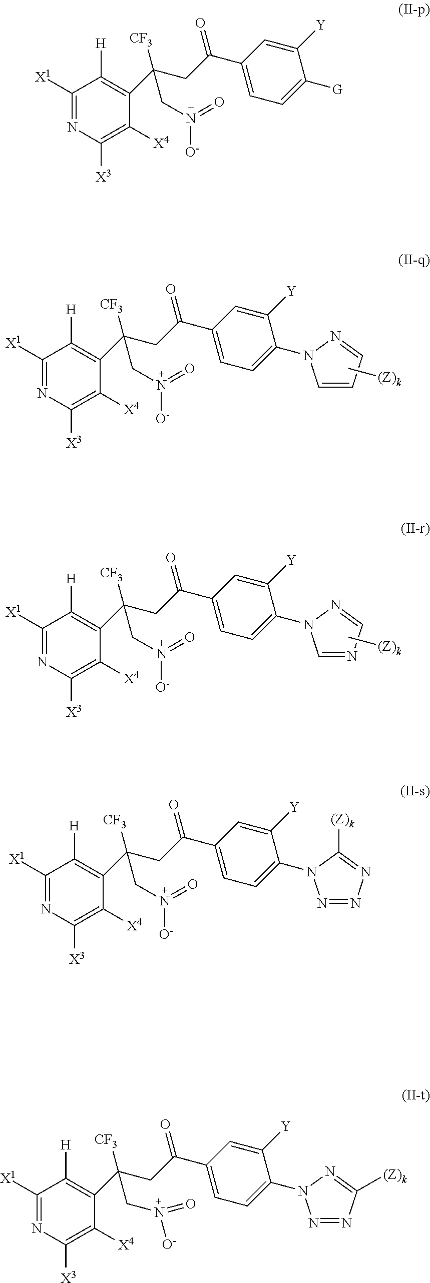

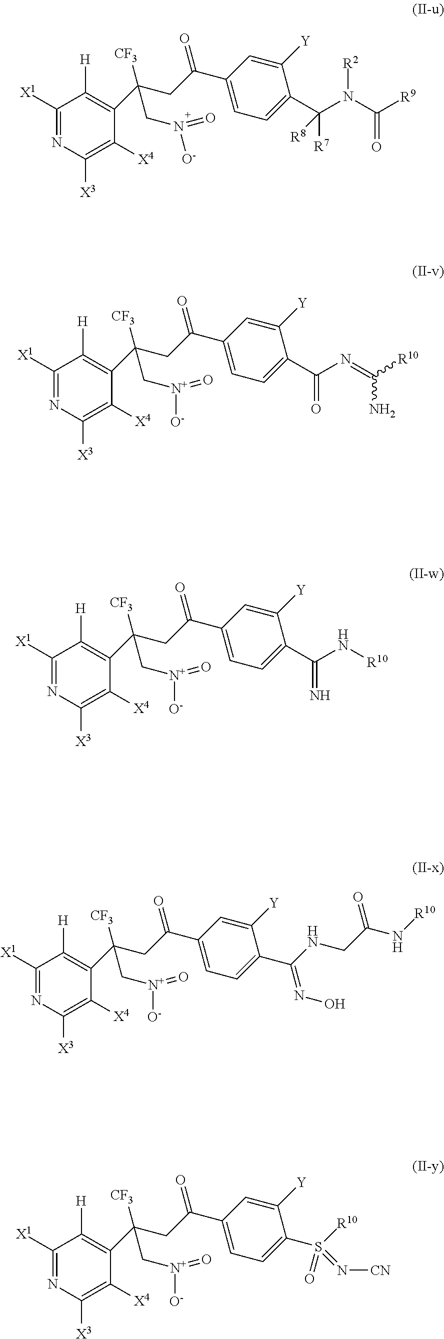

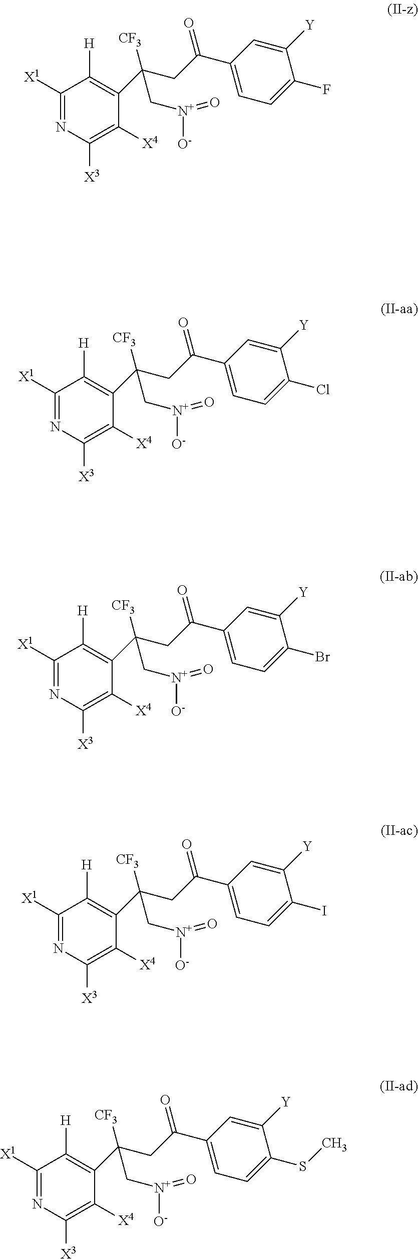

### EMBODIMENT [D]

Preferred are nitroketones of formula (II) to be used or employed according to the invention (e.g in the manufacturing method or for combating invertebrate pests) having one of the following general structures (II-ae) and (II-af) wherein X¹, X², X³ and X⁴ are as defined for X herein, and R¹ and R² are as defined and given herein.

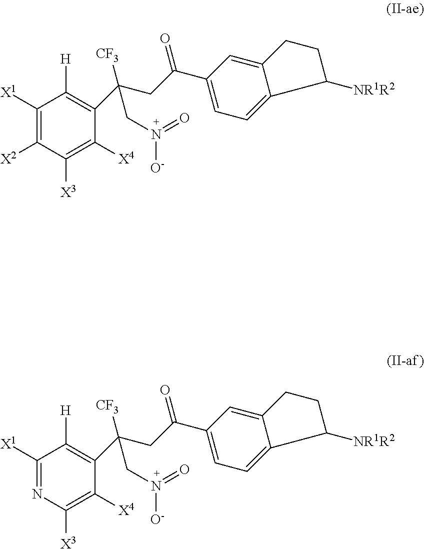

### EMBODIMENT [E]

Preferred are nitroketones having one of the formulae (II-a) to (II-o), or (II-ae) to be used or employed according to the invention (e.g in the manufacturing method or for combating invertebrate pests) wherein X¹, X² and X³ is chlorine and X⁴ is hydrogen.

### EMBODIMENT [F]

Preferred are nitroketones having one of the formula (II-a) to (II-o) or (II-ae) to be used or employed according to the invention (e.g in the manufacturing method or for combating invertebrate pests) wherein X¹ and X² are chlorine, X³ is trifluoromethyl and X⁴ is hydrogen.

### EMBODIMENT [G]

Preferred are nitroketones having one of the formula (II-a) to (II-o) or (II-ae) to be used or employed according to the invention (e.g in the manufacturing method or for combating invertebrate pests) X¹ and X³ are chlorine and X² and X⁴ are fluorine.

### EMBODIMENT [H]

Preferred are nitroketones having one of the formula (II-a) to (II-o) or (II-ae) to be used or employed according to the invention (e.g in the manufacturing method or for combating invertebrate pests) wherein X¹ is trifluoromethyl and X², X³ and X⁴ are hydrogen.

### EMBODIMENT [I]

Preferred are nitroketones having one of the formula (II-a) to (II-o) or (II-ae) to be used or employed according to the invention (e.g in the manufacturing method or for combating invertebrate pests) wherein X¹ and X³ are trifluoromethyl and X² and X⁴ are hydrogen.

### EMBODIMENT [J]

Preferred are nitroketones having one of the formula (II-a) to (II-o) or (II-ae) to be used or employed according to the invention (e.g in the manufacturing method or for combating invertebrate pests) wherein X¹ and X³ are chlorine and X² and X⁴ are hydrogen.

### EMBODIMENT [K]

Preferred are nitroketones having one of the formula (II-a) to (II-o) or (II-ae) to be used or employed according to the invention (e.g in the manufacturing method or for combating invertebrate pests) wherein X¹ is chlorine, X³ is trifluoromethyl and X² and X⁴ are hydrogen.

### EMBODIMENT [L]

Preferred are nitroketones having one of the formula (II-a) to (II-o) or (II-ae) to be used or employed according to the invention (e.g in the manufacturing method or for combating invertebrate pests) wherein X¹ is trifluoromethyl, X² is fluorine and X³ and X⁴ are hydrogen.

### EMBODIMENT [M]

Preferred are nitroketones having one of the formula (II-a) to (II-o) or (II-ae) to be used or employed according to the invention (e.g in the manufacturing method or for combating invertebrate pests) wherein X¹ and X³ are bromine and X² and X⁴ are hydrogen.

### EMBODIMENT [N]

Preferred are nitroketones having one of the formula (II-a) to (II-o) or (II-ae) to be used or employed according to the invention (e.g in the manufacturing method or for combating invertebrate pests) wherein X¹ is trifluoromethyl, X³ is fluorine and X² and X⁴ are hydrogen.

### EMBODIMENT [O]

Preferred are nitroketones having one of the formula (II-p) to (II-ad) or (II-af) to be used or employed according to the invention (e.g in the manufacturing method or for combating invertebrate pests) wherein X¹ and X³ are chlorine and X⁴ is hydrogen.

### EMBODIMENT [P]

Preferred are nitroketones having one of the formula (II-p) to (II-ad) or (II-af) to be used or employed according to the invention (e.g in the manufacturing method or for combating invertebrate pests) wherein X¹ and X³ are trifluoromethyl and X⁴ is hydrogen.

### EMBODIMENT [Q]

Preferred are nitroketones having one of the formula (II-p) to (II-ad) or (II-af) to be used or employed according to the invention (e.g in the manufacturing method or for combating invertebrate pests) wherein X¹ is trifluoromethyl, X³ is chlorine and X⁴ is hydrogen.

### EMBODIMENT [R]

Preferred are nitroketones having one of the formula (II-p) to (II-ad) or (II-af) to be used or employed according to the invention (e.g in the manufacturing method or for combating invertebrate pests) wherein X¹ is trifluoromethyl and X³ and X⁴ are hydrogen.

### EMBODIMENT [S]

Preferred are nitroketones having one of the formula (II-p) to (II-ad) or (II-af) to be used or employed according to the invention (e.g in the manufacturing method or for combating invertebrate pests) wherein X¹ is chlorine and X³ and X⁴ are hydrogen.

If a nitroketone of the following formula (II-k,), (II-l), (II-m), (II-n), (II-o), (II-z), (II-aa), (II-ab), (II-ac), (II-ad) is used in the method according to the invention, then it is preferred that the method according to the invention comprises another step, namely the reaction of a compound of formula (I) wherein T is (T1) and G is halogen or CH₃S, and wherein all other groups are as defined herein, with an optionally substituted saturated or unsaturated 5- to 6-membered heterocycle, preferably with a heterocycle selected from the following heterocycles (G1-H) to (G9-H)

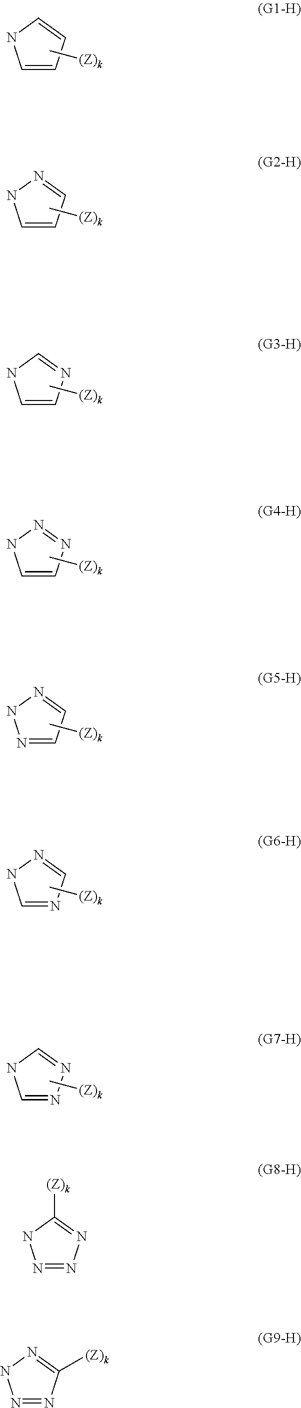

more preferably with a heterocycle (G2-H), (G6-H), (G8-H), (G9-H) most preferably with a heterocycle (G2-H) or (G6-H) under appropriate conditions to give compounds of formula (I) wherein T is (T1) and G is an optionally substituted saturated or unsaturated 5- to 6-membered heterocyclic group as defined herein.

If not mentioned otherwise, the following definitions shall apply throughout the application:

“Alkyl” represents linear or branched C₁₋₁₂ alkyl such as methyl, ethyl, n- or iso-propyl, n-, iso-, sec- or tert-butyl, n-pentyl, n-hexyl, n-heptyl, n-octyl, n-nonyl, n-decyl, n-undecyl or n-dodecyl, preferably C₁₋₆ alkyl, and more preferably C₁₋₄ alkyl. In addition, examples of an alkyl moiety included in other groups as a part of constitution, can be those described above for the “alkyl”.

“Acylamino” represents, for example, alkylcarbonylamino, cyclopropylcarbonylamino or benzoylamino, wherein examples of the alkyl moiety can also be those described above for the “alkyl”.

“Halogen” and a halogen moiety included in each group substituted with a halogen represent fluorine, chlorine, bromine or iodine, preferably fluorine, chlorine or bromine.

“Cycloalkyl” represents C₃₋₈ cycloalkyl, such as cyclopropyl, cyclobutyl, cyclopentyl, cyclohexyl, cycloheptyl or cyclooctyl, preferably C₃₋₇ cycloalkyl, and more preferably C₃₋₆ cycloalkyl. The cycloalkyl groups according to the invention may be substituted with at least one of the following groups C₁₋₆ alkyl, C₁₋₆ alkoxy, halogen, halogenalkyl and cyano. The term “cycloalkyl” also includes “heterocycloalkyl groups” i.e. C₃₋₆ cycloalkyl groups which are interrupted by oxygen or/and sulfur and which may be substituted with at least one of the following groups C₁₋₆ alkyl, C₁₋₆ alkoxy, halogen, halogenalkyl and cyano.

“Alkenyl” represents C₂₋₆ alkenyl, preferably C₂₋₅ alkenyl, such as vinyl, allyl, 1-propenyl, 1-(or 2-, or 3-) butenyl or 1-pentenyl, more preferably C₂₋₄ alkenyl.

“Alkynyl” represents C₂₋₆ alkynyl, preferably C₂₋₅ alkynyl, such as ethynyl, propargyl, 1-propynyl, butan-3-ynyl or pentan-4-ynyl, more preferably C₂₋₄ alkynyl.

“Aryl” represents a C₆₋₁₂ aromatic hydrocarbon group, for example, phenyl, naphthyl or biphenyl, preferably a C₆₋₁₀ aromatic hydrocarbon group, and more preferably a C₆ aromatic hydrocarbon group, or phenyl.

“Aralkyl” represents arylalkyl, for example, benzyl or phenethyl.

“Heterocycle” represents a 5- or 6-membered heterocyclic ring group comprising at least one of N, O and S as a hetero atom, and also represents a fused heterocyclic ring group which may be benzo-fused.

As specific examples of the heterocyclic ring, furyl, thienyl, pyrrolyl, isoxazolyl, pyrazolyl, oxazolyl, oxathiazolyl, imidazolyl, triazolyl, oxadiazolyl, thiadiazolyl, tetrazolyl, pyridyl, pyrimidinyl, pyridazinyl, pyrazinyl, triazinyl, indolyl, benzoxazolyl, quinolyl and the like can be mentioned.

As for the substituent which may be substituted on each “group which may be optionally substituted”, those selected from nitro, cyano, hydroxy, mercapto, isocyano, cyanate, isothiocyanate, carboxy, carbamoyl, aminosulfonyl, monoalkylamino, dialkylamino, N-alkylcarbonylamino, alkyl, alkenyl, alkynyl, cycloalkyl, cycloalkenyl, SF₅, alkoxy, alkenyloxy, alkynyloxy, cycloalkyloxy, cycloalkenyloxy, alkoxycarbonyl, alkenyloxycarbonyl, alkynyloxycarbonyl, aryloxycarbonyl, alkylcarbonyl, alkenylcarbonyl, alkynylcarbonyl, arylcarbonyl, alkylthio, cycloalkylthio, alkenylthio, cycloalkenylthio, alkynylthio, alkylsulfenyl, alkylsulfinyl including isomers, alkylsulfonyl, monoalkylaminosulfonyl, dialkylaminosulfonyl, alkylphosphinyl, alkylphosphonyl, alkylphosphinyl including isomers, alkylphosphonyl including isomers, N-alkyl-aminocarbonyl, N,N-dialkyl-aminocarbonyl, N-alkylcarbonyl-aminocarbonyl, N-alkylcarbonyl-N-alkylaminocarbonyl, aryl, aryloxy, benzyl, benzyloxy, benzylthio, arylthio, arylamino, benzylamino, trialkylsilyl, alkoxyalkyl, alkylthioalkyl, alkylthioalkoxy, alkoxyalkoxy, phenethyl, benzyloxy, haloalkyl, haloalkoxy, haloalkylthio, haloalkylcarbonyl, haloalkoxycarbonyl, haloalkoxyalkoxy, haloalkoxyalkylthio, haloalkoxyalkylcarbonyl or haloalkoxyalkyl, cycloalkylamino-carbonyl, alkylsulfinylimino, alkylsulfonylimino, alkoxyimino, a heterocyclic group and the like can be mentioned.

With the method according to the invention the desired pyrroline can be prepared in good yields and high purity, such that generally no complex purifications are required subsequently, and which at the same time is simple and inexpensive. This is surprising since it was expected that a reductive cyclization by employing a catalytic hydrogenation does lead rather to a pyrrolidine compound than to pyrrolin compound. Also, it was surprising that when halogen-substituted nitroketones according to the invention were used, in particular bromine-substituted nitroketones according to the invention, there was no significant depletion of the bromine.

Suitable catalysts to be used in the catalytic hydrogenation and thus in the reductive cyclization according to the invention comprise one or more transition metals of groups 8-11 of the Periodic Table, especially one or more metals selected from iron, ruthenium, copper, cobalt, rhodium, iridium, nickel, palladium and platinum. Besides their catalytic activity, suitable catalysts are under the selected reaction conditions inert. The metals may be present in any chemical form, for example in elemental, colloidal, salt or oxide form, together with complexing agents as chelates, or as alloys, in which case the alloys may also include other metals, for example aluminium, as well as the metals listed above. The metals may be present in supported form, i.e. applied to any support, preferably an inorganic support. Examples of suitable supports are carbon (charcoal or activated carbon), aluminium oxide, silicon dioxide, zirconium dioxide, titanium dioxide, calcium carbonate, and barium sulfate.

Suitable catalysts contain at least one precious metal, such as platinum and palladium, or cobalt or nickel. Suitable catalysts are moreover Raney-nickel catalysts, Raney-cobalt catalysts, Lindlar catalysts, platinium catalysts which are doped with vanadium or copper. Among the suitable catalyst Raney-cobalt catalysts and platinum containing catalysts (in particular platinum on carbon (Pt/C)) are preferred. If Raney-nickel catalysts are used in the method according to the invention, it is particularly advantageous to use Raney-nickel in the presence of an additive as defined herein. The catalysts can be used in any form, for example dry, or wet (water-wet). Preferably, the catalysts are used several times.

In the process according to the invention, the catalyst is used, based on the nitroketone used, in a concentration of about 0.01 to about 50% by weight. The catalyst is preferably used in a concentration of about 1 to about 50% by weight, more preferably the catalyst is used in a concentration of about 3% by weight to about 30% by weight.

The catalytic hydrogenation and thus reductive cyclization according to the invention is performed preferably at a temperature in the range from about 10° C. to about 200° C., more preferably at a temperature in the range from about 50° C. to about 110° C.

The catalytic hydrogenation and thus the reductive cyclization according to the invention is performed under elevated pressure (i.e. up to about 200 bar), preferably in an autoclave in a hydrogen gas atmosphere. The (additional) pressure increase can be brought about by supply of an inert gas, such as nitrogen or argon. The reductive cyclization according to the invention is effected preferably at a hydrogen pressure in the range from about 3.5 to about 100 bar, more preferably at a hydrogen pressure in the range from about 5 to about 50 bar, most preferably at a hydrogen pressure in the range from about 10 to 30 bar.

Suitable additives to be used in the method according to the invention are Lewis acids (e.g. ZnBr₂, ZnCl₂, MgO), Brønstedt acids (e.g. H₂SO₄, HCl, CH₃CO₂H, CF₃CO₂H, p-toluenesulfonic acid, methanesulfonic acid, trifluoromethanesulfonic acid, formic acid), organic sulfur-containing compounds (e.g. thiophene, tetrahydrothiophene, or 2,2′-thiobisethanol), organic bases (e.g. sodium acetate, potassium acetate, sodium formate, potassium formate, triethylamine, pyridine, N-methyl-morpholine, morpholine, piperidine), inorganic bases (e.g. sodium carbonate, potassium carbonate, sodium phosphate, potassium phosphate), and water scavengers (e.g. Na₂SO₄, MgSO₄, molecular sieves (such as zeolithes)).

When Raney-nickel is used, it is preferred that an additive is present in the method according to the invention. Preferably, organic sulfur-containing compounds are used.

If organic sulfur-containing compounds are used as an additive in the method according to the invention, preferred concentrations of such sulfur-containing compounds are in the range from about 0.001 mol % to about 20 mol % with respect to the amount of nitroketone used in the reaction, more preferably in the range of 0.01 mol % to 1.0 mol % and most preferably in the range of 0.01 mol % to 0.5 mol %.

If Brønstedt acids, organic or inorganic bases are used as an additive in the method according to the invention, preferred concentrations of such compounds are in the range from about 0.1 mol % to about 100 mol % with respect to the amount of nitroketone used, more preferably in the range from 1 to 20 mol % and most preferably in the range from 1 to 10 mol %.

If Lewis acids are used as an additive in the method according to the invention, preferred concentrations are in the range of about 0.1 mol % to about 100 mol % with respect to the amount of nitroketone used, more preferably in the range from 1.0 to 50 mol % and most preferably in the range from 1.0 to 20 mol %.

If water scavengers are used as an additive in the method according to the invention, preferred concentrations are in the range of about 1 wt % to 100 wt % with respect to the amount of nitroketone used, more preferably in the range from 5 wt % to 50 wt % and most preferably in the range from 10 wt % to 50 wt %.

It is generally advantageous to perform the process according to the invention in the presence of solvents (diluents). However, the catalytic hydrogenation can also be performed without a solvent. Solvents are advantageously used in such an amount that the reaction mixture remains efficiently stirrable over the entire process. Advantageously, based on the nitroketone used, 1 to 50 times the amount of solvent, preferably 2 to 40 times the amount of solvent and more preferably 2 to 30 times the amount of solvent is used.

Useful solvents for the performance of the process according to the invention include all organic solvents which are inert under the reaction conditions, the type of solvent used depending on the type of reaction procedure, more particularly on the type of catalyst used and/or the hydrogen source (introduction of gaseous hydrogen or generation in situ). Solvents are also understood in accordance with the invention to mean mixtures of pure solvents.

Solvents suitable in accordance to the invention are alcohols, such as methanol, ethanol, isopropanol, butanol; ethers, such as ethyl propyl ether, methyl tert-butyl ether, n-butyl ether, anisole, phenetole, cyclohexyl methyl ether, dimethyl ether, diethyl ether, dimethylglycol, diphenyl ether, dipropyl ether, diisopropyl ether, di-n-butyl ether, diisobutyl ether, diisoamyl ether, ethylene glycol dimethyl ether, isopropyl ethyl ether, tetrahydrofuran, methyl tetrahydrofuran, dioxane, dichlorodiethyl ether, and polyethers of ethylene oxide and/or propylene oxide; amines, such as trimethyl-, triethyl-, tripropyl-, and tributylamine, N-methyl morpholine, pyridine, alkylated pyridines and tetramethyl diamines, aliphatic, cycloaliphatic or aromatic hydrocarbons such as pentane, hexane, heptane, octane, nonane, and technical-grade hydrocarbons which may be substituted by fluorine and chlorine atoms, such as methylene chloride, dichloromethane, trichloromethane, carbon tetrachloride, fluorobenzene, chlorobenzene or dichlorobenzene; for example white spirits having components with boiling points in the range, for example, from 40° C. to 250° C., cymene, petroleum fractions within a boiling range from 70° C. to 190° C., cyclohexane, methylcyclohexane, petroleum ether, ligroin, octane, benzene, toluene, and xylene.

In the process according to the invention, it is preferred to use alcohols or cyclic ethers as solvent. Preferred is methanol, ethanol, isopropanol, tetrahydrofuran, or methyltetrahydrofuran. Among before-mentioned solvents tetrahydrofuran and isopropanol is preferred.

The nitroketones of formula (II) as defined herein, including the nitroketones of having the specific substructures as defined herein, as well as the specific embodiments as given herein (hereinafter also referred to as the “compounds of the present invention”) exhibit a very potent pesticidal activity. Thus, they can be used as pesticidal agents, preferably insecticide. In addition, the compounds of the present invention have a potent controlling effect against harmful insects without exhibiting any phytotoxicity to crop plants. Thus, the compounds of the present invention can be used for controlling a broad range of harmful invertebrate pests which occur in the agriculture, for instances, harmful sucking insects, chewing insects, other plant-parasitic insects, storage insects, hygienically harmful insects and the like, and also for combating and extermination thereof. As examples of the harmful invertebrate pests which occur in the agriculture, the following pests can be mentioned.

As an insect, Coleoptera, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; Lepidoptera, for example, Lymantria dispar, Malacosoma neustria, Pieris rapae, Spodoptera litura, Mamestra brassicae, Chilo suppressalis, Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella; Hemiptera, for example, Nephotettix cincticeps, Nilaparvata lugens, Pseudococcus comstocki, Unapsis yanonensis, Myzus persicas, Aphis pomi, Aphis gossypii, Rhopalosiphum pseudobrassicas, Stephanitis nashi, Nezara spp., Trialeurodes vaporariorm, Psylla spp.; Thysanoptera, for example, Thrips palmi, Franklinella occidental; Orthoptera, for example, Blatella germanica, Periplaneta americana, Gryllotalpa Africana, Locusta migratoria migratoriodes; Isoptera, for example, Reticulitermes speratus, Coptotermes formosanus; Diptera, for example, Musca domestica, Aedes aegypti, Hylemia platura, Culex pipiens, Anopheles sinensis, Culex tritaeniorhynchus, Liriomyza torifolii. As Acarina, Tetranychus cinnabarinus, Tetranychus urticae, Panonychus citri, Aculops pelekassi, Tarsonemus spp. can be mentioned. As nematodes, Meloidogyne incognita, Bursaphelenchus lignicolus Mamiya et Kiyohara, Aphelenchoides besseyi, Heterodera glycines, Pratylenchus spp. can be mentioned.

Further, the compounds of the present invention have excellent tolerability in plant and exhibit low toxicity which is desirable for warm-blooded animals. Still further, they are well tolerated in various environmental conditions, and therefore useful for protecting plants and plant parts.

The application of the compounds of the present invention may contribute to increase in harvest yield and improvement in harvested product quality. In addition, the compounds are suitable for the protection of preserved products and materials, and in hygienic field, for the control of harmful animals, in particular, insects, spider like animals, helminthes, nematodes and mollusks that are encountered in the field of agriculture, horticulture, veterinary medicine, forest, gardening and amusement facilities and the like.

The compounds of the present invention can be preferably used as agents for protecting plants. The compounds of the present invention are active for normally sensitive species and tolerant species, at all levels or several levels of growth of a plant. The above-described harmful organisms particularly include the followings.

As Anoplura (Phthiraptera), for example, Damalinia spp., Haematopinus, Linognathus spp., Pediculus spp., Trichodectes spp.

As Arachnid, for example, Acarus siro, Aceria sheldoni. Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranyctus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

As Bivalvia, for example, Dreissena spp.

As Chilopoda, for example, Geophilus spp., Scutigera spp.

As Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

As Collembola, for example, Onychiurus armatus.

As Dermaptera, for example, Forficula auricularia.

As Diplopoda, for example, Blaniulus guttulatus.

As Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp, Oscinella frit, Pegomyia hyoscyami, Phorbia spp., Stomoxys spp., Tabanus spp., Tannia spp., Tipula paludosa, Wohlfahrtia spp.

As Gastropoda, for example, Anion spp., Biomphalaria spp., Bulinus spp., Deroceras spp., Galba spp., Lymnaea spp., Oncomelania spp., Succinea spp.

As Helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medeinensis, Echinococcus granulosus, Echinococcus multiocularis, Enterobius vermicularis, Faciola spp., Haemonchus spp., Heterakis spp., Hymenolepis nana, Hyostrongulus spp., Loa loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

In addition, protozoa like Eimeria, etc. can be also controlled.

As Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus. spp., Euschistus spp., Eurygaster spp., Heliopeltis spp., Horchias nobiellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodonius spp., Sahlbergella singularis, Scotino phora spp., Stephanitis nashi, Tibraca spp., Triatoma spp.

As Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccu spp., Chryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchart, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratorioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesda gigas, Rastrococcus spp., Rhopalosiphum spp., Saissetia spp., Scaphoides Manus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolli.

As Hymenoptera, for example, Diprion spp., Hoplocampa spp., Lasius spp., Monomorium pharaonis and Vespa spp.

As Isopoda, for example, Armadillidium vulgare, Oniscus asellus, Porcellio scaber.

As Isoptera, for example, Reticulitermes spp., Odontotermes spp.

As Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias in sulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

As Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

As Siphonaptera, for example, Ceratophyllus spp., Xenopsylla cheopis.

As Symphyla, for example, Scutigerella immaculata.

As Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

As Thysanura, for example, Lepisma saccharina.

As plant parasitic nematodes, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp. are included.

According to the invention, it is possible to treat all plants and parts of plants. In the present invention, plants are to be understood as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' rights. Parts of plants are to be understood as meaning all above-ground and below-ground parts and organs of plants, such as shoot, leaf, flower, root and the like, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the compounds of the present invention is carried out directly or by application on their environment, habitat or storage area according to customary treatment methods, for example by dipping, spraying, evaporating, atomizing, dusting, coating, injection and, in the case of propagation material, in particular in the case of seeds, furthermore by one- or multi-layer coating.

The compounds of the present invention show a penetrating activity, suggesting that the compounds can penetrate plants to translocate from the under-ground part of the plants to the aboveground part of the plants.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof, are treated. The term “parts” or “parts of plants” or “plant parts” has been explained above.

Particularly preferably, plants of the plant cultivars which are in each case commercially available or in use are treated according to the present invention. Plant cultivars are to be understood as meaning plants having novel properties (“characters”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, nutrition), the treatment according to the present invention may also result in super-additive (“synergistic”) effects. Thus, for example, reduced application rates and/or a widening of the activity spectrum and/or an increase in the activity of substances and compositions which can be used according to the present invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects that were actually expected.

The transgenic plants or plant cultivars (i.e. those obtained by genetic engineering) which are preferably to be treated according to the present invention include all plants which, in the genetic modification, received genetic material which imparted particularly advantageous useful properties (“characters”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be raised are the important crop plants, such as cereal crops (barely, rice), maize, soya beans, potatoes, sugar beets, tomatoes, beans and other plant varieties, cotton, tobacco, rapeseed and the like, and also fruit plants (with the fruits like apples, pears, citrus fruits and grapes and the like), and particular emphasis is given to maize, soya beans, potatoes, cotton, tobacco and rapeseed. Characters that are emphasized are in particular increased defense of the plants against insects, spider-like animals, nematodes, slugs and snails, by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinafterreferred to as “Bt plants”). Similarly, characters that are particularly emphasized are the increased defense of the plants to fungi, bacteria and viruses, exhibited by systemically acquired resistance (SAR), systemin, phytoallexin, elicitor and genes related to resistance, and corresponding proteins and toxins expressed by the genes. Characters that are furthermore particularly emphasized are the increased tolerance of the plants to certain herbicidally active compounds, for example, imidazolinones, sulphonylureas, glyphosate, or phosphinotricin (for example, the “PTA” gene). The genes which impart the desired characters in interest can also be present in combination with one another in the transgenic plants. Examples of “Bt plants” which may be mentioned are maize varieties, cotton varieties, soya bean varieties and potato varieties which are sold under the trade names YIELD GARD® (for example, maize, cotton, soya beans), Knockout® (for example maize), StarLink® (for example, maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example rapeseed), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the trade name Clearfield® (for example maize). It is a matter of course that these statements also apply to plant cultivars which have these genetic characters or genetic characters still to be developed, and which will be developed and/or marketed in the future.

The listed plants can be particularly advantageously treated with the compounds of the present invention in an appropriate concentration.

Further, in the field of veterinary medicine, the compounds of the present invention can be effectively used against various harmful parasitic animals (i.e., endoparasites and ectoparasites), for example, insects and helminthes. As an example of harmful parasitic animals, the harmful organisms that are described below are included. Examples of insect include Gasterophilus spp., Stomoxys spp., Trichodectes spp., Rhodonius spp., Ctenocephalides canis, Cimx lecturius, Ctenocephalides felis, Lucilia cuprina and the like. Examples of order acarina include Ornithodoros spp., Ixodes spp., Boophilus spp. and the like.

In the field of veterinary medicine, i.e., in the veterinary medicine, the active compounds of the present invention are effective against various harmful animal parasites, in particular ectoparasites and endoparasites. The term “endoparasite” includes, in particular, a helminth (a tapeworm, a nematode, a sucking worm and the like) and a protozoa (coccidia and the like). Ectoparasite generally and preferably includes an anthropod, in particular insects [a fly (biting or sucking fly), larva of parasitic fly, lice, phthiriasis, blood-sucking lice, flea and the like], order acarina (hard tick or soft tick) or mites (sarcoptes scarbei, tsutsugamushi, bird mite and the like).

The followings are included in those parasitic organisms.

The parasitic organisms include those described below.

from Anoplurida, for example, Haematopinus spp., Linognathus spp., Pediculus spp., Phtirus spp., Solenopotes spp.; particularly, for representative examples, Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;
from Mallophagida, Amblycerina, and Ischnocerina, for example, Trimenopon spp., Menopon spp., Trinoton spp., Bovicola spp., Werneckiella spp., Lepikentron spp., Damalina spp., Trichodectes spp., Felicola spp.; particularly, for representative examples, Bovicola bovis, Bovicola ovis, Bovicola limbata, Damalina bovis, Trichodectes canis, Felicola subrostratus, Bovicola caprae, Lepikentron ovis, Werneckfella equi;
from Diptera, Nematocerina, and Brachycerina, for example, Aedes spp., Anopheles ssp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particularly, for representative examples, Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricornis, Gasterophilus pecorum, Braula coeca;
from Siphonapterida, for example, Pulex spp., Ctenocephalides spp., Tunga spp., Xenopsylla spp., Ceratophyllus spp.; particularly, for representative examples, Ctenocephalides canis, Ctenocephalides felis, Pulex irritans, Tunga penetrans, Xenopsylla cheopis;
from Heteropterida, for example, Cimex spp., Triatoma spp., Rhodnius spp., Panstrongylus spp.;
from Blattarida, for example, Blatta orientalis, Periplaneta americana, Blattela germanica, Supella spp. (for example, Suppella longipalpa);
from Acari(Acarina), Metastigmata, and Mesostigmata, for example, Argas spp., Ornithodorus spp., Otobius spp., Ixodes spp., Amblyomma spp., Rhipicephalus(Boophilus) spp., Dermacentor spp., Haemophysalis spp., Hyalomma spp., Dermanyssus spp., Rhipicephalus spp. (original genus of heteroxenous mites), Ornithonyssus spp., Pneumonyssus spp., Raillietia spp., Pneumonyssus spp., Sternostoma spp., Varroa spp., Acarapis spp.); particularly, for representative examples, Argas persicus, Argas reflexus, Ornithodorus moubata, Otobius megnini, Rhipicephalus(Boophilus) microplus, Rhipicephalus(Boophilus) decoloratus, Rhipicephalus(Boophilus) annulatus, Rhipicephalus(Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsconi;
from Actinedida(Prostigmata), and Acaridida(Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particularly, Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleli, Neoschonegastia xerothermobia, Trombicula akamushi, Otodectes cynotis, Notoedres cati, Sarcoptis canis, Sarcoptes bovis, Sarcoptes ovis, Sarcoptes rupicaprae(=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

The active compounds of the present invention are also suitable for controlling arthropods, helminths and protozoas which attack an animal. The animal includes an agricultural livestock like a cow, a sheep, a goat, a horse, a pig, a donkey, a camel, a buffalo, a rabbit, a chicken, a turkey, a duck, a goose, a nursery fish, a honey bee and the like. In addition, the animal also includes a pet (i.e., companion animal) like a dog, a cat, a pet bird, an aquarium fish and the like and an animal known as a test animal like a hamster, a guinea pig, a rat, a mouse and the like.

With the control of these arthropods, helminths and/or protozoas by using the active compounds of the present invention, death ratio of the host animal is reduced, productivity (for obtaining meat, milk, wool, leather, eggs and honey, etc.) and health of the host animal are expected to be improved, and also economically more favorable and convenient breeding of the animal can be achieved.

For example, (when applicable) it is preferable that blood uptake from a host via parasites is inhibited or interrupted. In addition, control of parasite can be useful for inhibiting transfer of infectious factors.

The term “control” used in the present specification in relation to a veterinary field means that the active compounds of the present invention are effective for reducing the occurrence of parasites in the animal infected with each parasite to a harmless level. More specifically, the term “control” used in the present specification means that the active compounds of the present invention are effective for eradicating each parasite or for inhibiting its growth or proliferation.

In general, when used for an animal treatment, the compounds of the present invention can be directly applied. Preferably, the compounds of the present invention are applied as pharmaceutical compositions which may contain vehicles and/or auxiliary agents that are known in the field and pharmaceutically acceptable.

In a veterinary medicine field and livestock farming, the active compounds can be applied (administered) in various known ways, such as via enteral administration in form of a tablet, a capsule, a drink, a syrup, a granule, a paste, a bolus and a feed stuff, or a suppository; via parenteral administration based on injection (intramuscular, subcutaneous, intravenous, intraperitoneal, etc.), implant, intranasal administration, etc.; by administration on skin in form of impregnation, liquid impregnation, spray, pouring on, spotting on, washing and powder spray; or with an aid of an molded article containing the active compounds, such as a neck tag, an ear tag, a tail tag, a leg tag, a horse rein, an identification tag, etc. The active compounds also can be prepared as shampoo, an appropriate preparation usable in aerosol, or as an unpressurized spray, for example a pump spray and a sprayer.

When used for livestock, poultry, pet and the like, the active compounds of the present invention can be prepared as a formulation containing them in an amount of 1 to 80% of weight (for example, powder, wettable preparation (WP), an emulsion, an emulsified concentrate (EC), a flowable, a homogenous solution and a suspension concentrate (SC)), and then can be applied directly or after dilution (for example, 100 to 10,000 times dilution), or they can be also applied as impregnation solution.

When used in a field of veterinary medicine, the active compounds of the present invention can be used in combination with appropriate synergists such as acaricidal agents, pesticides, anti-helminth agents or anti-protozoa agents or with other active compounds.

In the present invention, the compounds which have a pesticidal activity against the harmful pests encompassing all of the above are referred to as pesticides.

When used as pesticides, the active compounds of the present invention can be prepared in a form of common preparation. Such preparation form may includes, for example, a solution, an emulsion, wettable powder, granulated wettable powder, a suspension, powder, a foam, a paste, a tablet, a granule, an aerosol, a natural or synthetic agent impregnated with the active compounds, a microcapsule, a coating agent for seeds, a formulation equipped with a combustion device (the combustion device can be a smoke or fog cartridge, a can or a coil, etc.) and ULV (cold mist, warm mist), and the like.

These formulations may be prepared by methods known per se. For example, they can be prepared by mixing the active compounds together with spreading agents, i.e. liquid diluents or carriers; liquefied gas diluents or carriers; solid diluents or carriers, and, optionally, with surfactants i.e. emulsifiers and/or dispersants and/or foam-forming agents.

When water is used as the spreading agent, for example, organic solvents may be used as auxiliary solvents.

The liquid diluents or carriers may include, for example, aromatic hydrocarbons (e.g. xylene, toluene, alkylnaphthalene etc.), chlorinated aromatic or chlorinated aliphatic hydrocarbons (e.g. chlorobenzenes, ethylene chlorides, methylene chlorides etc.), aliphatic hydrocarbons (e.g. cyclohexanes) or paraffins (e.g. mineral oil fractions), alcohols (e.g. butanol, glycol and ethers or esters thereof, etc.), ketones (e.g. acetone, methyl ethyl ketone, methyl isobutyl ketone, cyclohexanone etc.), strong polar solvents (e.g. dimethylformamide, dimethylsulfoxide etc.), water and the like.

The liquefied gas dilution agents or carriers may include those present as gas at atmospheric temperature and by evaporation, for example, butane, propane, nitrogen gas, carbon dioxide, and an aerosol propellant such as halogenated hydrocarbons.

Examples of the solid dilution agents include ground natural minerals (for example, kaolins, clay, talc, chalk, quartz, attapulgite, montmorillonite, diatomaceous earth, etc.) and finely-ground synthetic minerals (for example, highly dispersed silicic acid, alumina and silicate, etc.) and the like.

Examples of the solid carriers for granules may include finely pulverized and sifted rocks (for example, calcite, marble, pumice, sepiolite and dolomite, etc.), synthetic granules of inorganic or organic powders, and fine granules of organic materials (for example, sawdust, coconut shells, corn cobs and tobacco stalks, etc.) and the like.

Examples of the emulsifiers and/or foam formers may include nonionic and anionic emulsifiers, for example, polyoxyethylene fatty acid esters, polyoxyethylene fatty acid alcohol ethers (for example, alkylaryl polyglycol ether), alkyl sulfonates, alkyl sulfates and aryl sulfonates, and albumin hydrolysates and the like.

Examples of the dispersants include lignin sulfite waste liquor and methylcellulose.

Binders may also be used in the formulation (powder, granule and emulsion). Examples of the binders may include carboxymethyl cellulose, natural or synthetic polymers (for example, gum arabic, polyvinyl alcohol and polyvinyl acetate, etc.).

Colorants may also be used. Examples of the colorants may include inorganic pigments (for example, iron oxide, titanium oxide and Prussian blue, etc.), organic dyes such as Alizarin dyes, azo dyes or metal phthalocyanine dyes, and further, trace elements such as salts of iron, manganese, boron, copper, cobalt, molybdenum or zinc.

In general, the formulation may include the above active components in an amount of 0.1 to 95% by weight, preferably 0.5 to 90% by weight.

The compounds of the present invention can be provided as mixtures with other active compounds such as pesticides, poison baits, sterilizing agents, acaricidal agents, nematocides, fungicides, growth regulating agents, and herbicides in a form of commercially useful formulation or an application form modified from formulation thereof.

The amount of the compounds of the present invention in commercially useful application form may vary over a broad range.

The concentration of the active compounds of the present invention for actual use may be, for example, between 0.0000001 and 100% by weight, preferably between 0.00001 and 1% by weight.

The compounds of the present invention can be used according to any common methods suitable for each application form.

The compounds of the present invention have stability that is effective for alkaline substances present on lime materials when the compounds are used against hygienic pests and other stored product pests. In addition, they exhibit excellent residual effectiveness on woods and soils.

Nitroketones according to the invention can be prepared by the preparation method (a) or (b) as given herein:

### Preparation Method (a) to Synthesize the Nitroketones to be Used in the Method According to the Invention:

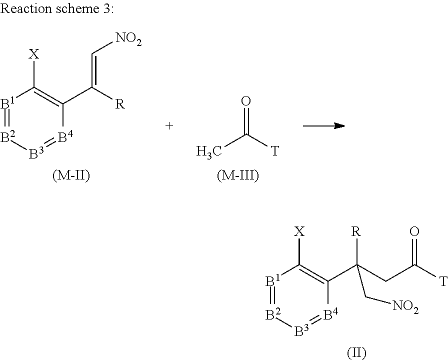

This reaction can be exemplified when 1,2,3-trichloro-5-(3,3,3-trifluoro-1-nitroprop-1-en-2-yl)benzene and 3-bromo-4-fluoroacetophenone are used:

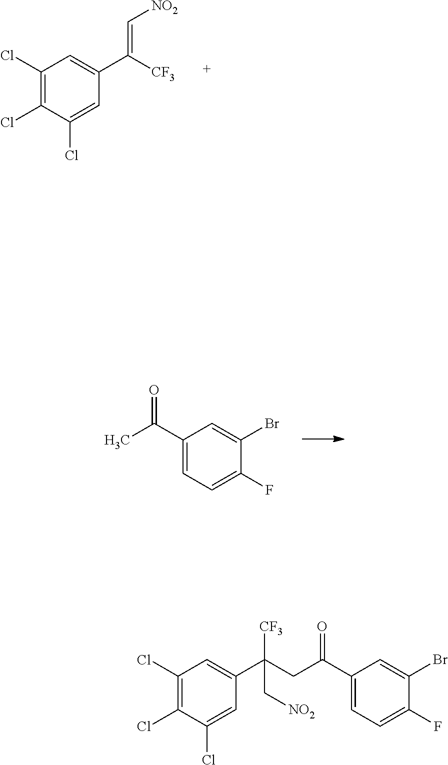 [SMILES: O=[N+]([O-])/C=C(/c1cc(Cl)c(Cl)c(Cl)c1)C(F)(F)F ; CC(=O)c1ccc(F)c(Br)c1 ; O=C(CC(C[N+](=O)[O-])(c1cc(Cl)c(Cl)c(Cl)c1)C(F)(F)F)c1ccc(F)c(Br)c1]

### Preparation Method (b) to Synthesize the Nitroketones to be Used in the Method According to the Invention:

Nitroketones according to the invention wherein T is

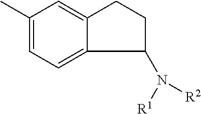

can be prepared by reacting a compound of formula (M-IV) with nitromethane in the presence of a base

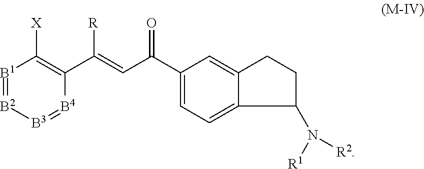

This reaction can be exemplified when N-{5-[4,4,4-trifluoro-3-(3,4,5-trichlorophenyl)but-2-enoyl]-2,3-dihydro-1H-inden-1-yl}propanamide and nitromethane are used:

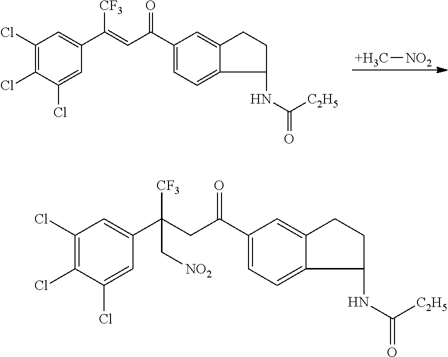

Compounds having the formula (M-II) can be prepared by reacting a compound of formula (M-V) with thionyl chloride, or by reacting a compound of formula (M-VI) with nitromethane in the presense of a suitable base

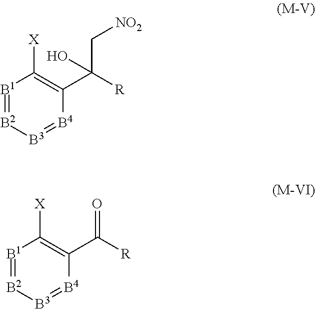

Representative compounds of formula (M-II) are for example: 1,3-dichloro-5-(3,3,3-trifluoro-1-nitropropen-2-yl)benzene, 1,2,3-trichloro-5-(3,3,3-trifluorol-nitropropen-2-yl)benzene, 1-trifluoromethyl-3-(3,3,3-trifluoro-1-nitropropen-2-yl)benzene, 1,3-bis(trifluoromethyl)-5-(3,3,3-trifluoro-1-nitropropen-2-yl)benzene, 1-chloro-3-trifluoromethyl-5-(3,3,3-trifluoro-1-nitropropen-2-yl)benzene, 1-fluoro-2-trifluoromethyl-4-(3,3,3-trifluoro-1-nitropropen-1-yl)benzene, 1,2-dichloro-3-trifluoromethyl-5-(3,3,3-trifluoro-1-nitropropen-2-yl)benzene, 2,6-dichloro-4-(3,3,3-trifluoromethyl-1-nitropropen-2-yl)pyridine, 2-trifluoromethyl-4-(3,3,3-trifluoro-1-nitropropen-2-yl)pyridine, and 2,6-bis(trifluoromethyl)-4-(3,3,3-trifluoro-1-nitropropen-2-yl)pyridine.

With respect to the compounds represented by Formula (M-III) in the preparation method (a), the compound in which T is the following group:

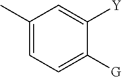

and G is a heterocyclic group as defined herein can be obtained by reacting the compounds having fluoro as the moiety that corresponds to G in Formula (M-III) (e.g. methyl-4-fluorophenyl ketone) with corresponding heterocyclic compounds (G-H), for example.

Similarly, a compound of formula (M-III) wherein G represents the following group:

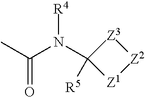

can be obtained by reacting a corresponding 2-substituted-4-acetylbenzoic acid with a compound represented by the following formula (M-VII):

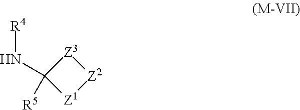

Beforementioned benzoic acid can be obtained by reacting a corresponding tert-butylbenzoic acid ester with trifluoroacetic acid. Compounds of formula (M-VII) are known.

Representative compounds of formula (M-III) are, for example tert-butyl 5-acetyl-2,3-dihydro-1H-inden-1-yl)carbamate, N-(5-acetyl-2,3-dihydro-1H-inden-1-yl)acetamide, N-(5-acetyl-2,3-dihydro-1H-inden-1-yl)propanamide, N-(5-acetyl-2,3-dihydro-1H-inden-1-yl)butanamide, N-(5-acetyl-2,3-dihydro-1H-inden-1-yl)cyclopropanecarboxamide, N-(5-acetyl-2,3-dihydro-1H-inden-1-yl)-2-cyclopropylacetamide, N-(5-acetyl-2,3-dihydro-1H-inden-1-yl)-2-(methylsulfanyl)acetamide, N-(5-acetyl-2,3-dihydro-1H-inden-1-yl)-2-(methylsulfonyl)acetamide and N-(5-acetyl-2,3-dihydro-1H-inden-1-yl)-2-methoxyacetamide.

The preparation method (a) to synthesize the nitroketones to be used in the method according to the invention can be carried out in the presence of an appropriate diluent, such as aliphatic, alicyclic and aromatic hydrocarbons (which may be chlorinated), for example, pentane, hexane, cyclohexane, petroleum ether, ligroin, benzene, toluene, xylene, dichloromethane, chloroform, carbon tetrachloride, 1,2-dichloroethane, chlorobenzene, dichlorobenzene and the like; ethers, for example, ethyl ether, methyl ethyl ether, isopropyl ether, butyl ether, dioxane, dimethoxyethane (DME), tetrahydrofuran (THF), diethylene glycol dimethyl ether (DGM) and the like; ketones, for example, acetone, methyl ethyl ketone (MEK), methyl-isopropyl ketone, methyl isobutyl ketone (MIBK) and the like; nitriles, for example, acetonitrile, propionitrile, acrylonitrile and the like; esters, for example, ethyl acetate, amyl acetate and the like; acid amides, for example, dimethylformamide (DMF), dimethylacetamide (DMA), N-methylpyrrolidone, 1,3-dimethyl-2-imidazolidinone, hexamethylphosphoric triamide (HMPA) and the like; sulfones and sulfoxides, for example, dimethyl sulfoxide (DMSO), sulfolane and the like; and bases, for example, pyridine. The preparation method (a) can be carried out in the presence of a base. As for a base, inorganic bases such as hydrides, hydroxides, carbonates and bicarbonates of alkali metals or alkali earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide and the like; amides of an inorganic alkali metal, for example, lithium amide, sodium amide, potassium amide and the like; organic bases such as alcoholates, tertiarly amines, dialkylaminoanilines, and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2.2.2]octane (DABCO) and 1,8-diazabicyclo[5.4.0]undec-7-ene (DBU) and the like; organo lithium compounds, for example, methyllithium, n-butyllithium, sec-butyllithium, tert-butyllithium, phenyllithium, dimethyl copper-lithium, lithium diisopropylamide, lithium cyclohexyl isopropyl amide, lithium dicyclohexylamide, can be mentioned.

The preparation method (a) can be carried out in a substantially broad range of temperatures. In general, it can be carried out within the range of about 10 to about 150° C., preferably within the range of about 30 to about 120° C. Furthermore, the reaction is preferably carried out at normal pressure, although it can also be carried out under reduced or elevated pressure. In carrying out the preparation method (a), the desired compound can be obtained by reacting, for example, 1 to 10 molar amounts of a compound represented by Formula (III) per mole of a compound represented by Formula (II) in a diluent, such as tetrahydrofuran, in the presence of a base.

Compounds of formula (M-IV) to be used in the preparation method (b) as well as their preparation method are described in WO2009/112275.

As an example of a general preparation method of the compounds of formula (M-IV), the compounds represented by the following formula (M-VIII) can be reacted with the compounds represented by the formula (M-IX)

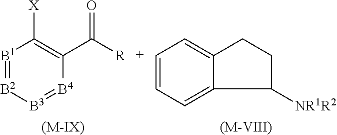

The compounds represented by formula (M-VIII) described above correspond to the compounds represented by formula (M-III) for the above preparation method (a) in which T is a group as follows:

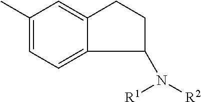

In addition, the specific processes for synthesizing the compounds represented by formula (M-VIII) are those described in the examples below.

Representative compounds of formula (M-IX) are, for example, 1-(3,5-dichlorophenyl)-2,2,2-trifluoroethanone, 1-(3,5-dibromophenyl)-2,2,2-trifluoroethanone, 2,2,2-trifluoro-1-(3,4,5-trichlorophenyl)ethanone, 1-[3,4-dichloro-5-(trifluoromethyl)phenyl]-2,2,2-trifluoroethane, 1-[3-chloro-5-(trifluoromethyl)phenyl]-2,2,2-trifluoroethanone, 1-[3,5-bis(trifluoromethyl)phenyl]-2,2,2-trifluoroethanone, 2,2,2-trifluoro-1-[3-(trifluoromethyl)phenyl]ethanone and 2,2,2-trifluoro-1-[4-fluoro-3-(trifluoromethyl)phenyl]ethanone.

Representative compounds of formula (M-IV) are, for example tert-butyl {5-[4,4,4-trifluoro-3-(3,4,5-trichlorophenyl)but-2-enoyl]-2,3-dihydro-1H-inden-1-yl}carbamate, N-{5-[4,4,4-trifluoro-3-(3,4,5-trichlorophenyl)but-2-enoyl]-2,3-dihydro-1H-inden-1-yl}acetamide, N-{5-[4,4,4-trifluoro-3-(3,4,5-trichlorophenyl)but-2-enoyl]-2,3-dihydro-1H-inden-1-yl}propanamide, N-{5-[4,4,4-trifluoro-3-(3,4,5-trichlorophenyl)but-2-enoyl]-2,3-dihydro-1H-inden-1-yl}butanamide, N-{5-[4,4,4-trifluoro-3-(3,4,5-trichlorophenyl)but-2-enoyl]-2,3-dihydro-1H-inden-1-yl}cyclopropanecarboxyamide, 2-methylsulfonyl-N-{5-[4,4,4-trifluoro-3-(3,4,5-trichlorophenyl)but-2-enoyl]-2,3-dihydro-1H-inden-1-yl}acetamide, N-{5-[3-(3,5-dichlorophenyl)-4,4,4-trifluorobut-2-enoyl]-2,3-dihydro-1H-inden-1-yl}propanamide, N{5-[3-(3-chlorophenyl)-5-(trifluoromethyl)-4,4,4-trifluorobut-2-enoyl]-2,3-dihydro-1H-inden-1-yl}propanamide, N-(5-[3-{3,5-bis(trifluoromethyl)phenyl]-4,4,4-trifluorobut-2-enoyl}-2,3-dihydro-1H-inden-1-yl]propanamide and N-(5-[3-{3,6-bis(trifluoromethyl)pyridin-4-yl]-4,4,4-trifluorobut-2-enoyl}-2,3-dihydro-1H-inden-1-yl)propanamide.

The preparation method (b) to synthesis the nitroketones to be used in the method according to the invention can be carried out in the presence of an appropriate diluent. As examples of the diluent which can be used, aliphatic, alicyclic and aromatic hydrocarbons (which may be chlorinated), for example, pentane, hexane, cyclohexane, petroleum ether, ligroin, benzene, toluene, xylene, dichloromethane, chloroform, carbon tetrachloride, 1,2-dichloroethane, chlorobenzene, dichlorobenzene; ethers, for example, ethyl ether, methyl ethyl ether, isopropyl ether, butyl ether, dioxane, dimethoxyethane (DME), tetrahydrofuran (THF), diethylene glycol dimethyl ether (DGM); ketones, for example, acetone, methyl ethyl ketone (MEK), methyl-isopropyl ketone, methyl isobutyl ketone (MIBK); nitriles, for example, acetonitrile, propionitrile, acrylonitrile; alcohols, for example, methanol, ethanol, isopropanol, butanol, ethylene glycol; esters, for example, ethyl acetate, amyl acetate; acid amides, for example, dimethylformamide (DMF), dimethylacetamide (DMA), N-methylpyrrolidone, 1,3-dimethyl-2-imidazolidinone, hexamethylphosphoric triamide (HMPA); sulfones and sulfoxides, for example, dimethyl sulfoxide (DMSO), sulfolane; and bases such as pyridine, can be mentioned.

The preparation method (b) can be carried out in the presence of a base, for example, alkali metal bases such as sodium carbonate, potassium carbonate, sodium hydrogen carbonate, sodium acetate, potassium acetate, sodium methoxide, sodium ethoxide, potassium tert-butoxide, lithium hydride, and organic bases such as triethylamine, diisopropylethylamine, tributylamine, N-methylmorpholine, N,N-dimethylaniline, N,N-diethylaniline, 4-tert-butyl-N,N-dimethylaniline, pyridine, picoline, lutidine, diazabicycloundecene, diazabicyclooctane, imidazole.

The preparation method (b) can be carried out in a substantially broad range of temperatures. In general, it can be carried out within the range of about −78 to about 200° C., preferably within the range of about −10 to about 100° C. Furthermore, the reaction is preferably carried out at normal pressure, although it can also be carried out under reduced or elevated pressure.

The reaction time is 0.1 to 72 hours, and preferably 1 to 24 hours. In carrying out the preparation method (b), the desired compound represented by formula (M-II) can be obtained by reacting, for example, one molar amount to slightly excess molar amounts of nitromethane per mole of a compound represented by formula (M-IV) in a diluent, e.g., DMF.

The compounds of the present invention, which can be obtained in accordance with the same method as the method of the above Synthetic examples, are exemplified in the following tables. Some of the compounds of the above Synthetic examples are also included in the tables. In the tables, Me=methyl, Et=ethyl, Bu=butyl, and Pr=propyl.

TABLE 1

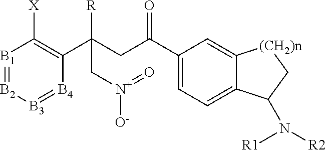

| Compound | | | X | B1 | B2 | B3 | B4 | R | R1 | R2 | n |
|---|---|---|---|---|---|---|---|---|---|---|---|
| T1 | — | 1 | H | C—Cl | C—H | C—Cl | C—H | CF3 | H | H | 1 |
| T1 | — | 2 | H | C—Cl | C—H | C—Cl | C—H | CF3 | tert-BuOC(═O) | H | 1 |
| T1 | — | 3 | H | C—Cl | C—H | C—Cl | C—H | CF3 | MeCO | H | 1 |
| T1 | — | 4 | H | C—Cl | C—H | C—Cl | C—H | CF3 | EtCO | H | 1 |
| T1 | — | 5 | H | C—Cl | C—H | C—Cl | C—H | CF3 | n-PrCO | H | 1 |
| T1 | — | 6 | H | C—Cl | C—H | C—Cl | C—H | CF3 | cyclo-PrCO | H | 1 |
| T1 | — | 7 | H | C—Cl | C—H | C—Cl | C—H | CF3 | cyclo-PrCH2CO | H | 1 |
| T1 | — | 8 | H | C—Cl | C—H | C—Cl | C—H | CF3 | CF3CH2CO | H | 1 |
| T1 | — | 9 | H | C—Cl | C—H | C—Cl | C—H | CF3 | CH3SCH2CO | H | 1 |
| T1 | — | 10 | H | C—Cl | C—H | C—Cl | C—H | CF3 | CH3SOCH2CO | H | 1 |
| T1 | — | 11 | H | C—Cl | C—H | C—Cl | C—H | CF3 | CH3SO2CH2CO | H | 1 |
| T1 | — | 12 | H | C—Cl | C—H | C—Cl | C—H | CF3 | CH3OCH2CH2CO | H | 1 |
| T1 | — | 13 | H | C—Cl | C—H | C—Cl | C—H | CF3 | CH3OCH(Me)CH2CO | H | 1 |
| T1 | — | 14 | H | C—Br | C—H | C—Br | C—H | CF3 | H | H | 1 |
| T1 | — | 15 | H | C—Br | C—H | C—Br | C—H | CF3 | tert-BuOC(═O) | H | 1 |
| T1 | — | 16 | H | C—Br | C—H | C—Br | C—H | CF3 | MeCO | H | 1 |
| T1 | — | 17 | H | C—Br | C—H | C—Br | C—H | CF3 | EtCO | H | 1 |
| T1 | — | 18 | H | C—Br | C—H | C—Br | C—H | CF3 | n-PrCO | H | 1 |
| T1 | — | 19 | H | C—Br | C—H | C—Br | C—H | CF3 | cyclo-PrCO | H | 1 |
| T1 | — | 20 | H | C—Br | C—H | C—Br | C—H | CF3 | cyclo-PrCH2CO | H | 1 |
| T1 | — | 21 | H | C—Br | C—H | C—Br | C—H | CF3 | CF3CH2CO | H | 1 |
| T1 | — | 22 | H | C—Br | C—H | C—Br | C—H | CF3 | CH3SCH2CO | H | 1 |
| T1 | — | 23 | H | C—Br | C—H | C—Br | C—H | CF3 | CH3SOCH2CO | H | 1 |
| T1 | — | 24 | H | C—Br | C—H | C—Br | C—H | CF3 | CH3SO2CH2CO | H | 1 |
| T1 | — | 25 | H | C—Br | C—H | C—Br | C—H | CF3 | CH3OCH2CH2CO | H | 1 |
| T1 | — | 26 | H | C—Br | C—H | C—Br | C—H | CF3 | CH3OCH(Me)CH2CO | H | 1 |
| T1 | — | 27 | H | C—CF3 | C—H | C—H | C—H | CF3 | H | H | 1 |
| T1 | — | 28 | H | C—CF3 | C—H | C—H | C—H | CF3 | tert-BuOC(═O) | H | 1 |
| T1 | — | 29 | H | C—CF3 | C—H | C—H | C—H | CF3 | MeCO | H | 1 |
| T1 | — | 30 | H | C—CF3 | C—H | C—H | C—H | CF3 | EtCO | H | 1 |
| T1 | — | 31 | H | C—CF3 | C—H | C—H | C—H | CF3 | n-PrCO | H | 1 |
| T1 | — | 32 | H | C—CF3 | C—H | C—H | C—H | CF3 | cyclo-PrCO | H | 1 |
| T1 | — | 33 | H | C—CF3 | C—H | C—H | C—H | CF3 | cyclo-PrCH2CO | H | 1 |
| T1 | — | 34 | H | C—CF3 | C—H | C—H | C—H | CF3 | CF3CH2CO | H | 1 |
| T1 | — | 35 | H | C—CF3 | C—H | C—H | C—H | CF3 | CH3SCH2CO | H | 1 |
| T1 | — | 36 | H | C—CF3 | C—H | C—H | C—H | CF3 | CH3SOCH2CO | H | 1 |
| T1 | — | 37 | H | C—CF3 | C—H | C—H | C—H | CF3 | CH3SO2CH2CO | H | 1 |
| T1 | — | 38 | H | C—CF3 | C—H | C—H | C—H | CF3 | CH3OCH2CH2CO | H | 1 |
| T1 | — | 39 | H | C—CF3 | C—H | C—H | C—H | CF3 | CH3OCH(Me)CH2CO | H | 1 |
| T1 | — | 40 | H | C—CF3 | C—H | C—CF3 | C—H | CF3 | H | H | 1 |
| T1 | — | 41 | H | C—CF3 | C—H | C—CF3 | C—H | CF3 | tert-BuOC(═O) | H | 1 |
| T1 | — | 42 | H | C—CF3 | C—H | C—CF3 | C—H | CF3 | MeCO | H | 1 |
| T1 | — | 43 | H | C—CF3 | C—H | C—CF3 | C—H | CF3 | EtCO | H | 1 |
| T1 | — | 44 | H | C—CF3 | C—H | C—CF3 | C—H | CF3 | n-PrCO | H | 1 |
| T1 | — | 45 | H | C—CF3 | C—H | C—CF3 | C—H | CF3 | cyclo-PrCO | H | 1 |
| T1 | — | 46 | H | C—CF3 | C—H | C—CF3 | C—H | CF3 | cyclo-PrCH2CO | H | 1 |
| T1 | — | 47 | H | C—CF3 | C—H | C—CF3 | C—H | CF3 | CF3CH2CO | H | 1 |
| T1 | — | 48 | H | C—CF3 | C—H | C—CF3 | C—H | CF3 | CH3SCH2CO | H | 1 |
| T1 | — | 49 | H | C—CF3 | C—H | C—CF3 | C—H | CF3 | CH3SOCH2CO | H | 1 |
| T1 | — | 50 | H | C—CF3 | C—H | C—CF3 | C—H | CF3 | CH3SO2CH2CO | H | 1 |
| T1 | — | 51 | H | C—CF3 | C—H | C—CF3 | C—H | CF3 | CH3OCH2CH2CO | H | 1 |
| T1 | — | 52 | H | C—CF3 | C—H | C—CF3 | C—H | CF3 | CH3OCH(Me)CH2CO | H | 1 |
| T1 | — | 53 | H | C—CF3 | C—H | C—Cl | C—H | CF3 | H | H | 1 |
| T1 | — | 54 | H | C—CF3 | C—H | C—Cl | C—H | CF3 | tert-BuOC(═O) | H | 1 |
| T1 | — | 55 | H | C—CF3 | C—H | C—Cl | C—H | CF3 | MeCO | H | 1 |
| T1 | — | 56 | H | C—CF3 | C—H | C—Cl | C—H | CF3 | EtCO | H | 1 |
| T1 | — | 57 | H | C—CF3 | C—H | C—Cl | C—H | CF3 | n-PrCO | H | 1 |
| T1 | — | 58 | H | C—CF3 | C—H | C—Cl | C—H | CF3 | cyclo-PrCO | H | 1 |
| T1 | — | 59 | H | C—CF3 | C—H | C—Cl | C—H | CF3 | cyclo-PrCH2CO | H | 1 |
| T1 | — | 60 | H | C—CF3 | C—H | C—Cl | C—H | CF3 | CF3CH2CO | H | 1 |
| T1 | — | 61 | H | C—CF3 | C—H | C—Cl | C—H | CF3 | CH3SCH2CO | H | 1 |
| T1 | — | 62 | H | C—CF3 | C—H | C—Cl | C—H | CF3 | CH3SOCH2CO | H | 1 |
| T1 | — | 63 | H | C—CF3 | C—H | C—Cl | C—H | CF3 | CH3SO2CH2CO | H | 1 |
| T1 | — | 64 | H | C—CF3 | C—H | C—Cl | C—H | CF3 | CH3OCH2CH2CO | H | 1 |
| T1 | — | 65 | H | C—CF3 | C—H | C—Cl | C—H | CF3 | CH3OCH(Me)CH2CO | H | 1 |
| T1 | — | 66 | H | C—CF3 | C—F | C—H | C—H | CF3 | H | H | 1 |
| T1 | — | 67 | H | C—CF3 | C—F | C—H | C—H | CF3 | tert-BuOC(═O) | H | 1 |
| T1 | — | 68 | H | C—CF3 | C—F | C—H | C—H | CF3 | MeCO | H | 1 |
| T1 | — | 69 | H | C—CF3 | C—F | C—H | C—H | CF3 | EtCO | H | 1 |
| T1 | — | 70 | H | C—CF3 | C—F | C—H | C—H | CF3 | n-PrCO | H | 1 |
| T1 | — | 71 | H | C—CF3 | C—F | C—H | C—H | CF3 | cyclo-PrCO | H | 1 |
| T1 | — | 72 | H | C—CF3 | C—F | C—H | C—H | CF3 | cyclo-PrCH2CO | H | 1 |
| T1 | — | 73 | H | C—CF3 | C—F | C—H | C—H | CF3 | CF3CH2CO | H | 1 |
| T1 | — | 74 | H | C—CF3 | C—F | C—H | C—H | CF3 | CH3SCH2CO | H | 1 |
| T1 | — | 75 | H | C—CF3 | C—F | C—H | C—H | CF3 | CH3SOCH2CO | H | 1 |
| T1 | — | 76 | H | C—CF3 | C—F | C—H | C—H | CF3 | CH3SO2CH2CO | H | 1 |
| T1 | — | 77 | H | C—CF3 | C—F | C—H | C—H | CF3 | CH3OCH2CH2CO | H | 1 |
| T1 | — | 78 | H | C—CF3 | C—F | C—H | C—H | CF3 | CH3OCH(Me)CH2CO | H | 1 |
| T1 | — | 79 | H | C—CF3 | C—H | C—F | C—H | CF3 | H | H | 1 |
| T1 | — | 80 | H | C—CF3 | C—H | C—F | C—H | CF3 | tert-BuOC(═O) | H | 1 |
| T1 | — | 81 | H | C—CF3 | C—H | C—F | C—H | CF3 | MeCO | H | 1 |
| T1 | — | 82 | H | C—CF3 | C—H | C—F | C—H | CF3 | EtCO | H | 1 |
| T1 | — | 83 | H | C—CF3 | C—H | C—F | C—H | CF3 | n-PrCO | H | 1 |
| T1 | — | 84 | H | C—CF3 | C—H | C—F | C—H | CF3 | cyclo-PrCO | Me | 1 |
| T1 | — | 85 | H | C—CF3 | C—H | C—F | C—H | CF3 | cyclo-PrCH2CO | Me | 1 |
| T1 | — | 86 | H | C—CF3 | C—H | C—F | C—H | CF3 | CF3CH2CO | Me | 1 |
| T1 | — | 87 | H | C—CF3 | C—H | C—F | C—H | CF3 | CH3SCH2CO | Me | 1 |
| T1 | — | 88 | H | C—CF3 | C—H | C—F | C—H | CF3 | CH3SOCH2CO | Me | 1 |
| T1 | — | 89 | H | C—CF3 | C—H | C—F | C—H | CF3 | CH3SO2CH2CO | Me | 1 |
| T1 | — | 90 | H | C—CF3 | C—H | C—F | C—H | CF3 | CH3OCH2CH2CO | Me | 1 |
| T1 | — | 91 | H | C—CF3 | C—H | C—F | C—H | CF3 | CH3OCH(Me)CH2CO | H | 1 |
| T1 | — | 92 | H | C—Cl | C—Cl | C—Cl | C—H | CF3 | H | H | 1 |
| T1 | — | 93 | H | C—Cl | C—Cl | C—Cl | C—H | CF3 | tert-BuOC(═O) | H | 1 |
| T1 | — | 94 | H | C—Cl | C—Cl | C—Cl | C—H | CF3 | MeCO | H | 1 |
| T1 | — | 95 | H | C—Cl | C—Cl | C—Cl | C—H | CF3 | EtCO | H | 1 |
| T1 | — | 96 | H | C—Cl | C—Cl | C—Cl | C—H | CF3 | n-PrCO | H | 1 |
| T1 | — | 97 | H | C—Cl | C—Cl | C—Cl | C—H | CF3 | cyclo-PrCO | H | 1 |
| T1 | — | 98 | H | C—Cl | C—Cl | C—Cl | C—H | CF3 | cyclo-PrCH2CO | H | 1 |
| T1 | — | 99 | H | C—Cl | C—Cl | C—Cl | C—H | CF3 | CF3CH2CO | H | 1 |
| T1 | — | 100 | H | C—Cl | C—Cl | C—Cl | C—H | CF3 | CH3SCH2CO | H | 1 |
| T1 | — | 101 | H | C—Cl | C—Cl | C—Cl | C—H | CF3 | CH3SOCH2CO | H | 1 |
| T1 | — | 102 | H | C—Cl | C—Cl | C—Cl | C—H | CF3 | CH3SO2CH2CO | H | 1 |
| T1 | — | 103 | H | C—Cl | C—Cl | C—Cl | C—H | CF3 | CH3OCH2CH2CO | H | 1 |
| T1 | — | 104 | H | C—Cl | C—Cl | C—Cl | C—H | CF3 | CH3OCH(Me)CH2CO | H | 1 |
| T1 | — | 105 | H | C—Cl | C—Cl | C—CF3 | C—H | CF3 | H | H | 1 |
| T1 | — | 106 | H | C—Cl | C—Cl | C—CF3 | C—H | CF3 | tert-BuOC(═O) | H | 1 |
| T1 | — | 107 | H | C—Cl | C—Cl | C—CF3 | C—H | CF3 | MeCO | H | 1 |
| T1 | — | 108 | H | C—Cl | C—Cl | C—CF3 | C—H | CF3 | EtCO | H | 1 |
| T1 | — | 109 | H | C—Cl | C—Cl | C—CF3 | C—H | CF3 | n-PrCO | H | 1 |
| T1 | — | 110 | H | C—Cl | C—Cl | C—CF3 | C—H | CF3 | cyclo-PrCO | H | 1 |
| T1 | — | 111 | H | C—Cl | C—Cl | C—CF3 | C—H | CF3 | cyclo-PrCH2CO | H | 1 |
| T1 | — | 112 | H | C—Cl | C—Cl | C—CF3 | C—H | CF3 | CF3CH2CO | H | 1 |
| T1 | — | 113 | H | C—Cl | C—Cl | C—CF3 | C—H | CF3 | CH3SCH2CO | H | 1 |
| T1 | — | 114 | H | C—Cl | C—Cl | C—CF3 | C—H | CF3 | CH3SOCH2CO | H | 1 |
| T1 | — | 115 | H | C—Cl | C—Cl | C—CF3 | C—H | CF3 | CH3SO2CH2CO | H | 1 |
| T1 | — | 116 | H | C—Cl | C—Cl | C—CF3 | C—H | CF3 | CH3OCH2CH2CO | H | 1 |
| T1 | — | 117 | H | C—Cl | C—Cl | C—CF3 | C—H | CF3 | CH3OCH(Me)CH2CO | H | 1 |
| T1 | — | 118 | H | C—Cl | N | C—Cl | C—H | CF3 | H | H | 1 |
| T1 | — | 119 | H | C—Cl | N | C—Cl | C—H | CF3 | tert-BuOC(═O) | H | 1 |
| T1 | — | 120 | H | C—Cl | N | C—Cl | C—H | CF3 | MeCO | H | 1 |
| T1 | — | 121 | H | C—Cl | N | C—Cl | C—H | CF3 | EtCO | H | 1 |
| T1 | — | 122 | H | C—Cl | N | C—Cl | C—H | CF3 | n-PrCO | H | 1 |
| T1 | — | 123 | H | C—Cl | N | C—Cl | C—H | CF3 | cyclo-PrCO | H | 1 |
| T1 | — | 124 | H | C—Cl | N | C—Cl | C—H | CF3 | cyclo-PrCH2CO | H | 1 |
| T1 | — | 125 | H | C—Cl | N | C—Cl | C—H | CF3 | CF3CH2CO | H | 1 |
| T1 | — | 126 | H | C—Cl | N | C—Cl | C—H | CF3 | CH3SCH2CO | H | 1 |
| T1 | — | 127 | H | C—Cl | N | C—Cl | C—H | CF3 | CH3SOCH2CO | H | 1 |
| T1 | — | 128 | H | C—Cl | N | C—Cl | C—H | CF3 | CH3SO2CH2CO | H | 1 |
| T1 | — | 129 | H | C—Cl | N | C—Cl | C—H | CF3 | CH3OCH2CH2CO | H | 1 |
| T1 | — | 130 | H | C—Cl | N | C—Cl | C—H | CF3 | CH3OCH(Me)CH2CO | H | 1 |
| T1 | — | 131 | H | C—CF3 | N | C—CF3 | C—H | CF3 | H | H | 1 |
| T1 | — | 132 | H | C—CF3 | N | C—CF3 | C—H | CF3 | tert-BuOC(═O) | H | 1 |
| T1 | — | 133 | H | C—CF3 | N | C—CF3 | C—H | CF3 | MeCO | H | 1 |
| T1 | — | 134 | H | C—CF3 | N | C—CF3 | C—H | CF3 | EtCO | H | 1 |
| T1 | — | 135 | H | C—CF3 | N | C—CF3 | C—H | CF3 | n-PrCO | H | 1 |
| T1 | — | 136 | H | C—CF3 | N | C—CF3 | C—H | CF3 | cyclo-PrCO | H | 1 |
| T1 | — | 137 | H | C—CF3 | N | C—CF3 | C—H | CF3 | cyclo-PrCH2CO | H | 1 |
| T1 | — | 138 | H | C—CF3 | N | C—CF3 | C—H | CF3 | CF3CH2CO | H | 1 |
| T1 | — | 139 | H | C—CF3 | N | C—CF3 | C—H | CF3 | CH3SCH2CO | H | 1 |
| T1 | — | 140 | H | C—CF3 | N | C—CF3 | C—H | CF3 | CH3SOCH2CO | H | 1 |
| T1 | — | 141 | H | C—CF3 | N | C—CF3 | C—H | CF3 | CH3SO2CH2CO | H | 1 |
| T1 | — | 142 | H | C—CF3 | N | C—CF3 | C—H | CF3 | CH3OCH2CH2CO | H | 1 |
| T1 | — | 143 | H | C—CF3 | N | C—CF3 | C—H | CF3 | CH3OCH(Me)CH2CO | H | 1 |
| T1 | — | 144 | H | C—CF3 | N | C—Cl | C—H | CF3 | H | H | 1 |
| T1 | — | 145 | H | C—CF3 | N | C—Cl | C—H | CF3 | tert-BuOC(═O) | H | 1 |
| T1 | — | 146 | H | C—CF3 | N | C—Cl | C—H | CF3 | MeCO | H | 1 |
| T1 | — | 147 | H | C—CF3 | N | C—Cl | C—H | CF3 | EtCO | H | 1 |
| T1 | — | 148 | H | C—CF3 | N | C—Cl | C—H | CF3 | n-PrCO | H | 1 |
| T1 | — | 149 | H | C—CF3 | N | C—Cl | C—H | CF3 | cyclo-PrCO | H | 1 |
| T1 | — | 150 | H | C—CF3 | N | C—Cl | C—H | CF3 | cyclo-PrCH2CO | H | 1 |
| T1 | — | 151 | H | C—CF3 | N | C—Cl | C—H | CF3 | CF3CH2CO | H | 1 |
| T1 | — | 152 | H | C—CF3 | N | C—Cl | C—H | CF3 | CH3SCH2CO | H | 1 |
| T1 | — | 153 | H | C—CF3 | N | C—Cl | C—H | CF3 | CH3SOCH2CO | H | 1 |
| T1 | — | 154 | H | C—CF3 | N | C—Cl | C—H | CF3 | CH3SO2CH2CO | H | 1 |
| T1 | — | 155 | H | C—CF3 | N | C—Cl | C—H | CF3 | CH3OCH2CH2CO | H | 1 |
| T1 | — | 156 | H | C—CF3 | N | C—Cl | C—H | CF3 | CH3OCH(Me)CH2CO | H | 1 |
| T1 | — | 157 | H | C—Cl | C—Cl | C—Cl | C—H | CF3 | H | H | 1 |
| T1 | — | 158 | H | C—Cl | C—Cl | C—Cl | C—H | CF3 | tert-BuOC(═O) | H | 1 |
| T1 | — | 159 | H | C—Cl | C—Cl | C—Cl | C—H | CF3 | MeCO | H | 1 |
| T1 | — | 160 | H | C—Cl | C—Cl | C—Cl | C—H | CF3 | EtCO | H | 1 |
| T1 | — | 161 | H | C—Cl | C—Cl | C—Cl | C—H | CF3 | n-PrCO | H | 1 |
| T1 | — | 162 | H | C—Cl | C—Cl | C—Cl | C—H | CF3 | cyclo-PrCO | H | 1 |
| T1 | — | 163 | H | C—Cl | C—Cl | C—Cl | C—H | CF3 | cyclo-PrCH2CO | H | 1 |
| T1 | — | 164 | H | C—Cl | C—Cl | C—Cl | C—H | CF3 | CF3CH2CO | H | 1 |
| T1 | — | 165 | H | C—Cl | C—Cl | C—Cl | C—H | CF3 | CH3SCH2CO | H | 1 |
| T1 | — | 166 | H | C—Cl | C—Cl | C—Cl | C—H | CF3 | CH3SOCH2CO | H | 1 |
| T1 | — | 167 | H | C—Cl | C—Cl | C—Cl | C—H | CF3 | CH3SO2CH2CO | H | 1 |
| T1 | — | 168 | H | C—Cl | C—Cl | C—Cl | C—H | CF3 | CH3OCH2CH2CO | H | 1 |
| T1 | — | 169 | H | C—Cl | C—Cl | C—Cl | C—H | CF3 | CH3OCH(Me)CH2CO | H | 1 |
| T1 | — | 170 | H | C—CF3 | N | C—H | C—H | CF3 | H | H | 1 |
| T1 | — | 171 | H | C—CF3 | N | C—H | C—H | CF3 | tert-BuOC(═O) | H | 1 |
| T1 | — | 172 | H | C—CF3 | N | C—H | C—H | CF3 | MeCO | H | 1 |
| T1 | — | 173 | H | C—CF3 | N | C—H | C—H | CF3 | EtCO | H | 1 |
| T1 | — | 174 | H | C—CF3 | N | C—H | C—H | CF3 | n-PrCO | H | 1 |
| T1 | — | 175 | H | C—CF3 | N | C—H | C—H | CF3 | cyclo-PrCO | H | 1 |
| T1 | — | 176 | H | C—CF3 | N | C—H | C—H | CF3 | cyclo-PrCH2CO | H | 1 |
| T1 | — | 177 | H | C—CF3 | N | C—H | C—H | CF3 | CF3CH2CO | H | 1 |
| T1 | — | 178 | H | C—CF3 | N | C—H | C—H | CF3 | CH3SCH2CO | H | 1 |
| T1 | — | 179 | H | C—CF3 | N | C—H | C—H | CF3 | CH3SOCH2CO | H | 1 |
| T1 | — | 180 | H | C—CF3 | N | C—H | C—H | CF3 | CH3SO2CH2CO | H | 1 |
| T1 | — | 181 | H | C—CF3 | N | C—H | C—H | CF3 | CH3OCH2CH2CO | H | 1 |
| T1 | — | 182 | H | C—CF3 | N | C—H | C—H | CF3 | CH3OCH(Me)CH2CO | H | 1 |
| T1 | — | 183 | H | C—Cl | N | C—H | C—H | CF3 | H | H | 1 |
| T1 | — | 184 | H | C—Cl | N | C—H | C—H | CF3 | tert-BuOC(═O) | H | 1 |
| T1 | — | 185 | H | C—Cl | N | C—H | C—H | CF3 | MeCO | H | 1 |
| T1 | — | 186 | H | C—Cl | N | C—H | C—H | CF3 | EtCO | H | 1 |
| T1 | — | 187 | H | C—Cl | N | C—H | C—H | CF3 | n-PrCO | H | 1 |
| T1 | — | 188 | H | C—Cl | N | C—H | C—H | CF3 | cyclo-PrCO | H | 1 |
| T1 | — | 189 | H | C—Cl | N | C—H | C—H | CF3 | cyclo-PrCH2CO | H | 1 |
| T1 | — | 190 | H | C—Cl | N | C—H | C—H | CF3 | CF3CH2CO | H | 1 |
| T1 | — | 191 | H | C—Cl | N | C—H | C—H | CF3 | CH3SCH2CO | H | 1 |
| T1 | — | 192 | H | C—Cl | N | C—H | C—H | CF3 | CH3SOCH2CO | H | 1 |
| T1 | — | 193 | H | C—Cl | N | C—H | C—H | CF3 | CH3SO2CH2CO | H | 1 |
| T1 | — | 194 | H | C—Cl | N | C—H | C—H | CF3 | CH3OCH2CH2CO | H | 1 |
| T1 | — | 195 | H | C—Cl | N | C—H | C—H | CF3 | CH3OCH(Me)CH2CO | H | 1 |
| T1 | — | 196 | H | C—Cl | C—F | C—Cl | C—F | CF3 | H | H | 1 |
| T1 | — | 197 | H | C—Cl | C—F | C—Cl | C—F | CF3 | tert-BuOC(═O) | H | 1 |
| T1 | — | 198 | H | C—Cl | C—F | C—Cl | C—F | CF3 | MeCO | H | 1 |
| T1 | — | 199 | H | C—Cl | C—F | C—Cl | C—F | CF3 | EtCO | H | 1 |
| T1 | — | 200 | H | C—Cl | C—F | C—Cl | C—F | CF3 | n-PrCO | H | 1 |
| T1 | — | 201 | H | C—Cl | C—F | C—Cl | C—F | CF3 | cyclo-PrCO | H | 1 |
| T1 | — | 202 | H | C—Cl | C—F | C—Cl | C—F | CF3 | cyclo-PrCH2CO | H | 1 |
| T1 | — | 203 | H | C—Cl | C—F | C—Cl | C—F | CF3 | CF3CH2CO | H | 1 |
| T1 | — | 204 | H | C—Cl | C—F | C—Cl | C—F | CF3 | CH3SCH2CO | H | 1 |
| T1 | — | 205 | H | C—Cl | C—F | C—Cl | C—F | CF3 | CH3SOCH2CO | H | 1 |
| T1 | — | 206 | H | C—Cl | C—F | C—Cl | C—F | CF3 | CH3SO2CH2CO | H | 1 |
| T1 | — | 207 | H | C—Cl | C—F | C—Cl | C—F | CF3 | CH3OCH2CH2CO | H | 1 |
| T1 | — | 208 | H | C—Cl | C—F | C—Cl | C—F | CF3 | CH3OCH(Me)CH2CO | H | 1 |

TABLE 2

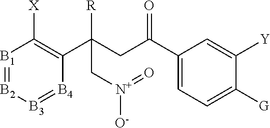

| Compound | | | X | B¹ | B² | B³ | B⁴ | R | Y | G | (Z)ₖ |
|---|---|---|---|---|---|---|---|---|---|---|---|
| T2 | — | 1 | H | C—Cl | C—H | C—Cl | C—H | CF3 | CN | G2 | H |
| T2 | — | 2 | H | C—Cl | C—H | C—Cl | C—H | CF3 | CN | G2 | 4-F |
| T2 | — | 3 | H | C—Cl | C—H | C—Cl | C—H | CF3 | CN | G2 | 4-Cl |
| T2 | — | 4 | H | C—Cl | C—H | C—Cl | C—H | CF3 | CN | G2 | 4-CN |
| T2 | — | 5 | H | C—Cl | C—H | C—Cl | C—H | CF3 | CN | G2 | 4-NO2 |
| T2 | — | 6 | H | C—Cl | C—H | C—Cl | C—H | CF3 | CN | G6 | H |
| T2 | — | 7 | H | C—Cl | C—H | C—Cl | C—H | CF3 | CN | G8 | H |
| T2 | — | 8 | H | C—Cl | C—H | C—Cl | C—H | CF3 | CN | G9 | H |
| T2 | — | 9 | H | C—Cl | C—H | C—Cl | C—H | CF3 | CN | F | H |
| T2 | — | 10 | H | C—Cl | C—H | C—Cl | C—H | CF3 | CN | Cl | H |
| T2 | — | 11 | H | C—Cl | C—H | C—Cl | C—H | CF3 | CN | Br | H |
| T2 | — | 12 | H | C—Br | C—H | C—Br | C—H | CF3 | CN | G2 | H |
| T2 | — | 13 | H | C—Br | C—H | C—Br | C—H | CF₃ | CN | G6 | H |
| T2 | — | 14 | H | C—Br | C—H | C—Br | C—H | CF₃ | CN | G8 | H |
| T2 | — | 15 | H | C—Br | C—H | C—Br | C—H | CF₃ | CN | G9 | H |
| T2 | — | 16 | H | C—Br | C—H | C—Br | C—H | CF₃ | CN | F | H |
| T2 | — | 17 | H | C—CF3 | C—H | C—H | C—H | CF₃ | CN | G2 | H |
| T2 | — | 18 | H | C—CF3 | C—H | C—H | C—H | CF₃ | CN | G6 | H |
| T2 | — | 19 | H | C—CF3 | C—H | C—H | C—H | CF₃ | CN | G8 | H |
| T2 | — | 20 | H | C—CF3 | C—H | C—H | C—H | CF3 | CN | G9 | H |
| T2 | — | 21 | H | C—CF3 | C—H | C—H | C—H | CF3 | CN | F | H |
| T2 | — | 22 | H | C—CF3 | C—H | C—CF3 | C—H | CF3 | CN | G2 | H |
| T2 | — | 23 | H | C—CF3 | C—H | C—CF3 | C—H | CF3 | CN | G2 | 4-F |
| T2 | — | 24 | H | C—CF3 | C—H | C—CF3 | C—H | CF3 | CN | G2 | 4-Cl |
| T2 | — | 25 | H | C—CF3 | C—H | C—CF3 | C—H | CF3 | CN | G2 | 4-CN |
| T2 | — | 26 | H | C—CF3 | C—H | C—CF3 | C—H | CF3 | CN | G6 | H |
| T2 | — | 27 | H | C—CF3 | C—H | C—CF3 | C—H | CF3 | CN | G8 | H |
| T2 | — | 28 | H | C—CF3 | C—H | C—CF3 | C—H | CF3 | CN | G9 | H |
| T2 | — | 29 | H | C—CF3 | C—H | C—CF3 | C—H | CF3 | CN | F | H |
| T2 | — | 30 | H | C—CF3 | C—H | C—CF3 | C—H | CF3 | CN | Cl | H |
| T2 | — | 31 | H | C—CF3 | C—H | C—CF3 | C—H | CF3 | CN | Br | H |
| T2 | — | 32 | H | C—CF3 | C—H | C—Cl | C—H | CF3 | CN | G2 | H |
| T2 | — | 33 | H | C—CF3 | C—H | C—Cl | C—H | CF3 | CN | G6 | H |
| T2 | — | 34 | H | C—CF3 | C—H | C—Cl | C—H | CF3 | CN | G8 | H |
| T2 | — | 35 | H | C—CF3 | C—H | C—Cl | C—H | CF3 | CN | G9 | H |
| T2 | — | 36 | H | C—CF3 | C—H | C—Cl | C—H | CF3 | CN | F | H |
| T2 | — | 37 | H | C—CF3 | C—F | C—H | C—H | CF3 | CN | G2 | H |
| T2 | — | 38 | H | C—CF3 | C—F | C—H | C—H | CF₃ | CN | G6 | H |
| T2 | — | 39 | H | C—CF3 | C—F | C—H | C—H | CF₃ | CN | G8 | H |
| T2 | — | 40 | H | C—CF3 | C—F | C—H | C—H | CF₃ | CN | G9 | H |
| T2 | — | 41 | H | C—CF3 | C—F | C—H | C—H | CF₃ | CN | F | H |
| T2 | — | 42 | H | C—CF3 | C—H | C—F | C—H | CF₃ | CN | G2 | H |
| T2 | — | 43 | H | C—CF3 | C—H | C—F | C—H | CF₃ | CN | G6 | H |
| T2 | — | 44 | H | C—CF3 | C—H | C—F | C—H | CF₃ | CN | G8 | H |
| T2 | — | 45 | H | C—CF3 | C—H | C—F | C—H | CF₃ | CN | G9 | H |
| T2 | — | 46 | H | C—CF3 | C—H | C—F | C—H | CF₃ | CN | F | H |
| T2 | — | 47 | H | C—Cl | C—Cl | C—Cl | C—H | CF₃ | CN | G2 | H |
| T2 | — | 48 | H | C—Cl | C—Cl | C—Cl | C—H | CF₃ | CN | G2 | 4-F |
| T2 | — | 49 | H | C—Cl | C—Cl | C—Cl | C—H | CF₃ | CN | G2 | 4-Cl |
| T2 | — | 50 | H | C—Cl | C—Cl | C—Cl | C—H | CF₃ | CN | G2 | 4-CN |
| T2 | — | 51 | H | C—Cl | C—Cl | C—Cl | C—H | CF₃ | CN | G2 | 4-NO2 |
| T2 | — | 52 | H | C—Cl | C—Cl | C—Cl | C—H | CF₃ | CN | G6 | H |
| T2 | — | 53 | H | C—Cl | C—Cl | C—Cl | C—H | CF₃ | CN | G8 | H |
| T2 | — | 54 | H | C—Cl | C—Cl | C—Cl | C—H | CF₃ | CN | G9 | H |
| T2 | — | 55 | H | C—Cl | C—Cl | C—Cl | C—H | CF₃ | CN | F | H |
| T2 | — | 56 | H | C—Cl | C—Cl | C—Cl | C—H | CF₃ | CN | Cl | H |
| T2 | — | 57 | H | C—Cl | C—Cl | C—Cl | C—H | CF₃ | CN | Br | H |
| T2 | — | 58 | H | C—Cl | C—Cl | C—CF3 | C—H | CF₃ | CN | G2 | H |
| T2 | — | 59 | H | C—Cl | C—Cl | C—CF3 | C—H | CF₃ | CN | G6 | H |
| T2 | — | 60 | H | C—Cl | C—Cl | C—CF3 | C—H | CF₃ | CN | G8 | H |
| T2 | — | 61 | H | C—Cl | C—Cl | C—CF3 | C—H | CF₃ | CN | G9 | H |
| T2 | — | 62 | H | C—Cl | C—Cl | C—CF3 | C—H | CF₃ | CN | F | H |
| T2 | — | 63 | H | C—Cl | N | C—Cl | C—H | CF₃ | CN | G2 | H |
| T2 | — | 64 | H | C—Cl | N | C—Cl | C—H | CF₃ | CN | G2 | 4-F |
| T2 | — | 65 | H | C—Cl | N | C—Cl | C—H | CF₃ | CN | G2 | 4-Cl |
| T2 | — | 66 | H | C—Cl | N | C—Cl | C—H | CF₃ | CN | G2 | 4-CN |
| T2 | — | 67 | H | C—Cl | N | C—Cl | C—H | CF₃ | CN | G2 | 4-NO2 |
| T2 | — | 68 | H | C—Cl | N | C—Cl | C—H | CF₃ | CN | G6 | H |
| T2 | — | 69 | H | C—Cl | N | C—Cl | C—H | CF₃ | CN | G8 | H |
| T2 | — | 70 | H | C—Cl | N | C—Cl | C—H | CF₃ | CN | G9 | H |
| T2 | — | 71 | H | C—Cl | N | C—Cl | C—H | CF₃ | CN | F | H |
| T2 | — | 72 | H | C—CF3 | N | C—CF3 | C—H | CF₃ | CN | G2 | H |
| T2 | — | 73 | H | C—CF3 | N | C—CF3 | C—H | CF₃ | CN | G6 | H |
| T2 | — | 74 | H | C—CF3 | N | C—CF3 | C—H | CF₃ | CN | G8 | H |
| T2 | — | 75 | H | C—CF3 | N | C—CF3 | C—H | CF₃ | CN | G9 | H |
| T2 | — | 76 | H | C—CF3 | N | C—CF3 | C—H | CF₃ | CN | F | H |
| T2 | — | 77 | H | C—CF3 | N | C—CF3 | C—H | CF₃ | CN | Cl | H |
| T2 | — | 78 | H | C—CF3 | N | C—CF3 | C—H | CF₃ | CN | Br | H |
| T2 | — | 79 | H | C—CF3 | N | C—Cl | C—H | CF₃ | CN | G2 | H |
| T2 | — | 80 | H | C—CF3 | N | C—Cl | C—H | CF₃ | CN | G2 | 4-Cl |
| T2 | — | 81 | H | C—CF3 | N | C—Cl | C—H | CF₃ | CN | G2 | 4-CN |
| T2 | — | 82 | H | C—CF3 | N | C—Cl | C—H | CF₃ | CN | G2 | 4-NO2 |
| T2 | — | 83 | H | C—CF3 | N | C—Cl | C—H | CF₃ | CN | G6 | H |
| T2 | — | 84 | H | C—CF3 | N | C—Cl | C—H | CF₃ | CN | G8 | H |
| T2 | — | 85 | H | C—CF3 | N | C—Cl | C—H | CF₃ | CN | G9 | H |
| T2 | — | 86 | H | C—CF3 | N | C—Cl | C—H | CF₃ | CN | F | H |
| T2 | — | 87 | H | C—Cl | C—H | C—Cl | C—H | CF₃ | Br | G2 | H |
| T2 | — | 88 | H | C—Cl | C—H | C—Cl | C—H | CF₃ | Br | G2 | 4-F |
| T2 | — | 89 | H | C—Cl | C—H | C—Cl | C—H | CF₃ | Br | G2 | 4-Cl |
| T2 | — | 90 | H | C—Cl | C—H | C—Cl | C—H | CF₃ | Br | G2 | 4-CN |
| T2 | — | 91 | H | C—Cl | C—H | C—Cl | C—H | CF₃ | Br | G2 | 4-NO2 |
| T2 | — | 92 | H | C—Cl | C—H | C—Cl | C—H | CF3 | Br | G6 | H |
| T2 | — | 93 | H | C—Cl | C—H | C—Cl | C—H | CF3 | Br | G8 | H |
| T2 | — | 94 | H | C—Cl | C—H | C—Cl | C—H | CF3 | Br | G9 | H |
| T2 | — | 95 | H | C—Cl | C—H | C—Cl | C—H | CF3 | Br | F | H |
| T2 | — | 96 | H | C—Cl | C—H | C—Cl | C—H | CF3 | Br | Cl | H |
| T2 | — | 97 | H | C—CF3 | C—H | C—CF3 | C—H | CF3 | Br | G2 | H |
| T2 | — | 98 | H | C—CF3 | C—H | C—CF3 | C—H | CF3 | Br | G2 | 4-F |
| T2 | — | 99 | H | C—CF3 | C—H | C—CF3 | C—H | CF3 | Br | G2 | 4-Cl |
| T2 | — | 100 | H | C—CF3 | C—H | C—CF3 | C—H | CF3 | Br | G2 | 4-CN |
| T2 | — | 101 | H | C—CF3 | C—H | C—CF3 | C—H | CF3 | Br | G6 | H |
| T2 | — | 102 | H | C—CF3 | C—H | C—CF3 | C—H | CF3 | Br | G8 | H |
| T2 | — | 103 | H | C—CF3 | C—H | C—CF3 | C—H | CF3 | Br | G9 | H |
| T2 | — | 104 | H | C—CF3 | C—H | C—CF3 | C—H | CF3 | Br | F | H |
| T2 | — | 105 | H | C—CF3 | C—H | C—CF3 | C—H | CF3 | Br | Cl | H |
| T2 | — | 106 | H | C—CF3 | C—H | C—Cl | C—H | CF3 | Br | G2 | H |
| T2 | — | 107 | H | C—CF3 | C—H | C—Cl | C—H | CF3 | Br | G6 | H |
| T2 | — | 108 | H | C—CF3 | C—H | C—Cl | C—H | CF3 | Br | G8 | H |
| T2 | — | 109 | H | C—CF3 | C—H | C—Cl | C—H | CF3 | Br | G9 | H |
| T2 | — | 110 | H | C—CF3 | C—H | C—Cl | C—H | CF3 | Br | F | H |
| T2 | — | 111 | H | C—CF3 | C—F | C—H | C—H | CF3 | Br | G2 | H |
| T2 | — | 112 | H | C—CF3 | C—F | C—H | C—H | CF₃ | Br | G6 | H |
| T2 | — | 113 | H | C—CF3 | C—F | C—H | C—H | CF₃ | Br | G8 | H |
| T2 | — | 114 | H | C—CF3 | C—F | C—H | C—H | CF₃ | Br | G9 | H |
| T2 | — | 115 | H | C—CF3 | C—F | C—H | C—H | CF₃ | Br | F | H |
| T2 | — | 116 | H | C—CF3 | C—H | C—F | C—H | CF₃ | Br | G2 | H |
| T2 | — | 117 | H | C—CF3 | C—H | C—F | C—H | CF₃ | Br | G6 | H |
| T2 | — | 118 | H | C—CF3 | C—H | C—F | C—H | CF₃ | Br | G8 | H |
| T2 | — | 119 | H | C—CF3 | C—H | C—F | C—H | CF₃ | Br | G9 | H |
| T2 | — | 120 | H | C—CF3 | C—H | C—F | C—H | CF₃ | Br | F | H |
| T2 | — | 121 | H | C—Cl | C—Cl | C—Cl | C—H | CF₃ | Br | G2 | H |
| T2 | — | 122 | H | C—Cl | C—Cl | C—Cl | C—H | CF₃ | Br | G2 | 4-F |
| T2 | — | 123 | H | C—Cl | C—Cl | C—Cl | C—H | CF₃ | Br | G2 | 4-Cl |
| T2 | — | 124 | H | C—Cl | C—Cl | C—Cl | C—H | CF₃ | Br | G2 | 4-CN |
| T2 | — | 125 | H | C—Cl | C—Cl | C—Cl | C—H | CF₃ | Br | G2 | 4-NO2 |
| T2 | — | 126 | H | C—Cl | C—Cl | C—Cl | C—H | CF₃ | Br | G4 | H |
| T2 | — | 127 | H | C—Cl | C—Cl | C—Cl | C—H | CF₃ | Br | G6 | H |
| T2 | — | 128 | H | C—Cl | C—Cl | C—Cl | C—H | CF₃ | Br | G8 | H |
| T2 | — | 129 | H | C—Cl | C—Cl | C—Cl | C—H | CF₃ | Br | G9 | H |
| T2 | — | 130 | H | C—Cl | C—Cl | C—Cl | C—H | CF₃ | Br | F | H |
| T2 | — | 131 | H | C—Cl | C—Cl | C—Cl | C—H | CF₃ | Br | Cl | H |
| T2 | — | 132 | H | C—Cl | N | C—Cl | C—H | CF₃ | Br | G2 | H |
| T2 | — | 133 | H | C—Cl | N | C—Cl | C—H | CF₃ | Br | G2 | 4-F |
| T2 | — | 134 | H | C—Cl | N | C—Cl | C—H | CF₃ | Br | G2 | 4-Cl |
| T2 | — | 135 | H | C—Cl | N | C—Cl | C—H | CF₃ | Br | G2 | 4-CN |
| T2 | — | 136 | H | C—Cl | N | C—Cl | C—H | CF₃ | Br | G2 | 4-NO2 |
| T2 | — | 137 | H | C—Cl | N | C—Cl | C—H | CF₃ | Br | G6 | H |
| T2 | — | 138 | H | C—Cl | N | C—Cl | C—H | CF₃ | Br | G8 | H |
| T2 | — | 139 | H | C—Cl | N | C—Cl | C—H | CF₃ | Br | G9 | H |
| T2 | — | 140 | H | C—Cl | N | C—Cl | C—H | CF₃ | Br | F | H |
| T2 | — | 141 | H | C—CF3 | N | C—CF3 | C—H | CF₃ | Br | G2 | H |
| T2 | — | 142 | H | C—CF3 | N | C—CF3 | C—H | CF₃ | Br | G6 | H |
| T2 | — | 143 | H | C—CF3 | N | C—CF3 | C—H | CF₃ | Br | G8 | H |
| T2 | — | 144 | H | C—CF3 | N | C—CF3 | C—H | CF₃ | Br | G9 | H |
| T2 | — | 145 | H | C—CF3 | N | C—CF3 | C—H | CF₃ | Br | F | H |
| T2 | — | 146 | H | C—CF3 | N | C—CF3 | C—H | CF₃ | Br | Cl | H |
| T2 | — | 147 | H | C—CF3 | N | C—CF3 | C—H | CF₃ | Br | Br | H |
| T2 | — | 148 | H | C—CF3 | N | C—Cl | C—H | CF₃ | Br | G2 | H |
| T2 | — | 149 | H | C—CF3 | N | C—Cl | C—H | CF₃ | Br | G2 | 4-Cl |
| T2 | — | 150 | H | C—CF3 | N | C—Cl | C—H | CF₃ | Br | G2 | 4-CN |
| T2 | — | 151 | H | C—CF3 | N | C—Cl | C—H | CF₃ | Br | G2 | 4-NO2 |
| T2 | — | 152 | H | C—CF3 | N | C—Cl | C—H | CF₃ | Br | G6 | H |
| T2 | — | 153 | H | C—CF3 | N | C—Cl | C—H | CF₃ | Br | G8 | H |
| T2 | — | 154 | H | C—CF3 | N | C—Cl | C—H | CF₃ | Br | G9 | H |
| T2 | — | 155 | H | C—CF3 | N | C—Cl | C—H | CF₃ | Br | F | H |
| T2 | — | 156 | H | C—CF3 | N | C—H | C—H | CF₃ | Br | G2 | H |
| T2 | — | 157 | H | C—CF3 | N | C—H | C—H | CF₃ | Br | G6 | H |
| T2 | — | 158 | H | C—CF3 | N | C—H | C—H | CF₃ | Br | G8 | H |
| T2 | — | 159 | H | C—CF3 | N | C—H | C—H | CF₃ | Br | G9 | H |
| T2 | — | 160 | H | C—CF3 | N | C—H | C—H | CF₃ | Br | F | H |
| T2 | — | 161 | H | C—CF3 | N | C—H | C—H | CF₃ | Br | Cl | H |
| T2 | — | 162 | H | C—CF3 | N | C—H | C—H | CF₃ | CN | G2 | H |
| T2 | — | 163 | H | C—CF3 | N | C—H | C—H | CF₃ | CN | G6 | H |
| T2 | — | 164 | H | C—CF3 | N | C—H | C—H | CF₃ | CN | G8 | H |
| T2 | — | 165 | H | C—CF3 | N | C—H | C—H | CF₃ | CN | G9 | H |
| T2 | — | 166 | H | C—CF3 | N | C—H | C—H | CF₃ | CN | F | H |
| T2 | — | 167 | H | C—CF3 | N | C—H | C—H | CF₃ | CN | Cl | H |
| T2 | — | 168 | H | C—CF3 | N | C—H | C—H | CF₃ | CN | Br | H |
| T2 | — | 169 | H | C—Cl | N | C—H | C—H | CF₃ | Br | G2 | H |
| T2 | — | 170 | H | C—Cl | N | C—H | C—H | CF₃ | Br | G2 | 4-Cl |
| T2 | — | 171 | H | C—Cl | N | C—H | C—H | CF₃ | Br | G2 | 4-CN |
| T2 | — | 172 | H | C—Cl | N | C—H | C—H | CF₃ | Br | G2 | 4-NO2 |
| T2 | — | 173 | H | C—Cl | N | C—H | C—H | CF₃ | Br | G6 | H |
| T2 | — | 174 | H | C—Cl | N | C—H | C—H | CF₃ | Br | G8 | H |
| T2 | — | 175 | H | C—Cl | N | C—H | C—H | CF₃ | Br | G9 | H |
| T2 | — | 176 | H | C—Cl | N | C—H | C—H | CF₃ | Br | F | H |
| T2 | — | 177 | H | C—Cl | N | C—H | C—H | CF₃ | CN | G2 | H |
| T2 | — | 178 | H | C—Cl | N | C—H | C—H | CF₃ | CN | G2 | 4-Cl |
| T2 | — | 179 | H | C—Cl | N | C—H | C—H | CF₃ | CN | G2 | 4-CN |
| T2 | — | 180 | H | C—Cl | N | C—H | C—H | CF₃ | CN | G2 | 4-NO2 |
| T2 | — | 181 | H | C—Cl | N | C—H | C—H | CF₃ | CN | G6 | H |
| T2 | — | 182 | H | C—Cl | N | C—H | C—H | CF₃ | CN | G8 | H |
| T2 | — | 183 | H | C—Cl | N | C—H | C—H | CF₃ | CN | G9 | H |
| T2 | — | 184 | H | C—Cl | N | C—H | C—H | CF₃ | CN | F | H |
| T2 | — | 185 | H | C—Cl | C—F | C—Cl | C—F | CF₃ | Br | G2 | H |
| T2 | — | 186 | H | C—Cl | C—F | C—Cl | C—F | CF₃ | Br | G2 | 4-Cl |
| T2 | — | 187 | H | C—Cl | C—F | C—Cl | C—F | CF₃ | Br | G2 | 4-CN |
| T2 | — | 188 | H | C—Cl | C—F | C—Cl | C—F | CF₃ | Br | G2 | 4-NO2 |
| T2 | — | 189 | H | C—Cl | C—F | C—Cl | C—F | CF₃ | Br | G6 | H |
| T2 | — | 190 | H | C—Cl | C—F | C—Cl | C—F | CF₃ | Br | G8 | H |
| T2 | — | 191 | H | C—Cl | C—F | C—Cl | C—F | CF₃ | Br | G9 | H |
| T2 | — | 192 | H | C—Cl | C—F | C—Cl | C—F | CF₃ | Br | F | H |
| T2 | — | 193 | H | C—Cl | C—F | C—Cl | C—F | CF₃ | CN | G2 | H |
| T2 | — | 194 | H | C—Cl | C—F | C—Cl | C—F | CF₃ | CN | G2 | 4-Cl |
| T2 | — | 195 | H | C—Cl | C—F | C—Cl | C—F | CF₃ | CN | G2 | 4-CN |
| T2 | — | 196 | H | C—Cl | C—F | C—Cl | C—F | CF₃ | CN | G2 | 4-NO2 |
| T2 | — | 197 | H | C—Cl | C—F | C—Cl | C—F | CF₃ | CN | G6 | H |
| T2 | — | 198 | H | C—Cl | C—F | C—Cl | C—F | CF₃ | CN | G8 | H |
| T2 | — | 199 | H | C—Cl | C—F | C—Cl | C—F | CF₃ | CN | G9 | H |
| T2 | — | 200 | H | C—Cl | C—F | C—Cl | C—F | CF₃ | CN | F | H |

TABLE 3

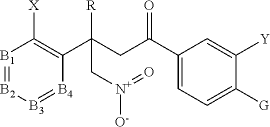

| Compound | | | X | B¹ | B² | B³ | B⁴ | R | Y | G |
|---|---|---|---|---|---|---|---|---|---|---|
| T3 | — | 1 | H | C—Cl | C—H | C—Cl | C—H | CF3 | Me | CN |
| T3 | — | 2 | H | C—Cl | C—H | C—Cl | C—H | CF3 | Cl | CN |
| T3 | — | 3 | H | C—Cl | C—H | C—Cl | C—H | CF3 | Me | CO2H |
| T3 | — | 4 | H | C—Cl | C—H | C—Cl | C—H | CF3 | Me | CO2Me |
| T3 | — | 5 | H | C—Cl | C—H | C—Cl | C—H | CF3 | Me | CO2Et |
| T3 | — | 6 | H | C—Cl | C—H | C—Cl | C—H | CF3 | Cl | CO2H |
| T3 | — | 7 | H | C—Cl | C—H | C—Cl | C—H | CF3 | Cl | CO2Me |
| T3 | — | 8 | H | C—Cl | C—H | C—Cl | C—H | CF3 | Br | CO2H |
| T3 | — | 9 | H | C—Cl | C—H | C—Cl | C—H | CF3 | Br | CO2Me |
| T3 | — | 10 | H | C—Br | C—H | C—Br | C—H | CF3 | Me | CN |
| T3 | — | 11 | H | C—Br | C—H | C—Br | C—H | CF3 | Me | CO2H |
| T3 | — | 12 | H | C—Br | C—H | C—Br | C—H | CF3 | Me | CO2Me |
| T3 | — | 13 | H | C—Br | C—H | C—Br | C—H | CF3 | Me | CO2Et |
| T3 | — | 14 | H | C—CF3 | C—H | C—H | C—H | CF3 | Me | CN |
| T3 | — | 15 | H | C—CF3 | C—H | C—H | C—H | CF3 | Me | CO2H |
| T3 | — | 16 | H | C—CF3 | C—H | C—H | C—H | CF3 | Me | CO2Me |
| T3 | — | 17 | H | C—CF3 | C—H | C—H | C—H | CF3 | Me | CO2Et |
| T3 | — | 18 | H | C—CF3 | C—H | C—CF3 | C—H | CF3 | H | CN |
| T3 | — | 19 | H | C—CF3 | C—H | C—CF3 | C—H | CF3 | Me | CN |
| T3 | — | 20 | H | C—CF3 | C—H | C—CF3 | C—H | CF3 | Cl | CN |
| T3 | — | 21 | H | C—CF3 | C—H | C—CF3 | C—H | CF3 | H | CO2H |
| T3 | — | 22 | H | C—CF3 | C—H | C—CF3 | C—H | CF3 | Me | CO2H |
| T3 | — | 23 | H | C—CF3 | C—H | C—CF3 | C—H | CF3 | Me | CO2Me |
| T3 | — | 24 | H | C—CF3 | C—H | C—CF3 | C—H | CF3 | Me | CO2Et |
| T3 | — | 25 | H | C—CF3 | C—H | C—CF3 | C—H | CF3 | Cl | CO2H |
| T3 | — | 26 | H | C—CF3 | C—H | C—CF3 | C—H | CF3 | Cl | CO2Me |
| T3 | — | 27 | H | C—CF3 | C—H | C—CF3 | C—H | CF3 | Br | CO2H |
| T3 | — | 28 | H | C—CF3 | C—H | C—CF3 | C—H | CF3 | Br | CO2Me |
| T3 | — | 29 | H | C—CF3 | C—H | C—Cl | C—H | CF3 | Me | CN |
| T3 | — | 30 | H | C—CF3 | C—H | C—Cl | C—H | CF3 | Me | CO2H |
| T3 | — | 31 | H | C—CF3 | C—H | C—Cl | C—H | CF3 | Me | CO2Me |
| T3 | — | 32 | H | C—CF3 | C—F | C—H | C—H | CF3 | Me | CN |
| T3 | — | 33 | H | C—CF3 | C—F | C—H | C—H | CF3 | Me | CO2H |
| T3 | — | 34 | H | C—CF3 | C—F | C—H | C—H | CF3 | Me | CO2Me |
| T3 | — | 35 | H | C—CF3 | C—H | C—F | C—H | CF3 | Me | CN |
| T3 | — | 36 | H | C—CF3 | C—H | C—F | C—H | CF3 | Me | CO2H |
| T3 | — | 37 | H | C—CF3 | C—H | C—F | C—H | CF3 | Me | CO2Me |
| T3 | — | 38 | H | C—Cl | C—Cl | C—Cl | C—H | CF3 | H | CN |
| T3 | — | 39 | H | C—Cl | C—Cl | C—Cl | C—H | CF3 | Me | CN |
| T3 | — | 40 | H | C—Cl | C—Cl | C—Cl | C—H | CF3 | Cl | CN |
| T3 | — | 41 | H | C—Cl | C—Cl | C—Cl | C—H | CF3 | H | CO2H |
| T3 | — | 42 | H | C—Cl | C—Cl | C—Cl | C—H | CF3 | Me | CO2H |
| T3 | — | 43 | H | C—Cl | C—Cl | C—Cl | C—H | CF3 | Me | CO2Me |
| T3 | — | 44 | H | C—Cl | C—Cl | C—Cl | C—H | CF3 | Me | CO2Et |
| T3 | — | 45 | H | C—Cl | C—Cl | C—Cl | C—H | CF3 | Cl | CO2H |
| T3 | — | 46 | H | C—Cl | C—Cl | C—Cl | C—H | CF3 | Cl | CO2Me |
| T3 | — | 47 | H | C—Cl | C—Cl | C—Cl | C—H | CF3 | Br | CO2H |
| T3 | — | 48 | H | C—Cl | C—Cl | C—Cl | C—H | CF3 | Br | CO2Me |
| T3 | — | 49 | H | C—Cl | C—Cl | C—CF3 | C—H | CF3 | Me | CN |
| T3 | — | 50 | H | C—Cl | C—Cl | C—CF3 | C—H | CF3 | Me | CO2H |
| T3 | — | 51 | H | C—Cl | C—Cl | C—CF3 | C—H | CF3 | Me | CO2Me |
| T3 | — | 52 | H | C—Cl | N | C—Cl | C—H | CF3 | H | CN |
| T3 | — | 53 | H | C—Cl | N | C—Cl | C—H | CF3 | Me | CN |
| T3 | — | 54 | H | C—Cl | N | C—Cl | C—H | CF3 | Cl | CN |
| T3 | — | 55 | H | C—Cl | N | C—Cl | C—H | CF3 | H | CO2H |
| T3 | — | 56 | H | C—Cl | N | C—Cl | C—H | CF3 | Me | CO2H |
| T3 | — | 57 | H | C—Cl | N | C—Cl | C—H | CF3 | Me | CO2Me |
| T3 | — | 58 | H | C—Cl | N | C—Cl | C—H | CF3 | Me | CO2Et |
| T3 | — | 59 | H | C—Cl | N | C—Cl | C—H | CF3 | Cl | CO2H |
| T3 | — | 60 | H | C—Cl | N | C—Cl | C—H | CF3 | Cl | CO2Me |
| T3 | — | 61 | H | C—Cl | N | C—Cl | C—H | CF3 | Br | CO2H |
| T3 | — | 62 | H | C—Cl | N | C—Cl | C—H | CF3 | Br | CO2Me |
| T3 | — | 63 | H | C—CF3 | N | C—CF3 | C—H | CF3 | H | CO2H |
| T3 | — | 64 | H | C—CF3 | N | C—CF3 | C—H | CF3 | Me | CO2H |
| T3 | — | 65 | H | C—CF3 | N | C—CF3 | C—H | CF3 | Me | CO2Me |
| T3 | — | 66 | H | C—CF3 | N | C—CF3 | C—H | CF3 | Me | CO2Et |
| T3 | — | 67 | H | C—CF3 | N | C—CF3 | C—H | CF3 | Cl | CO2H |
| T3 | — | 68 | H | C—CF3 | N | C—CF3 | C—H | CF3 | Cl | CO2Me |
| T3 | — | 69 | H | C—CF3 | N | C—CF3 | C—H | CF3 | Br | CO2H |
| T3 | — | 70 | H | C—CF3 | N | C—CF3 | C—H | CF3 | Br | CO2Me |
| T3 | — | 71 | H | C—CF3 | N | C—H | C—H | CF3 | Me | CN |
| T3 | — | 72 | H | C—CF3 | N | C—H | C—H | CF3 | Me | CO2H |
| T3 | — | 73 | H | C—CF3 | N | C—H | C—H | CF3 | Me | CO2Me |
| T3 | — | 74 | H | C—CF3 | N | C—Cl | C—H | CF3 | H | CO2H |
| T3 | — | 75 | H | C—CF3 | N | C—Cl | C—H | CF3 | Me | CO2H |
| T3 | — | 76 | H | C—CF3 | N | C—Cl | C—H | CF3 | Me | CO2Me |
| T3 | — | 77 | H | C—CF3 | N | C—Cl | C—H | CF3 | Me | CO2Et |
| T3 | — | 78 | H | C—CF3 | N | C—Cl | C—H | CF3 | Cl | CO2H |
| T3 | — | 79 | H | C—CF3 | N | C—Cl | C—H | CF3 | Cl | CO2Me |
| T3 | — | 80 | H | C—CF3 | N | C—Cl | C—H | CF3 | Br | CO2H |
| T3 | — | 81 | H | C—CF3 | N | C—Cl | C—H | CF3 | Br | CO2Me |
| T3 | — | 82 | H | C—Cl | N | C—H | C—H | CF3 | H | CN |
| T3 | — | 83 | H | C—Cl | N | C—H | C—H | CF3 | Me | CN |
| T3 | — | 84 | H | C—Cl | N | C—H | C—H | CF3 | Cl | CN |
| T3 | — | 85 | H | C—Cl | N | C—H | C—H | CF3 | H | CO2H |
| T3 | — | 86 | H | C—Cl | N | C—H | C—H | CF3 | Me | CO2H |
| T3 | — | 87 | H | C—Cl | N | C—H | C—H | CF3 | Me | CO2Me |
| T3 | — | 88 | H | C—Cl | N | C—H | C—H | CF3 | Me | CO2Et |
| T3 | — | 89 | H | C—Cl | N | C—H | C—H | CF3 | Cl | CO2H |
| T3 | — | 90 | H | C—Cl | N | C—H | C—H | CF3 | Cl | CO2Me |
| T3 | — | 91 | H | C—Cl | N | C—H | C—H | CF3 | Br | CO2H |
| T3 | — | 92 | H | C—Cl | N | C—H | C—H | CF3 | Br | CO2Me |
| T3 | — | 93 | H | C—Cl | C—F | C—Cl | C—F | CF3 | Me | CN |
| T3 | — | 94 | H | C—Cl | C—F | C—Cl | C—F | CF3 | Cl | CN |
| T3 | — | 95 | H | C—Cl | C—F | C—Cl | C—F | CF3 | Me | CO2H |
| T3 | — | 96 | H | C—Cl | C—F | C—Cl | C—F | CF3 | Me | CO2Me |
| T3 | — | 97 | H | C—Cl | C—F | C—Cl | C—F | CF3 | Me | CO2Et |
| T3 | — | 98 | H | C—Cl | C—F | C—Cl | C—F | CF3 | Cl | CO2H |
| T3 | — | 99 | H | C—Cl | C—F | C—Cl | C—F | CF3 | Cl | CO2Me |
| T3 | — | 100 | H | C—Cl | C—F | C—Cl | C—F | CF3 | Br | CO2H |
| T3 | — | 101 | H | C—Cl | C—F | C—Cl | C—F | CF3 | Br | CO2Me |

TABLE 4

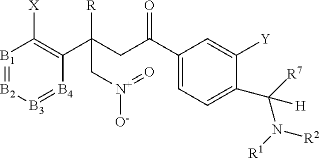

| Compound | | | X | B¹ | B² | B³ | B⁴ | R | R¹ | R² | R³ | Y |
|---|---|---|---|---|---|---|---|---|---|---|---|---|
| T4 | — | 1 | H | C—Cl | C—H | C—Cl | C—H | CF₃ | H | H | H | H |
| T4 | — | 2 | H | C—Cl | C—H | C—Cl | C—H | CF₃ | MeCO | H | H | H |
| T4 | — | 3 | H | C—Cl | C—H | C—Cl | C—H | CF3 | EtCO | H | H | H |
| T4 | — | 4 | H | C—Cl | C—H | C—Cl | C—H | CF3 | n-PrCO | H | H | H |
| T4 | — | 5 | H | C—Cl | C—H | C—Cl | C—H | CF3 | cyclo-PrCO | H | H | H |
| T4 | — | 6 | H | C—Cl | C—H | C—Cl | C—H | CF3 | cyclo-PrCH2CO | H | H | H |
| T4 | — | 7 | H | C—Cl | C—H | C—Cl | C—H | CF3 | CF3CH2CO | H | H | H |
| T4 | — | 8 | H | C—Br | C—H | C—Br | C—H | CF3 | H | H | H | H |
| T4 | — | 9 | H | C—Br | C—H | C—Br | C—H | CF₃ | MeCO | H | H | H |
| T4 | — | 10 | H | C—Br | C—H | C—Br | C—H | CF₃ | EtCO | H | H | H |
| T4 | — | 11 | H | C—Br | C—H | C—Br | C—H | CF₃ | n-PrCO | H | H | H |
| T4 | — | 12 | H | C—Br | C—H | C—Br | C—H | CF₃ | cyclo-PrCO | H | H | H |
| T4 | — | 13 | H | C—Br | C—H | C—Br | C—H | CF₃ | cyclo-PrCH2CO | H | H | H |
| T4 | — | 14 | H | C—Br | C—H | C—Br | C—H | CF₃ | CF3CH2CO | H | H | H |
| T4 | — | 15 | H | C—CF3 | C—H | C—H | C—H | CF3 | H | H | H | H |
| T4 | — | 16 | H | C—CF3 | C—H | C—H | C—H | CF3 | MeCO | H | H | H |
| T4 | — | 17 | H | C—CF3 | C—H | C—H | C—H | CF3 | EtCO | H | H | H |
| T4 | — | 18 | H | C—CF3 | C—H | C—H | C—H | CF3 | n-PrCO | H | H | H |
| T4 | — | 19 | H | C—CF3 | C—H | C—H | C—H | CF3 | cyclo-PrCO | H | H | H |
| T4 | — | 20 | H | C—CF3 | C—H | C—H | C—H | CF3 | cyclo-PrCH2CO | H | H | H |
| T4 | — | 21 | H | C—CF3 | C—H | C—H | C—H | CF3 | CF3CH2CO | H | H | H |
| T4 | — | 22 | H | C—CF3 | C—H | C—CF3 | C—H | CF3 | H | H | H | H |
| T4 | — | 23 | H | C—CF3 | C—H | C—CF3 | C—H | CF3 | MeCO | H | H | H |
| T4 | — | 24 | H | C—CF3 | C—H | C—CF3 | C—H | CF3 | EtCO | H | H | H |
| T4 | — | 25 | H | C—CF3 | C—H | C—CF3 | C—H | CF3 | n-PrCO | H | H | H |
| T4 | — | 26 | H | C—CF3 | C—H | C—CF3 | C—H | CF3 | cyclo-PrCO | H | H | H |
| T4 | — | 27 | H | C—CF3 | C—H | C—CF3 | C—H | CF3 | cyclo-PrCH2CO | H | H | H |
| T4 | — | 28 | H | C—CF3 | C—H | C—CF3 | C—H | CF3 | CF3CH2CO | H | H | H |
| T4 | — | 29 | H | C—CF3 | C—H | C—Cl | C—H | CF3 | H | H | H | H |
| T4 | — | 30 | H | C—CF3 | C—H | C—Cl | C—H | CF3 | MeCO | H | H | H |
| T4 | — | 31 | H | C—CF3 | C—H | C—Cl | C—H | CF3 | EtCO | H | H | H |
| T4 | — | 32 | H | C—CF3 | C—H | C—Cl | C—H | CF3 | n-PrCO | H | H | H |
| T4 | — | 33 | H | C—CF3 | C—H | C—Cl | C—H | CF3 | cyclo-PrCO | H | H | H |
| T4 | — | 34 | H | C—CF3 | C—H | C—Cl | C—H | CF3 | cyclo-PrCH2CO | H | H | H |
| T4 | — | 35 | H | C—CF3 | C—H | C—Cl | C—H | CF3 | CF3CH2CO | H | H | H |
| T4 | — | 36 | H | C—CF3 | C—F | C—H | C—H | CF3 | H | H | H | H |
| T4 | — | 37 | H | C—CF3 | C—F | C—H | C—H | CF₃ | MeCO | H | H | H |
| T4 | — | 38 | H | C—CF3 | C—F | C—H | C—H | CF₃ | EtCO | H | H | H |
| T4 | — | 39 | H | C—CF3 | C—F | C—H | C—H | CF₃ | n-PrCO | H | H | H |
| T4 | — | 40 | H | C—CF3 | C—F | C—H | C—H | CF₃ | cyclo-PrCO | H | H | H |
| T4 | — | 41 | H | C—CF3 | C—F | C—H | C—H | CF₃ | cyclo-PrCH2CO | H | H | H |
| T4 | — | 42 | H | C—CF3 | C—F | C—H | C—H | CF₃ | CF3CFH2CO | H | H | H |
| T4 | — | 43 | H | C—CF3 | C—H | C—F | C—H | CF₃ | H | H | H | H |
| T4 | — | 44 | H | C—CF3 | C—H | C—F | C—H | CF₃ | MeCO | H | H | H |
| T4 | — | 45 | H | C—CF3 | C—H | C—F | C—H | CF₃ | EtCO | H | H | H |
| T4 | — | 46 | H | C—CF3 | C—H | C—F | C—H | CF₃ | n-PrCO | H | H | H |
| T4 | — | 47 | H | C—CF3 | C—H | C—F | C—H | CF₃ | cyclo-PrCO | H | H | H |
| T4 | — | 48 | H | C—CF3 | C—H | C—F | C—H | CF₃ | cyclo-PrCH2CO | H | H | H |
| T4 | — | 49 | H | C—CF3 | C—H | C—F | C—H | CF₃ | CF3CH2CO | H | H | H |
| T4 | — | 50 | H | C—Cl | C—Cl | C—Cl | C—H | CF₃ | H | H | H | H |
| T4 | — | 51 | H | C—Cl | C—Cl | C—Cl | C—H | CF₃ | MeCO | H | H | H |
| T4 | — | 52 | H | C—Cl | C—Cl | C—Cl | C—H | CF₃ | n-PrCO | H | H | H |
| T4 | — | 53 | H | C—Cl | C—Cl | C—Cl | C—H | CF₃ | cyclo-PrCO | H | H | H |
| T4 | — | 54 | H | C—Cl | C—Cl | C—Cl | C—H | CF₃ | cyclo-PrCH2CO | H | H | H |
| T4 | — | 55 | H | C—Cl | C—Cl | C—Cl | C—H | CF₃ | CF3CH2CO | H | H | H |
| T4 | — | 56 | H | C—Cl | C—Cl | C—CF3 | C—H | CF₃ | H | H | H | H |
| T4 | — | 57 | H | C—Cl | C—Cl | C—CF3 | C—H | CF₃ | MeCO | H | H | H |
| T4 | — | 58 | H | C—Cl | C—Cl | C—CF3 | C—H | CF₃ | EtCO | H | H | H |
| T4 | — | 59 | H | C—Cl | C—Cl | C—CF3 | C—H | CF₃ | n-PrCO | H | H | H |
| T4 | — | 60 | H | C—Cl | C—Cl | C—CF3 | C—H | CF₃ | cyclo-PrCO | H | H | H |
| T4 | — | 61 | H | C—Cl | C—Cl | C—CF3 | C—H | CF₃ | cyclo-PrCH2CO | H | H | H |
| T4 | — | 62 | H | C—Cl | C—Cl | C—CF3 | C—H | CF₃ | CF3CH2CO | H | H | H |
| T4 | — | 63 | H | C—Cl | N | C—Cl | C—H | CF₃ | H | H | H | H |
| T4 | — | 64 | H | C—Cl | N | C—Cl | C—H | CF₃ | MeCO | H | H | H |
| T4 | — | 65 | H | C—Cl | N | C—Cl | C—H | CF₃ | EtCO | H | H | H |
| T4 | — | 66 | H | C—Cl | N | C—Cl | C—H | CF₃ | n-PrCO | H | H | H |
| T4 | — | 67 | H | C—Cl | N | C—Cl | C—H | CF₃ | cyclo-PrCO | H | H | H |
| T4 | — | 68 | H | C—Cl | N | C—Cl | C—H | CF₃ | cyclo-PrCH2CO | H | H | H |
| T4 | — | 69 | H | C—Cl | N | C—Cl | C—H | CF₃ | CF3CH2CO | H | H | H |
| T4 | — | 70 | H | C—CF3 | N | C—CF3 | C—H | CF₃ | H | H | H | H |
| T4 | — | 71 | H | C—CF3 | N | C—CF3 | C—H | CF₃ | MeCO | H | H | H |
| T4 | — | 72 | H | C—CF3 | N | C—CF3 | C—H | CF₃ | EtCO | H | H | H |
| T4 | — | 73 | H | C—CF3 | N | C—CF3 | C—H | CF₃ | n-PrCO | H | H | H |
| T4 | — | 74 | H | C—CF3 | N | C—CF3 | C—H | CF₃ | cyclo-PrCO | H | H | H |
| T4 | — | 75 | H | C—CF3 | N | C—CF3 | C—H | CF₃ | cyclo-PrCH2CO | H | H | H |
| T4 | — | 76 | H | C—CF3 | N | C—CF3 | C—H | CF₃ | CF3CH2CO | H | H | H |
| T4 | — | 77 | H | C—CF3 | N | C—CF3 | C—H | CF₃ | H | H | H | H |
| T4 | — | 78 | H | C—CF3 | N | C—Cl | C—H | CF₃ | MeCO | H | H | H |
| T4 | — | 79 | H | C—CF3 | N | C—Cl | C—H | CF₃ | EtCO | H | H | H |
| T4 | — | 80 | H | C—CF3 | N | C—Cl | C—H | CF₃ | n-PrCO | H | H | H |
| T4 | — | 81 | H | C—CF3 | N | C—Cl | C—H | CF₃ | cyclo-PrCO | H | H | H |
| T4 | — | 82 | H | C—CF3 | N | C—Cl | C—H | CF₃ | cyclo-PrCH2CO | H | H | H |
| T4 | — | 83 | H | C—CF3 | N | C—Cl | C—H | CF₃ | CF3CH2CO | H | H | H |
| T4 | — | 84 | H | C—Cl | C—Cl | C—Cl | C—H | CF₃ | H | Me | H | H |
| T4 | — | 85 | H | C—Cl | C—Cl | C—Cl | C—H | CF₃ | MeCO | Me | H | H |
| T4 | — | 86 | H | C—Cl | C—Cl | C—Cl | C—H | CF₃ | EtCO | Me | H | H |
| T4 | — | 87 | H | C—Cl | C—Cl | C—Cl | C—H | CF₃ | n-PrCO | Me | H | H |
| T4 | — | 88 | H | C—Cl | C—Cl | C—Cl | C—H | CF₃ | cyclo-PrCO | Me | H | H |
| T4 | — | 89 | H | C—Cl | C—Cl | C—Cl | C—H | CF₃ | cyclo-PrCH2CO | Me | H | H |
| T4 | — | 90 | H | C—Cl | C—Cl | C—Cl | C—H | CF₃ | CF3CH2CO | Me | H | H |
| T4 | | 91 | H | C—CF3 | N | C—H | C—H | CF₃ | H | H | H | H |
| T4 | | 92 | H | C—CF3 | N | C—H | C—H | CF₃ | MeCO | H | H | H |
| T4 | | 93 | H | C—CF3 | N | C—H | C—H | CF₃ | EtCO | H | H | H |
| T4 | | 94 | H | C—CF3 | N | C—H | C—H | CF₃ | n-PrCO | H | H | H |
| T4 | | 95 | H | C—CF3 | N | C—H | C—H | CF₃ | cyclo-PrCO | H | H | H |
| T4 | | 96 | H | C—CF3 | N | C—H | C—H | CF₃ | cyclo-PrCH2CO | H | H | H |
| T4 | | 97 | H | C—CF3 | N | C—H | C—H | CF₃ | CF3CH2CO | H | H | H |
| T4 | | 98 | H | C—Cl | N | C—H | C—H | CF₃ | H | H | H | H |
| T4 | | 99 | H | C—Cl | N | C—H | C—H | CF₃ | MeCO | H | H | H |
| T4 | | 100 | H | C—Cl | N | C—H | C—H | CF₃ | EtCO | H | H | H |
| T4 | | 101 | H | C—Cl | N | C—H | C—H | CF₃ | n-PrCO | H | H | H |
| T4 | | 102 | H | C—Cl | N | C—H | C—H | CF₃ | cyclo-PrCO | H | H | H |
| T4 | | 103 | H | C—Cl | N | C—H | C—H | CF₃ | cyclo-PrCH2CO | H | H | H |
| T4 | | 104 | H | C—Cl | N | C—H | C—H | CF₃ | CF3CH2CO | H | H | H |
| T4 | | 105 | H | C—Cl | C—F | C—Cl | C—F | CF₃ | H | H | H | H |
| T4 | | 106 | H | C—Cl | C—F | C—Cl | C—F | CF₃ | MeCO | H | H | H |
| T4 | | 107 | H | C—Cl | C—F | C—Cl | C—F | CF₃ | EtCO | H | H | H |
| T4 | | 108 | H | C—Cl | C—F | C—Cl | C—F | CF₃ | n-PrCO | H | H | H |
| T4 | | 109 | H | C—Cl | C—F | C—Cl | C—F | CF₃ | cyclo-PrCO | H | H | H |
| T4 | | 110 | H | C—Cl | C—F | C—Cl | C—F | CF₃ | cyclo-PrCH2CO | H | H | H |
| T4 | | 111 | H | C—Cl | C—F | C—Cl | C—F | CF₃ | CF3CH2CO | H | H | H |
| T4 | — | 112 | H | C—Cl | C—H | C—Cl | C—H | CF₃ | H | H | H | F |
| T4 | — | 113 | H | C—Cl | C—H | C—Cl | C—H | CF₃ | MeCO | H | H | F |
| T4 | — | 114 | H | C—Cl | C—H | C—Cl | C—H | CF3 | EtCO | H | H | F |
| T4 | — | 115 | H | C—Cl | C—H | C—Cl | C—H | CF3 | n-PrCO | H | H | F |
| T4 | — | 116 | H | C—Cl | C—H | C—Cl | C—H | CF3 | cyclo-PrCO | H | H | F |
| T4 | — | 117 | H | C—Cl | C—H | C—Cl | C—H | CF3 | cyclo-PrCH2CO | H | H | F |
| T4 | — | 118 | H | C—Cl | C—H | C—Cl | C—H | CF3 | CF3CH2CO | H | H | F |
| T4 | — | 119 | H | C—Br | C—H | C—Br | C—H | CF3 | H | H | H | F |
| T4 | — | 120 | H | C—Br | C—H | C—Br | C—H | CF₃ | MeCO | H | H | F |
| T4 | — | 121 | H | C—Br | C—H | C—Br | C—H | CF₃ | EtCO | H | H | F |
| T4 | — | 122 | H | C—Br | C—H | C—Br | C—H | CF₃ | n-PrCO | H | H | F |
| T4 | — | 123 | H | C—Br | C—H | C—Br | C—H | CF₃ | cyclo-PrCO | H | H | F |
| T4 | — | 124 | H | C—Br | C—H | C—Br | C—H | CF₃ | cyclo-PrCH2CO | H | H | F |
| T4 | — | 125 | H | C—Br | C—H | C—Br | C—H | CF₃ | CF3CH2CO | H | H | F |
| T4 | — | 126 | H | C—CF3 | C—H | C—H | C—H | CF3 | H | H | H | F |
| T4 | — | 127 | H | C—CF3 | C—H | C—H | C—H | CF3 | MeCO | H | H | F |
| T4 | — | 128 | H | C—CF3 | C—H | C—H | C—H | CF3 | EtCO | H | H | F |
| T4 | — | 129 | H | C—CF3 | C—H | C—H | C—H | CF3 | n-PrCO | H | H | F |
| T4 | — | 130 | H | C—CF3 | C—H | C—H | C—H | CF3 | cyclo-PrCO | H | H | F |
| T4 | — | 131 | H | C—CF3 | C—H | C—H | C—H | CF3 | cyclco-PrCH2CO | H | H | F |
| T4 | — | 132 | H | C—CF3 | C—H | C—H | C—H | CF3 | CF3CH2CO | H | H | F |
| T4 | — | 133 | H | C—CF3 | C—H | C—CF3 | C—H | CF3 | H | H | H | F |
| T4 | — | 134 | H | C—CF3 | C—H | C—CF3 | C—H | CF3 | MeCO | H | H | F |
| T4 | — | 135 | H | C—CF3 | C—H | C—CF3 | C—H | CF3 | EtCO | H | H | F |
| T4 | — | 136 | H | C—CF3 | C—H | C—CF3 | C—H | CF3 | n-PrCO | H | H | F |
| T4 | — | 137 | H | C—CF3 | C—H | C—CF3 | C—H | CF3 | cyclo-PrCO | H | H | F |
| T4 | — | 138 | H | C—CF3 | C—H | C—CF3 | C—H | CF3 | cyclo-PrCH2CO | H | H | F |
| T4 | — | 139 | H | C—CF3 | C—H | C—CF3 | C—H | CF3 | CF3CH2CO | H | H | F |
| T4 | — | 140 | H | C—CF3 | C—H | C—Cl | C—H | CF3 | H | H | H | F |
| T4 | — | 141 | H | C—CF3 | C—H | C—Cl | C—H | CF3 | MeCO | H | H | F |
| T4 | — | 142 | H | C—CF3 | C—H | C—Cl | C—H | CF3 | EtCO | H | H | F |
| T4 | — | 143 | H | C—CF3 | C—H | C—Cl | C—H | CF3 | n-PrCO | H | H | F |
| T4 | — | 144 | H | C—CF3 | C—H | C—Cl | C—H | CF3 | cyclo-PrCO | H | H | F |
| T4 | — | 145 | H | C—CF3 | C—H | C—Cl | C—H | CF3 | cyclo-PrCH2CO | H | H | F |
| T4 | — | 146 | H | C—CF3 | C—H | C—Cl | C—H | CF3 | CF3CH2CO | H | H | F |
| T4 | — | 147 | H | C—CF3 | C—F | C—H | C—H | CF3 | H | H | H | F |
| T4 | — | 148 | H | C—CF3 | C—F | C—H | C—H | CF₃ | MeCO | H | H | F |
| T4 | — | 149 | H | C—CF3 | C—F | C—H | C—H | CF₃ | EtCO | H | H | F |
| T4 | — | 150 | H | C—CF3 | C—F | C—H | C—H | CF₃ | n-PrCO | H | H | F |
| T4 | — | 151 | H | C—CF3 | C—F | C—H | C—H | CF₃ | cyclo-PrCO | H | H | F |
| T4 | — | 152 | H | C—CF3 | C—F | C—H | C—H | CF₃ | cyclo-PrCH2CO | H | H | F |
| T4 | — | 153 | H | C—CF3 | C—F | C—H | C—H | CF₃ | CF3CH2CO | H | H | F |
| T4 | — | 154 | H | C—CF3 | C—H | C—F | C—H | CF₃ | H | H | H | F |
| T4 | — | 155 | H | C—CF3 | C—H | C—F | C—H | CF₃ | MeCO | H | H | F |
| T4 | — | 156 | H | C—CF3 | C—H | C—F | C—H | CF₃ | EtCO | H | H | F |
| T4 | — | 157 | H | C—CF3 | C—H | C—F | C—H | CF₃ | n-PrCO | H | H | F |
| T4 | — | 158 | H | C—CF3 | C—H | C—F | C—H | CF₃ | cyclo-PrCO | H | H | F |
| T4 | — | 159 | H | C—CF3 | C—H | C—F | C—H | CF₃ | cyclo-PrCH2CO | H | H | F |
| T4 | — | 160 | H | C—CF3 | C—H | C—F | C—H | CF₃ | CF3CH2CO | H | H | F |
| T4 | — | 161 | H | C—Cl | C—Cl | C—Cl | C—H | CF₃ | H | H | H | F |
| T4 | — | 162 | H | C—Cl | C—Cl | C—Cl | C—H | CF₃ | MeCO | H | H | F |
| T4 | — | 163 | H | C—Cl | C—Cl | C—Cl | C—H | CF₃ | n-PrCO | H | H | F |
| T4 | — | 164 | H | C—Cl | C—Cl | C—Cl | C—H | CF₃ | cyclo-PrCO | H | H | F |
| T4 | — | 165 | H | C—Cl | C—Cl | C—Cl | C—H | CF₃ | cyclo-PrCH2CO | H | H | F |
| T4 | — | 166 | H | C—Cl | C—Cl | C—Cl | C—H | CF₃ | CF3CH2CO | H | H | F |
| T4 | — | 167 | H | C—Cl | C—Cl | C—CF3 | C—H | CF₃ | H | H | H | F |
| T4 | — | 168 | H | C—Cl | C—Cl | C—CF3 | C—H | CF₃ | MeCO | H | H | F |
| T4 | — | 169 | H | C—Cl | C—Cl | C—CF3 | C—H | CF₃ | EtCO | H | H | F |
| T4 | — | 170 | H | C—Cl | C—Cl | C—CF3 | C—H | CF₃ | n-PrCO | H | H | F |
| T4 | — | 171 | H | C—Cl | C—Cl | C—CF3 | C—H | CF₃ | cyclo-PrCO | H | H | F |
| T4 | — | 172 | H | C—Cl | C—Cl | C—CF3 | C—H | CF₃ | cyclo-PrCH2CO | H | H | F |
| T4 | — | 173 | H | C—Cl | C—Cl | C—CF3 | C—H | CF₃ | CF3CH2CO | H | H | F |
| T4 | — | 174 | H | C—Cl | N | C—Cl | C—H | CF₃ | H | H | H | F |
| T4 | — | 175 | H | C—Cl | N | C—Cl | C—H | CF₃ | MeCO | H | H | F |
| T4 | — | 176 | H | C—Cl | N | C—Cl | C—H | CF₃ | EtCO | H | H | F |
| T4 | — | 177 | H | C—Cl | N | C—Cl | C—H | CF₃ | n-PrCO | H | H | F |
| T4 | — | 178 | H | C—Cl | N | C—Cl | C—H | CF₃ | cyclo-PrCO | H | H | F |
| T4 | — | 179 | H | C—Cl | N | C—Cl | C—H | CF₃ | cyclo-PrCH2CO | H | H | F |
| T4 | — | 180 | H | C—Cl | N | C—Cl | C—H | CF₃ | CF3CH2CO | H | H | F |
| T4 | — | 181 | H | C—CF3 | N | C—CF3 | C—H | CF₃ | H | H | H | F |
| T4 | — | 182 | H | C—CF3 | N | C—CF3 | C—H | CF₃ | MeCO | H | H | F |
| T4 | — | 183 | H | C—CF3 | N | C—CF3 | C—H | CF₃ | EtCO | H | H | F |
| T4 | — | 184 | H | C—CF3 | N | C—CF3 | C—H | CF₃ | n-PrCO | H | H | F |
| T4 | — | 185 | H | C—CF3 | N | C—CF3 | C—H | CF₃ | cyclo-PrCO | H | H | F |
| T4 | — | 186 | H | C—CF3 | N | C—CF3 | C—H | CF₃ | cyclo-PrCH2CO | H | H | F |
| T4 | — | 187 | H | C—CF3 | N | C—CF3 | C—H | CF₃ | CF3CH2CO | H | H | F |
| T4 | — | 188 | H | C—CF3 | N | C—Cl | C—H | CF₃ | H | H | H | F |
| T4 | — | 189 | H | C—CF3 | N | C—Cl | C—H | CF₃ | MeCO | H | H | F |
| T4 | — | 190 | H | C—CF3 | N | C—Cl | C—H | CF₃ | EtCO | H | H | F |
| T4 | — | 191 | H | C—CF3 | N | C—Cl | C—H | CF₃ | n-PrCO | H | H | F |
| T4 | — | 192 | H | C—CF3 | N | C—Cl | C—H | CF₃ | cyclo-PrCO | H | H | F |
| T4 | — | 193 | H | C—CF3 | N | C—Cl | C—H | CF₃ | cyclo-PrCH2CO | H | H | F |
| T4 | — | 194 | H | C—CF3 | N | C—Cl | C—H | CF₃ | CF3CH2CO | H | H | F |
| T4 | — | 195 | H | C—Cl | C—Cl | C—Cl | C—H | CF₃ | H | Me | H | F |
| T4 | — | 196 | H | C—Cl | C—Cl | C—Cl | C—H | CF₃ | MeCO | Me | H | F |
| T4 | — | 197 | H | C—Cl | C—Cl | C—Cl | C—H | CF₃ | EtCO | Me | H | F |
| T4 | — | 198 | H | C—Cl | C—Cl | C—Cl | C—H | CF₃ | n-PrCO | Me | H | F |
| T4 | — | 199 | H | C—Cl | C—Cl | C—Cl | C—H | CF₃ | cyclo-PrCO | Me | H | F |
| T4 | — | 200 | H | C—Cl | C—Cl | C—Cl | C—H | CF₃ | cyclo-PrCH2CO | Me | H | F |
| T4 | — | 201 | H | C—Cl | C—Cl | C—Cl | C—H | CF₃ | CF3CH2CO | Me | H | F |
| T4 | | 202 | H | C—CF3 | N | C—H | C—H | CF₃ | H | Me | H | F |
| T4 | | 203 | H | C—CF3 | N | C—H | C—H | CF₃ | MeCO | Me | H | F |
| T4 | | 204 | H | C—CF3 | N | C—H | C—H | CF₃ | EtCO | H | H | F |
| T4 | | 205 | H | C—CF3 | N | C—H | C—H | CF₃ | n-PrCO | H | H | F |
| T4 | | 206 | H | C—CF3 | N | C—H | C—H | CF₃ | cyclo-PrCO | H | H | F |
| T4 | | 207 | H | C—CF3 | N | C—H | C—H | CF₃ | cyclo-PrCH2CO | H | H | F |
| T4 | | 208 | H | C—CF3 | N | C—H | C—H | CF₃ | CF3CH2CO | H | H | F |
| T4 | | 209 | H | C—Cl | N | C—H | C—H | CF₃ | H | H | H | F |
| T4 | | 210 | H | C—Cl | N | C—H | C—H | CF₃ | MeCO | H | H | F |
| T4 | | 211 | H | C—Cl | N | C—H | C—H | CF₃ | EtCO | H | H | F |
| T4 | | 212 | H | C—Cl | N | C—H | C—H | CF₃ | n-PrCO | H | H | F |
| T4 | | 213 | H | C—Cl | N | C—H | C—H | CF₃ | cyclo-PrCO | H | H | F |
| T4 | | 214 | H | C—Cl | N | C—H | C—H | CF₃ | cyclo-PrCH2CO | H | H | F |
| T4 | | 215 | H | C—Cl | N | C—H | C—H | CF₃ | CF3CH2CO | H | H | F |
| T4 | | 216 | H | C—Cl | C—F | C—Cl | C—F | CF₃ | H | H | H | F |
| T4 | | 217 | H | C—Cl | C—F | C—Cl | C—F | CF₃ | MeCO | H | H | F |
| T4 | | 218 | H | C—Cl | C—F | C—Cl | C—F | CF₃ | EtCO | H | H | F |
| T4 | | 219 | H | C—Cl | C—F | C—Cl | C—F | CF₃ | n-PrCO | H | H | F |
| T4 | | 220 | H | C—Cl | C—F | C—Cl | C—F | CF₃ | cyclo-PrCO | H | H | F |
| T4 | | 221 | H | C—Cl | C—F | C—Cl | C—F | CF₃ | cyclo-PrCH2CO | H | H | F |
| T4 | | 222 | H | C—Cl | C—F | C—Cl | C—F | CF₃ | CF3CH2CO | H | H | F |
| T4 | — | 223 | H | C—Cl | C—H | C—Cl | C—H | CF₃ | H | H | H | Cl |
| T4 | — | 224 | H | C—Cl | C—H | C—Cl | C—H | CF₃ | MeCO | H | H | Cl |
| T4 | — | 225 | H | C—Cl | C—H | C—Cl | C—H | CF3 | EtCO | H | H | Cl |
| T4 | — | 226 | H | C—Cl | C—H | C—Cl | C—H | CF3 | n-PrCO | H | H | Cl |
| T4 | — | 227 | H | C—Cl | C—H | C—Cl | C—H | CF3 | cyclo-PrCO | H | H | Cl |
| T4 | — | 228 | H | C—Cl | C—H | C—Cl | C—H | CF3 | cyclo-PrCH2CO | H | H | Cl |
| T4 | — | 229 | H | C—Cl | C—H | C—Cl | C—H | CF3 | CF3CH2CO | H | H | Cl |
| T4 | — | 230 | H | C—Br | C—H | C—Br | C—H | CF3 | H | H | H | Cl |
| T4 | — | 231 | H | C—Br | C—H | C—Br | C—H | CF₃ | MeOC | H | H | Cl |
| T4 | — | 232 | H | C—Br | C—H | C—Br | C—H | CF₃ | EtCO | H | H | Cl |
| T4 | — | 233 | H | C—Br | C—H | C—Br | C—H | CF₃ | n-PrCO | H | H | Cl |
| T4 | — | 234 | H | C—Br | C—H | C—Br | C—H | CF₃ | cyclo-PrCO | H | H | Cl |
| T4 | — | 235 | H | C—Br | C—H | C—Br | C—H | CF₃ | cyclo-PrCH2CO | H | H | Cl |
| T4 | — | 236 | H | C—Br | C—H | C—Br | C—H | CF₃ | CF3CH2CO | H | H | Cl |
| T4 | — | 237 | H | C—CF3 | C—H | C—H | C—H | CF3 | H | H | H | Cl |
| T4 | — | 238 | H | C—CF3 | C—H | C—H | C—H | CF3 | MeCO | H | H | Cl |
| T4 | — | 239 | H | C—CF3 | C—H | C—H | C—H | CF3 | EtCO | H | H | Cl |
| T4 | — | 240 | H | C—CF3 | C—H | C—H | C—H | CF3 | n-PrCO | H | H | Cl |
| T4 | — | 241 | H | C—CF3 | C—H | C—H | C—H | CF3 | cyclo-PrCO | H | H | Cl |
| T4 | — | 242 | H | C—CF3 | C—H | C—H | C—H | CF3 | cyclo-PrCH2CO | H | H | Cl |
| T4 | — | 243 | H | C—CF3 | C—H | C—H | C—H | CF3 | CF3CH2CO | H | H | Cl |
| T4 | — | 244 | H | C—CF3 | C—H | C—CF3 | C—H | CF3 | H | H | H | Cl |
| T4 | — | 245 | H | C—CF3 | C—H | C—CF3 | C—H | CF3 | MeCO | H | H | Cl |
| T4 | — | 246 | H | C—CF3 | C—H | C—CF3 | C—H | CF3 | EtCO | H | H | Cl |
| T4 | — | 247 | H | C—CF3 | C—H | C—CF3 | C—H | CF3 | n-PrCO | H | H | Cl |
| T4 | — | 248 | H | C—CF3 | C—H | C—CF3 | C—H | CF3 | cyclo-PrCO | H | H | Cl |
| T4 | — | 249 | H | C—CF3 | C—H | C—CF3 | C—H | CF3 | cyclo-PrCH2CO | H | H | Cl |
| T4 | — | 250 | H | C—CF3 | C—H | C—CF3 | C—H | CF3 | CF3CH2CO | H | H | Cl |
| T4 | — | 251 | H | C—CF3 | C—H | C—Cl | C—H | CF3 | H | H | H | Cl |
| T4 | — | 252 | H | C—CF3 | C—H | C—Cl | C—H | CF3 | MeCO | H | H | Cl |
| T4 | — | 253 | H | C—CF3 | C—H | C—Cl | C—H | CF3 | EtCO | H | H | Cl |
| T4 | — | 254 | H | C—CF3 | C—H | C—Cl | C—H | CF3 | n-PrCO | H | H | Cl |
| T4 | — | 255 | H | C—CF3 | C—H | C—Cl | C—H | CF3 | cyclo-PrCO | H | H | Cl |
| T4 | — | 256 | H | C—CF3 | C—H | C—Cl | C—H | CF3 | cyclo-PrCH2CO | H | H | Cl |
| T4 | — | 257 | H | C—CF3 | C—H | C—Cl | C—H | CF3 | CF3CH2CO | H | H | Cl |
| T4 | — | 258 | H | C—CF3 | C—F | C—H | C—H | CF3 | H | H | H | Cl |
| T4 | — | 259 | H | C—CF3 | C—F | C—H | C—H | CF₃ | MeCO | H | H | Cl |
| T4 | — | 260 | H | C—CF3 | C—F | C—H | C—H | CF₃ | EtCO | H | H | Cl |
| T4 | — | 261 | H | C—CF3 | C—F | C—H | C—H | CF₃ | n-PrCO | H | H | Cl |
| T4 | — | 262 | H | C—CF3 | C—F | C—H | C—H | CF₃ | cyclo-PrCO | H | H | Cl |
| T4 | — | 263 | H | C—CF3 | C—F | C—H | C—H | CF₃ | cyclo-PrCH2CO | H | H | Cl |
| T4 | — | 264 | H | C—CF3 | C—F | C—H | C—H | CF₃ | CF3CH2CO | H | H | Cl |
| T4 | — | 265 | H | C—CF3 | C—H | C—F | C—H | CF₃ | H | H | H | Cl |
| T4 | — | 266 | H | C—CF3 | C—H | C—F | C—H | CF₃ | MeCO | H | H | Cl |
| T4 | — | 267 | H | C—CF3 | C—H | C—F | C—H | CF₃ | EtCO | H | H | Cl |
| T4 | — | 268 | H | C—CF3 | C—H | C—F | C—H | CF₃ | n-PrCO | H | H | Cl |
| T4 | — | 269 | H | C—CF3 | C—H | C—F | C—H | CF₃ | cyclo-PrCO | H | H | Cl |
| T4 | — | 270 | H | C—CF3 | C—H | C—F | C—H | CF₃ | cyclo-PrCH2CO | H | H | Cl |
| T4 | — | 271 | H | C—CF3 | C—H | C—F | C—H | CF₃ | CF3CH2CO | H | H | Cl |
| T4 | — | 272 | H | C—Cl | C—Cl | C—Cl | C—H | CF₃ | H | H | H | Cl |
| T4 | — | 273 | H | C—Cl | C—Cl | C—Cl | C—H | CF₃ | MeCO | H | H | Cl |
| T4 | — | 274 | H | C—Cl | C—Cl | C—Cl | C—H | CF₃ | n-PrCO | H | H | Cl |
| T4 | — | 275 | H | C—Cl | C—Cl | C—Cl | C—H | CF₃ | cyclo-PrCO | H | H | Cl |
| T4 | — | 276 | H | C—Cl | C—Cl | C—Cl | C—H | CF₃ | cyclo-PrCH2CO | H | H | Cl |
| T4 | — | 277 | H | C—Cl | C—Cl | C—Cl | C—H | CF₃ | CF3CH2CO | H | H | Cl |
| T4 | — | 278 | H | C—Cl | C—Cl | C—CF3 | C—H | CF₃ | H | H | H | Cl |
| T4 | — | 279 | H | C—Cl | C—Cl | C—CF3 | C—H | CF₃ | MeCO | H | H | Cl |
| T4 | — | 280 | H | C—Cl | C—Cl | C—CF3 | C—H | CF₃ | EtCO | H | H | Cl |
| T4 | — | 281 | H | C—Cl | C—Cl | C—CF3 | C—H | CF₃ | n-PrCO | H | H | Cl |
| T4 | — | 282 | H | C—Cl | C—Cl | C—CF3 | C—H | CF₃ | cyclo-PrCO | H | H | Cl |
| T4 | — | 283 | H | C—Cl | C—Cl | C—CF3 | C—H | CF₃ | cyclo-PrCH2CO | H | H | Cl |
| T4 | — | 284 | H | C—Cl | C—Cl | C—CF3 | C—H | CF₃ | CF3CH2CO | H | H | Cl |
| T4 | — | 285 | H | C—Cl | N | C—Cl | C—H | CF₃ | H | H | H | Cl |
| T4 | — | 286 | H | C—Cl | N | C—Cl | C—H | CF₃ | MeCO | H | H | Cl |
| T4 | — | 287 | H | C—Cl | N | C—Cl | C—H | CF₃ | EtCO | H | H | Cl |
| T4 | — | 288 | H | C—Cl | N | C—Cl | C—H | CF₃ | n-PrCO | H | H | Cl |
| T4 | — | 289 | H | C—Cl | N | C—Cl | C—H | CF₃ | cyclo-PrCO | H | H | Cl |
| T4 | — | 290 | H | C—Cl | N | C—Cl | C—H | CF₃ | cyclo-PrCH2CO | H | H | Cl |
| T4 | — | 291 | H | C—Cl | N | C—Cl | C—H | CF₃ | CF3CH2CO | H | H | Cl |
| T4 | — | 292 | H | C—CF3 | N | C—CF3 | C—H | CF₃ | H | H | H | Cl |
| T4 | — | 293 | H | C—CF3 | N | C—CF3 | C—H | CF₃ | MeCO | H | H | Cl |
| T4 | — | 294 | H | C—CF3 | N | C—CF3 | C—H | CF₃ | EtCO | H | H | Cl |
| T4 | — | 295 | H | C—CF3 | N | C—CF3 | C—H | CF₃ | n-PrCO | H | H | Cl |
| T4 | — | 296 | H | C—CF3 | N | C—CF3 | C—H | CF₃ | cyclo-PrCO | H | H | Cl |
| T4 | — | 297 | H | C—CF3 | N | C—CF3 | C—H | CF₃ | cyclo-PrCH2CO | H | H | Cl |
| T4 | — | 298 | H | C—CF3 | N | C—CF3 | C—H | CF₃ | CF3CH2CO | H | H | Cl |
| T4 | — | 299 | H | C—CF3 | N | C—Cl | C—H | CF₃ | H | H | H | Cl |
| T4 | — | 300 | H | C—CF3 | N | C—Cl | C—H | CF₃ | MeCO | H | H | Cl |
| T4 | — | 301 | H | C—CF3 | N | C—Cl | C—H | CF₃ | EtCO | H | H | Cl |
| T4 | — | 302 | H | C—CF3 | N | C—Cl | C—H | CF₃ | n-PrCO | H | H | Cl |
| T4 | — | 303 | H | C—CF3 | N | C—Cl | C—H | CF₃ | cyclo-PrCO | H | H | Cl |
| T4 | — | 304 | H | C—CF3 | N | C—Cl | C—H | CF₃ | cyclo-PrCH2CO | H | H | Cl |
| T4 | — | 305 | H | C—CF3 | N | C—Cl | C—H | CF₃ | CF3CH2CO | H | H | Cl |
| T4 | — | 306 | H | C—Cl | C—Cl | C—Cl | C—H | CF₃ | H | Me | H | Cl |
| T4 | — | 307 | H | C—Cl | C—Cl | C—Cl | C—H | CF₃ | MeCO | Me | H | Cl |
| T4 | — | 308 | H | C—Cl | C—Cl | C—Cl | C—H | CF₃ | EtCO | Me | H | Cl |
| T4 | — | 309 | H | C—Cl | C—Cl | C—Cl | C—H | CF₃ | n-PrCO | Me | H | Cl |
| T4 | — | 310 | H | C—Cl | C—Cl | C—Cl | C—H | CF₃ | cyclo-PrCO | Me | H | Cl |
| T4 | — | 311 | H | C—Cl | C—Cl | C—Cl | C—H | CF₃ | cyclo-PrCH2CO | Me | H | Cl |
| T4 | — | 312 | H | C—Cl | C—Cl | C—Cl | C—H | CF₃ | CF3CH2CO | Me | H | Cl |
| T4 | | 313 | H | C—CF3 | N | C—H | C—H | CF₃ | H | H | H | Cl |
| T4 | | 314 | H | C—CF3 | N | C—H | C—H | CF₃ | MeCO | H | H | Cl |
| T4 | | 315 | H | C—CF3 | N | C—H | C—H | CF₃ | EtCO | H | H | Cl |
| T4 | | 316 | H | C—CF3 | N | C—H | C—H | CF₃ | n-PrCO | H | H | Cl |
| T4 | | 317 | H | C—CF3 | N | C—H | C—H | CF₃ | cyclo-PrCO | H | H | Cl |
| T4 | | 318 | H | C—CF3 | N | C—H | C—H | CF₃ | cyclo-PrCH2CO | H | H | Cl |
| T4 | | 319 | H | C—CF3 | N | C—H | C—H | CF₃ | CF3CH2CO | H | H | Cl |
| T4 | | 320 | H | C—Cl | N | C—H | C—H | CF₃ | H | H | H | Cl |
| T4 | | 321 | H | C—Cl | N | C—H | C—H | CF₃ | MeCO | H | H | Cl |
| T4 | | 322 | H | C—Cl | N | C—H | C—H | CF₃ | EtCO | H | H | Cl |
| T4 | | 323 | H | C—Cl | N | C—H | C—H | CF₃ | n-PrCO | H | H | Cl |
| T4 | | 324 | H | C—Cl | N | C—H | C—H | CF₃ | cyclo-PrCO | H | H | Cl |
| T4 | | 325 | H | C—Cl | N | C—H | C—H | CF₃ | cyclo-PrCH2CO | H | H | Cl |
| T4 | | 326 | H | C—Cl | N | C—H | C—H | CF₃ | CF3CH2CO | H | H | Cl |
| T4 | | 327 | H | C—Cl | C—F | C—Cl | C—F | CF₃ | H | H | H | Cl |
| T4 | | 328 | H | C—Cl | C—F | C—Cl | C—F | CF₃ | MeCO | H | H | Cl |
| T4 | | 329 | H | C—Cl | C—F | C—Cl | C—F | CF₃ | EtCO | H | H | Cl |
| T4 | | 330 | H | C—Cl | C—F | C—Cl | C—F | CF₃ | n-PrCO | H | H | Cl |
| T4 | | 331 | H | C—Cl | C—F | C—Cl | C—F | CF₃ | cyclo-PrCO | H | H | Cl |
| T4 | | 332 | H | C—Cl | C—F | C—Cl | C—F | CF₃ | cyclo-PrCH2CO | H | H | Cl |
| T4 | | 333 | H | C—Cl | C—F | C—Cl | C—F | CF₃ | CF3CH2CO | H | H | Cl |
| T4 | — | 334 | H | C—Cl | C—H | C—Cl | C—H | CF₃ | H | H | H | Br |
| T4 | — | 335 | H | C—Cl | C—H | C—Cl | C—H | CF₃ | MeCO | H | H | Br |
| T4 | — | 336 | H | C—Cl | C—H | C—Cl | C—H | CF3 | EtCO | H | H | Br |
| T4 | — | 337 | H | C—Cl | C—H | C—Cl | C—H | CF3 | n-PrCO | H | H | Br |
| T4 | — | 338 | H | C—Cl | C—H | C—Cl | C—H | CF3 | cyclo-PrCO | H | H | Br |
| T4 | — | 339 | H | C—Cl | C—H | C—Cl | C—H | CF3 | cyclo-PrCH2CO | H | H | Br |
| T4 | — | 340 | H | C—Cl | C—H | C—Cl | C—H | CF3 | CF3CH2CO | H | H | Br |
| T4 | — | 341 | H | C—Br | C—H | C—Br | C—H | CF3 | H | H | H | Br |
| T4 | — | 342 | H | C—Br | C—H | C—Br | C—H | CF₃ | MeCO | H | H | Br |
| T4 | — | 343 | H | C—Br | C—H | C—Br | C—H | CF₃ | EtCO | H | H | Br |
| T4 | — | 344 | H | C—Br | C—H | C—Br | C—H | CF₃ | n-PrCO | H | H | Br |
| T4 | — | 345 | H | C—Br | C—H | C—Br | C—H | CF₃ | cyclo-PrCO | H | H | Br |
| T4 | — | 346 | H | C—Br | C—H | C—Br | C—H | CF₃ | cyclo-PrCH2CO | H | H | Br |
| T4 | — | 347 | H | C—Br | C—H | C—Br | C—H | CF₃ | CF3CH2CO | H | H | Br |
| T4 | — | 348 | H | C—CF3 | C—H | C—H | C—H | CF3 | H | H | H | Br |
| T4 | — | 349 | H | C—CF3 | C—H | C—H | C—H | CF3 | MeCO | H | H | Br |
| T4 | — | 350 | H | C—CF3 | C—H | C—H | C—H | CF3 | EtCO | H | H | Br |
| T4 | — | 351 | H | C—CF3 | C—H | C—H | C—H | CF3 | n-PrCO | H | H | Br |
| T4 | — | 352 | H | C—CF3 | C—H | C—H | C—H | CF3 | cyclo-PrCO | H | H | Br |
| T4 | — | 353 | H | C—CF3 | C—H | C—H | C—H | CF3 | cyclo-PrCH2CO | H | H | Br |
| T4 | — | 354 | H | C—CF3 | C—H | C—H | C—H | CF3 | CF3CH2CO | H | H | Br |
| T4 | — | 355 | H | C—CF3 | C—H | C—CF3 | C—H | CF3 | H | H | H | Br |
| T4 | — | 356 | H | C—CF3 | C—H | C—CF3 | C—H | CF3 | MeCO | H | H | Br |
| T4 | — | 357 | H | C—CF3 | C—H | C—CF3 | C—H | CF3 | EtCO | H | H | Br |
| T4 | — | 358 | H | C—CF3 | C—H | C—CF3 | C—H | CF3 | n-PrCO | H | H | Br |
| T4 | — | 359 | H | C—CF3 | C—H | C—CF3 | C—H | CF3 | cyclo-PrCO | H | H | Br |
| T4 | — | 360 | H | C—CF3 | C—H | C—CF3 | C—H | CF3 | cyclo-PrCH2CO | H | H | Br |
| T4 | — | 361 | H | C—CF3 | C—H | C—CF3 | C—H | CF3 | CF3CH2CO | H | H | Br |
| T4 | — | 362 | H | C—CF3 | C—H | C—Cl | C—H | CF3 | H | H | H | Br |
| T4 | — | 363 | H | C—CF3 | C—H | C—Cl | C—H | CF3 | MeCO | H | H | Br |
| T4 | — | 364 | H | C—CF3 | C—H | C—Cl | C—H | CF3 | EtCO | H | H | Br |
| T4 | — | 365 | H | C—CF3 | C—H | C—Cl | C—H | CF3 | n-PrCO | H | H | Br |
| T4 | — | 366 | H | C—CF3 | C—H | C—Cl | C—H | CF3 | cyclo-PrCO | H | H | Br |
| T4 | — | 367 | H | C—CF3 | C—H | C—Cl | C—H | CF3 | cyclo-PrCH2CO | H | H | Br |
| T4 | — | 368 | H | C—CF3 | C—H | C—Cl | C—H | CF3 | CF3CH2CO | H | H | Br |
| T4 | — | 369 | H | C—CF3 | C—F | C—H | C—H | CF3 | H | H | H | Br |
| T4 | — | 370 | H | C—CF3 | C—F | C—H | C—H | CF₃ | MeCO | H | H | Br |
| T4 | — | 371 | H | C—CF3 | C—F | C—H | C—H | CF₃ | EtCO | H | H | Br |
| T4 | — | 372 | H | C—CF3 | C—F | C—H | C—H | CF₃ | n-PrCO | H | H | Br |
| T4 | — | 373 | H | C—CF3 | C—F | C—H | C—H | CF₃ | cyclo-PrCO | H | H | Br |
| T4 | — | 374 | H | C—CF3 | C—F | C—H | C—H | CF₃ | cyclo-PrCH2CO | H | H | Br |
| T4 | — | 375 | H | C—CF3 | C—F | C—H | C—H | CF₃ | CF3CH2CO | H | H | Br |
| T4 | — | 376 | H | C—CF3 | C—H | C—F | C—H | CF₃ | H | H | H | Br |
| T4 | — | 377 | H | C—CF3 | C—H | C—F | C—H | CF₃ | MeCO | H | H | Br |
| T4 | — | 378 | H | C—CF3 | C—H | C—F | C—H | CF₃ | EtCO | H | H | Br |
| T4 | — | 379 | H | C—CF3 | C—H | C—F | C—H | CF₃ | n-PrCO | H | H | Br |
| T4 | — | 380 | H | C—CF3 | C—H | C—F | C—H | CF₃ | cyclo-PrCO | H | H | Br |
| T4 | — | 381 | H | C—CF3 | C—H | C—F | C—H | CF₃ | cyclo-PrCH2CO | H | H | Br |
| T4 | — | 382 | H | C—CF3 | C—H | C—F | C—H | CF₃ | CF3CH2CO | H | H | Br |
| T4 | — | 383 | H | C—Cl | C—Cl | C—Cl | C—H | CF₃ | H | H | H | Br |
| T4 | — | 384 | H | C—Cl | C—Cl | C—Cl | C—H | CF₃ | MeCO | H | H | Br |
| T4 | — | 385 | H | C—Cl | C—Cl | C—Cl | C—H | CF₃ | n-PrCO | H | H | Br |
| T4 | — | 386 | H | C—Cl | C—Cl | C—Cl | C—H | CF₃ | cyclo-PrCO | H | H | Br |
| T4 | — | 387 | H | C—Cl | C—Cl | C—Cl | C—H | CF₃ | cyclo-PrCH2CO | H | H | Br |
| T4 | — | 388 | H | C—Cl | C—Cl | C—Cl | C—H | CF₃ | CF3CH2CO | H | H | Br |
| T4 | — | 389 | H | C—Cl | C—Cl | C—CF3 | C—H | CF₃ | H | H | H | Br |
| T4 | — | 390 | H | C—Cl | C—Cl | C—CF3 | C—H | CF₃ | MeCO | H | H | Br |
| T4 | — | 391 | H | C—Cl | C—Cl | C—CF3 | C—H | CF₃ | EtCO | H | H | Br |
| T4 | — | 392 | H | C—Cl | C—Cl | C—CF3 | C—H | CF₃ | n-PrCO | H | H | Br |
| T4 | — | 393 | H | C—Cl | C—Cl | C—CF3 | C—H | CF₃ | cyclo-PrCO | H | H | Br |
| T4 | — | 394 | H | C—Cl | C—Cl | C—CF3 | C—H | CF₃ | cyclo-PrCH2CO | H | H | Br |
| T4 | — | 395 | H | C—Cl | C—Cl | C—CF3 | C—H | CF₃ | CF3CH2CO | H | H | Br |
| T4 | — | 396 | H | C—Cl | N | C—Cl | C—H | CF₃ | H | H | H | Br |
| T4 | — | 397 | H | C—Cl | N | C—Cl | C—H | CF₃ | MeCO | H | H | Br |
| T4 | — | 398 | H | C—Cl | N | C—Cl | C—H | CF₃ | EtCO | H | H | Br |
| T4 | — | 399 | H | C—Cl | N | C—Cl | C—H | CF₃ | n-PrCO | H | H | Br |
| T4 | — | 400 | H | C—Cl | N | C—Cl | C—H | CF₃ | cyclo-PrCO | H | H | Br |
| T4 | — | 401 | H | C—Cl | N | C—Cl | C—H | CF₃ | cyclo-PrCH2CO | H | H | Br |
| T4 | — | 402 | H | C—Cl | N | C—Cl | C—H | CF₃ | CF3CH2CO | H | H | Br |
| T4 | — | 403 | H | C—CF3 | N | C—CF3 | C—H | CF₃ | H | H | H | Br |
| T4 | — | 404 | H | C—CF3 | N | C—CF3 | C—H | CF₃ | MeCO | H | H | Br |
| T4 | — | 405 | H | C—CF3 | N | C—CF3 | C—H | CF₃ | EtCO | H | H | Br |
| T4 | — | 406 | H | C—CF3 | N | C—CF3 | C—H | CF₃ | n-PrCO | H | H | Br |
| T4 | — | 407 | H | C—CF3 | N | C—CF3 | C—H | CF₃ | cyclo-PrCO | H | H | Br |
| T4 | — | 408 | H | C—CF3 | N | C—CF3 | C—H | CF₃ | cyclo-PrCH2CO | H | H | Br |
| T4 | — | 409 | H | C—CF3 | N | C—CF3 | C—H | CF₃ | CF3CH2CO | H | H | Br |
| T4 | — | 410 | H | C—CF3 | N | C—Cl | C—H | CF₃ | H | H | H | Br |
| T4 | — | 411 | H | C—CF3 | N | C—Cl | C—H | CF₃ | MeCO | H | H | Br |
| T4 | — | 412 | H | C—CF3 | N | C—Cl | C—H | CF₃ | EtCO | H | H | Br |
| T4 | — | 413 | H | C—CF3 | N | C—Cl | C—H | CF₃ | n-PrCO | H | H | Br |
| T4 | — | 414 | H | C—CF3 | N | C—Cl | C—H | CF₃ | cyclo-PrCO | H | H | Br |
| T4 | — | 415 | H | C—CF3 | N | C—Cl | C—H | CF₃ | cyclo-PrCH2CO | H | H | Br |
| T4 | — | 416 | H | C—CF3 | N | C—Cl | C—H | CF₃ | CF3CH2CO | H | H | Br |
| T4 | — | 417 | H | C—Cl | C—Cl | C—Cl | C—H | CF₃ | H | Me | H | Br |
| T4 | — | 418 | H | C—Cl | C—Cl | C—Cl | C—H | CF₃ | MeCO | Me | H | Br |
| T4 | — | 419 | H | C—Cl | C—Cl | C—Cl | C—H | CF₃ | EtCO | Me | H | Br |
| T4 | — | 420 | H | C—Cl | C—Cl | C—Cl | C—H | CF₃ | n-PrCO | Me | H | Br |
| T4 | — | 421 | H | C—Cl | C—Cl | C—Cl | C—H | CF₃ | cyclo-PrCO | Me | H | Br |
| T4 | — | 422 | H | C—Cl | C—Cl | C—Cl | C—H | CF₃ | cyclo-PrCH2CO | Me | H | Br |
| T4 | — | 423 | H | C—Cl | C—Cl | C—Cl | C—H | CF₃ | CF3CH2CO | Me | H | Br |
| T4 | | 424 | H | C—CF3 | N | C—H | C—H | CF₃ | H | H | H | Br |
| T4 | | 425 | H | C—CF3 | N | C—H | C—H | CF₃ | MeCO | H | H | Br |
| T4 | | 426 | H | C—CF3 | N | C—H | C—H | CF₃ | EtCO | H | H | Br |
| T4 | | 427 | H | C—CF3 | N | C—H | C—H | CF₃ | n-PrCO | H | H | Br |
| T4 | | 428 | H | C—CF3 | N | C—H | C—H | CF₃ | cyclo-PrCO | H | H | Br |
| T4 | | 429 | H | C—CF3 | N | C—H | C—H | CF₃ | cyclo-PrCH2CO | H | H | Br |
| T4 | | 430 | H | C—CF3 | N | C—H | C—H | CF₃ | CF3CH2CO | H | H | Br |
| T4 | | 431 | H | C—Cl | N | C—H | C—H | CF₃ | H | H | H | Br |
| T4 | | 432 | H | C—Cl | N | C—H | C—H | CF₃ | MeCO | H | H | Br |
| T4 | | 433 | H | C—Cl | N | C—H | C—H | CF₃ | EtCO | H | H | Br |
| T4 | | 434 | H | C—Cl | N | C—H | C—H | CF₃ | n-PrCO | H | H | Br |
| T4 | | 435 | H | C—Cl | N | C—H | C—H | CF₃ | cyclo-PrCO | H | H | Br |
| T4 | | 436 | H | C—Cl | N | C—H | C—H | CF₃ | cyclo-PrCH2CO | H | H | Br |
| T4 | | 437 | H | C—Cl | N | C—H | C—H | CF₃ | CF3CH2CO | H | H | Br |
| T4 | | 438 | H | C—Cl | C—F | C—Cl | C—F | CF₃ | H | H | H | Br |
| T4 | | 439 | H | C—Cl | C—F | C—Cl | C—F | CF₃ | MeCO | H | H | Br |
| T4 | | 440 | H | C—Cl | C—F | C—Cl | C—F | CF₃ | EtCO | H | H | Br |
| T4 | | 441 | H | C—Cl | C—F | C—Cl | C—F | CF₃ | n-PrCO | H | H | Br |
| T4 | | 442 | H | C—Cl | C—F | C—Cl | C—F | CF₃ | cyclo-PrCO | H | H | Br |
| T4 | | 443 | H | C—Cl | C—H | C—Cl | C—F | CF₃ | cyclo-PrCH2CO | H | H | Br |
| T4 | | 444 | H | C—Cl | C—H | C—Cl | C—F | CF₃ | CF3CH2CO | H | H | Br |
| T4 | — | 445 | H | C—Cl | C—H | C—Cl | C—H | CF₃ | H | H | H | CH₃ |
| T4 | — | 446 | H | C—Cl | C—H | C—Cl | C—H | CF₃ | MeCO | H | H | CH₃ |
| T4 | — | 447 | H | C—Cl | C—H | C—Cl | C—H | CF3 | EtCO | H | H | CH₃ |
| T4 | — | 448 | H | C—Cl | C—H | C—Cl | C—H | CF3 | n-PrCO | H | H | CH₃ |
| T4 | — | 449 | H | C—Cl | C—H | C—Cl | C—H | CF3 | cyclo-PrCO | H | H | CH₃ |
| T4 | — | 450 | H | C—Cl | C—H | C—Cl | C—H | CF3 | cyclo-PrCH2CO | H | H | CH₃ |
| T4 | — | 451 | H | C—Cl | C—H | C—Cl | C—H | CF3 | CF3CH2CO | H | H | CH₃ |
| T4 | — | 452 | H | C—Br | C—H | C—Br | C—H | CF3 | H | H | H | CH₃ |
| T4 | — | 453 | H | C—Br | C—H | C—Br | C—H | CF₃ | MeCO | H | H | CH₃ |
| T4 | — | 454 | H | C—Br | C—H | C—Br | C—H | CF₃ | EtCO | H | H | CH₃ |
| T4 | — | 455 | H | C—Br | C—H | C—Br | C—H | CF₃ | n-PrCO | H | H | CH₃ |
| T4 | — | 456 | H | C—Br | C—H | C—Br | C—H | CF₃ | cyclo-PrCO | H | H | CH₃ |
| T4 | — | 457 | H | C—Br | C—H | C—Br | C—H | CF₃ | cyclo-PrCH2CO | H | H | CH₃ |
| T4 | — | 458 | H | C—Br | C—H | C—Br | C—H | CF₃ | CF3CH2CO | H | H | CH₃ |
| T4 | — | 459 | H | C—CF3 | C—H | C—H | C—H | CF3 | H | H | H | CH₃ |
| T4 | — | 460 | H | C—CF3 | C—H | C—H | C—H | CF3 | MeCO | H | H | CH₃ |
| T4 | — | 461 | H | C—CF3 | C—H | C—H | C—H | CF3 | EtCO | H | H | CH₃ |
| T4 | — | 462 | H | C—CF3 | C—H | C—H | C—H | CF3 | n-PrCO | H | H | CH₃ |
| T4 | — | 463 | H | C—CF3 | C—H | C—H | C—H | CF3 | cyclo-PrCO | H | H | CH₃ |
| T4 | — | 464 | H | C—CF3 | C—H | C—H | C—H | CF3 | cyclo-PrCH2CO | H | H | CH₃ |
| T4 | — | 465 | H | C—CF3 | C—H | C—H | C—H | CF3 | CF3CH2CO | H | H | CH₃ |
| T4 | — | 466 | H | C—CF3 | C—H | C—CF3 | C—H | CF3 | H | H | H | CH₃ |
| T4 | — | 467 | H | C—CF3 | C—H | C—CF3 | C—H | CF3 | MeCO | H | H | CH₃ |
| T4 | — | 468 | H | C—CF3 | C—H | C—CF3 | C—H | CF3 | EtCO | H | H | CH₃ |
| T4 | — | 469 | H | C—CF3 | C—H | C—CF3 | C—H | CF3 | n-PrCO | H | H | CH₃ |
| T4 | — | 470 | H | C—CF3 | C—H | C—CF3 | C—H | CF3 | cyclo-PrCO | H | H | CH₃ |
| T4 | — | 471 | H | C—CF3 | C—H | C—CF3 | C—H | CF3 | cyclo-PrCH2CO | H | H | CH₃ |
| T4 | — | 472 | H | C—CF3 | C—H | C—CF3 | C—H | CF3 | CF3CH2CO | H | H | CH₃ |
| T4 | — | 473 | H | C—CF3 | C—H | C—Cl | C—H | CF3 | H | H | H | CH₃ |
| T4 | — | 474 | H | C—CF3 | C—H | C—Cl | C—H | CF3 | MeCO | H | H | CH₃ |
| T4 | — | 475 | H | C—CF3 | C—H | C—Cl | C—H | CF3 | EtCO | H | H | CH₃ |
| T4 | — | 476 | H | C—CF3 | C—H | C—Cl | C—H | CF3 | n-PrCO | H | H | CH₃ |
| T4 | — | 477 | H | C—CF3 | C—H | C—Cl | C—H | CF3 | cyclo-PrCO | H | H | CH₃ |
| T4 | — | 478 | H | C—CF3 | C—H | C—Cl | C—H | CF3 | cyclo-PrCH2CO | H | H | CH₃ |
| T4 | — | 479 | H | C—CF3 | C—H | C—Cl | C—H | CF3 | CF3CH2CO | H | H | CH₃ |
| T4 | — | 480 | H | C—CF3 | C—F | C—H | C—H | CF3 | H | H | H | CH₃ |
| T4 | — | 481 | H | C—CF3 | C—F | C—H | C—H | CF₃ | MeCO | H | H | CH₃ |
| T4 | — | 482 | H | C—CF3 | C—F | C—H | C—H | CF₃ | EtCO | H | H | CH₃ |
| T4 | — | 483 | H | C—CF3 | C—F | C—H | C—H | CF₃ | n-PrCO | H | H | CH₃ |
| T4 | — | 484 | H | C—CF3 | C—F | C—H | C—H | CF₃ | cyclo-PrCO | H | H | CH₃ |
| T4 | — | 485 | H | C—CF3 | C—F | C—H | C—H | CF₃ | cyclo-PrCH2CO | H | H | CH₃ |
| T4 | — | 486 | H | C—CF3 | C—F | C—H | C—H | CF₃ | CF3CH2CO | H | H | CH₃ |
| T4 | — | 487 | H | C—CF3 | C—H | C—F | C—H | CF₃ | H | H | H | CH₃ |
| T4 | — | 488 | H | C—CF3 | C—H | C—F | C—H | CF₃ | MeCO | H | H | CH₃ |
| T4 | — | 489 | H | C—CF3 | C—H | C—F | C—H | CF₃ | EtCO | H | H | CH₃ |
| T4 | — | 490 | H | C—CF3 | C—H | C—F | C—H | CF₃ | n-PrCO | H | H | CH₃ |
| T4 | — | 491 | H | C—CF3 | C—H | C—F | C—H | CF₃ | cyclo-PrCO | H | H | CH₃ |
| T4 | — | 492 | H | C—CF3 | C—H | C—F | C—H | CF₃ | cyclo-PrCH2CO | H | H | CH₃ |
| T4 | — | 493 | H | C—CF3 | C—H | C—F | C—H | CF₃ | CF3CH2CO | H | H | CH₃ |
| T4 | — | 494 | H | C—Cl | C—Cl | C—Cl | C—H | CF₃ | H | H | H | CH₃ |
| T4 | — | 495 | H | C—Cl | C—Cl | C—Cl | C—H | CF₃ | MeCO | H | H | CH₃ |
| T4 | — | 496 | H | C—Cl | C—Cl | C—Cl | C—H | CF₃ | n-PrCO | H | H | CH₃ |
| T4 | — | 497 | H | C—Cl | C—Cl | C—Cl | C—H | CF₃ | cyclo-PrCO | H | H | CH₃ |
| T4 | — | 498 | H | C—Cl | C—Cl | C—Cl | C—H | CF₃ | cyclo-PrCH2CO | H | H | CH₃ |
| T4 | — | 499 | H | C—Cl | C—Cl | C—Cl | C—H | CF₃ | CF3CH2CO | H | H | CH₃ |
| T4 | — | 500 | H | C—Cl | C—Cl | C—CF3 | C—H | CF₃ | H | H | H | CH₃ |
| T4 | — | 501 | H | C—Cl | C—Cl | C—CF3 | C—H | CF₃ | MeCO | H | H | CH₃ |
| T4 | — | 502 | H | C—Cl | C—Cl | C—CF3 | C—H | CF₃ | EtCO | H | H | CH₃ |
| T4 | — | 503 | H | C—Cl | C—Cl | C—CF3 | C—H | CF₃ | n-PrCO | H | H | CH₃ |
| T4 | — | 504 | H | C—Cl | C—Cl | C—CF3 | C—H | CF₃ | cyclo-PrCO | H | H | CH₃ |
| T4 | — | 505 | H | C—Cl | C—Cl | C—CF3 | C—H | CF₃ | cyclo-PrCH2CO | H | H | CH₃ |
| T4 | — | 506 | H | C—Cl | C—Cl | C—CF3 | C—H | CF₃ | CF3CH2CO | H | H | CH₃ |
| T4 | — | 507 | H | C—Cl | N | C—Cl | C—H | CF₃ | H | H | H | CH₃ |
| T4 | — | 508 | H | C—Cl | N | C—Cl | C—H | CF₃ | MeCO | H | H | CH₃ |
| T4 | — | 509 | H | C—Cl | N | C—Cl | C—H | CF₃ | EtCO | H | H | CH₃ |
| T4 | — | 510 | H | C—Cl | N | C—Cl | C—H | CF₃ | n-PrCO | H | H | CH₃ |
| T4 | — | 511 | H | C—Cl | N | C—Cl | C—H | CF₃ | cyclo-PrCO | H | H | CH₃ |
| T4 | — | 512 | H | C—Cl | N | C—Cl | C—H | CF₃ | cyclo-PrCH2CO | H | H | CH₃ |
| T4 | — | 513 | H | C—Cl | N | C—Cl | C—H | CF₃ | CF3CH2CO | H | H | CH₃ |
| T4 | — | 514 | H | C—CF3 | N | C—CF3 | C—H | CF₃ | H | H | H | CH₃ |
| T4 | — | 515 | H | C—CF3 | N | C—CF3 | C—H | CF₃ | MeCO | H | H | CH₃ |
| T4 | — | 516 | H | C—CF3 | N | C—CF3 | C—H | CF₃ | EtCO | H | H | CH₃ |
| T4 | — | 517 | H | C—CF3 | N | C—CF3 | C—H | CF₃ | n-PrCO | H | H | CH₃ |
| T4 | — | 518 | H | C—CF3 | N | C—CF3 | C—H | CF₃ | cyclo-PrCO | H | H | CH₃ |
| T4 | — | 519 | H | C—CF3 | N | C—CF3 | C—H | CF₃ | cyclo-PrCH2CO | H | H | CH₃ |
| T4 | — | 520 | H | C—CF3 | N | C—CF3 | C—H | CF₃ | CF3CH2CO | H | H | CH₃ |
| T4 | — | 521 | H | C—CF3 | N | C—Cl | C—H | CF₃ | H | H | H | CH₃ |
| T4 | — | 522 | H | C—CF3 | N | C—Cl | C—H | CF₃ | MeCO | H | H | CH₃ |
| T4 | — | 523 | H | C—CF3 | N | C—Cl | C—H | CF₃ | EtCO | H | H | CH₃ |
| T4 | — | 524 | H | C—CF3 | N | C—Cl | C—H | CF₃ | n-PrCO | H | H | CH₃ |
| T4 | — | 525 | H | C—CF3 | N | C—Cl | C—H | CF₃ | cyclo-PrCO | H | H | CH₃ |
| T4 | — | 526 | H | C—CF3 | N | C—Cl | C—H | CF₃ | cyclo-PrCH2CO | H | H | CH₃ |
| T4 | — | 527 | H | C—CF3 | N | C—Cl | C—H | CF₃ | CF3CH2CO | H | H | CH₃ |
| T4 | — | 528 | H | C—Cl | C—Cl | C—Cl | C—H | CF₃ | H | Me | H | CH₃ |
| T4 | — | 529 | H | C—Cl | C—Cl | C—Cl | C—H | CF₃ | MeCO | Me | H | CH₃ |
| T4 | — | 530 | H | C—Cl | C—Cl | C—Cl | C—H | CF₃ | EtCO | Me | H | CH₃ |
| T4 | — | 531 | H | C—Cl | C—Cl | C—Cl | C—H | CF₃ | n-PrCO | Me | H | CH₃ |
| T4 | — | 532 | H | C—Cl | C—Cl | C—Cl | C—H | CF₃ | cyclo-PrCO | Me | H | CH₃ |
| T4 | — | 533 | H | C—Cl | C—Cl | C—Cl | C—H | CF₃ | cyclo-PrCH2CO | Me | H | CH₃ |
| T4 | — | 534 | H | C—Cl | C—Cl | C—Cl | C—H | CF₃ | CF3CH2CO | Me | H | CH₃ |
| T4 | | 535 | H | C—CF3 | N | C—H | C—H | CF₃ | H | H | H | CH₃ |
| T4 | | 536 | H | C—CF3 | N | C—H | C—H | CF₃ | MeCO | H | H | CH₃ |
| T4 | | 537 | H | C—CF3 | N | C—H | C—H | CF₃ | EtCO | H | H | CH₃ |
| T4 | | 538 | H | C—CF3 | N | C—H | C—H | CF₃ | n-PrCO | H | H | CH₃ |
| T4 | | 539 | H | C—CF3 | N | C—H | C—H | CF₃ | cyclo-PrCO | H | H | CH₃ |
| T4 | | 540 | H | C—CF3 | N | C—H | C—H | CF₃ | cyclo-PrCH2CO | H | H | CH₃ |
| T4 | | 541 | H | C—CF3 | N | C—H | C—H | CF₃ | CF3CH2CO | H | H | CH₃ |
| T4 | | 542 | H | C—Cl | N | C—H | C—H | CF₃ | H | H | H | CH₃ |
| T4 | | 543 | H | C—Cl | N | C—H | C—H | CF₃ | MeCO | H | H | CH₃ |
| T4 | | 544 | H | C—Cl | N | C—H | C—H | CF₃ | EtCO | H | H | CH₃ |
| T4 | | 545 | H | C—Cl | N | C—H | C—H | CF₃ | n-PrCO | H | H | CH₃ |
| T4 | | 546 | H | C—Cl | N | C—H | C—H | CF₃ | cyclo-PrCO | H | H | CH₃ |
| T4 | | 547 | H | C—Cl | N | C—H | C—H | CF₃ | cyclo-PrCH2CO | H | H | CH₃ |
| T4 | | 548 | H | C—Cl | N | C—H | C—H | CF₃ | CF3CH2CO | H | H | CH₃ |
| T4 | | 549 | H | C—Cl | C—F | C—Cl | C—F | CF₃ | H | H | H | CH₃ |
| T4 | | 550 | H | C—Cl | C—F | C—Cl | C—F | CF₃ | MeCO | H | H | CH₃ |
| T4 | | 551 | H | C—Cl | C—F | C—Cl | C—F | CF₃ | EtCO | H | H | CH₃ |
| T4 | | 552 | H | C—Cl | C—F | C—Cl | C—F | CF₃ | n-PrCO | H | H | CH₃ |
| T4 | | 553 | H | C—Cl | C—F | C—Cl | C—F | CF₃ | cyclo-PrCO | H | H | CH₃ |
| T4 | | 554 | H | C—Cl | C—F | C—Cl | C—F | CF₃ | cyclo-PrCH2CO | H | H | CH₃ |
| T4 | | 555 | H | C—Cl | C—F | C—Cl | C—F | CF₃ | CF3CH2CO | H | H | CH₃ |
| T4 | — | 556 | H | C—Cl | C—H | C—Cl | C—H | CF₃ | H | H | H | CF₃ |
| T4 | — | 557 | H | C—Cl | C—H | C—Cl | C—H | CF₃ | MeCO | H | H | CF₃ |
| T4 | — | 558 | H | C—Cl | C—H | C—Cl | C—H | CF3 | EtCO | H | H | CF₃ |
| T4 | — | 559 | H | C—Cl | C—H | C—Cl | C—H | CF3 | n-PrCO | H | H | CF₃ |
| T4 | — | 560 | H | C—Cl | C—H | C—Cl | C—H | CF3 | cyclo-PrCO | H | H | CF₃ |
| T4 | — | 561 | H | C—Cl | C—H | C—Cl | C—H | CF3 | cyclo-PrCH2CO | H | H | CF₃ |
| T4 | — | 562 | H | C—Cl | C—H | C—Cl | C—H | CF3 | CF3CH2CO | H | H | CF₃ |
| T4 | — | 563 | H | C—Br | C—H | C—Br | C—H | CF3 | H | H | H | CF₃ |
| T4 | — | 564 | H | C—Br | C—H | C—Br | C—H | CF₃ | MeCO | H | H | CF₃ |
| T4 | — | 565 | H | C—Br | C—H | C—Br | C—H | CF₃ | EtCO | H | H | CF₃ |
| T4 | — | 566 | H | C—Br | C—H | C—Br | C—H | CF₃ | n-PrCO | H | H | CF₃ |
| T4 | — | 567 | H | C—Br | C—H | C—Br | C—H | CF₃ | cyclo-PrCO | H | H | CF₃ |
| T4 | — | 568 | H | C—Br | C—H | C—Br | C—H | CF₃ | cyclo-PrCH2CO | H | H | CF₃ |
| T4 | — | 569 | H | C—Br | C—H | C—Br | C—H | CF₃ | CF3CH2CO | H | H | CF₃ |
| T4 | — | 570 | H | C—CF3 | C—H | C—H | C—H | CF3 | H | H | H | CF₃ |
| T4 | — | 571 | H | C—CF3 | C—H | C—H | C—H | CF3 | MeCO | H | H | CF₃ |
| T4 | — | 572 | H | C—CF3 | C—H | C—H | C—H | CF3 | EtCO | H | H | CF₃ |
| T4 | — | 573 | H | C—CF3 | C—H | C—H | C—H | CF3 | n-PrCO | H | H | CF₃ |
| T4 | — | 574 | H | C—CF3 | C—H | C—H | C—H | CF3 | cyclo-PrCO | H | H | CF₃ |
| T4 | — | 575 | H | C—CF3 | C—H | C—H | C—H | CF3 | cyclo-PrCH2CO | H | H | CF₃ |
| T4 | — | 576 | H | C—CF3 | C—H | C—H | C—H | CF3 | CF3CH2CO | H | H | CF₃ |
| T4 | — | 577 | H | C—CF3 | C—H | C—CF3 | C—H | CF3 | H | H | H | CF₃ |
| T4 | — | 578 | H | C—CF3 | C—H | C—CF3 | C—H | CF3 | MeCO | H | H | CF₃ |
| T4 | — | 579 | H | C—CF3 | C—H | C—CF3 | C—H | CF3 | EtCO | H | H | CF₃ |
| T4 | — | 580 | H | C—CF3 | C—H | C—CF3 | C—H | CF3 | n-PrCO | H | H | CF₃ |
| T4 | — | 581 | H | C—CF3 | C—H | C—CF3 | C—H | CF3 | cyclo-PrCO | H | H | CF₃ |
| T4 | — | 582 | H | C—CF3 | C—H | C—CF3 | C—H | CF3 | cyclo-PrCH2CO | H | H | CF₃ |
| T4 | — | 583 | H | C—CF3 | C—H | C—CF3 | C—H | CF3 | CF3CH2CO | H | H | CF₃ |
| T4 | — | 584 | H | C—CF3 | C—H | C—Cl | C—H | CF3 | H | H | H | CF₃ |
| T4 | — | 585 | H | C—CF3 | C—H | C—Cl | C—H | CF3 | MeCO | H | H | CF₃ |
| T4 | — | 586 | H | C—CF3 | C—H | C—Cl | C—H | CF3 | EtCO | H | H | CF₃ |
| T4 | — | 587 | H | C—CF3 | C—H | C—Cl | C—H | CF3 | n-PrCO | H | H | CF₃ |
| T4 | — | 588 | H | C—CF3 | C—H | C—Cl | C—H | CF3 | cyclo-PrCO | H | H | CF₃ |
| T4 | — | 589 | H | C—CF3 | C—H | C—Cl | C—H | CF3 | cyclo-PrCH2CO | H | H | CF₃ |
| T4 | — | 590 | H | C—CF3 | C—H | C—Cl | C—H | CF3 | CF3CH2CO | H | H | CF₃ |
| T4 | — | 591 | H | C—CF3 | C—F | C—H | C—H | CF3 | H | H | H | CF₃ |
| T4 | — | 592 | H | C—CF3 | C—F | C—H | C—H | CF₃ | MeCO | H | H | CF₃ |
| T4 | — | 593 | H | C—CF3 | C—F | C—H | C—H | CF₃ | EtCO | H | H | CF₃ |
| T4 | — | 594 | H | C—CF3 | C—F | C—H | C—H | CF₃ | n-PrCO | H | H | CF₃ |
| T4 | — | 595 | H | C—CF3 | C—F | C—H | C—H | CF₃ | cyclo-PrCO | H | H | CF₃ |
| T4 | — | 596 | H | C—CF3 | C—F | C—H | C—H | CF₃ | cyclo-PrCH2CO | H | H | CF₃ |
| T4 | — | 597 | H | C—CF3 | C—F | C—H | C—H | CF₃ | CF3CH2CO | H | H | CF₃ |
| T4 | — | 598 | H | C—CF3 | C—H | C—F | C—H | CF₃ | H | H | H | CF₃ |
| T4 | — | 599 | H | C—CF3 | C—H | C—F | C—H | CF₃ | MeCO | H | H | CF₃ |
| T4 | — | 600 | H | C—CF3 | C—H | C—F | C—H | CF₃ | EtCO | H | H | CF₃ |
| T4 | — | 601 | H | C—CF3 | C—H | C—F | C—H | CF₃ | n-PrCO | H | H | CF₃ |
| T4 | — | 602 | H | C—CF3 | C—H | C—F | C—H | CF₃ | cyclo-PrCO | H | H | CF₃ |
| T4 | — | 603 | H | C—CF3 | C—H | C—F | C—H | CF₃ | cyclo-PrCH2CO | H | H | CF₃ |
| T4 | — | 604 | H | C—CF3 | C—H | C—F | C—H | CF₃ | CF3CH2CO | H | H | CF₃ |
| T4 | — | 605 | H | C—Cl | C—Cl | C—Cl | C—H | CF₃ | H | H | H | CF₃ |
| T4 | — | 606 | H | C—Cl | C—Cl | C—Cl | C—H | CF₃ | MeCO | H | H | CF₃ |
| T4 | — | 607 | H | C—Cl | C—Cl | C—Cl | C—H | CF₃ | n-PrCO | H | H | CF₃ |
| T4 | — | 608 | H | C—Cl | C—Cl | C—Cl | C—H | CF₃ | cyclo-PrCO | H | H | CF₃ |
| T4 | — | 609 | H | C—Cl | C—Cl | C—Cl | C—H | CF₃ | cyclo-PrCH2CO | H | H | CF₃ |
| T4 | — | 610 | H | C—Cl | C—Cl | C—Cl | C—H | CF₃ | CF3CH2CO | H | H | CF₃ |
| T4 | — | 611 | H | C—Cl | C—Cl | C—CF3 | C—H | CF₃ | H | H | H | CF₃ |
| T4 | — | 612 | H | C—Cl | C—Cl | C—CF3 | C—H | CF₃ | MeCO | H | H | CF₃ |
| T4 | — | 613 | H | C—Cl | C—Cl | C—CF3 | C—H | CF₃ | EtCO | H | H | CF₃ |
| T4 | — | 614 | H | C—Cl | C—Cl | C—CF3 | C—H | CF₃ | n-PrCO | H | H | CF₃ |
| T4 | — | 615 | H | C—Cl | C—Cl | C—CF3 | C—H | CF₃ | cyclo-PrCO | H | H | CF₃ |
| T4 | — | 616 | H | C—Cl | C—Cl | C—CF3 | C—H | CF₃ | cyclo-PrCH2CO | H | H | CF₃ |
| T4 | — | 617 | H | C—Cl | C—Cl | C—CF3 | C—H | CF₃ | CF3CH2CO | H | H | CF₃ |
| T4 | — | 618 | H | C—Cl | N | C—Cl | C—H | CF₃ | H | H | H | CF₃ |
| T4 | — | 619 | H | C—Cl | N | C—Cl | C—H | CF₃ | MeCO | H | H | CF₃ |
| T4 | — | 620 | H | C—Cl | N | C—Cl | C—H | CF₃ | EtCO | H | H | CF₃ |
| T4 | — | 621 | H | C—Cl | N | C—Cl | C—H | CF₃ | n-PrCO | H | H | CF₃ |
| T4 | — | 622 | H | C—Cl | N | C—Cl | C—H | CF₃ | cyclo-PrCO | H | H | CF₃ |
| T4 | — | 623 | H | C—Cl | N | C—Cl | C—H | CF₃ | cyclo-PrCH2CO | H | H | CF₃ |
| T4 | — | 624 | H | C—Cl | N | C—Cl | C—H | CF₃ | CF3CH2CO | H | H | CF₃ |
| T4 | — | 625 | H | C—CF3 | N | C—CF3 | C—H | CF₃ | H | H | H | CF₃ |
| T4 | — | 626 | H | C—CF3 | N | C—CF3 | C—H | CF₃ | MeCO | H | H | CF₃ |
| T4 | — | 627 | H | C—CF3 | N | C—CF3 | C—H | CF₃ | EtCO | H | H | CF₃ |
| T4 | — | 628 | H | C—CF3 | N | C—CF3 | C—H | CF₃ | n-PrCO | H | H | CF₃ |
| T4 | — | 629 | H | C—CF3 | N | C—CF3 | C—H | CF₃ | cyclo-PrCO | H | H | CF₃ |
| T4 | — | 630 | H | C—CF3 | N | C—CF3 | C—H | CF₃ | cyclo-PrCH2CO | H | H | CF₃ |
| T4 | — | 631 | H | C—CF3 | N | C—CF3 | C—H | CF₃ | CF3CH2CO | H | H | CF₃ |
| T4 | — | 632 | H | C—CF3 | N | C—Cl | C—H | CF₃ | H | H | H | CF₃ |
| T4 | — | 633 | H | C—CF3 | N | C—Cl | C—H | CF₃ | MeCO | H | H | CF₃ |
| T4 | — | 634 | H | C—CF3 | N | C—Cl | C—H | CF₃ | EtCO | H | H | CF₃ |
| T4 | — | 635 | H | C—CF3 | N | C—Cl | C—H | CF₃ | n-PrCO | H | H | CF₃ |
| T4 | — | 636 | H | C—CF3 | N | C—Cl | C—H | CF₃ | cyclo-PrCO | H | H | CF₃ |
| T4 | — | 637 | H | C—CF3 | N | C—Cl | C—H | CF₃ | cyclo-PrCH2CO | H | H | CF₃ |
| T4 | — | 638 | H | C—CF3 | N | C—Cl | C—H | CF₃ | CF3CH2CO | H | H | CF₃ |
| T4 | — | 639 | H | C—Cl | C—Cl | C—Cl | C—H | CF₃ | H | Me | H | CF₃ |
| T4 | — | 640 | H | C—Cl | C—Cl | C—Cl | C—H | CF₃ | MeCO | Me | H | CF₃ |
| T4 | — | 641 | H | C—Cl | C—Cl | C—Cl | C—H | CF₃ | EtCO | Me | H | CF₃ |
| T4 | — | 642 | H | C—Cl | C—Cl | C—Cl | C—H | CF₃ | n-PrCO | Me | H | CF₃ |
| T4 | — | 643 | H | C—Cl | C—Cl | C—Cl | C—H | CF₃ | cyclo-PrCO | Me | H | CF₃ |
| T4 | — | 644 | H | C—Cl | C—Cl | C—Cl | C—H | CF₃ | cyclo-PrCH2CO | Me | H | CF₃ |
| T4 | — | 645 | H | C—Cl | C—Cl | C—Cl | C—H | CF₃ | CF3CH2CO | Me | H | CF₃ |
| T4 | | 646 | H | C—CF3 | N | C—H | C—H | CF₃ | H | H | H | CF₃ |
| T4 | | 647 | H | C—CF3 | N | C—H | C—H | CF₃ | MeCO | H | H | CF₃ |
| T4 | | 648 | H | C—CF3 | N | C—H | C—H | CF₃ | EtCO | H | H | CF₃ |
| T4 | | 649 | H | C—CF3 | N | C—H | C—H | CF₃ | n-PrCO | H | H | CF₃ |
| T4 | | 650 | H | C—CF3 | N | C—H | C—H | CF₃ | cyclo-PrCO | H | H | CF₃ |
| T4 | | 651 | H | C—CF3 | N | C—H | C—H | CF₃ | cyclo-PrCH2CO | H | H | CF₃ |
| T4 | | 652 | H | C—CF3 | N | C—H | C—H | CF₃ | CF3CH2CO | H | H | CF₃ |
| T4 | | 653 | H | C—Cl | N | C—H | C—H | CF₃ | H | H | H | CF₃ |
| T4 | | 654 | H | C—Cl | N | C—H | C—H | CF₃ | MeCO | H | H | CF₃ |
| T4 | | 655 | H | C—Cl | N | C—H | C—H | CF₃ | EtCO | H | H | CF₃ |
| T4 | | 656 | H | C—Cl | N | C—H | C—H | CF₃ | n-PrCO | H | H | CF₃ |
| T4 | | 657 | H | C—Cl | N | C—H | C—H | CF₃ | cyclo-PrCO | H | H | CF₃ |
| T4 | | 658 | H | C—Cl | N | C—H | C—H | CF₃ | cyclo-PrCH2CO | H | H | CF₃ |
| T4 | | 659 | H | C—Cl | N | C—H | C—H | CF₃ | CF3CH2CO | H | H | CF₃ |
| T4 | | 660 | H | C—Cl | C—F | C—Cl | C—F | CF₃ | H | H | H | CF₃ |
| T4 | | 661 | H | C—Cl | C—F | C—Cl | C—F | CF₃ | MeCO | H | H | CF₃ |
| T4 | | 662 | H | C—Cl | C—F | C—Cl | C—F | CF₃ | EtCO | H | H | CF₃ |
| T4 | | 663 | H | C—Cl | C—F | C—Cl | C—F | CF₃ | n-PrCO | H | H | CF₃ |
| T4 | | 664 | H | C—Cl | C—F | C—Cl | C—F | CF₃ | cyclo-PrCO | H | H | CF₃ |
| T4 | | 665 | H | C—Cl | C—F | C—Cl | C—F | CF₃ | cyclo-PrCH2CO | H | H | CF₃ |
| T4 | | 666 | H | C—Cl | C—F | C—Cl | C—F | CF₃ | CF3CH2CO | H | H | CF₃ |
| T4 | — | 667 | H | C—Cl | C—H | C—Cl | C—H | CF₃ | H | H | Me | H |
| T4 | — | 668 | H | C—Cl | C—H | C—Cl | C—H | CF₃ | MeCO | H | Me | H |
| T4 | — | 669 | H | C—Cl | C—H | C—Cl | C—H | CF3 | EtCO | H | Me | H |
| T4 | — | 670 | H | C—Cl | C—H | C—Cl | C—H | CF3 | n-PrCO | H | Me | H |
| T4 | — | 671 | H | C—Cl | C—H | C—Cl | C—H | CF3 | cyclo-PrCO | H | Me | H |
| T4 | — | 672 | H | C—Cl | C—H | C—Cl | C—H | CF3 | cyclo-PrCH2CO | H | Me | H |
| T4 | — | 673 | H | C—Cl | C—H | C—Cl | C—H | CF3 | CF3CH2CO | H | Me | H |
| T4 | — | 674 | H | C—Br | C—H | C—Br | C—H | CF3 | H | H | Me | H |
| T4 | — | 675 | H | C—Br | C—H | C—Br | C—H | CF₃ | MeCO | H | Me | H |
| T4 | — | 676 | H | C—Br | C—H | C—Br | C—H | CF₃ | EtCO | H | Me | H |
| T4 | — | 677 | H | C—Br | C—H | C—Br | C—H | CF₃ | n-PrCO | H | Me | H |
| T4 | — | 678 | H | C—Br | C—H | C—Br | C—H | CF₃ | cyclo-PrCO | H | Me | H |
| T4 | — | 679 | H | C—Br | C—H | C—Br | C—H | CF₃ | cyclo-PrCH2CO | H | Me | H |
| T4 | — | 680 | H | C—Br | C—H | C—Br | C—H | CF₃ | CF3CH2CO | H | Me | H |
| T4 | — | 681 | H | C—CF3 | C—H | C—H | C—H | CF3 | H | H | Me | H |
| T4 | — | 682 | H | C—CF3 | C—H | C—H | C—H | CF3 | MeCO | H | Me | H |
| T4 | — | 683 | H | C—CF3 | C—H | C—H | C—H | CF3 | EtCO | H | Me | H |
| T4 | — | 684 | H | C—CF3 | C—H | C—H | C—H | CF3 | n-PrCO | H | Me | H |
| T4 | — | 685 | H | C—CF3 | C—H | C—H | C—H | CF3 | cyclo-PrCO | H | Me | H |
| T4 | — | 686 | H | C—CF3 | C—H | C—H | C—H | CF3 | cyclo-PrCH2CO | H | Me | H |
| T4 | — | 687 | H | C—CF3 | C—H | C—H | C—H | CF3 | CF3CH2CO | H | Me | H |
| T4 | — | 688 | H | C—CF3 | C—H | C—CF3 | C—H | CF3 | H | H | Me | H |
| T4 | — | 689 | H | C—CF3 | C—H | C—CF3 | C—H | CF3 | MeCO | H | Me | H |
| T4 | — | 690 | H | C—CF3 | C—H | C—CF3 | C—H | CF3 | EtCO | H | Me | H |
| T4 | — | 691 | H | C—CF3 | C—H | C—CF3 | C—H | CF3 | n-PrCO | H | Me | H |
| T4 | — | 692 | H | C—CF3 | C—H | C—CF3 | C—H | CF3 | cyclo-PrCO | H | Me | H |
| T4 | — | 693 | H | C—CF3 | C—H | C—CF3 | C—H | CF3 | cyclo-PrCH2CO | H | Me | H |
| T4 | — | 694 | H | C—CF3 | C—H | C—CF3 | C—H | CF3 | CF3CH2CO | H | Me | H |
| T4 | — | 695 | H | C—CF3 | C—H | C—Cl | C—H | CF3 | H | H | Me | H |
| T4 | — | 696 | H | C—CF3 | C—H | C—Cl | C—H | CF3 | MeCO | H | Me | H |
| T4 | — | 697 | H | C—CF3 | C—H | C—Cl | C—H | CF3 | EtCO | H | Me | H |
| T4 | — | 698 | H | C—CF3 | C—H | C—Cl | C—H | CF3 | n-PrCO | H | Me | H |
| T4 | — | 699 | H | C—CF3 | C—H | C—Cl | C—H | CF3 | cyclo-PrCO | H | Me | H |
| T4 | — | 700 | H | C—CF3 | C—H | C—Cl | C—H | CF3 | cyclo-PrCH2CO | H | Me | H |
| T4 | — | 701 | H | C—CF3 | C—H | C—Cl | C—H | CF3 | CF3CH2CO | H | Me | H |
| T4 | — | 702 | H | C—CF3 | C—F | C—H | C—H | CF3 | H | H | Me | H |
| T4 | — | 703 | H | C—CF3 | C—F | C—H | C—H | CF₃ | MeCO | H | Me | H |
| T4 | — | 704 | H | C—CF3 | C—F | C—H | C—H | CF₃ | EtCO | H | Me | H |
| T4 | — | 705 | H | C—CF3 | C—F | C—H | C—H | CF₃ | n-PrCO | H | Me | H |
| T4 | — | 706 | H | C—CF3 | C—F | C—H | C—H | CF₃ | cyclo-PrCO | H | Me | H |
| T4 | — | 707 | H | C—CF3 | C—F | C—H | C—H | CF₃ | cyclo-PrCH2CO | H | Me | H |
| T4 | — | 708 | H | C—CF3 | C—F | C—H | C—H | CF₃ | CF3CH2CO | H | Me | H |
| T4 | — | 709 | H | C—CF3 | C—H | C—F | C—H | CF₃ | H | H | Me | H |
| T4 | — | 710 | H | C—CF3 | C—H | C—F | C—H | CF₃ | MeCO | H | Me | H |
| T4 | — | 711 | H | C—CF3 | C—H | C—F | C—H | CF₃ | EtCO | H | Me | H |
| T4 | — | 712 | H | C—CF3 | C—H | C—F | C—H | CF₃ | n-PrCO | H | Me | H |
| T4 | — | 713 | H | C—CF3 | C—H | C—F | C—H | CF₃ | cyclo-PrCO | H | Me | H |
| T4 | — | 714 | H | C—CF3 | C—H | C—F | C—H | CF₃ | cyclo-PrCH2CO | H | Me | H |
| T4 | — | 715 | H | C—CF3 | C—H | C—F | C—H | CF₃ | CF3CH2CO | H | Me | H |
| T4 | — | 716 | H | C—Cl | C—Cl | C—Cl | C—H | CF₃ | H | H | Me | H |
| T4 | — | 717 | H | C—Cl | C—Cl | C—Cl | C—H | CF₃ | MeCO | H | Me | H |
| T4 | — | 718 | H | C—Cl | C—Cl | C—Cl | C—H | CF₃ | n-PrCO | H | Me | H |
| T4 | — | 719 | H | C—Cl | C—Cl | C—Cl | C—H | CF₃ | cyclo-PrCO | H | Me | H |
| T4 | — | 720 | H | C—Cl | C—Cl | C—Cl | C—H | CF₃ | cyclo-PrCH2CO | H | Me | H |
| T4 | — | 721 | H | C—Cl | C—Cl | C—Cl | C—H | CF₃ | CF3CH2CO | H | Me | H |
| T4 | — | 722 | H | C—Cl | C—Cl | C—CF3 | C—H | CF₃ | H | H | Me | H |
| T4 | — | 723 | H | C—Cl | C—Cl | C—CF3 | C—H | CF₃ | MeCO | H | Me | H |
| T4 | — | 724 | H | C—Cl | C—Cl | C—CF3 | C—H | CF₃ | EtCO | H | Me | H |
| T4 | — | 725 | H | C—Cl | C—Cl | C—CF3 | C—H | CF₃ | n-PrCO | H | Me | H |
| T4 | — | 726 | H | C—Cl | C—Cl | C—CF3 | C—H | CF₃ | cyclo-PrCO | H | Me | H |
| T4 | — | 727 | H | C—Cl | C—Cl | C—CF3 | C—H | CF₃ | cyclo-PrCH2CO | H | Me | H |
| T4 | — | 728 | H | C—Cl | C—Cl | C—CF3 | C—H | CF₃ | CF3CH2CO | H | Me | H |
| T4 | — | 729 | H | C—Cl | N | C—Cl | C—H | CF₃ | H | H | Me | H |
| T4 | — | 730 | H | C—Cl | N | C—Cl | C—H | CF₃ | MeCO | H | Me | H |
| T4 | — | 731 | H | C—Cl | N | C—Cl | C—H | CF₃ | EtCO | H | Me | H |
| T4 | — | 732 | H | C—Cl | N | C—Cl | C—H | CF₃ | n-PrCO | H | Me | H |
| T4 | — | 733 | H | C—Cl | N | C—Cl | C—H | CF₃ | cyclo-PrCO | H | Me | H |
| T4 | — | 734 | H | C—Cl | N | C—Cl | C—H | CF₃ | cyclo-PrCH2CO | H | Me | H |
| T4 | — | 735 | H | C—Cl | N | C—Cl | C—H | CF₃ | CF3CH2CO | H | Me | H |
| T4 | — | 736 | H | C—CF3 | N | C—CF3 | C—H | CF₃ | H | H | Me | H |
| T4 | — | 737 | H | C—CF3 | N | C—CF3 | C—H | CF₃ | MeCO | H | Me | H |
| T4 | — | 738 | H | C—CF3 | N | C—CF3 | C—H | CF₃ | EtCO | H | Me | H |
| T4 | — | 739 | H | C—CF3 | N | C—CF3 | C—H | CF₃ | n-PrCO | H | Me | H |
| T4 | — | 740 | H | C—CF3 | N | C—CF3 | C—H | CF₃ | cyclo-PrCO | H | Me | H |
| T4 | — | 741 | H | C—CF3 | N | C—CF3 | C—H | CF₃ | cyclo-PrCH2CO | H | Me | H |
| T4 | — | 742 | H | C—CF3 | N | C—CF3 | C—H | CF₃ | CF3CH2CO | H | Me | H |
| T4 | — | 743 | H | C—CF3 | N | C—Cl | C—H | CF₃ | H | H | Me | H |
| T4 | — | 744 | H | C—CF3 | N | C—Cl | C—H | CF₃ | MeCO | H | Me | H |
| T4 | — | 745 | H | C—CF3 | N | C—Cl | C—H | CF₃ | EtCO | H | Me | H |
| T4 | — | 746 | H | C—CF3 | N | C—Cl | C—H | CF₃ | n-PrCO | H | Me | H |
| T4 | — | 747 | H | C—CF3 | N | C—Cl | C—H | CF₃ | cyclo-PrCO | H | Me | H |
| T4 | — | 748 | H | C—CF3 | N | C—Cl | C—H | CF₃ | cyclo-PrCH2CO | H | Me | H |
| T4 | — | 749 | H | C—CF3 | N | C—Cl | C—H | CF₃ | CF3CH2CO | H | Me | H |
| T4 | — | 750 | H | C—Cl | C—Cl | C—Cl | C—H | CF₃ | H | Me | Me | H |
| T4 | — | 751 | H | C—Cl | C—Cl | C—Cl | C—H | CF₃ | MeCO | Me | Me | H |
| T4 | — | 752 | H | C—Cl | C—Cl | C—Cl | C—H | CF₃ | EtCO | Me | Me | H |
| T4 | — | 753 | H | C—Cl | C—Cl | C—Cl | C—H | CF₃ | n-PrCO | Me | Me | H |
| T4 | — | 754 | H | C—Cl | C—Cl | C—Cl | C—H | CF₃ | cyclo-PrCO | Me | Me | H |
| T4 | — | 755 | H | C—Cl | C—Cl | C—Cl | C—H | CF₃ | cyclo-PrCH2CO | Me | Me | H |
| T4 | — | 756 | H | C—Cl | C—Cl | C—Cl | C—H | CF₃ | CF3CH2CO | Me | Me | H |
| T4 | | 757 | H | C—CF3 | N | C—H | C—H | CF₃ | H | H | Me | H |
| T4 | | 758 | H | C—CF3 | N | C—H | C—H | CF₃ | MeCO | H | Me | H |
| T4 | | 759 | H | C—CF3 | N | C—H | C—H | CF₃ | EtCO | H | Me | H |
| T4 | | 760 | H | C—CF3 | N | C—H | C—H | CF₃ | n-PrCO | H | Me | H |
| T4 | | 761 | H | C—CF3 | N | C—H | C—H | CF₃ | cyclo-PrCO | H | Me | H |
| T4 | | 762 | H | C—CF3 | N | C—H | C—H | CF₃ | cyclo-PrCH2CO | H | Me | H |
| T4 | | 763 | H | C—CF3 | N | C—H | C—H | CF₃ | CF3CH2CO | H | Me | H |
| T4 | | 764 | H | C—Cl | N | C—H | C—H | CF₃ | H | H | Me | H |
| T4 | | 765 | H | C—Cl | N | C—H | C—H | CF₃ | MeCO | H | Me | H |
| T4 | | 766 | H | C—Cl | N | C—H | C—H | CF₃ | EtCO | H | Me | H |
| T4 | | 767 | H | C—Cl | N | C—H | C—H | CF₃ | n-PrCO | H | Me | H |
| T4 | | 768 | H | C—Cl | N | C—H | C—H | CF₃ | cyclo-PrCO | H | Me | H |
| T4 | | 769 | H | C—Cl | N | C—H | C—H | CF₃ | cyclo-PrCH2CO | H | Me | H |
| T4 | | 770 | H | C—Cl | N | C—H | C—H | CF₃ | CF3CH2CO | H | Me | H |
| T4 | | 771 | H | C—Cl | C—F | C—Cl | C—F | CF₃ | H | H | Me | H |
| T4 | | 772 | H | C—Cl | C—F | C—Cl | C—F | CF₃ | MeCO | H | Me | H |
| T4 | | 773 | H | C—Cl | C—F | C—Cl | C—F | CF₃ | EtCO | H | Me | H |
| T4 | | 774 | H | C—Cl | C—F | C—Cl | C—F | CF₃ | n-PrCO | H | Me | H |
| T4 | | 775 | H | C—Cl | C—F | C—Cl | C—F | CF₃ | cyclo-PrCO | H | Me | H |
| T4 | | 776 | H | C—Cl | C—F | C—Cl | C—F | CF₃ | cyclo-PrCH2CO | H | Me | H |
| T4 | | 777 | H | C—Cl | C—F | C—Cl | C—F | CF₃ | CF3CH2CO | H | Me | H |
| T4 | — | 778 | H | C—Cl | C—H | C—Cl | C—H | CF₃ | H | H | Me | F |
| T4 | — | 779 | H | C—Cl | C—H | C—Cl | C—H | CF₃ | MeCO | H | Me | F |
| T4 | — | 780 | H | C—Cl | C—H | C—Cl | C—H | CF3 | EtCO | H | Me | F |
| T4 | — | 781 | H | C—Cl | C—H | C—Cl | C—H | CF3 | n-PrCO | H | Me | F |
| T4 | — | 782 | H | C—Cl | C—H | C—Cl | C—H | CF3 | cyclo-PrCO | H | Me | F |
| T4 | — | 783 | H | C—Cl | C—H | C—Cl | C—H | CF3 | cyclo-PrCH2O | H | Me | F |
| T4 | — | 784 | H | C—Cl | C—H | C—Cl | C—H | CF3 | CF3CH2CO | H | Me | F |
| T4 | — | 785 | H | C—Br | C—H | C—Br | C—H | CF3 | H | H | Me | F |
| T4 | — | 786 | H | C—Br | C—H | C—Br | C—H | CF₃ | MeCO | H | Me | F |
| T4 | — | 787 | H | C—Br | C—H | C—Br | C—H | CF₃ | EtCO | H | Me | F |
| T4 | — | 788 | H | C—Br | C—H | C—Br | C—H | CF₃ | n-PrCO | H | Me | F |
| T4 | — | 789 | H | C—Br | C—H | C—Br | C—H | CF₃ | cyclo-PrCO | H | Me | F |
| T4 | — | 790 | H | C—Br | C—H | C—Br | C—H | CF₃ | cyclo-PrCH2CO | H | Me | F |
| T4 | — | 791 | H | C—Br | C—H | C—Br | C—H | CF₃ | CF3CH2CO | H | Me | F |
| T4 | — | 792 | H | C—CF3 | C—H | C—H | C—H | CF3 | H | H | Me | F |
| T4 | — | 793 | H | C—CF3 | C—H | C—H | C—H | CF3 | MeCO | H | Me | F |
| T4 | — | 794 | H | C—CF3 | C—H | C—H | C—H | CF3 | EtCO | H | Me | F |
| T4 | — | 795 | H | C—CF3 | C—H | C—H | C—H | CF3 | n-PrCO | H | Me | F |
| T4 | — | 796 | H | C—CF3 | C—H | C—H | C—H | CF3 | cyclo-PrCO | H | Me | F |
| T4 | — | 797 | H | C—CF3 | C—H | C—H | C—H | CF3 | cyclo-PrCH2CO | H | Me | F |
| T4 | — | 798 | H | C—CF3 | C—H | C—H | C—H | CF3 | CF3CH2CO | H | Me | F |
| T4 | — | 799 | H | C—CF3 | C—H | C—CF3 | C—H | CF3 | H | H | Me | F |
| T4 | — | 800 | H | C—CF3 | C—H | C—CF3 | C—H | CF3 | MeCO | H | Me | F |
| T4 | — | 801 | H | C—CF3 | C—H | C—CF3 | C—H | CF3 | EtCO | H | Me | F |
| T4 | — | 802 | H | C—CF3 | C—H | C—CF3 | C—H | CF3 | n-PrCO | H | Me | F |
| T4 | — | 803 | H | C—CF3 | C—H | C—CF3 | C—H | CF3 | cyclo-PrCO | H | Me | F |
| T4 | — | 804 | H | C—CF3 | C—H | C—CF3 | C—H | CF3 | cyclo-PrCH2CO | H | Me | F |
| T4 | — | 805 | H | C—CF3 | C—H | C—CF3 | C—H | CF3 | CF3CH2CO | H | Me | F |
| T4 | — | 806 | H | C—CF3 | C—H | C—Cl | C—H | CF3 | H | H | Me | F |
| T4 | — | 807 | H | C—CF3 | C—H | C—Cl | C—H | CF3 | MeCO | H | Me | F |
| T4 | — | 808 | H | C—CF3 | C—H | C—Cl | C—H | CF3 | EtCO | H | Me | F |
| T4 | — | 809 | H | C—CF3 | C—H | C—Cl | C—H | CF3 | n-PrCO | H | Me | F |
| T4 | — | 810 | H | C—CF3 | C—H | C—Cl | C—H | CF3 | cyclo-PrCO | H | Me | F |
| T4 | — | 811 | H | C—CF3 | C—H | C—Cl | C—H | CF3 | cyclo-PrCH2CO | H | Me | F |
| T4 | — | 812 | H | C—CF3 | C—H | C—Cl | C—H | CF3 | CF3CH2CO | H | Me | F |
| T4 | — | 813 | H | C—CF3 | C—F | C—H | C—H | CF3 | H | H | Me | F |
| T4 | — | 814 | H | C—CF3 | C—F | C—H | C—H | CF₃ | MeCO | H | Me | F |
| T4 | — | 815 | H | C—CF3 | C—F | C—H | C—H | CF₃ | EtCO | H | Me | F |
| T4 | — | 816 | H | C—CF3 | C—F | C—H | C—H | CF₃ | n-PrCO | H | Me | F |
| T4 | — | 817 | H | C—CF3 | C—F | C—H | C—H | CF₃ | cyclo-PrCO | H | Me | F |
| T4 | — | 818 | H | C—CF3 | C—F | C—H | C—H | CF₃ | cyclo-PrCH2CO | H | Me | F |
| T4 | — | 819 | H | C—CF3 | C—F | C—H | C—H | CF₃ | CF3CH2CO | H | Me | F |
| T4 | — | 820 | H | C—CF3 | C—H | C—F | C—H | CF₃ | H | H | Me | F |
| T4 | — | 821 | H | C—CF3 | C—H | C—F | C—H | CF₃ | MeCO | H | Me | F |
| T4 | — | 822 | H | C—CF3 | C—H | C—F | C—H | CF₃ | EtCO | H | Me | F |
| T4 | — | 823 | H | C—CF3 | C—H | C—F | C—H | CF₃ | n-PrCO | H | Me | F |
| T4 | — | 824 | H | C—CF3 | C—H | C—F | C—H | CF₃ | cyclo-PrCO | H | Me | F |
| T4 | — | 825 | H | C—CF3 | C—H | C—F | C—H | CF₃ | cyclo-PrCH2CO | H | Me | F |
| T4 | — | 826 | H | C—CF3 | C—H | C—F | C—H | CF₃ | CF3CH2CO | H | Me | F |
| T4 | — | 827 | H | C—Cl | C—Cl | C—Cl | C—H | CF₃ | H | H | Me | F |
| T4 | — | 828 | H | C—Cl | C—Cl | C—Cl | C—H | CF₃ | MeCO | H | Me | F |
| T4 | — | 829 | H | C—Cl | C—Cl | C—Cl | C—H | CF₃ | n-PrCO | H | Me | F |
| T4 | — | 830 | H | C—Cl | C—Cl | C—Cl | C—H | CF₃ | cyclo-PrCO | H | Me | F |
| T4 | — | 831 | H | C—Cl | C—Cl | C—Cl | C—H | CF₃ | cyclo-PrCH2CO | H | Me | F |
| T4 | — | 832 | H | C—Cl | C—Cl | C—Cl | C—H | CF₃ | CF3CH2CO | H | Me | F |
| T4 | — | 833 | H | C—Cl | C—Cl | C—CF3 | C—H | CF₃ | H | H | Me | F |
| T4 | — | 834 | H | C—Cl | C—Cl | C—CF3 | C—H | CF₃ | MeCO | H | Me | F |
| T4 | — | 835 | H | C—Cl | C—Cl | C—CF3 | C—H | CF₃ | EtCO | H | Me | F |
| T4 | — | 836 | H | C—Cl | C—Cl | C—CF3 | C—H | CF₃ | n-PrCO | H | Me | F |
| T4 | — | 837 | H | C—Cl | C—Cl | C—CF3 | C—H | CF₃ | cyclo-PrCO | H | Me | F |
| T4 | — | 838 | H | C—Cl | C—Cl | C—CF3 | C—H | CF₃ | cyclo-PrCH2CO | H | Me | F |
| T4 | — | 839 | H | C—Cl | C—Cl | C—CF3 | C—H | CF₃ | CF3CH2CO | H | Me | F |
| T4 | — | 840 | H | C—Cl | N | C—Cl | C—H | CF₃ | H | H | Me | F |
| T4 | — | 841 | H | C—Cl | N | C—Cl | C—H | CF₃ | MeCO | H | Me | F |
| T4 | — | 842 | H | C—Cl | N | C—Cl | C—H | CF₃ | EtCO | H | Me | F |
| T4 | — | 843 | H | C—Cl | N | C—Cl | C—H | CF₃ | n-PrCO | H | Me | F |
| T4 | — | 844 | H | C—Cl | N | C—Cl | C—H | CF₃ | cyclo-PrCO | H | Me | F |
| T4 | — | 845 | H | C—Cl | N | C—Cl | C—H | CF₃ | cyclo-PrCH2CO | H | Me | F |
| T4 | — | 846 | H | C—Cl | N | C—Cl | C—H | CF₃ | CF3CH2CO | H | Me | F |
| T4 | — | 847 | H | C—CF3 | N | C—CF3 | C—H | CF₃ | H | H | Me | F |
| T4 | — | 848 | H | C—CF3 | N | C—CF3 | C—H | CF₃ | MeCO | H | Me | F |
| T4 | — | 849 | H | C—CF3 | N | C—CF3 | C—H | CF₃ | EtCO | H | Me | F |
| T4 | — | 850 | H | C—CF3 | N | C—CF3 | C—H | CF₃ | n-PrCO | H | Me | F |
| T4 | — | 851 | H | C—CF3 | N | C—CF3 | C—H | CF₃ | cyclo-PrCO | H | Me | F |
| T4 | — | 852 | H | C—CF3 | N | C—CF3 | C—H | CF₃ | cyclo-PrCH2CO | H | Me | F |
| T4 | — | 853 | H | C—CF3 | N | C—CF3 | C—H | CF₃ | CF3CH2CO | H | Me | F |
| T4 | — | 854 | H | C—CF3 | N | C—Cl | C—H | CF₃ | H | H | Me | F |
| T4 | — | 855 | H | C—CF3 | N | C—Cl | C—H | CF₃ | MeCO | H | Me | F |
| T4 | — | 856 | H | C—CF3 | N | C—Cl | C—H | CF₃ | EtCO | H | Me | F |
| T4 | — | 857 | H | C—CF3 | N | C—Cl | C—H | CF₃ | n-PrCO | H | Me | F |
| T4 | — | 858 | H | C—CF3 | N | C—Cl | C—H | CF₃ | cyclo-PrCO | H | Me | F |
| T4 | — | 859 | H | C—CF3 | N | C—Cl | C—H | CF₃ | cyclo-PrCH2CO | H | Me | F |
| T4 | — | 860 | H | C—CF3 | N | C—Cl | C—H | CF₃ | CF3CH2CO | H | Me | F |
| T4 | — | 861 | H | C—Cl | C—Cl | C—Cl | C—H | CF₃ | H | H | Me | F |
| T4 | — | 862 | H | C—Cl | C—Cl | C—Cl | C—H | CF₃ | MeCO | H | Me | F |
| T4 | — | 863 | H | C—Cl | C—Cl | C—Cl | C—H | CF₃ | EtCO | H | Me | F |
| T4 | — | 864 | H | C—Cl | C—Cl | C—Cl | C—H | CF₃ | n-PrCO | H | Me | F |
| T4 | — | 865 | H | C—Cl | C—Cl | C—Cl | C—H | CF₃ | cyclo-PrCO | H | Me | F |
| T4 | — | 866 | H | C—Cl | C—Cl | C—Cl | C—H | CF₃ | cyclo-PrCH2CO | H | Me | F |
| T4 | — | 867 | H | C—Cl | C—Cl | C—Cl | C—H | CF₃ | CF3CH2CO | H | Me | F |
| T4 | | 868 | H | C—CF3 | N | C—H | C—H | CF₃ | H | H | Me | F |
| T4 | | 869 | H | C—CF3 | N | C—H | C—H | CF₃ | MeCO | H | Me | F |
| T4 | | 870 | H | C—CF3 | N | C—H | C—H | CF₃ | EtCO | H | Me | F |
| T4 | | 871 | H | C—CF3 | N | C—H | C—H | CF₃ | n-PrCO | H | Me | F |
| T4 | | 872 | H | C—CF3 | N | C—H | C—H | CF₃ | cyclo-PrCO | H | Me | F |
| T4 | | 873 | H | C—CF3 | N | C—H | C—H | CF₃ | cyclo-PrCH2CO | H | Me | F |
| T4 | | 874 | H | C—CF3 | N | C—H | C—H | CF₃ | CF3CH2CO | H | Me | F |
| T4 | | 875 | H | C—Cl | N | C—H | C—H | CF₃ | H | H | Me | F |
| T4 | | 876 | H | C—Cl | N | C—H | C—H | CF₃ | MeCO | H | Me | F |
| T4 | | 877 | H | C—Cl | N | C—H | C—H | CF₃ | EtCO | H | Me | F |
| T4 | | 878 | H | C—Cl | N | C—H | C—H | CF₃ | n-PrCO | H | Me | F |
| T4 | | 879 | H | C—Cl | N | C—H | C—H | CF₃ | cyclo-PrCO | H | Me | F |
| T4 | | 880 | H | C—Cl | N | C—H | C—H | CF₃ | cyclo-PrCH2CO | H | Me | F |
| T4 | | 881 | H | C—Cl | N | C—H | C—H | CF₃ | CF3CH2CO | H | Me | F |
| T4 | | 882 | H | C—Cl | C—F | C—Cl | C—F | CF₃ | H | H | Me | F |
| T4 | | 883 | H | C—Cl | C—F | C—Cl | C—F | CF₃ | MeCO | H | Me | F |
| T4 | | 884 | H | C—Cl | C—F | C—Cl | C—F | CF₃ | EtCO | H | Me | F |
| T4 | | 885 | H | C—Cl | C—F | C—Cl | C—F | CF₃ | n-PrCO | H | Me | F |
| T4 | | 886 | H | C—Cl | C—F | C—Cl | C—F | CF₃ | cyclo-PrCO | H | Me | F |
| T4 | | 887 | H | C—Cl | C—F | C—Cl | C—F | CF₃ | cyclo-PrCH2CO | H | Me | F |
| T4 | | 888 | H | C—Cl | C—F | C—Cl | C—F | CF₃ | CF3CH2CO | H | Me | F |
| T4 | — | 889 | H | C—Cl | C—H | C—Cl | C—H | CF₃ | H | H | Me | Cl |
| T4 | — | 890 | H | C—Cl | C—H | C—Cl | C—H | CF₃ | MeCO | H | Me | Cl |
| T4 | — | 891 | H | C—Cl | C—H | C—Cl | C—H | CF3 | EtCO | H | Me | Cl |
| T4 | — | 892 | H | C—Cl | C—H | C—Cl | C—H | CF3 | n-PrCO | H | Me | Cl |
| T4 | — | 893 | H | C—Cl | C—H | C—Cl | C—H | CF3 | cyclo-PrCO | H | Me | Cl |
| T4 | — | 894 | H | C—Cl | C—H | C—Cl | C—H | CF3 | cyclo-PrCH2CO | H | Me | Cl |
| T4 | — | 895 | H | C—Cl | C—H | C—Cl | C—H | CF3 | CF3CH2CO | H | Me | Cl |
| T4 | — | 896 | H | C—Br | C—H | C—Br | C—H | CF3 | H | H | Me | Cl |
| T4 | — | 897 | H | C—Br | C—H | C—Br | C—H | CF₃ | MeCO | H | Me | Cl |
| T4 | — | 898 | H | C—Br | C—H | C—Br | C—H | CF₃ | EtCO | H | Me | Cl |
| T4 | — | 899 | H | C—Br | C—H | C—Br | C—H | CF₃ | n-PrCO | H | Me | Cl |
| T4 | — | 900 | H | C—Br | C—H | C—Br | C—H | CF₃ | cyclo-PrCO | H | Me | Cl |
| T4 | — | 901 | H | C—Br | C—H | C—Br | C—H | CF₃ | cyclo-PrCH2CO | H | Me | Cl |
| T4 | — | 902 | H | C—Br | C—H | C—Br | C—H | CF₃ | CF3CH2CO | H | Me | Cl |
| T4 | — | 903 | H | C—CF3 | C—H | C—H | C—H | CF3 | H | H | Me | Cl |
| T4 | — | 904 | H | C—CF3 | C—H | C—H | C—H | CF3 | MeCO | H | Me | Cl |
| T4 | — | 905 | H | C—CF3 | C—H | C—H | C—H | CF3 | EtCO | H | Me | Cl |
| T4 | — | 906 | H | C—CF3 | C—H | C—H | C—H | CF3 | n-PrCO | H | Me | Cl |
| T4 | — | 907 | H | C—CF3 | C—H | C—H | C—H | CF3 | cyclo-PrCO | H | Me | Cl |
| T4 | — | 908 | H | C—CF3 | C—H | C—H | C—H | CF3 | cyclo-PrCH2CO | H | Me | Cl |
| T4 | — | 909 | H | C—CF3 | C—H | C—H | C—H | CF3 | CF3CH2CO | H | Me | Cl |
| T4 | — | 910 | H | C—CF3 | C—H | C—CF3 | C—H | CF3 | H | H | Me | Cl |
| T4 | — | 911 | H | C—CF3 | C—H | C—CF3 | C—H | CF3 | MeCO | H | Me | Cl |
| T4 | — | 912 | H | C—CF3 | C—H | C—CF3 | C—H | CF3 | EtCO | H | Me | Cl |
| T4 | — | 913 | H | C—CF3 | C—H | C—CF3 | C—H | CF3 | n-PrCO | H | Me | Cl |
| T4 | — | 914 | H | C—CF3 | C—H | C—CF3 | C—H | CF3 | cyclo-PrCO | H | Me | Cl |
| T4 | — | 915 | H | C—CF3 | C—H | C—CF3 | C—H | CF3 | cylco-PrCH2CO | H | Me | Cl |
| T4 | — | 916 | H | C—CF3 | C—H | C—CF3 | C—H | CF3 | CF3CH2CO | H | Me | Cl |
| T4 | — | 917 | H | C—CF3 | C—H | C—Cl | C—H | CF3 | H | H | Me | Cl |
| T4 | — | 918 | H | C—CF3 | C—H | C—Cl | C—H | CF3 | MeCO | H | Me | Cl |
| T4 | — | 919 | H | C—CF3 | C—H | C—Cl | C—H | CF3 | EtCO | H | Me | Cl |
| T4 | — | 920 | H | C—CF3 | C—H | C—Cl | C—H | CF3 | n-PrCO | H | Me | Cl |
| T4 | — | 921 | H | C—CF3 | C—H | C—Cl | C—H | CF3 | cyclo-PrCO | H | Me | Cl |
| T4 | — | 922 | H | C—CF3 | C—H | C—Cl | C—H | CF3 | cyclo-PrCH2CO | H | Me | Cl |
| T4 | — | 923 | H | C—CF3 | C—H | C—Cl | C—H | CF3 | CF3CH2CO | H | Me | Cl |
| T4 | — | 924 | H | C—CF3 | C—F | C—H | C—H | CF3 | H | H | Me | Cl |
| T4 | — | 925 | H | C—CF3 | C—F | C—H | C—H | CF₃ | MeCO | H | Me | Cl |
| T4 | — | 926 | H | C—CF3 | C—F | C—H | C—H | CF₃ | EtCO | H | Me | Cl |
| T4 | — | 927 | H | C—CF3 | C—F | C—H | C—H | CF₃ | n-PrCO | H | Me | Cl |
| T4 | — | 928 | H | C—CF3 | C—F | C—H | C—H | CF₃ | cyclo-PrCO | H | Me | Cl |
| T4 | — | 929 | H | C—CF3 | C—F | C—H | C—H | CF₃ | cyclo-PrCH2CO | H | Me | Cl |
| T4 | — | 930 | H | C—CF3 | C—F | C—H | C—H | CF₃ | CF3CH2CO | H | Me | Cl |
| T4 | — | 931 | H | C—CF3 | C—H | C—F | C—H | CF₃ | H | H | Me | Cl |
| T4 | — | 932 | H | C—CF3 | C—H | C—F | C—H | CF₃ | MeCO | H | Me | Cl |
| T4 | — | 933 | H | C—CF3 | C—H | C—F | C—H | CF₃ | EtCO | H | Me | Cl |
| T4 | — | 934 | H | C—CF3 | C—H | C—F | C—H | CF₃ | n-PrCO | H | Me | Cl |
| T4 | — | 935 | H | C—CF3 | C—H | C—F | C—H | CF₃ | cyclo-PrCO | H | Me | Cl |
| T4 | — | 936 | H | C—CF3 | C—H | C—F | C—H | CF₃ | cyclo-PrCH2CO | H | Me | Cl |
| T4 | — | 937 | H | C—CF3 | C—H | C—F | C—H | CF₃ | CF3CH2CO | H | Me | Cl |
| T4 | — | 938 | H | C—Cl | C—Cl | C—Cl | C—H | CF₃ | H | H | Me | Cl |
| T4 | — | 939 | H | C—Cl | C—Cl | C—Cl | C—H | CF₃ | MeCO | H | Me | Cl |
| T4 | — | 940 | H | C—Cl | C—Cl | C—Cl | C—H | CF₃ | n-PrCO | H | Me | Cl |
| T4 | — | 941 | H | C—Cl | C—Cl | C—Cl | C—H | CF₃ | cyclo-PrCO | H | Me | Cl |
| T4 | — | 942 | H | C—Cl | C—Cl | C—Cl | C—H | CF₃ | cyclo-PrCH2CO | H | Me | Cl |
| T4 | — | 943 | H | C—Cl | C—Cl | C—Cl | C—H | CF₃ | CF3CH2CO | H | Me | Cl |
| T4 | — | 944 | H | C—Cl | C—Cl | C—CF3 | C—H | CF₃ | H | H | Me | Cl |
| T4 | — | 945 | H | C—Cl | C—Cl | C—CF3 | C—H | CF₃ | MeCO | H | Me | Cl |
| T4 | — | 946 | H | C—Cl | C—Cl | C—CF3 | C—H | CF₃ | EtCO | H | Me | Cl |
| T4 | — | 947 | H | C—Cl | C—Cl | C—CF3 | C—H | CF₃ | n-PrCO | H | Me | Cl |
| T4 | — | 948 | H | C—Cl | C—Cl | C—CF3 | C—H | CF₃ | cyclo-PrCO | H | Me | Cl |
| T4 | — | 949 | H | C—Cl | C—Cl | C—CF3 | C—H | CF₃ | cyclo-PrCH2CO | H | Me | Cl |
| T4 | — | 950 | H | C—Cl | C—Cl | C—CF3 | C—H | CF₃ | CF3CH2CO | H | Me | Cl |
| T4 | — | 951 | H | C—Cl | N | C—Cl | C—H | CF₃ | H | H | Me | Cl |
| T4 | — | 952 | H | C—Cl | N | C—Cl | C—H | CF₃ | MeCO | H | Me | Cl |
| T4 | — | 953 | H | C—Cl | N | C—Cl | C—H | CF₃ | EtCO | H | Me | Cl |
| T4 | — | 954 | H | C—Cl | N | C—Cl | C—H | CF₃ | n-PrCO | H | Me | Cl |
| T4 | — | 955 | H | C—Cl | N | C—Cl | C—H | CF₃ | cyclo-PrCO | H | Me | Cl |
| T4 | — | 956 | H | C—Cl | N | C—Cl | C—H | CF₃ | cyclo-PrCH2CO | H | Me | Cl |
| T4 | — | 957 | H | C—Cl | N | C—Cl | C—H | CF₃ | CF3CH2CO | H | Me | Cl |
| T4 | — | 958 | H | C—CF3 | N | C—CF3 | C—H | CF₃ | H | H | Me | Cl |
| T4 | — | 959 | H | C—CF3 | N | C—CF3 | C—H | CF₃ | MeCO | H | Me | Cl |
| T4 | — | 960 | H | C—CF3 | N | C—CF3 | C—H | CF₃ | EtCO | H | Me | Cl |
| T4 | — | 961 | H | C—CF3 | N | C—CF3 | C—H | CF₃ | n-PrCO | H | Me | Cl |
| T4 | — | 962 | H | C—CF3 | N | C—CF3 | C—H | CF₃ | cyclo-PrCO | H | Me | Cl |
| T4 | — | 963 | H | C—CF3 | N | C—CF3 | C—H | CF₃ | cyclo-PrCH2CO | H | Me | Cl |
| T4 | — | 964 | H | C—CF3 | N | C—CF3 | C—H | CF₃ | CF3CH2CO | H | Me | Cl |
| T4 | — | 965 | H | C—CF3 | N | C—Cl | C—H | CF₃ | H | H | Me | Cl |
| T4 | — | 966 | H | C—CF3 | N | C—Cl | C—H | CF₃ | MeCO | H | Me | Cl |
| T4 | — | 967 | H | C—CF3 | N | C—Cl | C—H | CF₃ | EtCO | H | Me | Cl |
| T4 | — | 968 | H | C—CF3 | N | C—Cl | C—H | CF₃ | n-PrCO | H | Me | Cl |
| T4 | — | 969 | H | C—CF3 | N | C—Cl | C—H | CF₃ | cyclo-PrCO | H | Me | Cl |
| T4 | — | 970 | H | C—CF3 | N | C—Cl | C—H | CF₃ | cyclo-PrCH2CO | H | Me | Cl |
| T4 | — | 971 | H | C—CF3 | N | C—Cl | C—H | CF₃ | CF3CH2CO | H | Me | Cl |
| T4 | — | 972 | H | C—Cl | C—Cl | C—Cl | C—H | CF₃ | H | Me | Me | Cl |
| T4 | — | 973 | H | C—Cl | C—Cl | C—Cl | C—H | CF₃ | MeCO | Me | Me | Cl |
| T4 | — | 974 | H | C—Cl | C—Cl | C—Cl | C—H | CF₃ | EtCO | Me | Me | Cl |
| T4 | — | 975 | H | C—Cl | C—Cl | C—Cl | C—H | CF₃ | n-PrCO | Me | Me | Cl |
| T4 | — | 976 | H | C—Cl | C—Cl | C—Cl | C—H | CF₃ | cyclo-PrCO | Me | Me | Cl |
| T4 | — | 977 | H | C—Cl | C—Cl | C—Cl | C—H | CF₃ | cyclo-PrCH2CO | Me | Me | Cl |
| T4 | — | 978 | H | C—Cl | C—Cl | C—Cl | C—H | CF₃ | CF3CH2CO | Me | Me | Cl |
| T4 | | 979 | H | C—CF3 | N | C—H | C—H | CF₃ | H | H | Me | Cl |
| T4 | | 980 | H | C—CF3 | N | C—H | C—H | CF₃ | MeCO | H | Me | Cl |
| T4 | | 981 | H | C—CF3 | N | C—H | C—H | CF₃ | EtCO | H | Me | Cl |
| T4 | | 982 | H | C—CF3 | N | C—H | C—H | CF₃ | n-PrCO | H | Me | Cl |
| T4 | | 983 | H | C—CF3 | N | C—H | C—H | CF₃ | cyclo-PrCO | H | Me | Cl |
| T4 | | 984 | H | C—CF3 | N | C—H | C—H | CF₃ | cyclo-PrCH2CO | H | Me | Cl |
| T4 | | 985 | H | C—CF3 | N | C—H | C—H | CF₃ | CF3CH2CO | H | Me | Cl |
| T4 | | 986 | H | C—Cl | N | C—H | C—H | CF₃ | H | H | Me | Cl |
| T4 | | 987 | H | C—Cl | N | C—H | C—H | CF₃ | MeCO | H | Me | Cl |
| T4 | | 988 | H | C—Cl | N | C—H | C—H | CF₃ | EtCO | H | Me | Cl |
| T4 | | 989 | H | C—Cl | N | C—H | C—H | CF₃ | n-PrCO | H | Me | Cl |
| T4 | | 990 | H | C—Cl | N | C—H | C—H | CF₃ | cyclo-PrCO | H | Me | Cl |
| T4 | | 991 | H | C—Cl | N | C—H | C—H | CF₃ | cyclo-PrCH2CO | H | Me | Cl |
| T4 | | 992 | H | C—Cl | N | C—H | C—H | CF₃ | CF3CH2CO | H | Me | Cl |
| T4 | | 993 | H | C—Cl | C—F | C—Cl | C—F | CF₃ | H | H | Me | Cl |
| T4 | | 994 | H | C—Cl | C—F | C—Cl | C—F | CF₃ | MeCO | H | Me | Cl |
| T4 | | 995 | H | C—Cl | C—F | C—Cl | C—F | CF₃ | EtCO | H | Me | Cl |
| T4 | | 996 | H | C—Cl | C—F | C—Cl | C—F | CF₃ | n-PrCO | H | Me | Cl |
| T4 | | 997 | H | C—Cl | C—F | C—Cl | C—F | CF₃ | cyclo-PrCO | H | Me | Cl |
| T4 | | 998 | H | C—Cl | C—F | C—Cl | C—F | CF₃ | cyclo-PrCH2CO | H | Me | Cl |
| T4 | | 999 | H | C—Cl | C—F | C—Cl | C—F | CF₃ | CF3CH2CO | H | Me | Cl |
| T4 | | 1000 | H | C—Cl | C—H | C—Cl | C—H | CF₃ | H | H | Me | Br |
| T4 | | 1001 | H | C—Cl | C—H | C—Cl | C—H | CF₃ | MeCO | H | Me | Br |
| T4 | — | 1002 | H | C—Cl | C—H | C—Cl | C—H | CF3 | EtCO | H | Me | Br |
| T4 | — | 1003 | H | C—Cl | C—H | C—Cl | C—H | CF3 | n-PrCO | H | Me | Br |
| T4 | — | 1004 | H | C—Cl | C—H | C—Cl | C—H | CF3 | cyclo-PrCO | H | Me | Br |
| T4 | — | 1005 | H | C—Cl | C—H | C—Cl | C—H | CF3 | cyclo-PrCH2CO | H | Me | Br |
| T4 | — | 1006 | H | C—Cl | C—H | C—Cl | C—H | CF3 | CF3CH2CO | H | Me | Br |
| T4 | — | 1007 | H | C—Br | C—H | C—Br | C—H | CF3 | H | H | Me | Br |
| T4 | — | 1008 | H | C—Br | C—H | C—Br | C—H | CF₃ | MeCO | H | Me | Br |
| T4 | — | 1009 | H | C—Br | C—H | C—Br | C—H | CF₃ | EtCO | H | Me | Br |
| T4 | — | 1010 | H | C—Br | C—H | C—Br | C—H | CF₃ | n-PrCO | H | Me | Br |
| T4 | — | 1011 | H | C—Br | C—H | C—Br | C—H | CF₃ | cyclo-PrCO | H | Me | Br |
| T4 | — | 1012 | H | C—Br | C—H | C—Br | C—H | CF₃ | cyclo-PrCH2CO | H | Me | Br |
| T4 | — | 1013 | H | C—Br | C—H | C—Br | C—H | CF₃ | CF3CH2CO | H | Me | Br |
| T4 | — | 1014 | H | C—CF3 | C—H | C—H | C—H | CF3 | H | H | Me | Br |
| T4 | — | 1015 | H | C—CF3 | C—H | C—H | C—H | CF3 | MeCO | H | Me | Br |
| T4 | — | 1016 | H | C—CF3 | C—H | C—H | C—H | CF3 | EtCO | H | Me | Br |
| T4 | — | 1017 | H | C—CF3 | C—H | C—H | C—H | CF3 | n-PrCO | H | Me | Br |
| T4 | — | 1018 | H | C—CF3 | C—H | C—H | C—H | CF3 | cyclo-PrCO | H | Me | Br |
| T4 | — | 1019 | H | C—CF3 | C—H | C—H | C—H | CF3 | cyclo-PrCH2CO | H | Me | Br |
| T4 | — | 1020 | H | C—CF3 | C—H | C—H | C—H | CF3 | CF3CH2CO | H | Me | Br |
| T4 | — | 1021 | H | C—CF3 | C—H | C—CF3 | C—H | CF3 | H | H | Me | Br |
| T4 | — | 1022 | H | C—CF3 | C—H | C—CF3 | C—H | CF3 | MeCO | H | Me | Br |
| T4 | — | 1023 | H | C—CF3 | C—H | C—CF3 | C—H | CF3 | EtCO | H | Me | Br |
| T4 | — | 1024 | H | C—CF3 | C—H | C—CF3 | C—H | CF3 | n-PrCO | H | Me | Br |
| T4 | — | 1025 | H | C—CF3 | C—H | C—CF3 | C—H | CF3 | cyclo-PrCO | H | Me | Br |
| T4 | — | 1026 | H | C—CF3 | C—H | C—CF3 | C—H | CF3 | cyclo-PrCH2CO | H | Me | Br |
| T4 | — | 1027 | H | C—CF3 | C—H | C—CF3 | C—H | CF3 | CF3CH2CO | H | Me | Br |
| T4 | — | 1028 | H | C—CF3 | C—H | C—Cl | C—H | CF3 | H | H | Me | Br |
| T4 | — | 1029 | H | C—CF3 | C—H | C—Cl | C—H | CF3 | MeCO | H | Me | Br |
| T4 | — | 1030 | H | C—CF3 | C—H | C—Cl | C—H | CF3 | EtCO | H | Me | Br |
| T4 | — | 1031 | H | C—CF3 | C—H | C—Cl | C—H | CF3 | n-PrCO | H | Me | Br |
| T4 | — | 1032 | H | C—CF3 | C—H | C—Cl | C—H | CF3 | cyclo-PrCO | H | Me | Br |
| T4 | — | 1033 | H | C—CF3 | C—H | C—Cl | C—H | CF3 | cyclo-PrCH2CO | H | Me | Br |
| T4 | — | 1034 | H | C—CF3 | C—H | C—Cl | C—H | CF3 | CF3CH2CO | H | Me | Br |
| T4 | — | 1035 | H | C—CF3 | C—F | C—H | C—H | CF3 | H | H | Me | Br |
| T4 | — | 1036 | H | C—CF3 | C—F | C—H | C—H | CF₃ | MeCO | H | Me | Br |
| T4 | — | 1037 | H | C—CF3 | C—F | C—H | C—H | CF₃ | EtCO | H | Me | Br |
| T4 | — | 1038 | H | C—CF3 | C—F | C—H | C—H | CF₃ | n-PrCO | H | Me | Br |
| T4 | — | 1039 | H | C—CF3 | C—F | C—H | C—H | CF₃ | cyclo-PrCO | H | Me | Br |
| T4 | — | 1040 | H | C—CF3 | C—F | C—H | C—H | CF₃ | cyclo-PrCH2CO | H | Me | Br |
| T4 | — | 1041 | H | C—CF3 | C—F | C—H | C—H | CF₃ | CF3CH2CO | H | Me | Br |
| T4 | — | 1042 | H | C—CF3 | C—H | C—F | C—H | CF₃ | H | H | Me | Br |
| T4 | — | 1043 | H | C—CF3 | C—H | C—F | C—H | CF₃ | MeCO | H | Me | Br |
| T4 | — | 1044 | H | C—CF3 | C—H | C—F | C—H | CF₃ | EtCO | H | Me | Br |
| T4 | — | 1045 | H | C—CF3 | C—H | C—F | C—H | CF₃ | n-PrCO | H | Me | Br |
| T4 | — | 1046 | H | C—CF3 | C—H | C—F | C—H | CF₃ | cyclo-PrCO | H | Me | Br |
| T4 | — | 1047 | H | C—CF3 | C—H | C—F | C—H | CF₃ | cyclo-PrCH2CO | H | Me | Br |
| T4 | — | 1048 | H | C—CF3 | C—H | C—F | C—H | CF₃ | CF3CH2CO | H | Me | Br |
| T4 | — | 1049 | H | C—Cl | C—Cl | C—Cl | C—H | CF₃ | H | H | Me | Br |
| T4 | — | 1050 | H | C—Cl | C—Cl | C—Cl | C—H | CF₃ | MeCO | H | Me | Br |
| T4 | — | 1051 | H | C—Cl | C—Cl | C—Cl | C—H | CF₃ | n-PrCO | H | Me | Br |
| T4 | — | 1052 | H | C—Cl | C—Cl | C—Cl | C—H | CF₃ | cyclo-PrCO | H | Me | Br |
| T4 | — | 1053 | H | C—Cl | C—Cl | C—Cl | C—H | CF₃ | cyclo-PrCH2CO | H | Me | Br |
| T4 | — | 1054 | H | C—Cl | C—Cl | C—Cl | C—H | CF₃ | CF3CH2CO | H | Me | Br |
| T4 | — | 1055 | H | C—Cl | C—Cl | C—CF3 | C—H | CF₃ | H | H | Me | Br |
| T4 | — | 1056 | H | C—Cl | C—Cl | C—CF3 | C—H | CF₃ | MeCO | H | Me | Br |
| T4 | — | 1057 | H | C—Cl | C—Cl | C—CF3 | C—H | CF₃ | EtCO | H | Me | Br |
| T4 | — | 1058 | H | C—Cl | C—Cl | C—CF3 | C—H | CF₃ | n-PrCO | H | Me | Br |
| T4 | — | 1059 | H | C—Cl | C—Cl | C—CF3 | C—H | CF₃ | cyclo-PrCO | H | Me | Br |
| T4 | — | 1060 | H | C—Cl | C—Cl | C—CF3 | C—H | CF₃ | cyclo-PrCH2CO | H | Me | Br |
| T4 | — | 1061 | H | C—Cl | C—Cl | C—CF3 | C—H | CF₃ | CF3HC2CO | H | Me | Br |
| T4 | — | 1062 | H | C—Cl | N | C—Cl | C—H | CF₃ | H | H | Me | Br |
| T4 | — | 1063 | H | C—Cl | N | C—Cl | C—H | CF₃ | MeCO | H | Me | Br |
| T4 | — | 1064 | H | C—Cl | N | C—Cl | C—H | CF₃ | EtCO | H | Me | Br |
| T4 | — | 1065 | H | C—Cl | N | C—Cl | C—H | CF₃ | n-PrCO | H | Me | Br |
| T4 | — | 1066 | H | C—Cl | N | C—Cl | C—H | CF₃ | cyclo-PrCO | H | Me | Br |
| T4 | — | 1067 | H | C—Cl | N | C—Cl | C—H | CF₃ | cyclo-PrCH2CO | H | Me | Br |
| T4 | — | 1068 | H | C—Cl | N | C—Cl | C—H | CF₃ | CF3CH2CO | H | Me | Br |
| T4 | — | 1069 | H | C—CF3 | N | C—CF3 | C—H | CF₃ | H | H | Me | Br |
| T4 | — | 1070 | H | C—CF3 | N | C—CF3 | C—H | CF₃ | MeCO | H | Me | Br |
| T4 | — | 1071 | H | C—CF3 | N | C—CF3 | C—H | CF₃ | EtCO | H | Me | Br |
| T4 | — | 1072 | H | C—CF3 | N | C—CF3 | C—H | CF₃ | n-PrCO | H | Me | Br |
| T4 | — | 1073 | H | C—CF3 | N | C—CF3 | C—H | CF₃ | cyclo-PrCO | H | Me | Br |
| T4 | — | 1074 | H | C—CF3 | N | C—CF3 | C—H | CF₃ | cyclo-PrCH2CO | H | Me | Br |
| T4 | — | 1075 | H | C—CF3 | N | C—CF3 | C—H | CF₃ | CF3CH2CO | H | Me | Br |
| T4 | — | 1076 | H | C—CF3 | N | C—Cl | C—H | CF₃ | H | H | Me | Br |
| T4 | — | 1077 | H | C—CF3 | N | C—Cl | C—H | CF₃ | MeCO | H | Me | Br |
| T4 | — | 1078 | H | C—CF3 | N | C—Cl | C—H | CF₃ | EtCO | H | Me | Br |
| T4 | — | 1079 | H | C—CF3 | N | C—Cl | C—H | CF₃ | n-PrCO | H | Me | Br |
| T4 | — | 1080 | H | C—CF3 | N | C—Cl | C—H | CF₃ | cyclo-PrCO | H | Me | Br |
| T4 | — | 1081 | H | C—CF3 | N | C—Cl | C—H | CF₃ | cyclo-PrCH2CO | H | Me | Br |
| T4 | — | 1082 | H | C—CF3 | N | C—Cl | C—H | CF₃ | CF3CH2CO | H | Me | Br |
| T4 | — | 1083 | H | C—Cl | C—Cl | C—Cl | C—H | CF₃ | H | Me | Me | Br |
| T4 | — | 1084 | H | C—Cl | C—Cl | C—Cl | C—H | CF₃ | MeCO | Me | Me | Br |
| T4 | — | 1085 | H | C—Cl | C—Cl | C—Cl | C—H | CF₃ | EtCO | Me | Me | Br |
| T4 | — | 1086 | H | C—Cl | C—Cl | C—Cl | C—H | CF₃ | n-PrCO | Me | Me | Br |
| T4 | — | 1087 | H | C—Cl | C—Cl | C—Cl | C—H | CF₃ | cyclo-PrCO | Me | Me | Br |
| T4 | — | 1088 | H | C—Cl | C—Cl | C—Cl | C—H | CF₃ | cyclo-PrCH2CO | Me | Me | Br |
| T4 | — | 1089 | H | C—Cl | C—Cl | C—Cl | C—H | CF₃ | CF3CH2CO | Me | Me | Br |
| T4 | | 1090 | H | C—CF3 | N | C—H | C—H | CF₃ | H | H | Me | Br |
| T4 | | 1091 | H | C—CF3 | N | C—H | C—H | CF₃ | MeCO | H | Me | Br |
| T4 | | 1092 | H | C—CF3 | N | C—H | C—H | CF₃ | EtCO | H | Me | Br |
| T4 | | 1093 | H | C—CF3 | N | C—H | C—H | CF₃ | n-PrCO | H | Me | Br |
| T4 | | 1094 | H | C—CF3 | N | C—H | C—H | CF₃ | cyclo-PrCO | H | Me | Br |
| T4 | | 1095 | H | C—CF3 | N | C—H | C—H | CF₃ | cyclo-PrCH2CO | H | Me | Br |
| T4 | | 1096 | H | C—CF3 | N | C—H | C—H | CF₃ | CF3CH2CO | H | Me | Br |
| T4 | | 1097 | H | C—Cl | N | C—H | C—H | CF₃ | H | H | Me | Br |
| T4 | | 1098 | H | C—Cl | N | C—H | C—H | CF₃ | MeCO | H | Me | Br |
| T4 | | 1099 | H | C—Cl | N | C—H | C—H | CF₃ | EtCO | H | Me | Br |
| T4 | | 1100 | H | C—Cl | N | C—H | C—H | CF₃ | n-PrCO | H | Me | Br |
| T4 | | 1101 | H | C—Cl | N | C—H | C—H | CF₃ | cyclo-PrCO | H | Me | Br |
| T4 | | 1102 | H | C—Cl | N | C—H | C—H | CF₃ | cyclo-PrCH2CO | H | Me | Br |
| T4 | | 1103 | H | C—Cl | N | C—H | C—H | CF₃ | CF3CH2CO | H | Me | Br |
| T4 | | 1104 | H | C—Cl | C—F | C—Cl | C—F | CF₃ | H | H | Me | Br |
| T4 | | 1105 | H | C—Cl | C—F | C—Cl | C—F | CF₃ | MeCO | H | Me | Br |
| T4 | | 1106 | H | C—Cl | C—F | C—Cl | C—F | CF₃ | EtCO | H | Me | Br |
| T4 | | 1107 | H | C—Cl | C—F | C—Cl | C—F | CF₃ | n-PrCO | H | Me | Br |
| T4 | | 1108 | H | C—Cl | C—F | C—Cl | C—F | CF₃ | cyclo-PrCO | H | Me | Br |
| T4 | | 1109 | H | C—Cl | C—F | C—Cl | C—F | CF₃ | cyclo-PrCH2CO | H | Me | Br |
| T4 | | 1110 | H | C—Cl | C—F | C—Cl | C—F | CF₃ | CF3CH2CO | H | Me | Br |
| T4 | — | 1111 | H | C—Cl | C—H | C—Cl | C—H | CF₃ | H | H | Me | CH₃ |
| T4 | — | 1112 | H | C—Cl | C—H | C—Cl | C—H | CF₃ | MeCO | H | Me | CH₃ |
| T4 | — | 1113 | H | C—Cl | C—H | C—Cl | C—H | CF3 | EtCO | H | Me | CH₃ |
| T4 | — | 1114 | H | C—Cl | C—H | C—Cl | C—H | CF3 | n-PrCO | H | Me | CH₃ |
| T4 | — | 1115 | H | C—Cl | C—H | C—Cl | C—H | CF3 | cyclo-PrCO | H | Me | CH₃ |
| T4 | — | 1116 | H | C—Cl | C—H | C—Cl | C—H | CF3 | cylco-PrCH2CO | H | Me | CH₃ |
| T4 | — | 1117 | H | C—Cl | C—H | C—Cl | C—H | CF3 | CF3CH2CO | H | Me | CH₃ |
| T4 | — | 1118 | H | C—Br | C—H | C—Br | C—H | CF3 | H | H | Me | CH₃ |
| T4 | — | 1119 | H | C—Br | C—H | C—Br | C—H | CF₃ | MeCO | H | Me | CH₃ |
| T4 | — | 1120 | H | C—Br | C—H | C—Br | C—H | CF₃ | EtCO | H | Me | CH₃ |
| T4 | — | 1121 | H | C—Br | C—H | C—Br | C—H | CF₃ | n-PrCO | H | Me | CH₃ |
| T4 | — | 1122 | H | C—Br | C—H | C—Br | C—H | CF₃ | cyclo-PrCO | H | Me | CH₃ |
| T4 | — | 1123 | H | C—Br | C—H | C—Br | C—H | CF₃ | cyclo-PrCH2CO | H | Me | CH₃ |
| T4 | — | 1124 | H | C—Br | C—H | C—Br | C—H | CF₃ | CF3CH2CO | H | Me | CH₃ |
| T4 | — | 1125 | H | C—CF3 | C—H | C—H | C—H | CF3 | H | H | Me | CH₃ |
| T4 | — | 1126 | H | C—CF3 | C—H | C—H | C—H | CF3 | MeCO | H | Me | CH₃ |
| T4 | — | 1127 | H | C—CF3 | C—H | C—H | C—H | CF3 | EtCO | H | Me | CH₃ |
| T4 | — | 1128 | H | C—CF3 | C—H | C—H | C—H | CF3 | n-PrCO | H | Me | CH₃ |
| T4 | — | 1129 | H | C—CF3 | C—H | C—H | C—H | CF3 | cyclo-PrCO | H | Me | CH₃ |
| T4 | — | 1130 | H | C—CF3 | C—H | C—H | C—H | CF3 | cyclo-PrCH2CO | H | Me | CH₃ |
| T4 | — | 1131 | H | C—CF3 | C—H | C—H | C—H | CF3 | CF3CH2CO | H | Me | CH₃ |
| T4 | — | 1132 | H | C—CF3 | C—H | C—CF3 | C—H | CF3 | H | H | Me | CH₃ |
| T4 | — | 1133 | H | C—CF3 | C—H | C—CF3 | C—H | CF3 | MeCO | H | Me | CH₃ |
| T4 | — | 1134 | H | C—CF3 | C—H | C—CF3 | C—H | CF3 | EtCO | H | Me | CH₃ |
| T4 | — | 1135 | H | C—CF3 | C—H | C—CF3 | C—H | CF3 | n-PrCO | H | Me | CH₃ |
| T4 | — | 1136 | H | C—CF3 | C—H | C—CF3 | C—H | CF3 | cyclo-PrCO | H | Me | CH₃ |
| T4 | — | 1137 | H | C—CF3 | C—H | C—CF3 | C—H | CF3 | cyclo-PrCH2CO | H | Me | CH₃ |
| T4 | — | 1138 | H | C—CF3 | C—H | C—CF3 | C—H | CF3 | CF3CH2CO | H | Me | CH₃ |
| T4 | — | 1139 | H | C—CF3 | C—H | C—Cl | C—H | CF3 | H | H | Me | CH₃ |
| T4 | — | 1140 | H | C—CF3 | C—H | C—Cl | C—H | CF3 | MeCO | H | Me | CH₃ |
| T4 | — | 1141 | H | C—CF3 | C—H | C—Cl | C—H | CF3 | EtCO | H | Me | CH₃ |
| T4 | — | 1142 | H | C—CF3 | C—H | C—Cl | C—H | CF3 | n-PrCO | H | Me | CH₃ |
| T4 | — | 1143 | H | C—CF3 | C—H | C—Cl | C—H | CF3 | cyclo-PrCO | H | Me | CH₃ |
| T4 | — | 1144 | H | C—CF3 | C—H | C—Cl | C—H | CF3 | cyclo-PrCH2CO | H | Me | CH₃ |
| T4 | — | 1145 | H | C—CF3 | C—H | C—Cl | C—H | CF3 | CF3CH2CO | H | Me | CH₃ |
| T4 | — | 1146 | H | C—CF3 | C—F | C—H | C—H | CF3 | H | H | Me | CH₃ |
| T4 | — | 1147 | H | C—CF3 | C—F | C—H | C—H | CF₃ | MeCO | H | Me | CH₃ |
| T4 | — | 1148 | H | C—CF3 | C—F | C—H | C—H | CF₃ | EtCO | H | Me | CH₃ |
| T4 | — | 1149 | H | C—CF3 | C—F | C—H | C—H | CF₃ | n-PrCO | H | Me | CH₃ |
| T4 | — | 1150 | H | C—CF3 | C—F | C—H | C—H | CF₃ | cyclo-PrCO | H | Me | CH₃ |
| T4 | — | 1151 | H | C—CF3 | C—F | C—H | C—H | CF₃ | cyclo-PrCH2CO | H | Me | CH₃ |
| T4 | — | 1152 | H | C—CF3 | C—F | C—H | C—H | CF₃ | CF3CH2CO | H | Me | CH₃ |
| T4 | — | 1153 | H | C—CF3 | C—H | C—F | C—H | CF₃ | H | H | Me | CH₃ |
| T4 | — | 1154 | H | C—CF3 | C—H | C—F | C—H | CF₃ | MeCO | H | Me | CH₃ |
| T4 | — | 1155 | H | C—CF3 | C—H | C—F | C—H | CF₃ | EtCO | H | Me | CH₃ |
| T4 | — | 1156 | H | C—CF3 | C—H | C—F | C—H | CF₃ | n-PrCO | H | Me | CH₃ |
| T4 | — | 1157 | H | C—CF3 | C—H | C—F | C—H | CF₃ | cyclo-PrCO | H | Me | CH₃ |
| T4 | — | 1158 | H | C—CF3 | C—H | C—F | C—H | CF₃ | cyclo-PrCH2CO | H | Me | CH₃ |
| T4 | — | 1159 | H | C—CF3 | C—H | C—F | C—H | CF₃ | CF3CH2CO | H | Me | CH₃ |
| T4 | — | 1160 | H | C—Cl | C—Cl | C—Cl | C—H | CF₃ | H | H | Me | CH₃ |
| T4 | — | 1161 | H | C—Cl | C—Cl | C—Cl | C—H | CF₃ | MeCO | H | Me | CH₃ |
| T4 | — | 1162 | H | C—Cl | C—Cl | C—Cl | C—H | CF₃ | n-PrCO | H | Me | CH₃ |
| T4 | — | 1163 | H | C—Cl | C—Cl | C—Cl | C—H | CF₃ | cyclo-PrCO | H | Me | CH₃ |
| T4 | — | 1164 | H | C—Cl | C—Cl | C—Cl | C—H | CF₃ | cyclo-PrCH2CO | H | Me | CH₃ |
| T4 | — | 1165 | H | C—Cl | C—Cl | C—Cl | C—H | CF₃ | CF3CH2CO | H | Me | CH₃ |
| T4 | — | 1166 | H | C—Cl | C—Cl | C—CF3 | C—H | CF₃ | H | H | Me | CH₃ |
| T4 | — | 1167 | H | C—Cl | C—Cl | C—CF3 | C—H | CF₃ | MeCO | H | Me | CH₃ |
| T4 | — | 1168 | H | C—Cl | C—Cl | C—CF3 | C—H | CF₃ | EtCO | H | Me | CH₃ |
| T4 | — | 1169 | H | C—Cl | C—Cl | C—CF3 | C—H | CF₃ | n-PrCO | H | Me | CH₃ |
| T4 | — | 1170 | H | C—Cl | C—Cl | C—CF3 | C—H | CF₃ | cyclo-PrCO | H | Me | CH₃ |
| T4 | — | 1171 | H | C—Cl | C—Cl | C—CF3 | C—H | CF₃ | cyclo-PrCH2CO | H | Me | CH₃ |
| T4 | — | 1172 | H | C—Cl | C—Cl | C—CF3 | C—H | CF₃ | CF3CH2CO | H | Me | CH₃ |
| T4 | — | 1173 | H | C—Cl | N | C—Cl | C—H | CF₃ | H | H | Me | CH₃ |
| T4 | — | 1174 | H | C—Cl | N | C—Cl | C—H | CF₃ | MeCO | H | Me | CH₃ |
| T4 | — | 1175 | H | C—Cl | N | C—Cl | C—H | CF₃ | EtCO | H | Me | CH₃ |
| T4 | — | 1176 | H | C—Cl | N | C—Cl | C—H | CF₃ | n-PrCO | H | Me | CH₃ |
| T4 | — | 1177 | H | C—Cl | N | C—Cl | C—H | CF₃ | cyclo-PrCO | H | Me | CH₃ |
| T4 | — | 1178 | H | C—Cl | N | C—Cl | C—H | CF₃ | cyclo-PrCH2CO | H | Me | CH₃ |
| T4 | — | 1179 | H | C—Cl | N | C—Cl | C—H | CF₃ | CF3CH2CO | H | Me | CH₃ |
| T4 | — | 1180 | H | C—CF3 | N | C—CF3 | C—H | CF₃ | H | H | Me | CH₃ |
| T4 | — | 1181 | H | C—CF3 | N | C—CF3 | C—H | CF₃ | MeCO | H | Me | CH₃ |
| T4 | — | 1182 | H | C—CF3 | N | C—CF3 | C—H | CF₃ | EtCO | H | Me | CH₃ |
| T4 | — | 1183 | H | C—CF3 | N | C—CF3 | C—H | CF₃ | n-PrCO | H | Me | CH₃ |
| T4 | — | 1184 | H | C—CF3 | N | C—CF3 | C—H | CF₃ | cyclo-PrCO | H | Me | CH₃ |
| T4 | — | 1185 | H | C—CF3 | N | C—CF3 | C—H | CF₃ | cyclo-PrCH2CO | H | Me | CH₃ |
| T4 | — | 1186 | H | C—CF3 | N | C—CF3 | C—H | CF₃ | CF3CH2CO | H | Me | CH₃ |
| T4 | — | 1187 | H | C—CF3 | N | C—Cl | C—H | CF₃ | H | H | Me | CH₃ |
| T4 | — | 1188 | H | C—CF3 | N | C—Cl | C—H | CF₃ | MeCO | H | Me | CH₃ |
| T4 | — | 1189 | H | C—CF3 | N | C—Cl | C—H | CF₃ | EtCO | H | Me | CH₃ |
| T4 | — | 1190 | H | C—CF3 | N | C—Cl | C—H | CF₃ | n-PrCO | H | Me | CH₃ |
| T4 | — | 1191 | H | C—CF3 | N | C—Cl | C—H | CF₃ | cyclo-PrCO | H | Me | CH₃ |
| T4 | — | 1192 | H | C—CF3 | N | C—Cl | C—H | CF₃ | cyclo-PrCH2CO | H | Me | CH₃ |
| T4 | — | 1193 | H | C—CF3 | N | C—Cl | C—H | CF₃ | CF3CH2CO | H | Me | CH₃ |
| T4 | — | 1194 | H | C—Cl | C—Cl | C—Cl | C—H | CF₃ | H | Me | Me | CH₃ |
| T4 | — | 1195 | H | C—Cl | C—Cl | C—Cl | C—H | CF₃ | MeCO | Me | Me | CH₃ |
| T4 | — | 1196 | H | C—Cl | C—Cl | C—Cl | C—H | CF₃ | EtCO | Me | Me | CH₃ |
| T4 | — | 1197 | H | C—Cl | C—Cl | C—Cl | C—H | CF₃ | n-PrCO | Me | Me | CH₃ |
| T4 | — | 1198 | H | C—Cl | C—Cl | C—Cl | C—H | CF₃ | cyclo-PrCO | Me | Me | CH₃ |
| T4 | — | 1199 | H | C—Cl | C—Cl | C—Cl | C—H | CF₃ | cyclo-PrCH2CO | Me | Me | CH₃ |
| T4 | — | 1200 | H | C—Cl | C—Cl | C—Cl | C—H | CF₃ | CF3CH2CO | Me | Me | CH₃ |
| T4 | | 1201 | H | C—CF3 | N | C—H | C—H | CF₃ | H | H | Me | CH₃ |
| T4 | | 1202 | H | C—CF3 | N | C—H | C—H | CF₃ | MeCO | H | Me | CH₃ |
| T4 | | 1203 | H | C—CF3 | N | C—H | C—H | CF₃ | EtCO | H | Me | CH₃ |
| T4 | | 1204 | H | C—CF3 | N | C—H | C—H | CF₃ | n-PrCO | H | Me | CH₃ |
| T4 | | 1205 | H | C—CF3 | N | C—H | C—H | CF₃ | cyclo-PrCO | H | Me | CH₃ |
| T4 | | 1206 | H | C—CF3 | N | C—H | C—H | CF₃ | cyclo-PrCH2CO | H | Me | CH₃ |
| T4 | | 1207 | H | C—CF3 | N | C—H | C—H | CF₃ | CF3CH2CO | H | Me | CH₃ |
| T4 | | 1208 | H | C—Cl | N | C—H | C—H | CF₃ | H | H | Me | CH₃ |
| T4 | | 1209 | H | C—Cl | N | C—H | C—H | CF₃ | MeCO | H | Me | CH₃ |
| T4 | | 1210 | H | C—Cl | N | C—H | C—H | CF₃ | EtCO | H | Me | CH₃ |
| T4 | | 1211 | H | C—Cl | N | C—H | C—H | CF₃ | n-PrCO | H | Me | CH₃ |
| T4 | | 1212 | H | C—Cl | N | C—H | C—H | CF₃ | cyclo-PrCO | H | Me | CH₃ |
| T4 | | 1213 | H | C—Cl | N | C—H | C—H | CF₃ | cyclo-PrCH2CO | H | Me | CH₃ |
| T4 | | 1214 | H | C—Cl | N | C—H | C—H | CF₃ | CF3CH2CO | H | Me | CH₃ |
| T4 | | 1215 | H | C—Cl | C—F | C—Cl | C—F | CF₃ | H | H | Me | CH₃ |
| T4 | | 1216 | H | C—Cl | C—F | C—Cl | C—F | CF₃ | MeCO | H | Me | CH₃ |
| T4 | | 1217 | H | C—Cl | C—F | C—Cl | C—F | CF₃ | EtCO | H | Me | CH₃ |
| T4 | | 1218 | H | C—Cl | C—F | C—Cl | C—F | CF₃ | n-PrCO | H | Me | CH₃ |
| T4 | | 1219 | H | C—Cl | C—F | C—Cl | C—F | CF₃ | cyclo-PrCO | H | Me | CH₃ |
| T4 | | 1220 | H | C—Cl | C—F | C—Cl | C—F | CF₃ | cyclo-PrCH2CO | H | Me | CH₃ |
| T4 | | 1221 | H | C—Cl | C—F | C—Cl | C—F | CF₃ | CF3CH2CO | H | Me | CH₃ |
| T4 | — | 1222 | H | C—Cl | C—H | C—Cl | C—H | CF₃ | H | H | Me | CF₃ |
| T4 | — | 1223 | H | C—Cl | C—H | C—Cl | C—H | CF₃ | MeCO | H | Me | CF₃ |
| T4 | — | 1224 | H | C—Cl | C—H | C—Cl | C—H | CF3 | EtCO | H | Me | CF₃ |
| T4 | — | 1225 | H | C—Cl | C—H | C—Cl | C—H | CF3 | n-PrCO | H | Me | CF₃ |
| T4 | — | 1226 | H | C—Cl | C—H | C—Cl | C—H | CF3 | cyclo-PrCO | H | Me | CF₃ |
| T4 | — | 1227 | H | C—Cl | C—H | C—Cl | C—H | CF3 | cyclo-PrCH2CO | H | Me | CF₃ |
| T4 | — | 1228 | H | C—Cl | C—H | C—Cl | C—H | CF3 | CF3CH2CO | H | Me | CF₃ |
| T4 | — | 1229 | H | C—Br | C—H | C—Br | C—H | CF3 | H | H | Me | CF₃ |
| T4 | — | 1230 | H | C—Br | C—H | C—Br | C—H | CF₃ | MeCO | H | Me | CF₃ |
| T4 | — | 1231 | H | C—Br | C—H | C—Br | C—H | CF₃ | EtCO | H | Me | CF₃ |
| T4 | — | 1232 | H | C—Br | C—H | C—Br | C—H | CF₃ | n-PrCO | H | Me | CF₃ |
| T4 | — | 1233 | H | C—Br | C—H | C—Br | C—H | CF₃ | cyclo-PrCO | H | Me | CF₃ |
| T4 | — | 1234 | H | C—Br | C—H | C—Br | C—H | CF₃ | cyclo-PrCH2CO | H | Me | CF₃ |
| T4 | — | 1235 | H | C—Br | C—H | C—Br | C—H | CF₃ | CF3CH2CO | H | Me | CF₃ |
| T4 | — | 1236 | H | C—CF3 | C—H | C—H | C—H | CF3 | H | H | Me | CF₃ |
| T4 | — | 1237 | H | C—CF3 | C—H | C—H | C—H | CF3 | MeCO | H | Me | CF₃ |
| T4 | — | 1238 | H | C—CF3 | C—H | C—H | C—H | CF3 | EtCO | H | Me | CF₃ |
| T4 | — | 1239 | H | C—CF3 | C—H | C—H | C—H | CF3 | n-PrCO | H | Me | CF₃ |
| T4 | — | 1240 | H | C—CF3 | C—H | C—H | C—H | CF3 | cyclo-PrCO | H | Me | CF₃ |
| T4 | — | 1241 | H | C—CF3 | C—H | C—H | C—H | CF3 | cyclo-PrCH2CO | H | Me | CF₃ |
| T4 | — | 1242 | H | C—CF3 | C—H | C—H | C—H | CF3 | CF3CH2CO | H | Me | CF₃ |
| T4 | — | 1243 | H | C—CF3 | C—H | C—CF3 | C—H | CF3 | H | H | Me | CF₃ |
| T4 | — | 1244 | H | C—CF3 | C—H | C—CF3 | C—H | CF3 | MeCO | H | Me | CF₃ |
| T4 | — | 1245 | H | C—CF3 | C—H | C—CF3 | C—H | CF3 | EtCO | H | Me | CF₃ |
| T4 | — | 1246 | H | C—CF3 | C—H | C—CF3 | C—H | CF3 | n-PrCO | H | Me | CF₃ |
| T4 | — | 1247 | H | C—CF3 | C—H | C—CF3 | C—H | CF3 | cyclo-PrCO | H | Me | CF₃ |
| T4 | — | 1248 | H | C—CF3 | C—H | C—CF3 | C—H | CF3 | cyclo-PrCH2CO | H | Me | CF₃ |
| T4 | — | 1249 | H | C—CF3 | C—H | C—CF3 | C—H | CF3 | CF3CH2CO | H | Me | CF₃ |
| T4 | — | 1250 | H | C—CF3 | C—H | C—Cl | C—H | CF3 | H | H | Me | CF₃ |
| T4 | — | 1251 | H | C—CF3 | C—H | C—Cl | C—H | CF3 | MeCO | H | Me | CF₃ |
| T4 | — | 1252 | H | C—CF3 | C—H | C—Cl | C—H | CF3 | EtCO | H | Me | CF₃ |
| T4 | — | 1253 | H | C—CF3 | C—H | C—Cl | C—H | CF3 | n-PrCO | H | Me | CF₃ |
| T4 | — | 1254 | H | C—CF3 | C—H | C—Cl | C—H | CF3 | cyclo-PrCO | H | Me | CF₃ |
| T4 | — | 1255 | H | C—CF3 | C—H | C—Cl | C—H | CF3 | cyclo-PrCH2CO | H | Me | CF₃ |
| T4 | — | 1256 | H | C—CF3 | C—H | C—Cl | C—H | CF3 | CF3CH2CO | H | Me | CF₃ |
| T4 | — | 1257 | H | C—CF3 | C—F | C—H | C—H | CF3 | H | H | Me | CF₃ |
| T4 | — | 1258 | H | C—CF3 | C—F | C—H | C—H | CF₃ | MeCO | H | Me | CF₃ |
| T4 | — | 1259 | H | C—CF3 | C—F | C—H | C—H | CF₃ | EtCO | H | Me | CF₃ |
| T4 | — | 1260 | H | C—CF3 | C—F | C—H | C—H | CF₃ | n-PrCO | H | Me | CF₃ |
| T4 | — | 1261 | H | C—CF3 | C—F | C—H | C—H | CF₃ | cyclo-PrCO | H | Me | CF₃ |
| T4 | — | 1262 | H | C—CF3 | C—F | C—H | C—H | CF₃ | cyclo-PrCH2CO | H | Me | CF₃ |
| T4 | — | 1263 | H | C—CF3 | C—F | C—H | C—H | CF₃ | CF3CH2CO | H | Me | CF₃ |
| T4 | — | 1264 | H | C—CF3 | C—H | C—F | C—H | CF₃ | H | H | Me | CF₃ |
| T4 | — | 1265 | H | C—CF3 | C—H | C—F | C—H | CF₃ | MeCO | H | Me | CF₃ |
| T4 | — | 1266 | H | C—CF3 | C—H | C—F | C—H | CF₃ | EtCO | H | Me | CF₃ |
| T4 | — | 1267 | H | C—CF3 | C—H | C—F | C—H | CF₃ | n-PrCO | H | Me | CF₃ |
| T4 | — | 1268 | H | C—CF3 | C—H | C—F | C—H | CF₃ | cyclo-PrCO | H | Me | CF₃ |
| T4 | — | 1269 | H | C—CF3 | C—H | C—F | C—H | CF₃ | cyclo-PrCH2CO | H | Me | CF₃ |
| T4 | — | 1270 | H | C—CF3 | C—H | C—F | C—H | CF₃ | CF3CH2CO | H | Me | CF₃ |
| T4 | — | 1271 | H | C—Cl | C—Cl | C—Cl | C—H | CF₃ | H | H | Me | CF₃ |
| T4 | — | 1272 | H | C—Cl | C—Cl | C—Cl | C—H | CF₃ | MeCO | H | Me | CF₃ |
| T4 | — | 1273 | H | C—Cl | C—Cl | C—Cl | C—H | CF₃ | n-PrCO | H | Me | CF₃ |
| T4 | — | 1274 | H | C—Cl | C—Cl | C—Cl | C—H | CF₃ | cyclo-PrCO | H | Me | CF₃ |
| T4 | — | 1275 | H | C—Cl | C—Cl | C—Cl | C—H | CF₃ | cyclo-PrCH2CO | H | Me | CF₃ |
| T4 | — | 1276 | H | C—Cl | C—Cl | C—Cl | C—H | CF₃ | CF3CH2CO | H | Me | CF₃ |
| T4 | — | 1277 | H | C—Cl | C—Cl | C—CF3 | C—H | CF₃ | H | H | Me | CF₃ |
| T4 | — | 1278 | H | C—Cl | C—Cl | C—CF3 | C—H | CF₃ | MeCO | H | Me | CF₃ |
| T4 | — | 1279 | H | C—Cl | C—Cl | C—CF3 | C—H | CF₃ | EtCO | H | Me | CF₃ |
| T4 | — | 1280 | H | C—Cl | C—Cl | C—CF3 | C—H | CF₃ | n-PrCO | H | Me | CF₃ |
| T4 | — | 1281 | H | C—Cl | C—Cl | C—CF3 | C—H | CF₃ | cyclo-PrCO | H | Me | CF₃ |
| T4 | — | 1282 | H | C—Cl | C—Cl | C—CF3 | C—H | CF₃ | cyclo-PrCH2CO | H | Me | CF₃ |
| T4 | — | 1283 | H | C—Cl | C—Cl | C—CF3 | C—H | CF₃ | CF3CH2CO | H | Me | CF₃ |
| T4 | — | 1284 | H | C—Cl | N | C—Cl | C—H | CF₃ | H | H | Me | CF₃ |
| T4 | — | 1285 | H | C—Cl | N | C—Cl | C—H | CF₃ | MeCO | H | Me | CF₃ |
| T4 | — | 1286 | H | C—Cl | N | C—Cl | C—H | CF₃ | EtCO | H | Me | CF₃ |
| T4 | — | 1287 | H | C—Cl | N | C—Cl | C—H | CF₃ | n-PrCO | H | Me | CF₃ |
| T4 | — | 1288 | H | C—Cl | N | C—Cl | C—H | CF₃ | cyclo-PrCO | H | Me | CF₃ |
| T4 | — | 1289 | H | C—Cl | N | C—Cl | C—H | CF₃ | cyclo-PrCH2CO | H | Me | CF₃ |
| T4 | — | 1290 | H | C—Cl | N | C—Cl | C—H | CF₃ | CF3CH2CO | H | Me | CF₃ |
| T4 | — | 1291 | H | C—CF3 | N | C—CF3 | C—H | CF₃ | H | H | Me | CF₃ |
| T4 | — | 1292 | H | C—CF3 | N | C—CF3 | C—H | CF₃ | MeCO | H | Me | CF₃ |
| T4 | — | 1293 | H | C—CF3 | N | C—CF3 | C—H | CF₃ | EtCO | H | Me | CF₃ |
| T4 | — | 1294 | H | C—CF3 | N | C—CF3 | C—H | CF₃ | n-PrCO | H | Me | CF₃ |
| T4 | — | 1295 | H | C—CF3 | N | C—CF3 | C—H | CF₃ | cyclo-PrCO | H | Me | CF₃ |
| T4 | — | 1296 | H | C—CF3 | N | C—CF3 | C—H | CF₃ | cyclo-PrCH2CO | H | Me | CF₃ |
| T4 | — | 1297 | H | C—CF3 | N | C—CF3 | C—H | CF₃ | CF3CH2CO | H | Me | CF₃ |
| T4 | — | 1298 | H | C—CF3 | N | C—Cl | C—H | CF₃ | H | H | Me | CF₃ |
| T4 | — | 1299 | H | C—CF3 | N | C—Cl | C—H | CF₃ | MeCO | H | Me | CF₃ |
| T4 | — | 1300 | H | C—CF3 | N | C—Cl | C—H | CF₃ | EtCO | H | Me | CF₃ |
| T4 | — | 1301 | H | C—CF3 | N | C—Cl | C—H | CF₃ | n-PrCO | H | Me | CF₃ |
| T4 | — | 1302 | H | C—CF3 | N | C—Cl | C—H | CF₃ | cyclo-PrCO | H | Me | CF₃ |
| T4 | — | 1303 | H | C—CF3 | N | C—Cl | C—H | CF₃ | cyclo-PrCH2CO | H | Me | CF₃ |
| T4 | — | 1304 | H | C—CF3 | N | C—Cl | C—H | CF₃ | CF3CH2CO | H | Me | CF₃ |
| T4 | — | 1305 | H | C—Cl | C—Cl | C—Cl | C—H | CF₃ | H | Me | Me | CF₃ |
| T4 | — | 1306 | H | C—Cl | C—Cl | C—Cl | C—H | CF₃ | MeCO | Me | Me | CF₃ |
| T4 | — | 1307 | H | C—Cl | C—Cl | C—Cl | C—H | CF₃ | EtCO | Me | Me | CF₃ |
| T4 | — | 1308 | H | C—Cl | C—Cl | C—Cl | C—H | CF₃ | n-PrCO | Me | Me | CF₃ |
| T4 | — | 1309 | H | C—Cl | C—Cl | C—Cl | C—H | CF₃ | cyclo-PrCO | Me | Me | CF₃ |
| T4 | — | 1310 | H | C—Cl | C—Cl | C—Cl | C—H | CF₃ | cyclo-PrCH2CO | Me | Me | CF₃ |
| T4 | — | 1311 | H | C—Cl | C—Cl | C—Cl | C—H | CF₃ | CF3CH2CO | Me | Me | CF₃ |
| T4 | — | 1312 | H | C—CF3 | N | C—H | C—H | CF₃ | H | H | Me | CF₃ |
| T4 | — | 1313 | H | C—CF3 | N | C—H | C—H | CF₃ | MeCO | H | Me | CF₃ |
| T4 | — | 1314 | H | C—CF3 | N | C—H | C—H | CF₃ | EtCO | H | Me | CF₃ |
| T4 | — | 1315 | H | C—CF3 | N | C—H | C—H | CF₃ | n-PrCO | H | Me | CF₃ |
| T4 | — | 1316 | H | C—CF3 | N | C—H | C—H | CF₃ | cyclo-PrCO | H | Me | CF₃ |
| T4 | — | 1317 | H | C—CF3 | N | C—H | C—H | CF₃ | cyclo-PrCH2CO | H | Me | CF₃ |
| T4 | — | 1318 | H | C—CF3 | N | C—H | C—H | CF₃ | CF3CH2CO | H | Me | CF₃ |
| T4 | — | 1319 | H | C—Cl | N | C—H | C—H | CF₃ | H | H | Me | CF₃ |
| T4 | — | 1320 | H | C—Cl | N | C—H | C—H | CF₃ | MeCO | H | Me | CF₃ |
| T4 | — | 1321 | H | C—Cl | N | C—H | C—H | CF₃ | EtCO | H | Me | CF₃ |
| T4 | — | 1322 | H | C—Cl | N | C—H | C—H | CF₃ | n-PrCO | H | Me | CF₃ |
| T4 | — | 1323 | H | C—Cl | N | C—H | C—H | CF₃ | cyclo-PrCO | H | Me | CF₃ |
| T4 | — | 1324 | H | C—Cl | N | C—H | C—H | CF₃ | cyclo-PrCH2CO | H | Me | CF₃ |
| T4 | — | 1325 | H | C—Cl | N | C—H | C—H | CF₃ | CF3CH2CO | H | Me | CF₃ |
| T4 | — | 1326 | H | C—Cl | C—F | C—Cl | C—F | CF₃ | H | H | Me | CF₃ |
| T4 | — | 1327 | H | C—Cl | C—F | C—Cl | C—F | CF₃ | MeCO | H | Me | CF₃ |
| T4 | — | 1328 | H | C—Cl | C—F | C—Cl | C—F | CF₃ | EtCO | H | Me | CF₃ |
| T4 | — | 1329 | H | C—Cl | C—F | C—Cl | C—F | CF₃ | n-PrCO | H | Me | CF₃ |
| T4 | — | 1330 | H | C—Cl | C—F | C—Cl | C—F | CF₃ | cyclo-PrCO | H | Me | CF₃ |
| T4 | — | 1331 | H | C—Cl | C—F | C—Cl | C—F | CF₃ | cyclo-PrCH2CO | H | Me | CF₃ |
| T4 | — | 1332 | H | C—Cl | C—F | C—Cl | C—F | CF₃ | CF3CH2CO | H | Me | CF₃ |
| T4 | — | 1333 | H | C—Cl | C—Cl | C—Cl | C—H | CF3 | EtCO | H | H | H |
| T4 | — | 1334 | H | C—Cl | C—Cl | C—Cl | C—H | CF3 | EtCO | H | H | F |
| T4 | — | 1335 | H | C—Cl | C—Cl | C—Cl | C—H | CF3 | EtCO | H | H | Cl |
| T4 | — | 1336 | H | C—Cl | C—Cl | C—Cl | C—H | CF3 | EtCO | H | H | Br |
| T4 | — | 1337 | H | C—Cl | C—Cl | C—Cl | C—H | CF3 | EtCO | H | H | Me |
| T4 | — | 1338 | H | C—Cl | C—Cl | C—Cl | C—H | CF3 | EtCO | H | H | CF₃ |
| T4 | — | 1339 | H | C—Cl | C—Cl | C—Cl | C—H | CF3 | EtCO | H | Me | H |
| T4 | — | 1340 | H | C—Cl | C—Cl | C—Cl | C—H | CF3 | EtCO | H | Me | F |
| T4 | — | 1341 | H | C—Cl | C—Cl | C—Cl | C—H | CF3 | EtCO | H | Me | Cl |
| T4 | — | 1342 | H | C—Cl | C—Cl | C—Cl | C—H | CF3 | EtCO | H | Me | Br |
| T4 | — | 1343 | H | C—Cl | C—Cl | C—Cl | C—H | CF3 | EtCO | H | Me | Me |
| T4 | — | 1344 | H | C—Cl | C—Cl | C—Cl | C—H | CF3 | EtCO | H | Me | CF₃ |

| Compound | | | 1H-NMR |
|---|---|---|---|
| T1 | — | 93 | 1H-NMR (CDCl3) δ: 1.50 (9H, s), 1.83-1.87 (1H, m), 2.64-2.65 (1H, m), |
| | | | 2.93-2.99 (2H, m), 4.05 (2H, dd), 4.75-4.77 (1H, m), 5.22-5.25 (1H, m), 5.52 |
| | | | (2H, dd), 7.33 (2H, s), 7.46 (1H, d), 7.81-7.84 (2H, m) |
| T1 | — | 95 | 1H-NMR (CDCl3) δ: 1.22 (3H, t), 1.83-1.86 (1H, m), 2.29 (2H, q), 2.65-2.68 |
| | | | (1H, m), 2.89-3.04 (2H, m), 4.05 (2H, dd), 5.46-5.58 (4H, m), 7.29-7.39 (3H, |
| | | | m), 7.69-7.78 (2H, m) |
| T2 | — | 52 | 1H-NMR (CDCl3) δ: 4.09 (2H, dd), 5.49 (2H, dd), 7.36 (2H, s), 8.06 (1H, d), |
| | | | 8.24 (1H, s), 8.33-8.36 (1H, m), 8.44 (1H, d), 8.99 (1H, s) |
| T2 | — | 55 | 1H-NMR (CDCl3) δ: 4.03 (2H, dd), 5.48 (2H, dd), 7.37-7.40 (3H, m), |
| | | | 8.23-8.28 (2H, m) |
| T2 | — | 127 | 1H-NMR (CDCl3) δ: 4.07 (2H, dd), 5.50 (2H, dd), 7.35 (2H, s), 7.75 (1H, d), |
| | | | 8.05-8.08 (1H, m), 8.18 (1H, s), 8.33 (1H, d), 8.70 (1H, s) |
| T2 | — | 130 | 1H-NMR (CDCl3) δ: 4.02 (2H, dd), 5.49 (2H, dd), 7.25-7.32 (3H, m), |
| | | | 7.92-7.95 (1H, m), 8.18-8.20 (1H, m) |

The present invention is illustrated in detail with reference to the examples which follow, though the examples should not be interpreted in such a manner as to restrict the invention.

### A: Preparation of the Starting Materials for the Manufacturing the Nitrooketones of Formula (II) According to the Invention

### Synthesis of Example A-1

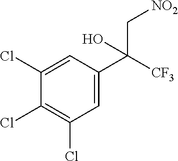 [SMILES: O=[N+]([O-])CC(O)(c1cc(Cl)c(Cl)c(Cl)c1)C(F)(F)F]

6 g 2,2,2-Trifluoro-1-(3,4,5-trichlorophenyl)ethanone, 13.2 g nitromethane and 3 g potassium carbonate were suspended in 100 ml dichloromethane. The reaction mixture was stirred at room temperature for 14 hours. After filtering off the crystals, 2 N hydrochloric acid was added and extracted with ethyl acetate. The organic layer was separated, washed with brine, and dried over anhydrous magnesium sulfate. The solvent was distilled off under reduced pressure, and the residue was purified by silica gel column chromatography to obtain 7.3 g 1,1,1-trifluoro-3-nitro-2-(3,4,5-trichlorophenyl)propan-2-ol.

¹H-NMR (CDCl₃) δ: 4.76 (1H, s), 5.00 (1H, s), 7.62 (2H, s).

### Synthesis of Example A-2

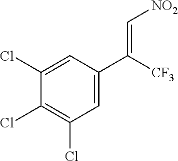 [SMILES: O=[N+]([O-])/C=C(/c1cc(Cl)c(Cl)c(Cl)c1)C(F)(F)F]

0.30 g 1,1,1-Trifluoro-3-nitro-2-(3,4,5-trichlorophenyl)propan-2-ol and 0.53 g thionyl chloride were dissolved in 10 ml toluene. The reaction mixture was cooled to 0° C., and then slowly added with 0.14 g pyridine. The resulting reaction mixture was stirred for 20 hours and refluxed under heating for 1 hour. 2 N Hydrochloric acid was added to the mixture at 0° C. and extracted with ethyl acetate. The organic layer was washed with brine, and dried over anhydrous magnesium sulfate. The solvent was distilled off under reduced pressure, the residue was purified by column chromatography (ethyl acetate/hexane) to obtain 0.1 g 1,2,3-trichloro-5-(3,3,3-trifluoro-1-nitroprop-1-en-2-yl)benzene.

¹H-NMR (CDCl₃) δ: 7.34 (2H, s), 7.56 (1H, m).

### Synthesis of Example A-3

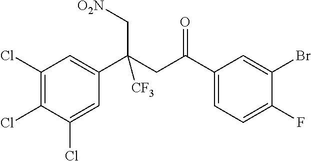 [SMILES: O=C(CC(C[N+](=O)[O-])(c1cc(Cl)c(Cl)c(Cl)c1)C(F)(F)F)c1ccc(F)c(Br)c1]

Under argon atmosphere, to a tetrahydrofuran solution (5 ml) of 0.07 g 3-bromo-4-fluoroacetophenone, 0.16 ml 2.0 M tetrahydrofuran solution of lithium diisopropylamide was added at −75° C. After stirring for 30 minutes, the reaction mixture was added with 0.1 g 1,2,3-trichloro-5-(3,3,3-trifluoro-1-nitroprop-1-en-2-yl)benzene, and the mixture was stirred for 5 hours. A saturated aqueous solution of ammonium chloride was added to the mixture and extracted with ethyl acetate. The organic layer was washed with brine, and dried over anhydrous magnesium sulfate. The solvent was distilled off under reduced pressure to obtain 0.19 g (83% purity) 1-(3-bromo-4-fluorophenyl)-4,4,4-trifluoro-3-(nitromethyl)-3-(3,4,5-trichlorophenyl)-butan-1-one.

¹H-NMR (CDCl₃) δ: 4.01 (2H, dd), 5.49 (2H, dd), 7.08-7.42 (3H, m), 7.92-7.95 (1H, m), 8.18-8.21 (1H, m).

### Synthesis of Example A-4

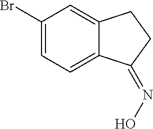 [SMILES: O/N=C1/CCc2cc(Br)ccc21]

5 g 5-Bromoindan-1-one, 1.8 g hydroxylamine hydrochloric acid salt and 2.7 g sodium acetate were added to 80 ml methanol, and the resulting reaction mixture was stirred at room temperature for 20 hours. The reaction solution was concentrated under reduced pressure, and the residue was dissolved into water and t-butyl methyl ether. The organic layer was separated, washed with saturated aqueous solution of sodium hydrogen carbonate and brine, and dried over anhydrous magnesium sulfate. The solvent was distilled off under reduced pressure to obtain 5.3 g 5-bromo-N-hydroxyindan-1-imine

¹H-NMR (CDCl₃) δ: 2.97-3.03 (4H, m), 7.37-7.53 (3H, m).

### Synthesis of Example A-5

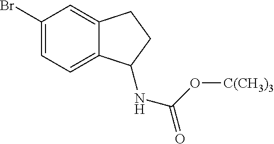 [SMILES: CC(C)(C)OC(=O)NC1CCc2cc(Br)ccc21]

5.3 g 5-Bromo-N-hydroxyindan-1-imine, 10.2 g di-tert-butyl bicarbonate, and 2.8 g nickel chloride hexahydrate were dissolved in 100 ml methanol and 20 ml dioxane. The reaction mixture was cooled to −20° C., slowly added with 3.5 g sodium borohydride. After stirring for 1 hour, the reaction mixture was added with 6.0 g diethylenetriamine, and then stirred for 30 minutes. The reaction mixture was diluted by adding water, and extracted twice with t-butyl methyl ether. The organic layers were dried over anhydrous magnesium sulfate. The solvent was distilled off under reduced pressure, the residue was purified by column chromatography (ethyl acetate/hexane) to 5.6 g obtain tert-butyl (5-bromo-2,3-dihydro-1H-inden-1-yl)carbamate.

¹H-NMR (CDCl₃) δ: 1.48 (9H, s), 1.75-1.80 (1H, m), 2.55-2.59 (1H, m), 2.76-2.98 (3H, m), 4.70-4.73 (1H, m), 5.11-5.14 (1H, m), 7.18-7.21 (1H, m), 7.32-7.35 (2H, m).

### Synthesis of Example A-6

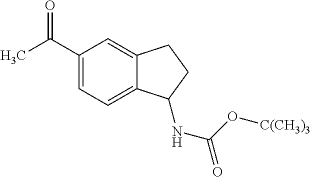 [SMILES: CC(=O)c1ccc2c(c1)CCC2NC(=O)OC(C)(C)C]

Under argon atmosphere, 4.0 g tert-butyl (5-bromo-2,3-dihydro-1H-inden-1-yl)-carbamate was added to a 1.6 M n-butyllithium hexane solution (20 ml) and tetrahydrofuran (100 ml) solution at −75° C. After stirring for 15 minutes, the reaction mixture was added with methyl acetate, and then stirred for 1 hour. The reaction liquid was diluted with t-butyl methyl ether and washed with water and brine. The organic layer was dried over anhydrous magnesium sulfate. The solvent was distilled off under reduced pressure, and the residue was purified by column chromatography (ethyl acetate/hexane) to 2.0 g obtain tert-butyl (5-acetyl-2,3-dihydro-1H-inden-1-yl)carbamate.

¹H-NMR (CDCl₃) δ: 1.50 (9H, s), 1.79-1.85 (1H, m), 2.60-2.64 (4H, m), 2.80-3.05 (2H, m), 4.73-4.76 (1H, m), 5.18-5.30 (1H, m), 7.40 (1H, d), 7.81-7.83 (2H, m).

### Synthesis of Example A-7

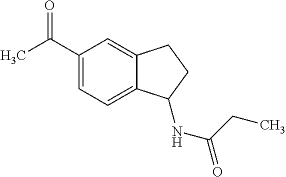 [SMILES: CCC(=O)NC1CCc2cc(C(C)=O)ccc21]

1.8 g Trifluoroacetic acid was added to a 20 ml methylene chloride solution of tert-butyl (5-acetyl-2,3-dihydro-1H-inden-1-yl)carbamate (0.4 g), and stirred at room temperature for 2 hours. The reaction solution was concentrated under reduced pressure, and the residue was dissolved into t-butyl methyl ether and washed with saturated aqueous solution of sodium hydrogen carbonate and brine. The organic layer was dried over anhydrous magnesium sulfate, the solvent was distilled off under reduced pressure, and the residue was dissolved into 10 ml pyridine. 0.3 g acetic anhydride was added and the mixture was stirred at room temperature for 8 hours. The reaction liquid was concentrated under reduced pressure, and subjected to the azeotropic distillation with toluene. The residue was purified by column chromatography (ethyl acetate/hexane) to obtain 0.3 g N-(5-acetyl-2,3-dihydro-1H-inden-1-yl)propanamide.

¹H-NMR (CDCl₃) δ: 1.20 (3H, t), 1.79-1.85 (1H, m), 2.27 (2H, q), 2.51-2.67 (4H, m), 2.77-3.04 (2H, m), 5.48-5.51 (1H, m), 5.99-6.01 (1H, m), 7.29-7.31 (1H, m), 7.75-7.78 (2H, m).

### Synthesis of Example A-8

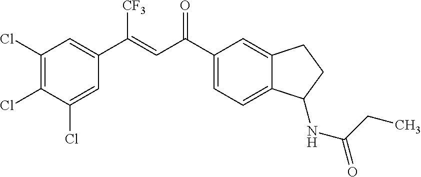 [SMILES: CCC(=O)NC1CCc2cc(C(=O)/C=C(/c3cc(Cl)c(Cl)c(Cl)c3)C(F)(F)F)ccc21]

0.1 g lithium hydride was added to a tetrahydrofuran (20 ml) solution of 2,2,2-trifluoro-1-(3,4,5-trichlorophenyl)ethanone (1.0 g) and N-(5-acetyl-2,3-dihydro-1H-inden-1-yl)propanamide (0.4 g), and the mixture was refluxed under heating for 8 hours. After diluting with t-butyl methyl ether, the reaction mixture was washed with a saturated aqueous solution of sodium hydrogen carbonate and brine. The organic layer was dried over anhydrous magnesium sulfate, the reaction liquid was concentrated under reduced pressure, and the residue was purified by column chromatography (ethyl acetate/hexane) to obtain 0.3 g N-{5-[4,4,4-trifluoro-3-(3,4,5-trichlorophenyl)but-2-enoyl]-2,3-dihydro-1H-inden-1-yl}propanamide.

¹H-NMR (CDCl₃) δ: 1.22 (3H, t), 1.78-1.88 (1H, m), 2.28 (2H, q), 2.63-2.69 (1H, m), 2.87-3.05 (2H, m), 5.51-5.62 (2H, m), 7.28-7.37 (4H, m), 7.65-7.67 (2H, m).

### Synthetic Example A-9

### Synthesis of a Nitroketone According to the Invention

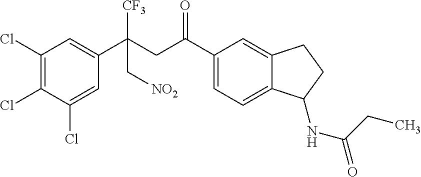 [SMILES: CCC(=O)NC1CCc2cc(C(=O)CC(C[N+](=O)[O-])(c3cc(Cl)c(Cl)c(Cl)c3)C(F)(F)F)ccc21]

To the N,N-dimethylformamide (60 ml) solution of N-{5-[4,4,4-trifluoro-3-(3,4,5-trichlorophenyl)but-2-enoyl]-2,3-dihydro-1H-inden-1-yl}propanamide (2.0 g) and nitromethane (0.5 g), diazabicycloundecene (0.6 g) was added and the mixture was stirred at room temperature for 10 hours. The reaction liquid was diluted with t-butyl methyl ether, and washed three times with brine. The organic layers were dried over anhydrous magnesium sulfate. The reaction solution was concentrated under reduced pressure, and the residue was purified by column chromatography (ethyl acetate/hexane) to obtain 1.0 g N-{5-[4,4,4-trifluoro-3-(nitromethyl)-3-(3,4,5-trichloro-phenyl)butanoyl]-2,3-dihydro-1H-inden-1-yl}propanamide. ¹H-NMR (CDCl₃) δ: 1.22 (3H, t), 1.83-1.86 (1H, m), 2.29 (2H, q), 2.65-2.68 (1H, m), 2.89-3.04 (2H, m), 4.05 (2H, dd), 5.46-5.58 (4H, m), 7.29-7.39 (3H, m), 7.69-7.78 (2H, m).

### B: Method According to the Invention

### Synthesis of 5-(3-bromo-4-fluorophenyl)-3-(3,4,5-trichlorophenyl)-3-(trifluoromethyl)-3,4-dihydro-2H-pyrrole

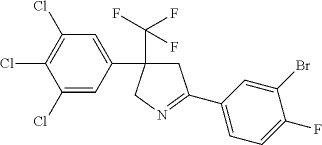 [SMILES: Fc1ccc(C2=NCC(c3cc(Cl)c(Cl)c(Cl)c3)(C(F)(F)F)C2)cc1Br]

In a hastelloy autoclave, 1.06 g 1-(3-bromo-4-fluorophenyl)-4,4,4-trifluoro-3-(nitromethyl)-3-(3,4,5-trichlorophenyl)butane-1-one were dissolved in 14 mL of THF. 300 mg of Raney-cobalt were added and the autoclave was purged with nitrogen. The mixture was stirred for three hours at 20 bar hydrogen pressure and 100° C. After filtration of the catalyst the residue was evaporated to dryness and then submitted to column chromatography. 690 mg (74% theoretical yield) of the target compound were obtained as a white solid.

¹H-NMR (CDCl₃) δ: 8.09-8.07 (m, 1H), 7.80-7.78 (m, 1H), 7.39 (s, 2H), 7.21-7.18 (m, 1H), 4.89-4.86 (m, 1H), 4.44-4.41 (m, 1H), 3.77-3.74 (m, 1H), 3.42-3.39 (m, 1H).

### C: Method According to Chem. Commun. 2009, 2136-2138

When stirring 300 mg of 1-(3-bromo-4-fluorophenyl)-4,4,4-trifluoro-3-(nitromethyl)-3-(3,4,5-trichlorophenyl)butane-1-one in 10 mL of Ethanol in the presence of 100 mg Raney-Nickel for 12 h at room temperature under atmospheric hydrogen pressure in analogy to the reaction conditions described in the prior art (Chem. Commun. 2009, 2136-2138), full consumption of the starting material was observed. Selectivity according to LC (liquid chromatography) was only 22%, thus limiting the maximum theoretical yield to 22%.

### D: Biological Test Data

The test preparations in Biological test examples 1 to 3 were prepared as follows.

Solvent: 3 parts by weight of dimethylformamide; Emulsifier: 1 part by weight of polyoxyethylene alkyl phenyl ether; To prepare a suitable preparation containing the active compound, 1 part by weight of the active compound was mixed with the above amount of the solvent containing the above amount of the emulsifier, and the resulting mixture was diluted with water to a predetermined concentration.

### Biological Test Example 1

### Test on Spodoptera litura Larvae

Leaves of sweet potato were dipped in a solution including the above-prepared active compound which had been diluted to a given concentration with water. The chemical preparation was air-dried and placed in a petri dish (9 cm diameter). Ten Spodoptera litura larvae at their 3rd-instar of metamorphosis were released in the petri dish, which was then placed in a constant temperature room (25° C.). Two and 4 days later, respectively, more sweet potato leaves were added. Seven days later, a pesticidal activity was calculated by counting the number of dead Spodoptera litura larvae. In this case, 100% pesticidal activity means death of all the larvae, while 0% means all surviving. In the present test, an average value was taken from the results obtained from a single zone of two petri dishes.

In the above biological test example 1, as representative examples, Examples Nos. T1-95, T2-52 and T2-127, T4-1335, T4-275, T4-1339 showed the pest controlling effect of 100% pesticidal rate at an effective component concentration of 500 ppm.

### Biological Test Example 2

### Test on Tetranychus Urticae

Two kidney bean leaves at unfolded leaf stage having two main leaves that have been grown in a pot (6 cm diameter), 50 to 100 adult Tetranychus urticae were placed. After 1 day, a generous amount of a solution including the above-prepared active compound that had been diluted to a given concentration with water was sprayed thereto using a spray gun. After keeping the pot in a green house for 7 days, an acaricidal activity was determined. In this case, 100% acaricidal activity means death of all the insects, while 0% means all surviving.

As a representative example, Examples Nos. T4-275, T4-1339 showed the pest controlling effect with 90% acaricidal rate at an effective component concentration of 500 ppm.

As a representative example, Examples Nos. T1-95, T4-1335, showed the pest controlling effect with 100% acaricidal rate at an effective component concentration of 500 ppm.

### Biological Test Example 3

### Test on Aulacophora femoralis

Cucumber leaves were dipped in a solution including the above-prepared active compound that had been diluted to a given concentration with water. The preparation was air-dried and then added to a plastic cup containing sterilized black soil. Five Aulacophora femoralis larvae at their 2nd-instar of metamorphosis were released in the cup, which was then placed in a constant temperature room (25° C.). Seven days later, a pesticidal activity was calculated by counting the number of dead Aulacophora femoralis larvae. In this case, 100% pesticidal activity means death of all the larvae, while 0% means all surviving.

As a representative example, Examples No. T1-95, T4-275, T4-1339 showed the controlling effect with 100% pesticidal rate at an effective component concentration of 500 ppm.

### Biological Test Example 4

### Test on Boophilus microplus

### Preparation of Test Preparations

### Solvent: Dimethylsulfoxide

To produce a suitable preparation of an active compound, 10 mg of the compound of the present invention were dissolved in 0.5 ml solvent, and the concentrate was diluted with animal blood of to the desired concentration.

Five female adult Boophilus microplus ticks with blood engorged stomach were injected in the abdomen with the above compound solution. The ticks were then transferred to a petri dish and bred in a breeder for a certain period of time. After the certain period of time has lapsed, mortality ratio of Boophilus microplus was determined. In this case, 100% indicates that none of the laid eggs were hatched while 0% indicates that all of eggs were hatched.

As a representative example, Example No. T1-95 showed the pesticidal activity of 100% at an effective component concentration of 100 ppm.

### Biological Test Example 6

### Test on Lucillia cuprina

To the test tube including minced horsemeat (1 cm³ size) and the aqueous solution containing the compound which had been prepared in the same manner as Biological test example 4 (0.5 ml), approximately 20 to 30 Lucillia cuprina larvae were added. After a certain period of time has lapsed, mortality ratio of Lucillia cuprina was determined. In this case, 100% indicates that none of the Lucillia cuprina survived while 0% indicates that all of them survived.

As a representative example, Example No. T1-95 showed the pesticidal activity of 100% at an effective component concentration of 100 ppm.

### E: Preparation Examples

### Preparation Example 1

### Granules

To a mixture containing 10 parts of the compound of the present invention, 30 parts of bentonite (montmorillonite), 58 parts of talc and 2 parts of lignin sulfonate is added 25 parts of water, and the mixture was well kneaded and granulated with 10 to 40 meshes by an extruding granulator and dried at 40 to 50° C. to obtain granules.

### Preparation Example 2

### Granules

95 parts of clay mineral granules having particle diameter distribution within the range of 0.2 to 2 mm are put into a rotary mixer, and then wetted evenly by spraying of 5 parts of the compound of the present invention together with a liquid diluent under rotating condition and dried at 40 to 50° C. to obtain granules.

### Preparation Example 3

### Emulsion

30 parts of the compound of the present invention, 55 parts of xylene, 8 parts of polyoxyethylene alkyl phenyl ether and 7 parts of calcium alkylbenzenesulfonate are mixed together to obtain the emulsion.

### Preparation Example 4

### Wettable Agent

15 parts of the compound of the present invention, 80 parts of a mixture of white carbon (hydrated amorphous silicon oxide fine powder) and powdered clay (1:5), formalin condensate of 2 parts of sodium alkylbenzenesulfonate and 3 parts of sodium alkylnaphthalenesulfonate is mixed together and the mixture is crushed to obtain a wettable agent.

### Preparation Example 5

### Wettable Granules

20 parts of the active compound of the present invention, 30 parts of lignin sodium sulfonate, 15 parts of bentonite and 35 parts of calcined diatomaceous earth powder are well mixed, and after addition of water, the mixture is then extruded with a screen of 0.3 mm and dried to obtain wettable granules.

1. A method for the preparation of a pyrroline of formula (I) 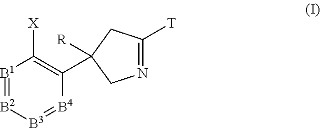
by catalytic hydration of a nitroketone of formula (II) 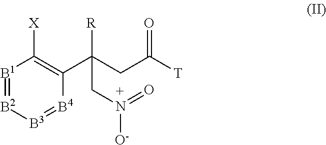
said method comprising employing a transition metal catalyst and gaseous hydrogen at an elevated pressure in a solvent, optionally in the presence of at least one additive selected from the group consisting of Lewis acids, Brønstedt acids, organic sulfur-containing compounds, organic or inorganic bases, and water scavengers
wherein in the formulae (I) and (II)
T is one of the following chemical groupings (T1), (T2), (T3), (T4) or (T5) 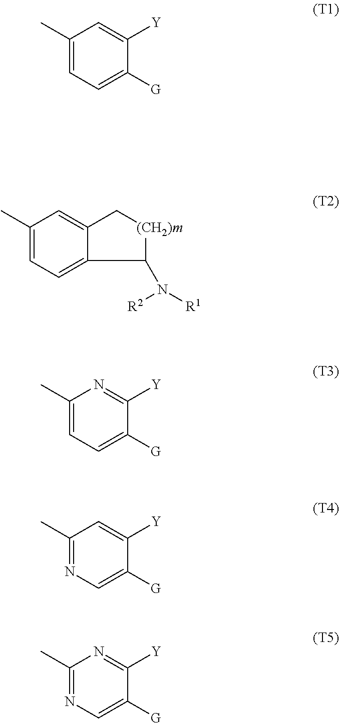
wherein
Y is hydrogen, halogen, nitro, cyano, amino, hydroxy, mercapto, alkyl, haloalkyl, cycloalkyl, halocycloalkyl, alkoxy, haloalkoxy, alkylthio, alkylsulfinyl, alkylsulfonyl, haloalkylthio, haloalkylsulfinyl, haloalkylsulfonyl, alkylsulfonyloxy, haloalkylsulfonyloxy, mono- or di-alkylaminosulfonyl, mono- or di-haloalkylamino-sulfonyl, mono- or di-alkylamino, acylamino, alkoxycarbonylamino, haloalkoxycarbonylamino, alkylsulfonylamino, haloalkylsulfonylamino, trialkylsilyl, alkoxyamino, haloalkoxyamino, alkoxyiminoalkyl, haloalkoxyiminoalkyl, alkylsulfinylimino, alkylsulfinyliminoalkyl, alkylsulfinyliminoalkylcarbonyl, alkylsulfoxyimino, alkylsulfoxyiminoalkyl, alkoxycarbonyl, alkylcarbonyl, aminocarbonyl, mono- or di-alkylaminocarbonyl, aminothiocarbonyl or mono- or di-alkylaminothiocarbonyl, and each group from alkyl to di-alkylaminothiocarbonyl among the definitions of Y may be optionally substituted;
G is a saturated or unsaturated 5- to 6-membered heterocyclic group which may be substituted, or
G is halogen, cyano, alkyl, alkoxycarbonyl, carboxy, or CH₃S, or
G is one of the following groups (G10a) or (G10b): 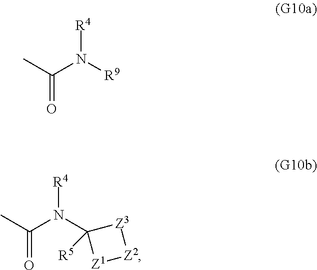
G is one of the following groups (G11) to (G16): 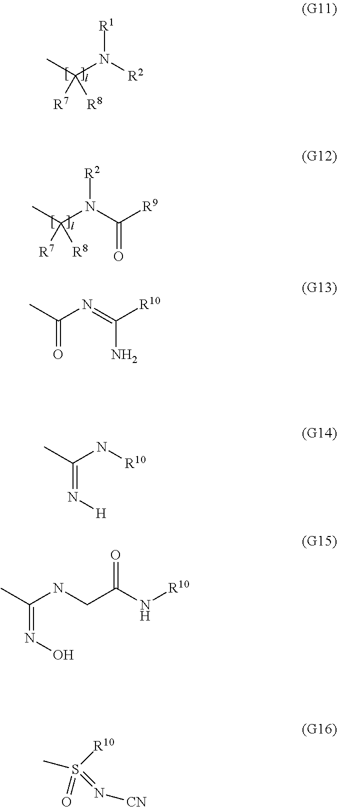
R¹ is hydrogen, cyano, formyl, thioformyl, alkylcarbonyl, haloalkylcarbonyl, alkyl-thiocarbonyl, haloalkyl-thiocarbonyl, mono- or di-alkylaminocarbonyl, mono- or di-alkylamino-thiocarbonyl, alkoxyaminocarbonyl, alkoxyamino-thiocarbonyl, alkoxycarbonyl, alkoxyalkylcarbonyl, alkoxy-thiocarbonyl, alkylthio-carbonyl, alkylthio-thiocarbonyl, alkylsulfonyl, haloalkylsulfonyl, cycloalkylcarbonyl, alkenylcarbonyl, alkynylcarbonyl, cycloalkyl-alkylcarbonyl, alkylthioalkylcarbonyl, alkylsulfinylalkylcarbonyl, alkylsulfonylalkylcarbonyl, alkylcarbonylalkylcarbonyl, cycloalkylaminocarbonyl, alkenylaminocarbonyl, alkynylaminocarbonyl, —CH₂—R³, —C(O)R³ or —C(S)R³, and each group from alkylcarbonyl to alkynylaminocarbonyl among the definitions of R¹ may be substituted;
R² is hydrogen, amino, hydroxy, cyano, alkyl, haloalkyl, cycloalkyl, alkenyl, alkynyl, alkylimino, alkoxy, alkylcarbonyl, alkylcarbonylamino, alkoxyalkyl, cyanoalkyl, alkoxycarbonylalkyl, —CH₂—R³, —C(O)R³ or —C(S)R³, and each group from alkyl to alkylcarbonylamino among the definitions of R² may be substituted; or
R¹ and R² together with the N atom to which they are bound, may form a 3- to 6-membered heterocyclic ring which may be substituted and may further comprise N, O or S;
R³ is phenyl which may be substituted, a 5- to 6-membered heterocyclic ring which may be substituted, optionally substituted alkyl, amino, or mono- or diaalkylamino;
m is 1 or 2;
R⁴ and R⁵ independently are hydrogen, halogen, alkyl which may be substituted or haloalkyl which may be substituted;
Z¹, Z² and Z³ independently is —CR⁴R⁵—, —C(O)—, —C(N—OR⁶)—, —NR⁶—, —S(O)ₙ—, —S(N—R⁶)— or —S(O)(N—R⁶)—, or oxygen with the proviso that Z¹, Z² and Z³ do not simultaneously represent —CR⁴R⁵— or do not simultaneously represent oxygen;
n is 0, 1 or 2;
R⁶ is hydrogen, cyano, nitro, alkyl, haloalkyl, cycloalkylalkyl, alkylcarbonyl, haloalkylcarbonyl, alkoxycarbonyl, haloalkoxycarbonyl, alkylsulfonyl, haloalkylsulfonyl, aralkyl (arylalkyl) or alkyl which is substituted with a heterocycle, and each group from alkyl to alkyl which is substituted with a heterocycle among the definitions of R⁶ may be substituted;
R⁷ and R⁸ each independently are hydrogen, cyano, alkyl, cycloalkyl, haloalkyl, cyclohaloalkyl, alkenyl, alkynyl, haloalkenyl, haloalkynyl, alkoxycarbony, or alkoxythiocarbonyl, and each of the groups from alkyl to alkoxythiocarbonyl among the definition of R⁷ and R⁸ may be substituted; or
R⁹ is hydrogen, alkyl, alkoxy, haloalkyl, mono- or di-alkylamino, alkoxyamino, alkoxyalkyl, alkylhio, cycloalkyl, alkenyl, alkynyl, cycloalkylalkyl, alkylthioalkyl, alkylsulfinylalkyl, alkylsulfonylalkyl, alkylcarbonylalkyl, cycloalkylamino, alkenylamino, alkynylamino, —CH₂R³, —C(O)R³ or —C(S)R³, and each group from alkyl to alkynylamino among the definitions of R⁹ may be substituted;
R¹⁰ is hydrogen, hydroxy, alkyl, haloalkyl, cycloalkyl, alkenyl, alkinyl, alkoxy, alkylcarbonyl, alkoxycarbonyl, alkoxyalkyl, cyanoalkyl, alkoxycarbonylalkyl, —CH₂R³, —C(O)R³ or —C(S)R³ and each group from alkyl to alkoxycarbonylalkyl among the definitions of R¹⁰ may be substituted;
l stands for 1, 2 or 3;
R is alkyl which may be substituted or haloalkyl which may be substituted;
X is hydrogen, halogen, nitro, cyano, hydroxy, mercapto, amino, alkyl, haloalkyl, alkoxy, haloalkoxy, alkylthio, alkylsulfinyl, alkylsulfonyl, haloalkylthio, haloalkylsulfinyl, haloalkylsulfonyl, acylamino, alkoxycarbonylamino, haloalkoxycarbonylamino, alkoxyimino, haloalkoxyimino, alkylsulfonylamino or sulfur pentafluoride, and each group from alkyl to alkylsulfonylamino among the definitions of X may be optionally substituted; and
B¹, B², B³ and B⁴ independently is C—X or N.

2. The method according to claim 1, wherein said elevated pressure is in a range from 2 to 100 bar.

3. The method according to claim 1, wherein the transition
metal catalyst used comprises at least one metal selected from the group consisting of platinum, palladium, cobalt and nickel.

4. The method according to claim 3, wherein Raney-Nickel is used as transition metal catalyst in the presence of an organic sulfur-containing compound.

5. The method according to claim 4, wherein said organic sulfur-containing compound is selected from the group consisting of thiophene, tetrahydrothiophene and 2,2′-thiobisethanol.

6. A nitroketone of formula (II) 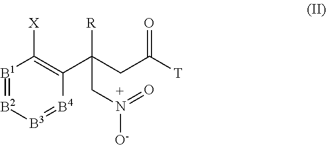
wherein
T is one of the following chemical groupings (T1), (T2), (T3), (T4) or (T5) 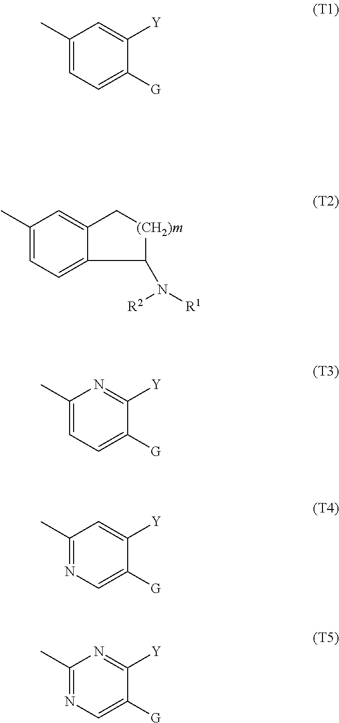
wherein
Y is hydrogen, halogen, nitro, cyano, amino, hydroxy, mercapto, alkyl, haloalkyl, cycloalkyl, halocycloalkyl, alkoxy, haloalkoxy, alkylthio, alkylsulfinyl, alkylsulfonyl, haloalkylthio, haloalkylsulfinyl, haloalkylsulfonyl, alkylsulfonyloxy, haloalkylsulfonyloxy, mono- or di-alkylaminosulfonyl, mono- or di-haloalkylamino-sulfonyl, mono- or di-alkylamino, acylamino, alkoxycarbonylamino, haloalkoxycarbonylamino, alkylsulfonylamino, haloalkylsulfonylamino, trialkylsilyl, alkoxyamino, haloalkoxyamino, alkoxyiminoalkyl, haloalkoxyiminoalkyl, alkylsulfinylimino, alkylsulfinyliminoalkyl, alkylsulfinyliminoalkylcarbonyl, alkylsulfoxyimino, alkylsulfoxyiminoalkyl, alkoxycarbonyl, alkylcarbonyl, aminocarbonyl, mono- or di-alkylaminocarbonyl, aminothiocarbonyl or mono- or di-alkylaminothiocarbonyl, and each group from alkyl to di-alkylaminothiocarbonyl among the definitions of Y may be optionally substituted;
G is a saturated or unsaturated 5- to 6-membered heterocyclic group which may be substituted, or
G is halogen, cyano, alkyl, alkoxycarbonyl, carboxy, or CH₃S, or
G is one of the following groups (G10a) or (G10b): 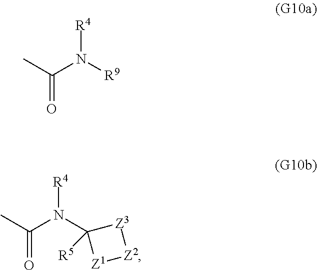
G is one of the following groups (G11) to (G16): 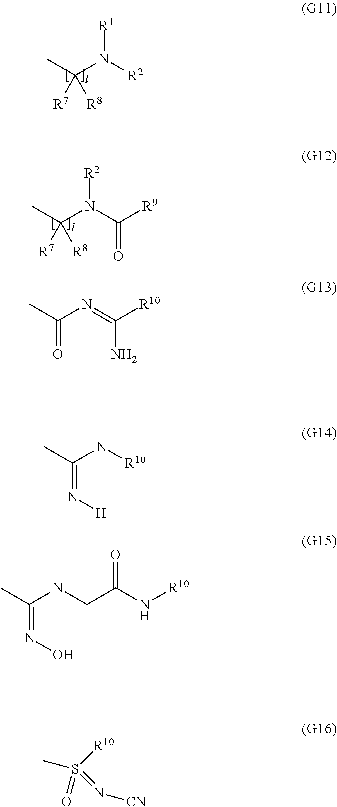
R¹ is hydrogen, cyano, formyl, thioformyl, alkylcarbonyl, haloalkylcarbonyl, alkyl-thiocarbonyl, haloalkyl-thiocarbonyl, mono- or di-alkylaminocarbonyl, mono- or di-alkylamino-thiocarbonyl, alkoxyaminocarbonyl, alkoxyamino-thiocarbonyl, alkoxycarbonyl, alkoxyalkylcarbonyl, alkoxy-thiocarbonyl, alkylthio-carbonyl, alkylthio-thiocarbonyl, alkylsulfonyl, haloalkylsulfonyl, cycloalkylcarbonyl, alkenylcarbonyl, alkynylcarbonyl, cycloalkyl-alkylcarbonyl, alkylthioalkylcarbonyl, alkylsulfinylalkylcarbonyl, alkylsulfonylalkylcarbonyl, alkylcarbonylalkylcarbonyl, cycloalkylaminocarbonyl, alkenylaminocarbonyl, alkenylaminocarbonyl, —CH₂—R³, —C(O)R³ or —C(S)R³, and each group from alkylcarbonyl to alkynylaminocarbonyl among the definitions of R¹ may be substituted;
R² is hydrogen, amino, hydroxy, cyano, alkyl, haloalkyl, cycloalkyl, alkenyl, alkenyl, alkylimino, alkoxy, alkylcarbonyl, alkylcarbonylamino, alkoxyalkyl, cyanoalkyl, alkoxycarbonylalkyl, —CH₂—R³, —C(O)R³ or —C(S)R³, and each group from alkyl to alkylcarbonylamino among the definitions of R² may be substituted; or
R¹ and R² together with the N atom to which they are bound, may form a 3- to 6-membered heterocyclic ring which may be substituted and may further comprise N, O or S;
R³ is phenyl which may be substituted, a 5- to 6-membered heterocyclic ring which may be substituted, optionally substituted alkyl, amino, or mono- or diaalkylamino;
m is 1 or 2;
R⁴ and R⁵ independently are hydrogen, halogen, alkyl which may be substituted or haloalkyl which may be substituted;
Z¹, Z² and Z³ independently is —CR⁴R⁵—, —C(O)—, —C(N—OR⁶)—, —NR⁶—, —S(O), —S(N—R⁶)— or —S(O)(N—R⁶)—, or oxygen with the proviso that Z¹, Z² and Z³ do not simultaneously represent —CR⁴R⁵— or do not simultaneously represent oxygen;
n is 0, 1 or 2;
R⁶ is hydrogen, cyano, nitro, alkyl, haloalkyl, cycloalkylalkyl, alkylcarbonyl, haloalkylcarbonyl, alkoxycarbonyl, haloalkoxycarbonyl, alkylsulfonyl, haloalkylsulfonyl, aralkyl (arylalkyl) or alkyl which is substituted with a heterocycle, and each group from alkyl to alkyl which is substituted with a heterocycle among the definitions of R⁶ may be substituted;
R⁷ and R⁸ each independently are hydrogen, cyano, alkyl, cycloalkyl, haloalkyl, cyclohaloalkyl, alkenyl, alkynyl, haloalkenyl, haloalkynyl, alkoxycarbony, or alkoxythiocarbonyl, and each of the groups from alkyl to alkoxythiocarbonyl among the definition of R⁷ and R⁸ may be substituted; or
R⁹ is hydrogen, alkyl, alkoxy, haloalkyl, mono- or di-alkylamino, alkoxyamino, alkoxyalkyl, alkylhio, cycloalkyl, alkenyl, alkynyl, cycloalkylalkyl, alkylthioalkyl, alkylsulfinylalkyl, alkylsulfonylalkyl, alkylcarbonylalkyl, cycloalkylamino, alkenylamino, alkynylamino, —CH₂R³, —C(O)R³ or —C(S)R³, and each group from alkyl to alkynylamino among the definitions of R⁹ may be substituted;
R¹⁰ is hydrogen, hydroxy, alkyl, haloalkyl, cycloalkyl, alkenyl, alkinyl, alkoxy, alkylcarbonyl, alkoxycarbonyl, alkoxyalkyl, cyanoalkyl, alkoxycarbonylalkyl, —CH₂R³, —C(O)R³ or —C(S)R³ and each group from alkyl to alkoxycarbonylalkyl among the definitions of R¹⁰ may be substituted;
l stands for 1, 2 or 3;
R is alkyl which may be substituted or haloalkyl which may be substituted;
X is hydrogen, halogen, nitro, cyano, hydroxy, mercapto, amino, alkyl, haloalkyl, alkoxy, haloalkoxy, alkylthio, alkylsulfinyl, alkylsulfonyl, haloalkylthio, haloalkylsulfinyl, haloalkylsulfonyl, acylamino, alkoxycarbonylamino, haloalkoxycarbonylamino, alkoxyimino, haloalkoxyimino, alkylsulfonylamino or sulfur pentafluoride, and each group from alkyl to alkylsulfonylamino among the definitions of X may be optionally substituted; and
B¹, B², B³ and B⁴ independently is C—X or N.

7. The nitroketone of formula (II) according to claim 6, in which
T is one of the following chemical groupings (T1), (T2), (T3), (T4) or (T5) 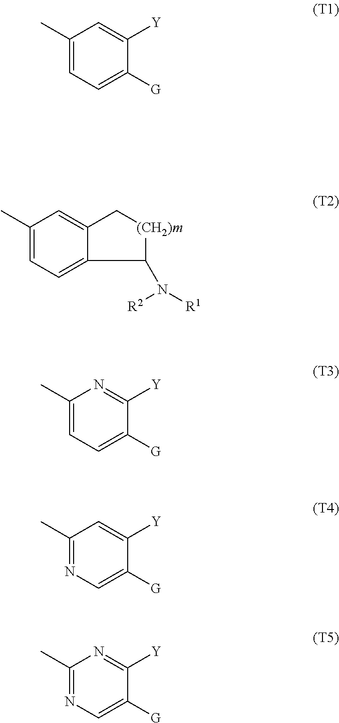
wherein
Y is hydrogen, halogen, nitro, cyano, amino, hydroxy, mercapto, C₁₋₁₂ alkyl, C₁₋₁₂ haloalkyl, C₃₋₈ cycloalkyl, C₃₋₈ halocycloalkyl, C₁₋₁₂ alkoxy, C₁₋₁₂ haloalkoxy, C₁₋₁₂ alkylthio, C₁₋₁₂ alkylsulfinyl, C₁₋₁₂ alkylsulfonyl, C₁₋₁₂ haloalkylthio, C₁₋₁₂ haloalkylsulfinyl, C₁₋₁₂ haloalkylsulfonyl, C₁₋₁₂ alkylsulfonyloxy, C₁₋₁₂ haloalkylsulfonyloxy, mono-(C₁₋₁₂) or di-(C₂₋₂₄) alkylaminosulfonyl, mono-(C₁₋₁₂) or di-(C₂₋₂₄) haloalkylaminosulfonyl, mono-(C₁₋₁₂) or di(C₂₋₂₄) alkylamino, C₁₋₁₂ alkyl-carbonylamino, C₃₋₈ cycloalkyl-carbonylamino, benzoylamino, C₁₋₁₂ alkoxy-carbonylamino, C₁₋₁₂ haloalkoxy-carbonylamino, C₁₋₁₂ alkylsulfonylamino, C₁₋₁₂ haloalkylsulfonylamino, C₃₋₃₆ trialkylsilyl, C₁₋₁₂ alkoxyamino, C₁₋₁₂ haloalkoxyamino, C₁₋₁₂ alkoxyimino-C₁₋₁₂ alkyl, C₁₋₁₂ haloalkoxyimino-C₁₋₁₂ alkyl, C₁₋₁₂ alkylsulfinylimino, C₁₋₁₂ alkylsulfinylimino-C₁₋₁₂ alkyl, C₁₋₁₂ alkylsulfinylimino-C₁₋₁₂ alkyl-carbonyl, C₁₋₁₂ alkylsulfoxyimino, C₁₋₁₂ alkylsulfoxyimino-C₁₋₁₂ alkyl, C₁₋₁₂ alkoxy-carbonyl, C₁₋₁₂ alkyl-carbonyl, aminocarbonyl, mono-(C₁₋₁₂) or di-(C₂₋₂₄) alkyl-aminocarbonyl, aminothiocarbonyl or mono-(C₁₋₁₂) or di-(C₂₋₂₄) alkylaminothio-carbonyl, and each group from C₁₋₁₂ alkyl to di-(C₂₋₂₄) alkylaminothio-carbonyl among the definitions of Y may be optionally substituted;
G is one of the following heterocyclic groups (G1) to (G9): 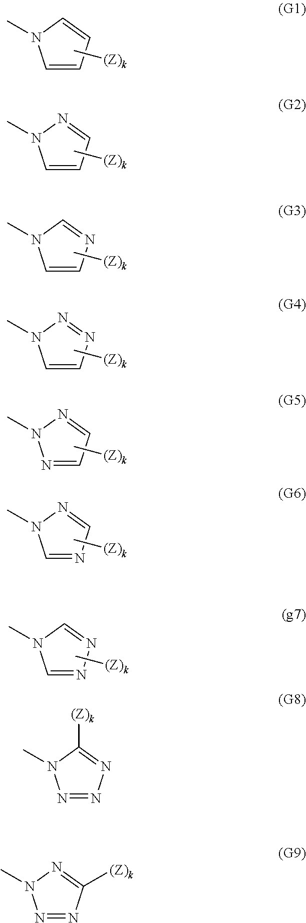
wherein in the groups (G1) to (G9), the substituent (Z) is C₁₋₆ alkyl, C₁₋₆ haloalkyl, or halogen, and k is 0, 1, 2, 3 or 4, or
G is halogen, cyano, C₁₋₁₂ alkyl, C₁₋₁₂ alkoxy-carbonyl, carboxy or CH₃S, or
G is one of the following groups (G10a) or (G10b): 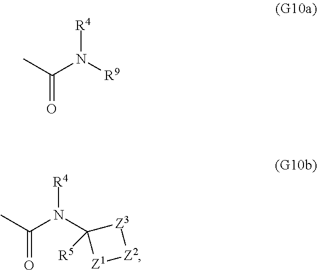
or
G is one of the following groups (G11) to (G16): 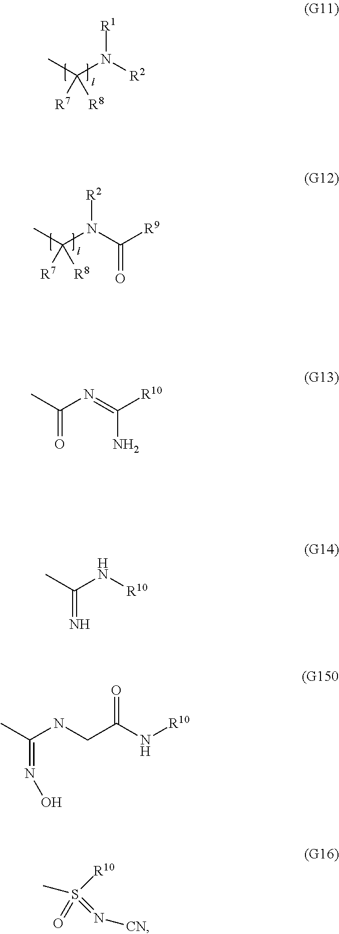
R¹ is hydrogen, cyano, formyl, thioformyl, C₁₋₁₂ alkyl-carbonyl, C₁₋₁₂ haloalkyl-carbonyl, C₁₋₁₂ alkyl-thiocarbonyl, C₁₋₁₂ haloalkyl-thiocarbonyl, mono-(C₁₋₁₂) or di-(C₂₋₂₄) alkyl-aminocarbonyl, mono-(C₁₋₁₂) or di-(C₂₋₂₄) alkylamino-thiocarbonyl, C₁₋₁₂ alkoxy-aminocarbonyl, C₁₋₁₂ alkoxyamino-thiocarbonyl, C₁₋₁₂ alkoxy-carbonyl, C₁₋₁₂ alkoxy-C₁₋₁₂ alkyl-carbonyl, C₁₋₁₂ alkoxy-thiocarbonyl, C₁₋₁₂ alkylthio-carbonyl, C₁₋₁₂ alkylthio-thiocarbonyl, C₁₋₁₂ alkylsulfonyl, C₁₋₁₂ haloalkylsulfonyl, C₃₋₈ cycloalkyl-carbonyl, C₂₋₆ alkenyl-carbonyl, C₂₋₆ alkynyl-carbonyl, C₃₋₈ cycloalkyl-C₁₋₄ alkyl-carbonyl, C₁₋₁₂ alkylthio-C₁₋₁₂ alkyl-carbonyl, C₁₋₁₂ alkylsulfinyl-C₁₋₁₂alkyl-carbonyl, C₁₋₁₂ alkylsulfonyl-C₁₋₁₂alkyl-carbonyl, C₁₋₁₂ alkylcarbonyl-C₁₋₁₂ alkyl-carbonyl, C₃₋₈ cycloalkylamino-carbonyl, C₂₋₆ alkenylamino-carbonyl, C₂₋₆ alkynylamino-carbonyl, —C(O)R³ or —C(S)R³, and each group from C₁₋₁₂ alkyl-carbonyl to C₂₋₆ alkynylamino-carbonyl among the definitions of R¹ may be optionally substituted;
R² is hydrogen, amino, hydroxy, cyano, C₁₋₁₂ alkyl, C₁₋₁₂ haloalkyl, C₃₋₈ cycloalkyl, C₂₋₆ alkenyl, C₂₋₆ alkynyl, C₁₋₁₂ alkylimino, C₁₋₁₂ alkoxy, C₁₋₁₂ alkyl-carbonyl, C₁₋₁₂ alkyl-carbonylamino, C₁₋₁₂ alkoxy-C₁₋₁₂ alkyl, C₁₋₁₂ cyanoalkyl, C₁₋₁₂ alkoxycarbonyl-C₁₋₁₂ alkyl, —CH₂—R³, —C(O)R³ or —C(S)R³, and each group from C₁₋₁₂ alkyl to C₁₋₁₂ alkyl-carbonylamino among the definitions of R² may be optionally substituted; or
R¹ and R², together with the N atom to which they are bound, may form a 3- to 6-membered heterocyclic ring which may be substituted and may further comprise N, O or S;
R³ is phenyl which may be substituted, a 5- to 6-membered heterocyclic group which may be substituted and comprises at least one of N, O and S, optionally substituted C₁₋₁₂ alkyl, amino, mono- or di(C₁₋₁₂)alkylamino;
m is 1 or 2;
R⁴ and R⁵ independently are hydrogen, halogen, C₁₋₁₂ alkyl which may be substituted or C₁₋₁₂ haloalkyl which may be substituted;
Z¹, Z² and Z³ independently are —CR⁴R⁵—, —C(O)—, —C(N—OR⁶)—, —NR⁶—, —S(O)ₙ—, —S(N—R⁶)—, —S(O)(N—R⁶)—, or oxygen, with the proviso that Z¹, Z² and Z³ do not simultaneously represent —CR⁴R⁵—, or do not simultaneously represent oxygen;
n is 0, 1 or 2;
R⁶ is hydrogen, cyano, nitro, C₁₋₁₂ alkyl, C₁₋₁₂ haloalkyl, C₃₋₈ cycloalkyl-C₁₋₄ alkyl, C₁₋₁₂ alkyl-carbonyl, C₁₋₁₂ haloalkyl-carbonyl, C₁₋₁₂ alkoxy-carbonyl, C₁₋₁₂ haloalkoxy-carbonyl, C₁₋₁₂ alkylsulfonyl, C₁₋₁₂ haloalkylsulfonyl, benzyl which may be substituted, phenethyl which may be substituted or C₁₋₄ alkyl which is substituted with a 6-membered heterocycle comprising 1 to 3 nitrogen, and each group from C₁₋₁₂ alkyl to C₁₋₄ alkyl which is substituted with a 6-membered heterocycle comprising 1 to 3 nitrogen among the definitions of R⁶ may be optionally substituted;
R⁷ and R⁸ each independently is hydrogen, cyano, C₁₋₁₂ alkyl, C₃₋₈ cycloalkyl, C₁₋₁₂ haloalkyl, C₃₋₈ cyclohaloalkyl, C₂₋₁₂ alkenyl, C₂₋₁₂ alkynyl, C₂₋₁₂ haloalkenyl, C₂₋₁₂ haloalkynyl, C₁₋₁₂ alkoxy-carbonyl, C₁₋₁₂ alkoxy-thiocarbonyl, and each of the groups from C₁₋₁₂ alkyl to C₁₋₁₂ alkoxy-thiocarbonyl among the groups of R⁷ and R⁸ may be substituted;
R⁷ and R⁸ together with the carbon atom to which they are bound may form a 3 to 6-membered carbocyclic ring;
R⁹ is hydrogen, C₁₋₁₂ alkyl, C₁₋₁₂ alkoxy, C₁₋₁₂ haloalkyl, mono- or di-(C₁₋₁₂)alkylamino, C₁₋₁₂ alkoxyamino, C₁₋₁₂ alkoxy(C₁₋₁₂)alkyl, C₁₋₁₂ alkylhio, C₃₋₈ cycloalkyl, C₂₋₁₂ alkenyl, C₂₋₁₂ alkynyl, C₃₋₈ cycloalkylalkyl, C₁₋₁₂ alkylthio(C₁₋₁₂)alkyl, C₁₋₁₂ alkylsulfinyl(C₁₋₁₂)alkyl, C₁₋₁₂ alkylsulfonyl(C₁₋₁₂)alkyl, C₁₋₁₂alkylcarbonyl(C₁₋₁₂)alkyl, C₃₋₈ cycloalkylamino, C₂₋₁₂ alkenylamino, C₂₋₁₂ alkynylamino, —CH₂R³, —C(O)R³ or —C(S)R³, and each group from C₁₋₁₂ alkyl to C₂₋₁₂ alkynylamino among the definitions of R⁹ may be substituted;
R¹⁰ is hydrogen, hydroxy, C₁₋₁₂ alkyl, C₁₋₁₂ haloalkyl, C₃₋₈ cycloalkyl, C₂₋₁₂ alkenyl, C₂₋₁₂ alkinyl, C₁₋₁₂ alkoxy, C₁₋₁₂ alkylcarbonyl, C₁₋₁₂ alkoxycarbonyl, C₁₋₁₂ alkoxy(C₁₋₁₂)alkyl, C₁₋₁₂ cyanoalkyl, C₁₋₁₂ alkoxycarbonyl(C₁₋₁₂)alkyl, —CH₂R³, —C(O)R³ or —C(S)R³ and each group from C₁₋₁₂ alkyl to C₁₋₁₂ alkoxycarbonyl(C₁₋₁₂)alkyl among the definitions of R¹⁰ may be substituted;
l stands for 1, 2 or 3;
R is C₁₋₁₂ alkyl which may be substituted or C₁₋₁₂ haloalkyl which may be substituted;
X is hydrogen, halogen, nitro, cyano, hydroxy, mercapto, amino, C₁₋₁₂ alkyl, C₁₋₁₂ haloalkyl, C₁₋₁₂ alkoxy, C₁₋₁₂ haloalkoxy, C₁₋₁₂ alkylthio, C₁₋₁₂ alkylsulfinyl, C₁₋₁₂ alkylsulfonyl, C₁₋₁₂ haloalkylthio, C₁₋₁₂ haloalkylsulfinyl, C₁₋₁₂ haloalkylsulfonyl, C₁₋₁₂ alkylcarbonylamino, benzoylamino, C₁₋₁₂ alkoxy-carbonylamino, C₁₋₁₂ haloalkoxy-carbonylamino, C₁₋₁₂ alkoxyimino, C₁₋₁₂ haloalkoxyimino, C₁₋₁₂ alkylsulfonylamino or sulfur pentafluoride, and each group from C₁₋₁₂ alkyl to C₁₋₁₂ alkylsulfonylamino among the definitions of X may be optionally substituted;
B¹, B², B³ and B⁴ independently are C—X or N;

8. The nitroketone of formula (II) according to claim 6, in which
T is one of the following chemical groupings (T1), (T2), (T3), (T4) or (T5) 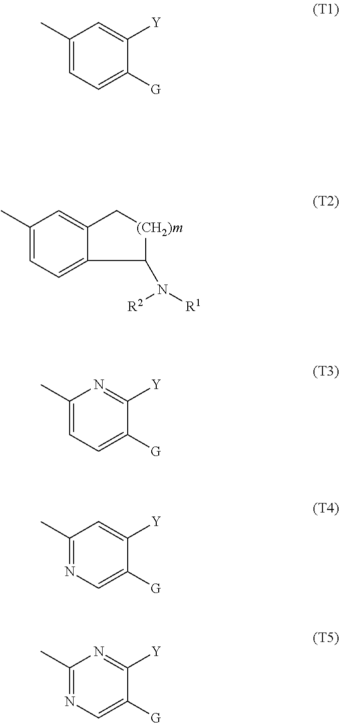
wherein
Y is hydrogen, halogen, nitro, cyano, amino, hydroxy, mercapto, C₁₋₆ alkyl, C₁₋₆ haloalkyl, C₃₋₆ cycloalkyl, C₃₋₆ halocycloalkyl, C₁₋₆ alkoxy, C₁₋₆ haloalkoxy, C₁₋₆ alkylthio, C₁₋₆ alkylsulfinyl, C₁₋₆ alkylsulfonyl, C₁₋₆ haloalkylthio, C₁₋₆ haloalkylsulfinyl, C₁₋₆ haloalkylsulfonyl, C₁₋₆ alkylsulfonyloxy, C₁₋₆ haloalkylsulfonyloxy, mono-(C₁₋₆) or di-(C₂₋₁₂) alkylaminosulfonyl, mono-(C₁₋₆) or di-(C₂₋₁₂) haloalkylaminosulfonyl, mono-(C₁₋₆) or di-(C₂₋₁₂) alkylamino, C₁₋₆ alkyl-carbonylamino, C₃₋₆ cycloalkyl-carbonylamino, benzoylamino, C₁₋₆ alkoxy-carbonylamino, C₁₋₆ haloalkoxy-carbonylamino, C₁₋₆ alkylsulfonylamino, C₁₋₆ haloalkylsulfonylamino, C₃₋₁₈ trialkylsilyl, C₁₋₆ alkoxyamino, C₁₋₆ haloalkoxyamino, C₁₋₆ alkoxyimino-C₁₋₆ alkyl, C₁₋₆ haloalkoxyimino-C₁₋₆ alkyl, C₁₋₆ alkylsulfinylimino, C₁₋₆ alkylsulfinylimino-C₁₋₆ alkyl, C₁₋₆ alkylsulfinylimino-C₁₋₆ alkyl-carbonyl, C₁₋₆ alkylsulfoxyimino, C₁₋₆ alkylsulfoxyimino-C₁₋₆ alkyl, C₁₋₆ alkoxy-carbonyl, C₁₋₆ alkyl-carbonyl, aminocarbonyl, mono-(C₁₋₆) or di-(C₂₋₁₂) alkyl-aminocarbonyl, aminothiocarbonyl or mono-(C₁₋₆) or di-(C₂₋₁₂) alkylaminothio-carbonyl, and each group from C₁₋₆ alkyl to di-(C₂₋₁₂) alkylaminothio-carbonyl among the definitions of Y may be optionally substituted;
G is one of the following heterocyclic groups (G1) to (G9): 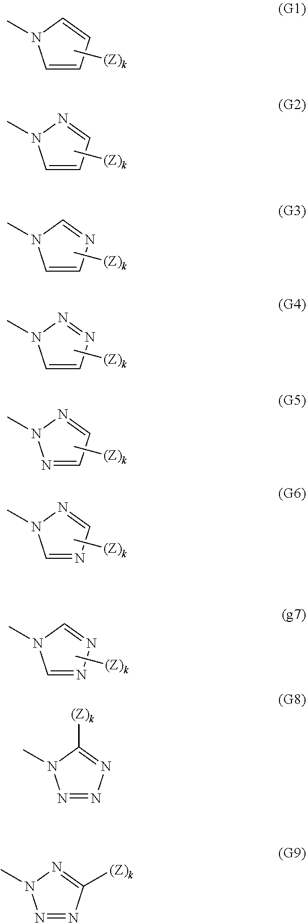
wherein in the groups (G1) to (G9), the substituent (Z) is C₁₋₆ alkyl, C₁₋₆ haloalkyl, halogen, cyano or nitro, and k is 0, 1, 2, 3 or 4, or
G is halogen, cyano, C₁₋₆ alkyl, C₁₋₆ alkoxy-carbonyl, carboxy or CH₃S, or
G is one of the following groups (G10a) or (G10b): 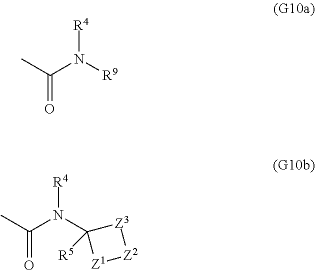
G is one of the following groups (G11) to (G16): 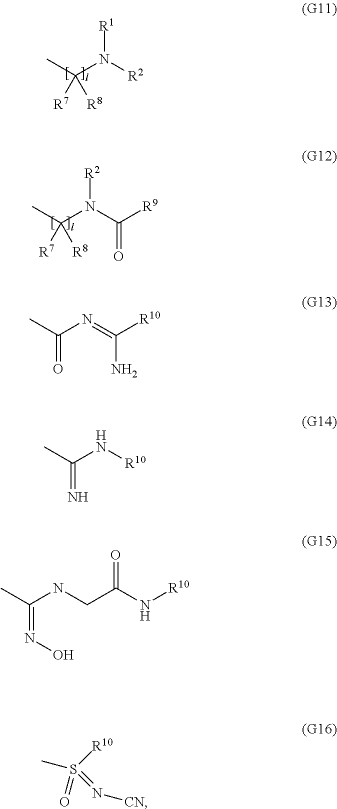
R¹ is hydrogen, cyano, carbonyl, thiocarbonyl, C₁₋₆ alkyl-carbonyl, C₁₋₆ haloalkyl-carbonyl, C₁₋₆ alkyl-thiocarbonyl, C₁₋₆ haloalkyl-thiocarbonyl, mono-(C₁₋₆) or di-(C₂₋₁₂) alkyl-aminocarbonyl, mono-(C₁₋₆) or di-(C₂₋₁₂) alkylamino-thiocarbonyl, C₁₋₆ alkoxy-aminocarbonyl, C₁₋₆ alkoxyamino-thiocarbonyl, C₁₋₆ alkoxy-carbonyl, C₁₋₆ alkoxy-C₁₋₆ alkyl-carbonyl, C₁₋₆ alkoxy-thiocarbonyl, C₁₋₆ alkylthio-carbonyl, C₁₋₆ alkylthio-thiocarbonyl, C₁₋₆ alkylsulfonyl, C₁₋₆ haloalkylsulfonyl, C₃₋₆ cycloalkyl-carbonyl, C₂₋₄ alkenyl-carbonyl, C₂₋₄ alkynyl-carbonyl, C₃₋₆ cycloalkyl-C₁₋₂ alkyl-carbonyl, C₁₋₆ alkylthio-C₁₋₆ alkyl-carbonyl, C₁₋₆ alkylsulfinyl-C₁₋₆ alkyl-carbonyl, C₁₋₆ alkylsulfonyl-C₁₋₆ alkyl-carbonyl, C₁₋₆ alkylcarbonyl-C₁₋₆ alkyl-carbonyl, C₃₋₆ cycloalkylamino-carbonyl, C₂₋₄ alkenylamino-carbonyl, C₂₋₄ alkynylamino-carbonyl, —CH₂—R³, —C(O)R³ or —C(S)R³, and each group from C₁₋₆ alkyl to C₁₋₆ alkynylamino-carbonyl among the definitions of R¹ may be optionally substituted;
R² is hydrogen, amino, hydroxy, cyano, C₁₋₆ alkyl, C₁₋₆ haloalkyl, C₃₋₆ cycloalkyl, C₂₋₄ alkenyl, C₂₋₄ alkynyl, C₁₋₆ alkylimino, C₁₋₆ alkoxy, C₁₋₆ alkyl-carbonyl, C₁₋₆ alkyl-carbonylamino, C₁₋₆ alkoxy-C₁₋₆ alkyl, C₁₋₆ cyanoalkyl, C₁₋₆ alkoxycarbonyl-C₁₋₆ alkyl, —CH₂—R³, —C(O)R³ or —C(S)R³, and each group from C₁₋₆ alkyl to C₁₋₆ alkyl-carbonylamino among the definitions of R² may be optionally substituted; or
R¹ and R², together with the N atom to which they are bound, may form a 3- to 6-membered heterocyclic ring which may be substituted and may further comprise N, O or S; preferably R¹ and R², together with the N atom to which they are bound, may form a 3- to 6-membered heterocyclic ring which may be substituted and may further comprise N, O or S;
R³ is optionally substituted phenyl, pyridyl, pyrimidinyl, or a group (G1) to (G9) as defined herein, or an optionally substituted C₁₋₆ alkyl, amino, mono- or di(C₁₋₁₂)alkylamino group;
m is 1 or 2;
R⁴ and R⁵ independently are hydrogen, halogen, C₁₋₆ alkyl which may be substituted or C₁₋₆haloalkyl which may be substituted;
Z¹, Z² and Z³ independently are —CR⁴R⁵—, —C(O)—, —C(N—OR⁶)—, —NR⁶—, —S(O)ₙ—, —S(N—R⁶)—, —S(O)(N—R⁶)—, or oxygen, with the proviso that Z¹, Z² and Z³ do not simultaneously represent —CR⁴R⁵—, or do not simultaneously represent oxygen;
n is 0, 1 or 2;
R⁶ is hydrogen, cyano, nitro, C₁₋₆ alkyl, C₁₋₆ haloalkyl, C₃₋₆ cycloalkyl-C₁₋₂ alkyl, C₁₋₆ alkyl-carbonyl, C₁₋₆ haloalkyl-carbonyl, C₁₋₆ alkoxy-carbonyl, C₁₋₆ haloalkoxy-carbonyl, C₁₋₆ alkylsulfonyl, C₁₋₆ haloalkylsulfonyl, benzyl which may be substituted, phenethyl which may be substituted or C₁₋₄ alkyl which is substituted with a 6-membered heterocycle comprising 1 to 3 nitrogen, and each group from C₁₋₆ alkyl to C₁₋₄ alkyl which is substituted with a 6-membered heterocycle comprising 1 to 3 nitrogen among the definitions of R⁶ may be optionally substituted;
R² and R⁸ each independently is hydrogen, cyano, C₁₋₆ alkyl, C₃₋₆ cycloalkyl, C₁₋₆ haloalkyl, C₃₋₆ cyclohaloalkyl, C₂₋₆ alkenyl, C₂₋₆ alkynyl, C₂₋₁₂ haloalkenyl, C₂₋₆ haloalkynyl, C₁₋₆ alkoxy-carbonyl, C₁₋₆ alkoxy-thiocarbonyl, and each of the groups from C₁₋₆ alkyl to C₁₋₆ alkoxy-thiocarbonyl among the groups of R² and R⁸ may be substituted; or
R² and R⁸ together with the carbon atom to which they are bound may form a 3 to 6-membered carbocyclic ring;
R⁹ is hydrogen, C₁₋₆ alkyl, C₁₋₆ alkoxy, C₁₋₆ haloalkyl, mono- or di-(C₁₋₆)alkylamino, C₁₋₆ alkoxyamino, C₁₋₆ alkoxy(C₁₋₆)alkyl, C₁₋₆ alkylhio, C₃₋₆ cycloalkyl, C₂₋₆ alkenyl, C₂₋₆ alkynyl, C₃₋₆ cycloalkylalkyl, C₁₋₆ alkylthio(C₁₋₆)alkyl, C₁₋₆ alkylsulfinyl(C₁₋₆)alkyl, C₁₋₆ alkylsulfonyl(C₁₋₆)alkyl, C₁₋₆ alkylcarbonyl(C₁₋₆)alkyl, C₃₋₆ cycloalkylamino, C₂₋₆ alkenylamino, C₂₋₆ alkynylamino, —CH₂R³, —C(O)R³ or —C(S)R³, and each group from C₁₋₆ alkyl to C₂₋₆ alkynylamino among the definitions of R⁹ may be substituted;
R¹⁰ is hydrogen, hydroxy, C₁₋₆ alkyl, C₁₋₆haloalkyl, C₃₋₆ cycloalkyl, C₂₋₆ alkenyl, C₂₋₆ alkinyl, C₁₋₆ alkoxy, C₁₋₆ alkylcarbonyl, C₁₋₆ alkoxycarbonyl, C₁₋₆ alkoxy(C₁₋₆)alkyl, C₁₋₆ cyanoalkyl, C₁₋₆ alkoxycarbonyl(C₁₋₆)alkyl, —CH₂R³, —C(O)R³ or —C(S)R³ and each group from C₁₋₆ alkyl to C₁₋₆ alkoxycarbonyl(C₁₋₆)alkyl among the definitions of R¹⁰ may be substituted;
l stands for 1, 2 or 3;
R is C₁₋₆ alkyl which may be substituted or C₁₋₆ haloalkyl which may be substituted;
X is hydrogen, halogen, nitro, cyano, hydroxy, mercapto, amino, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, C₁₋₆ haloalkylthio, C₁₋₆ haloalkylsulfinyl, C₁₋₆ haloalkylsulfonyl, C₁₋₆ alkylcarbonylamino, benzoylamino, C₁₋₆ alkoxy-carbonylamino, C₁₋₆ haloalkoxy-carbonylamino, C₁₋₆ alkoxyimino, C₁₋₆ haloalkoxyimino, C₁₋₆ alkylsulfonylamino or sulfur pentafluoride, and each group from C₁₋₆ alkyl to C₁₋₆ alkylsulfonylamino among the definitions of X may be optionally substituted; and
B¹, B², B³ and B⁴ independently are C—X or N.

9. The nitroketone compound of formula (II) according to claim 6, capable of being used for combating harmful invertebrate pests which occur in the agriculture or which occur in a veterinary field.

10. A method for combating one or more harmful invertebrate pests which occur in the agriculture or which occur in a veterinary field comprising using a compound of claim 6 to combat said pests.
